(19) United States
(12) Patent Application Publication
(10) Pub. No.: US 2024/0102018 A1
(43) Pub. Date: Mar. 28, 2024

(54) OLIGONUCLEOTIDE COMPOSITIONS AND METHODS THEREOF

(71) Applicant: Amylyx Pharmaceuticals, Inc., Cambridge, MA (US)

(72) Inventors: Joshua Barry Cohen, Somerville, MA (US); Justin Bernard Klee, Cambridge, MA (US); Duncan Brown, Berkeley, CA (US); Evan James Mizerak, Somerville, MA (US)

(21) Appl. No.: 18/356,591

(22) Filed: Jul. 21, 2023

Related U.S. Application Data
(60) Provisional application No. 63/391,161, filed on Jul. 21, 2022.

(51) Int. Cl. C12N 15/113

(52) U.S. Cl. CPC C12N 15/1137; C12N 2310/315; C12N 2310/321; C12N 2310/322; C12N 2320/32

(57) ABSTRACT

Among other things, the present disclosure provides oligonucleotides targeting SARM1 and compositions thereof. In some embodiments, the present disclosure provides methods for preventing or treating various conditions, disorders or diseases.

### CROSS-REFERENCE TO RELATED APPLICATIONS

This application claims priority to U.S. Provisional Application Ser. No. 63/391,161, filed Jul. 21, 2022, the entirety of which is incorporated herein by reference.

### SEQUENCE LISTING

The instant application contains a Sequence Listing which has been submitted electronically in XML file format and is hereby incorporated by reference in its entirety. Said XML copy, created on Sep. 28, 2023, is named 2014230-0010_SL.xml and is 1,673,007 bytes in size.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Among other things, the present disclosure provides technologies (e.g., oligonucleotides, compositions, methods, etc.) for treating various conditions, disorders or diseases associated with SARM1. In some embodiments, the present disclosure provides oligonucleotides that comprise various modifications, e.g., nucleobase modifications, sugar modifications, internucleotidic linkage modifications, etc. and can hybridize to a SARM1 transcript. In some embodiments, the present disclosure provides oligonucleotides and compositions thereof that when administered or delivered to a system comprising or expressing a SARM1 transcript can reduce the level of a SARM1 transcript. In some embodiments, a provided technology reduces levels of a SARM1 transcript and/or polypeptide in a system. In some embodiments, the present disclosure provides technologies for preventing and/or treating various conditions, disorders or diseases associated with SARM1.

In some embodiments, the present disclosure encompasses the recognition that oligonucleotides of certain base sequence may be more effective in reducing levels of SARM1 transcripts (e.g., SARM1 mRNA) and/or products thereof (e.g., SARM1 polypeptides). In some embodiments, a base sequence of an oligonucleotide is comprises about 5 or more (e.g., about 5, 6, 7, 8, 9, 10, 11, 12, 13, 14, 15, 16, 17, 18, 19, or 20) contiguous nucleobases of CCACTAGCCCTGGGAGCAAA (SEQ ID NO: 1), GCCATCTCCATCCATAGAGC (SEQ ID NO: 2), AGGAGAGCTGTGGGCTTGGG (SEQ ID NO: 3), CACCCATGCCTCCCAGCAGA (SEQ ID NO: 4), GCTGGCTGTACTCACTCTCC (SEQ ID NO: 5), GTGCTCTGTCCTTGGTCCTG (SEQ ID NO: 6), CCCATTCTCATGCAGCCTAC (SEQ ID NO: 7), CTGTGACCTAGGCTCCTTGA (SEQ ID NO: 8), GGTCTGAGAGGCTGTGGGTC (SEQ ID NO: 9), GCTCCCAGTTCTTCTGTGGT (SEQ ID NO: 10), GATGTCCTCCACAGGTGACA (SEQ ID NO: 11), GCTTCCTGCCTTACTGACCT (SEQ ID NO: 12), CTCTCCTTTGTCCCTGACCA (SEQ ID NO: 13), GCCTTGCCTTTTCCTCACTC (SEQ ID NO: 14), GCCTGGTCACTAACCCTCTC (SEQ ID NO: 15), CACCCACCTTGGTCTTGCCT (SEQ ID NO: 16), CACACTGATGTCCTGTCCCA (SEQ ID NO: 17), CACACCTCTGGGTCTTGGCC (SEQ ID NO: 18), GCTGCCCATCACTCCCAGTT (SEQ ID NO: 19), CTCTCCATCTGCCCTGGCCC (SEQ ID NO: 20), CAGTCCCTCTCCTTGTCTCT (SEQ ID NO: 21), ATCCACCTGCTGCTCCTGGG (SEQ ID NO: 22), CCCTTGTGTCTTGTGGGTGC (SEQ ID NO: 23), GCCCTAGGATTTTCCTGTTG (SEQ ID NO: 24), GCCTCAACTCCTGCCTCCCA (SEQ ID NO: 25), AGACACCTGGGTATCAGCCT (SEQ ID NO: 26), TCCTTCTTCCCTATTTCCCA (SEQ ID NO: 27), GCATCACTCACTGTCAGGTA (SEQ ID NO: 28), GTCAGTGCCACAGCCTTGTC (SEQ ID NO: 29), GGCACCTACCTTATGCACCC (SEQ ID NO: 30), ACTACTGCATCCCTCAGCCC (SEQ ID NO: 31), GCTTGTCTCATCCTGTCTCT (SEQ ID NO: 32), TGTCTCTGAGCTGACTGCTT (SEQ ID NO: 33), GGGCTTGACTCCACACTCCA (SEQ ID NO: 34), GGCATGGCATCTCAGCTTCA (SEQ ID NO: 35), TTCAGGATCACCTAGCTGGT (SEQ ID NO: 36), CCTCTTTGCCATCTGCTGGG (SEQ ID NO: 37), GAGTGCAGTTCACTTGTGGT (SEQ ID NO: 38), TGCCCACACTCTGCCTGTCA (SEQ ID NO: 39), CAGAGGGAGCTGCTAGTCAG (SEQ ID NO: 40), TTGGCAAAGGTGATGCAGGC (SEQ ID NO: 41), CCTCCACCAGTTGGAAGACC (SEQ ID NO: 42), GGTTCTCAGCCACCAGGATC (SEQ ID NO: 43), GTGCTCCAAGATGCCTGCCA (SEQ ID NO: 44), CCTTGCAGGCTCTTGATGGC (SEQ ID NO: 45), GTGCCATTGGTAGAGTAGGA (SEQ ID NO: 46), GTGAGCTCCCTAAAGAACCT (SEQ ID NO: 47), GGTTTGCCACCAGTACAGGG (SEQ ID NO: 48), TCCAGCTTCTCCACATCAAT (SEQ ID NO: 49), GAACTTGCCTGCTTCCAGCT (SEQ ID NO: 50), ACACTCTGGATGAGTTTGTC (SEQ ID NO: 51), GGGCACCCATGACACTCTGG (SEQ ID NO: 52), ACTTGTCCAGTGCTCCAGGT (SEQ ID NO: 53), CCCAATCCTTGCAGTCATGG (SEQ ID NO: 54), AGCACAGCCTGCATGTCCTC (SEQ ID NO: 55), CAAACTGGTGTCAGAGCCTG (SEQ ID NO: 56), GCAGCACCCTCCAAACTGGT (SEQ ID NO: 57), TGGTTAGGTTGGACCCATGG (SEQ ID NO: 58), GCCCAGGTTGTCTCAGCCCA (SEQ ID NO: 59), TCCCTCTCCAGATACTGAGG (SEQ ID NO: 60), ACAGACAACCCAATGGCAGG (SEQ ID NO: 61), GTCTCCAGAACTGAGCAGGG (SEQ ID NO: 62), CCTTAATTCCTGTCTGAGGC (SEQ ID NO: 63), CAGAATACAGTGCCCAGGCC (SEQ ID NO: 64), CCCAGGCCCTTGCTCAGAAT (SEQ ID NO: 65), GCACTCATCCCTGGCTGGCT (SEQ ID NO: 66), GATTACAGGGCAAGGCCACA (SEQ ID NO: 67), GCCCTGGATGTGGCAAAAGA (SEQ ID NO: 68), AAGGAAGTCAGAGGGAGGGC (SEQ ID NO: 69), CAGGCCCAAACAGGAGGCTC (SEQ ID NO: 70), ATGCCCAGACCCAGGCCCAA (SEQ ID NO: 71), CTGAGGCACAGCACCAAGGC (SEQ ID NO: 72), GCCAGACCAGGAAGGAGCCT (SEQ ID NO: 73), TCAGGACTTTGCCTCTTTCC (SEQ ID NO: 74), GCTTTAGAGATTTGCTACCC (SEQ ID NO: 75), GCCCAGCCTCAGAATGATTC (SEQ ID NO: 76), CCTCTGAACCCAGTGGAGGA (SEQ ID NO: 77), GCCTGGGTTTATTGGAGGGT (SEQ ID NO: 78), GCCAGCACAGCCAAGAGTGG (SEQ ID NO: 79), or GGGAGTGGAAGGAAGGAGCC (SEQ ID NO: 80), wherein each T is optionally and independently replaced with U. In some embodiments, a base sequence of an oligonucleotide is

(SEQ ID NO: 81)
CCACTAGCCCTGGGAGCAAA,
(SEQ ID NO: 82)
GCCATCTCCATCCATAGAGC,
(SEQ ID NO: 83)
AGGAGAGCTGTGGGCTTGGG,
(SEQ ID NO: 84)
CACCCATGCCTCCCAGCAGA,
(SEQ ID NO: 85)
GCTGGCTGTACTCACTCTCC,
(SEQ ID NO: 86)
GTGCTCTGTCCTTGGTCCTG,
(SEQ ID NO: 87)
CCCATTCTCATGCAGCCTAC,
(SEQ ID NO: 88)
CTGTGACCTAGGCTCCTTGA,
(SEQ ID NO: 89)
GGTCTGAGAGGCTGTGGGTC,
(SEQ ID NO: 90)
GCTCCCAGTTCTTCTGTGGT,
(SEQ ID NO: 91)
GATGTCCTCCACAGGTGACA,
(SEQ ID NO: 92)
GCTTCCTGCCTTACTGACCT,
(SEQ ID NO: 93)
CTCTCCTTTGTCCCTGACCA,
(SEQ ID NO: 94)
GCCTTGCCTTTTCCTCACTC,
(SEQ ID NO: 95)
GCCTGGTCACTAACCCTCTC,
(SEQ ID NO: 96)
CACCCACCTTGGTCTTGCCT,
(SEQ ID NO: 97)
CACACTGATGTCCTGTCCCA,
(SEQ ID NO: 98)
CACACCTCTGGGTCTTGGCC,
(SEQ ID NO: 99)
GCTGCCCATCACTCCCAGTT,
(SEQ ID NO: 100)
CTCTCCATCTGCCCTGGCCC,
(SEQ ID NO: 101)
CAGTCCCTCTCCTTGTCTCT,
(SEQ ID NO: 102)
ATCCACCTGCTGCTCCTGGG,
(SEQ ID NO: 103)
CCCTTGTGTCTTGTGGGTGC,
(SEQ ID NO: 104)
GCCCTAGGATTTTCCTGTTG,
(SEQ ID NO: 105)
GCCTCAACTCCTGCCTCCCA,
(SEQ ID NO: 106)
AGACACCTGGGTATCAGCCT,
(SEQ ID NO: 107)
TCCTTCTTCCCTATTTCCCA,
(SEQ ID NO: 108)
GCATCACTCACTGTCAGGTA,
(SEQ ID NO: 109)
GTCAGTGCCACAGCCTTGTC,
(SEQ ID NO: 110)
GGCACCTACCTTATGCACCC,
(SEQ ID NO: 111)
ACTACTGCATCCCTCAGCCC,
(SEQ ID NO: 112)
GCTTGTCTCATCCTGTCTCT,
(SEQ ID NO: 113)
TGTCTCTGAGCTGACTGCTT,
(SEQ ID NO: 114)
GGGCTTGACTCCACACTCCA,
(SEQ ID NO: 115)
GGCATGGCATCTCAGCTTCA,
(SEQ ID NO: 116)
TTCAGGATCACCTAGCTGGT,
(SEQ ID NO: 117)
CCTCTTTGCCATCTGCTGGG,
(SEQ ID NO: 118)
GAGTGCAGTTCACTTGTGGT,
(SEQ ID NO: 119)
TGCCCACACTCTGCCTGTCA,
(SEQ ID NO: 120)
CAGAGGGAGCTGCTAGTCAG,
(SEQ ID NO: 121)
TTGGCAAAGGTGATGCAGGC,
(SEQ ID NO: 122)
CCTCCACCAGTTGGAAGACC,
(SEQ ID NO: 123)
GGTTCTCAGCCACCAGGATC,
(SEQ ID NO: 124)
GTGCTCCAAGATGCCTGCCA,
(SEQ ID NO: 125)
CCTTGCAGGCTCTTGATGGC,
(SEQ ID NO: 126)
GTGCCATTGGTAGAGTAGGA,
(SEQ ID NO: 127)
GTGAGCTCCCTAAAGAACCT,
(SEQ ID NO: 128)
GGTTTGCCACCAGTACAGGG,
(SEQ ID NO: 129)
TCCAGCTTCTCCACATCAAT,
(SEQ ID NO: 130)
GAACTTGCCTGCTTCCAGCT,
(SEQ ID NO: 131)
ACACTCTGGATGAGTTTGTC,
(SEQ ID NO: 132)
GGGCACCCATGACACTCTGG,
(SEQ ID NO: 133)
ACTTGTCCAGTGCTCCAGGT,
(SEQ ID NO: 134)
CCCAATCCTTGCAGTCATGG,
(SEQ ID NO: 135)
AGCACAGCCTGCATGTCCTC,
(SEQ ID NO: 136)
CAAACTGGTGTCAGAGCCTG,
(SEQ ID NO: 137)
GCAGCACCCTCCAAACTGGT,
(SEQ ID NO: 138)
TGGTTAGGTTGGACCCATGG,
(SEQ ID NO: 139)
GCCCAGGTTGTCTCAGCCCA,
(SEQ ID NO: 140)
TCCCTCTCCAGATACTGAGG,
(SEQ ID NO: 141)
ACAGACAACCCAATGGCAGG,
(SEQ ID NO: 142)
GTCTCCAGAACTGAGCAGGG,
(SEQ ID NO: 143)
CCTTAATTCCTGTCTGAGGC,
(SEQ ID NO: 144)
CAGAATACAGTGCCCAGGCC,
(SEQ ID NO: 145)
CCCAGGCCCTTGCTCAGAAT,
(SEQ ID NO: 146)
GCACTCATCCCTGGCTGGCT,
(SEQ ID NO: 147)
GATTACAGGGCAAGGCCACA,
(SEQ ID NO: 148)
GCCCTGGATGTGGCAAAAGA,
(SEQ ID NO: 149)
AAGGAAGTCAGAGGGAGGGC,
(SEQ ID NO: 150)
CAGGCCCAAACAGGAGGCTC,
(SEQ ID NO: 151)
ATGCCCAGACCCAGGCCCAA,
(SEQ ID NO: 152)
CTGAGGCACAGCACCAAGGC,
(SEQ ID NO: 153)
GCCAGACCAGGAAGGAGCCT,
(SEQ ID NO: 154)
TCAGGACTTTGCCTCTTTCC,
(SEQ ID NO: 155)
GCTTTAGAGATTTGCTACCC,
(SEQ ID NO: 156)
GCCCAGCCTCAGAATGATTC,
(SEQ ID NO: 157)
CCTCTGAACCCAGTGGAGGA,
(SEQ ID NO: 158)
GCCTGGGTTTATTGGAGGGT,
(SEQ ID NO: 159)
GCCAGCACAGCCAAGAGTGG,
or
(SEQ ID NO: 160)
GGGAGTGGAAGGAAGGAGCC.

In some embodiments, provided oligonucleotides comprising various modifications, such as nucleobase modifications, sugar modifications, internucleotidic linkage modifications, etc. Various useful modifications are available in the art and may be utilized in accordance with the present disclosure. In some embodiments, modifications provide various benefits, e.g., improved stability, binding affinity, pharmacokinetic profiles, pharmacodynamic profiles, etc.

For example, in some embodiments, provided oligonucleotides comprise various sugar modifications. In some embodiments, a modified sugar is a natural RNA sugar with a 2′-OR^{s} modification, wherein R^{s} is an optionally substituted C₁₋₆ aliphatic, and —OR^{s} replace the 2′-OH group (a “2′-OR^{s} modified sugar”). In some embodiments, R^{s} is optionally substituted C₁₋₆ alkyl. In some embodiments, R^{s} is —CH₃. In some embodiments, R^{s} is —CH₂CH₂OCH₃.

In some embodiments, provided oligonucleotides comprises or consists of a wing-core-wing structure, wherein there are independently about 1-10 (e.g., about 1, 2, 3, 4, 5, 6, 7, 8, 9, or 10) nucleosides in each wing, there are about 5 or more (e.g., about 5-20, about 5, 6, 7, 8, 9, 10, 11, 12, 13, 14, 15, 16, 17, 18, 19, or 20, etc.) nucleosides in a core, and each wing independently comprises one or more (e.g., about 1, 2, 3, 4, 5, 6, 7, 8, 9, 10 or more) modified sugars. In some embodiments, each sugar in a wing is independently a modified sugar. In some embodiments, each sugar in a wing is independently a 2′-OR modified sugar. In some embodiments, a modified sugar is a 2′-MOE modified sugar (a 2′-OR modified sugar wherein R^{s} is —CH₂CH₂OCH₃). In some embodiments, each wing independently comprises one or more (e.g., about 1, 2, 3, 4, 5 or more) 2′-MOE modified sugar. In some embodiments, each sugar in a wing is independently a 2′-MOE modified sugar. In some embodiments, core regions comprise fewer modified sugars, and/or lower levels of modified sugars, compared to one or both wings. In some embodiments, there are no modified sugars in a core region. In some embodiments, each sugar in a core region is independently a nature DNA sugar.

Additionally or alternatively, in some embodiments, provided oligonucleotides comprise modified internucleotidic linkages. In some embodiments, modified internucleotidic linkages provide improved properties and/or activities compared to natural phosphate linkages. Various internucleotidic linkages are available in the art and can be utilized in accordance with the present disclosure. In some embodiments, a modified internucleotidic linkage is a phosphorothioate internucleotidic linkage (—O—P(O)(SH)—O—, which may exist in various salt forms). In some embodiments, each linkages in a provided oligonucleotide is a phosphorothioate internucleotidic linkage.

In some embodiments, the present disclosure provides technologies for preparing oligonucleotides and compositions thereof. In some embodiments, provided oligonucleotides and compositions thereof are of high purity. In some embodiments, oligonucleotides are provided as diastereomeric mixtures with respect to chiral linkage phosphorus, e.g., in phosphorothioate internucleotidic linkages. In some embodiments, one or more diastereomers with respect to chiral linkage phosphorus are enriched in provided compositions.

As described herein, oligonucleotides and compositions of the present disclosure may be provided/utilized in various forms. In some embodiments, the present disclosure provides compositions comprising one or more forms of oligonucleotides, e.g., acid forms (e.g., in which natural phosphate linkages exist as —O(P(O)(OH)—O—, phosphorothioate internucleotidic linkages exist as —O(P(O)(SH)—O—)), salt forms (e.g., in which one or more or all natural phosphate linkages independently exist as salt forms (e.g., sodium salt (—O(P(O)(O⁻Na⁺)—O—), one or more or all phosphorothioate internucleotidic linkages exist as salt forms (e.g., sodium salt (—O(P(O)(S⁻Na⁺)—O—), etc.), hydrates, etc. As appreciated by those skilled in the art, oligonucleotides can exist in various salt forms, including pharmaceutically acceptable salts, and in solutions (e.g., various aqueous buffering system), cations may dissociate from anions. In some embodiments, the present disclosure provides a pharmaceutical composition comprising a provided oligonucleotide and/or one or more pharmaceutically acceptable salts thereof, and a pharmaceutically acceptable carrier. In some embodiments, a pharmaceutically acceptable carrier is or comprises a buffer. In some embodiments, a pharmaceutically acceptable carrier is a buffered saline. In some embodiments, a pharmaceutically acceptable carrier is artificial cerebrospinal fluid (aCSF). In some embodiments, a pharmaceutically acceptable carrier is cerebrospinal fluid.

In some embodiments, the present disclosure describes useful technologies for assessing oligonucleotide and compositions thereof. Certain useful technologies are described in the Examples.

Provided technologies can be utilized for various purposes. For example, in some embodiments, provided technologies are useful for preventing and/or treating various conditions, disorders or diseases associated with SARM1. In some embodiments, the present disclosure provides a method for preventing a condition, disorder or disease, comprising administering or delivering to a subject susceptible thereto an effective amount of a provided oligonucleotide. In some embodiments, the present disclosure provides a method for treating a condition, disorder or disease, comprising administering or delivering to a subject suffering therefrom an effective amount of a provided oligonucleotide. In some embodiments, an oligonucleotide is administered or delivered in a pharmaceutical composition. In some embodiments, an oligonucleotide is administered or delivered in one or more forms, e.g., in some embodiments, in one or more pharmaceutically acceptable salt forms. In some embodiments, an oligonucleotide is administered or delivered in a solution, e.g., in an aCSF solution. Various technologies are available in the art and may be utilized to administer or deliver provided oligonucleotides and compositions thereof. For example, in some embodiments, oligonucleotides and compositions there are administered or delivered intrathecally.

In some embodiments, a condition, disorder or disease is a neurodegenerative condition, disorder or disease. In some embodiments, a condition, disorder or disease is or comprises Wallerian degeneration. In some embodiments, a condition, disorder or disease is associated with Wallerian degeneration. In some embodiments, a condition, disorder or disease is amyotrophic lateral sclerosis (ALS). In some embodiments, a condition, disorder or disease is neuropathy. In some embodiments, a condition, disorder or disease is peripheral neuropathy. In some embodiments, a condition, disorder or disease is peripheral neuropathy induced by chemotherapy. In some embodiments, a condition, disorder or disease is Parkinson's disease. In some embodiments, a condition, disorder or disease is Huntington's disease. In some embodiments, a condition, disorder or disease is Alzheimer's disease. In some embodiments, a condition, disorder or disease is frontotemporal dementia. In some embodiments, a condition, disorder or disease is brain injury. In some embodiments, a condition, disorder or disease is traumatic brain injury. In some embodiments, a condition, disorder or disease is progressive supranuclear palsy. In some embodiments, a condition, disorder or disease is corticobasal degeneration. In some embodiments, a condition, disorder or disease is Wolfram Syndrome. In some embodiments, a condition, disorder or disease is Friedreich's Ataxia. In some embodiments, a condition, disorder or disease is Multiple System Atrophy. In some embodiments, a condition, disorder or disease is Spinal Cerebellar Ataxia. In some embodiments, a condition, disorder or disease is Spinal Muscular Atrophy (SMA). In some embodiments, a condition, disorder or disease is Pick's Disease. In some embodiments, a condition, disorder or disease is progressive motor atrophy.

### BRIEF DESCRIPTION OF THE DRAWINGS

FIG. 1. Provided oligonucleotides can reduce levels of SARM1 mRNA. (A) and (B): Various oligonucleotides were demonstrated to knockdown SARM1. Human iPSC-derived glutamatergic neurons were treated with various oligonucleotides targeting SARM1 by gymnotic uptake for 72 hours. Cells were lysed and RNA collected. RNA was used in real time RT-qPCR to quantify fold-change of SARM1 expression. Average fold-change of SARM1 expression of biological replicates (black dots) is represented in the top panel, average SARM1 Cp value of technical replicates (black dots) is represented in the middle panel, and average RPLP0 Cp value of technical replicates (black dots) is represented in the bottom panel. The X-axis denotes oligonucleotide; the Y-axis depicts fold-change in SARM1 expression (top panel), SARM1 Cp value (middle panel), or RPLP0 Cp value (bottom panel). Dotted horizontal line indicates corresponding fold-change or Cp value from treatment with vehicle-only negative control. Error bars represent standard deviation. * indicates data from only 1 technical replicate displayed.

FIG. 2. Various provided oligonucleotides provide knockdown of SARM1. Various oligonucleotides were assessed to knockdown SARM1. Human iPSC-derived glutamatergic neurons were treated with various oligonucleotides targeting SARM1 or NEAT1 (positive and negative control) by gymnotic uptake for 72 hours. Cells were lysed and RNA collected. RNA was used in real time RT-qPCR to quantify fold-change of SARM1 or NEAT1 expression. Average fold-change of SARM1 or NEAT1 expression of biological replicates (black dots) is represented in the top panel, average SARM1 or NEAT1 Cp value of technical replicates (black dots) is represented in the middle panel, and average RPLP0 Cp value of technical replicates (black dots) is represented in the bottom panel. The X-axis denotes oligonucleotide; the Y-axis depicts fold-change in SARM1 or NEAT1 expression (top panel), SARM1 or NEAT1 Cp value (middle panel), or RPLP0 Cp value (bottom panel). Dotted horizontal line indicates corresponding fold-change or Cp value from treatment with vehicle-only negative control. Error bars represent standard deviation. For NEAT1, from left to right, vehicle, positive control, and negative control. For SARM1 and RPLP0, from left to right, vehicle, positive control of NEAT1, negative control, and various assessed oligonucleotides. * indicates data from only 1 technical replicate displayed.

FIG. 3. Various provided oligonucleotides display no obvious cytotoxicity as compared to vehicle treatment. (A) and (B): Various oligonucleotides were confirmed to not significantly change percentage of live cells. Human iPSC-derived glutamatergic neurons were treated with various oligonucleotides by gymnotic uptake for 48 hours. Following this incubation period, cells were harvested and underwent Hoechst staining (5 μg/mL). The number of live, dead, and total (live+dead) cells were counted and the percent of live cells was calculated. Average live cell percentage of biological replicates is represented in the top panel, and average live cell count of biological replicates is represented in the bottom panel. The X-axis denotes oligonucleotide; the Y-axis depicts live cells as percentage of total cells (top panel) or total live cell count (bottom panel). Dotted horizontal line indicates corresponding live cell percentage or total live cell count for treatment with vehicle-only. From left to right: vehicle, positive control of NEAT1, negative control, and various assessed oligonucleotides. For percentage of live cells, the lower dotted line indicates corresponding live cell percentage for treatment with the negative control oligonucleotide composition. Error bars represent standard deviation.

FIG. 4. Provided oligonucleotides can provide knockdown of SARM1 mRNA. Human iPSC-derived motor neurons were treated with oligonucleotide compositions targeting SARM1 or a scrambled (Scr) negative control oligonucleotide by gymnotic uptake for 48 hours. After 48 hours, media was refreshed to remove oligonucleotides. Cells were lysed at various time points (0, 3, 7, 10, 14, 21 days) following removal of oligonucleotides and RNA collected. RNA was used in RT-qPCR to quantify SARM1 expression. % knockdown of SARM1 was determined as compared to mean negative control % knockdown of SARM1 for the corresponding time point. Mean values are represented by bars and replicates (N=3) are indicated by a dot or triangle. The X-axis denotes days (DO, D3, D7, D10, D14, D21) after removal of oligonucleotides; the Y-axis depicts % knockdown of SARM1. Error bars represent standard deviation.

FIG. 5. SARM1 protein levels after oligonucleotide treatment. Human iPSC-derived motor neurons were treated with an oligonucleotide composition targeting SARM1 or scrambled (Scr) negative control oligonucleotide by gymnotic uptake for 48 hours. After 48 hours, media was refreshed to remove oligonucleotides. Cells were lysed at various time points (0, 3, 7, 10, 14, 21 days) following removal of oligonucleotides. Lysates were used in western blotting to quantify SARM1 protein levels. Shown are data for oligonucleotide 45 (A) and oligonucleotide 62 (B). Mean values are represented by bars and replicates (N=2) are indicated by a dot or triangle. The X-axis denotes days (DO, D3, D7, D10, D14, D21) after removal of oligonucleotides; the Y-axis depicts relative knockdown of SARM1 protein (% over mean negative control SARM1 protein level at corresponding time point). Error bars represent standard deviation.

FIG. 6. Cell morphology upon treatment with provided oligonucleotide compositions. Human iPSC-derived motor neurons were treated with an oligonucleotide composition targeting SARM1 or scrambled (Scr) negative control oligonucleotide by gymnotic uptake for 48 hours. After 48 hours, media was refreshed to remove oligonucleotides. Cell morphology was assessed by brightfield imaging at various time points (0, 3, 21 days) following removal of oligonucleotides. Representative images are shown. Time point (DO, D3, D21) for each row are indicated by labels at left; treatment (vehicle (H₂O), scrambled (Scr) negative control oligonucleotide, oligonucleotide composition 45, oligonucleotide composition 62) for each column of images is indicated by labels at top.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as various forms, e.g., salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different forms, e.g., salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a nucleic acid is one that contains a number of, in some embodiments, a continuous stretch of, nucleobases that are characteristic of that nucleic acid.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently —H or an optionally substituted group selected from C₁₋₁₀ aliphatic, C₆₋₁₄ aryl, C₁₋₁₀ heteroaliphatic having 1-5 heteroatoms, 5-10 membered heteroaryl having 1-5 heteroatoms and 3-10 membered heterocyclyl having 1-4 heteroatoms, or two or more R′ groups are taken together with their intervening atoms to from an optionally substituted 3-10 membered ring having 0-5 heteroatoms in addition to the intervening atoms. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. It is understood by a person of ordinary skill in the art that an internucleotidic linkage may exist as an anion or cation at a given pH due to the existence of acid or base moieties in the linkage.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral (e.g., as in phosphorothioate internucleotidic linkages). In some embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-F. In some embodiments, a 2′-modification is 2′-OR, wherein R is optionally substituted C₁₋₁₀ aliphatic. In some embodiments, a 2′-modification is 2′-OMe (2′-O-methyl). In some embodiments, a 2′-modification is 2′-MOE (2′-O-methoxyethyl). In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly—refers to a nucleic acid containing 2 to about 10,000 nucleotide monomer units and wherein the prefix oligo—refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, an oligonucleotide is from about 9 to about 39 nucleosides in length. In some embodiments, an oligonucleotide is from about 25 to about 70 nucleosides in length. In some embodiments, an oligonucleotide is from about 26 to about 70 nucleosides in length. In some embodiments, an oligonucleotide is from about 27 to about 70 nucleosides in length. In some embodiments, an oligonucleotide is from about 28 to about 70 nucleosides in length. In some embodiments, an oligonucleotide is from about 29 to about 70 nucleosides in length. In some embodiments, an oligonucleotide is from about 30 to about 70 nucleosides in length. In some embodiments, an oligonucleotide is from about 31 to about 70 nucleosides in length. In some embodiments, an oligonucleotide is from about 32 to about 70 nucleosides in length. In some embodiments, an oligonucleotide is from about 25 to about 60 nucleosides in length. In some embodiments, an oligonucleotide is from about 25 to about 50 nucleosides in length. In some embodiments, an oligonucleotide is from about 25 to about 40 nucleosides in length. In some embodiments, an oligonucleotide is from about 30 to about 40 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, an oligonucleotide is at least 4 nucleosides in length. In some embodiments, an oligonucleotide is at least 5 nucleosides in length. In some embodiments, an oligonucleotide is at least 6 nucleosides in length. In some embodiments, an oligonucleotide is at least 7 nucleosides in length. In some embodiments, an oligonucleotide is at least 8 nucleosides in length. In some embodiments, an oligonucleotide is at least 9 nucleosides in length. In some embodiments, an oligonucleotide is at least 10 nucleosides in length. In some embodiments, an oligonucleotide is at least 11 nucleosides in length. In some embodiments, an oligonucleotide is at least 12 nucleosides in length. In some embodiments, an oligonucleotide is at least 15 nucleosides in length. In some embodiments, an oligonucleotide is at least 15 nucleosides in length. In some embodiments, an oligonucleotide is at least 16 nucleosides in length. In some embodiments, an oligonucleotide is at least 17 nucleosides in length. In some embodiments, an oligonucleotide is at least 18 nucleosides in length. In some embodiments, an oligonucleotide is at least 19 nucleosides in length. In some embodiments, an oligonucleotide is at least 20 nucleosides in length. In some embodiments, an oligonucleotide is at least 25 nucleosides in length. In some embodiments, an oligonucleotide is at least 26 nucleosides in length. In some embodiments, an oligonucleotide is at least 27 nucleosides in length. In some embodiments, an oligonucleotide is at least 28 nucleosides in length. In some embodiments, an oligonucleotide is at least 29 nucleosides in length. In some embodiments, an oligonucleotide is at least 30 nucleosides in length. In some embodiments, an oligonucleotide is at least 31 nucleosides in length. In some embodiments, an oligonucleotide is at least 32 nucleosides in length. In some embodiments, an oligonucleotide is at least 33 nucleosides in length. In some embodiments, an oligonucleotide is at least 34 nucleosides in length. In some embodiments, an oligonucleotide is at least 35 nucleosides in length. In some embodiments, an oligonucleotide is at least 36 nucleosides in length. In some embodiments, an oligonucleotide is at least 37 nucleosides in length. In some embodiments, an oligonucleotide is at least 38 nucleosides in length. In some embodiments, an oligonucleotide is at least 39 nucleosides in length. In some embodiments, an oligonucleotide is at least 40 nucleosides in length. In some embodiments, an oligonucleotide is 25 nucleosides in length. In some embodiments, an oligonucleotide is 26 nucleosides in length. In some embodiments, an oligonucleotide is 27 nucleosides in length. In some embodiments, an oligonucleotide is 28 nucleosides in length. In some embodiments, an oligonucleotide is 29 nucleosides in length. In some embodiments, an oligonucleotide is 30 nucleosides in length. In some embodiments, an oligonucleotide is 31 nucleosides in length. In some embodiments, an oligonucleotide is 32 nucleosides in length. In some embodiments, an oligonucleotide is 33 nucleosides in length. In some embodiments, an oligonucleotide is 34 nucleosides in length. In some embodiments, an oligonucleotide is 35 nucleosides in length. In some embodiments, an oligonucleotide is 36 nucleosides in length. In some embodiments, an oligonucleotide is 37 nucleosides in length. In some embodiments, an oligonucleotide is 38 nucleosides in length. In some embodiments, an oligonucleotide is 39 nucleosides in length. In some embodiments, an oligonucleotide is 40 nucleosides in length. In some embodiments, each nucleoside counted in an oligonucleotide length independently comprises a nucleobase comprising a ring having at least one nitrogen ring atom. In some embodiments, each nucleoside counted in an oligonucleotide length independently comprises A, T, C, G, or U, or optionally substituted A, T, C, G, or U, or an optionally substituted tautomer of A, T, C, G or U.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{º}; —(CH₂)₀₋₄R^{º}; —O(CH₂)₀₋₄R^{º}, —O—(CH₂)₀₋₄C(O)OR^{º}; —(CH₂)₀₋₄CH(OR^{º})₂; —(CH₂)₀₋₄Ph, which may be substituted with R^{º}; —(CH₂)₀₋₄O(CH₂)₀₋₁Ph which may be substituted with R^{º}; —CH═CHPh, which may be substituted with R^{º}; —(CH₂)₀₋₄O(CH₂)₀₋₁-pyridyl which may be substituted with R^{º}; —NO₂; —CN; —N₃; —(CH₂)₀₋₄N(R^{º})₂; —(CH₂)₀₋₄N(R^{º})C(O)R^{º}; —N(R^{º})C(S)R^{º}; —(CH₂)₀₋₄N(R^{º})C(O)NR^{º}₂; —N(R^{º})C(S)NR^{º}₂; —(CH₂)₀₋₄N(R^{º})C(O)OR^{º}; —N(R^{º})N(R^{º})C(O)R^{º}; —N(R^{º})N(R^{º})C(O)NR^{º}₂; —N(R^{º})N(R^{º})C(O)OR^{º}; —(CH₂)₀₋₄C(O)R^{º}; —C(S)R^{º}; —(CH₂)₀₋₄C(O)OR^{º}; —(CH₂)₀₋₄C(O)SR^{º}; —(CH₂)₀₋₄C(O)OSiR^{º}₃; —(CH₂)₀₋₄₀C(O)R^{º}; —OC(O)(CH₂)₀₋₄SR^{º}, —SC(S)SR^{º}; —(CH₂)₀₋₄SC(O)R^{º}; —(CH₂)₀₋₄C(O)NR^{º}₂; —C(S)NR^{º}₂; —C(S)SR^{º}; —(CH₂)₀₋₄₀C(O)NR^{º}₂; —C(O)N(OR^{º})R^{º}; —C(O)C(O)R^{º}; —C(O)CH₂C(O)R^{º}; —C(NOR^{º})R^{º}; —(CH₂)₀₋₄SSR^{º}; —(CH₂)₀₋₄S(O)₂R^{º}; —(CH₂)₀₋₄S(O)₂OR^{º}; —(CH₂)₀₋₄S(O)₂R^{º}; —S(O)₂NR^{º}₂; —(CH₂)₀₋₄S(O)R^{º}; —N(R^{º})S(O)₂NR^{º}₂; —N(R^{º})S(O)₂R^{º}; —N(OR^{º})R^{º}; —C(NH)NR^{º}₂; —Si(R^{º})₃; —OSi(R^{º})₃; —B(R^{º})₂; —OB(R^{º})₂; —OB(OR^{º})₂; —P(R^{º})₂; —P(OR^{º})₂; —P(R^{º})(OR^{º}); —OP(R^{º})₂; —OP(OR^{º})₂; —OP(R^{º})(OR^{º}); —P(O)(R^{º})₂; —P(O)(OR^{º})₂; —OP(O)(R^{º})₂; —OP(O)(OR^{º})₂; —OP(O)(OR^{º})(SR^{º}); —SP(O)(R^{º})₂; —SP(O)(OR^{º})₂; —N(R^{º})P(O)(R^{º})₂; —N(R^{º})P(O)(OR^{º})₂; —P(R^{º})₂[B(R^{º})₃]; —P(OR^{º})₂[B(R^{º})₃]; —OP(R^{º})₂[B(R^{º})₃]; —OP(OR^{º})₂[B(R^{º})₃]; —(C₁₋₄ straight or branched alkylene)O—N(R^{º})₂; or —(C₁₋₄ straight or branched alkylene)C(O)O—N(R^{º})₂, wherein each R^{º} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{º}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{º} (or the ring formed by taking two independent occurrences of R^{º} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR′, —(CH₂)₀₋₂SH, —(CH₂)₀₋₂NH₂, —(CH₂)₀₋₂NHR^{●}, —(CH₂)₀₋₂NR′₂, —NO₂, —SiR^{●}3, —OSiR^{●}3, —C(O)SR′, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{º} include ═0 and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{s}, —O(haloR^{●}), —CN, —C(O)OH, —C(O)OR^{●}, —NH₂, —NHR^{●}, —NR′₂, or —NO₂, wherein each R′ is unsubstituted or where preceded by “halo” is substituted only with one or more halogens, and is independently C₁₋₄ aliphatic, —CH₂Ph, —O(CH₂)₀₋₁Ph, or a 3-6 (e.g., 3-5, 5-6, etc.)-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR′₂, or —NO₂, wherein each R′ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Protecting group: The term “protecting group,” as used herein, is well known in the art and includes those described in detail in Protecting Groups in Organic Synthesis, T. W. Greene and P. G. M. Wuts, 3rd edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

As used in the present disclosure, in some embodiments, “one or more” is 1-200, 1-150, 1-100, 1-90, 1-80, 1-70, 1-60, 1-50, 1-40, 1-30, or 1,2, 3,4, 5,6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is “one or more” as described herein.

### Description of Certain Embodiments

Among other things, the present disclosure provides various oligonucleotides and compositions thereof. In some embodiments, oligonucleotides of the present disclosure target SARM1 and can hybridize with a SARM1 transcript, e.g., a SARM1 mRNA. In some embodiments, provided technologies, e.g., oligonucleotide, compositions, methods, etc., reduce levels of SARM1 transcripts and/or products thereof. Use of naturally occurring nucleic acids is limited, for example, by their susceptibility to endo- and exo-nucleases. As such, various synthetic counterparts have been developed to circumvent these shortcomings and/or to further improve various properties and activities. In some embodiments, provided oligonucleotides comprise various chemical modifications, e.g., nucleobase modifications, sugar modifications, internucleotidic linkage modifications, etc., which, among other things, render these molecules less susceptible to degradation and improve other properties and/or activities. In some embodiments, an oligonucleotide comprises one or more features described herein, e.g., base sequence, length, wing, core, activity, etc. In some embodiments, an oligonucleotide has a base sequence described herein, and/or a wing-core-wing structure as described herein.

### Base Sequences

Base sequences of various oligonucleotides are of sufficient lengths so that they can form duplexes with complementary sequences in target nucleic acids for one or more biological functions. In some embodiments, oligonucleotides specifically target their target nucleic acids. In some embodiments, the base sequence of a provided oligonucleotide is or comprises a sequence that is complementary to a portion in a target nucleic acid (a “target portion”), e.g., a SARM1 gene or a transcript thereof. In some embodiments, a target portion comprises a portion of an exon and/or intron. In some embodiments, a target portion comprises a portion of exon 1, exon 2, exon 3, exon 4, exon 5, exon 6, exon 7, exon 8, or exon 9 of SARM1. In some embodiments, a target portion comprises a portion of intron 1, intron 2, intron 3, intron 4, intron 5, intron 6, intron 7, or intron 8 of SARM1. In some embodiments, a target portion comprises a portion of exon 1 of SARM1. In some embodiments, a target portion comprises a portion of exon 2 of SARM1. In some embodiments, a target portion comprises a portion of exon 3 of SARM1. In some embodiments, a target portion comprises a portion of exon 4 of SARM1. In some embodiments, a target portion comprises a portion of exon 5 of SARM1. a portion of In some embodiments, a target portion comprises a portion of exon 7 of SARM1. In some embodiments, a target portion comprises a portion of exon 8 of SARM1. In some embodiments, a target portion comprises a portion of exon 9 of SARM1. In some embodiments, a target portion comprises a portion of intron 1 of SARM1. In some embodiments, a target portion comprises a portion of intron 2 of SARM1. In some embodiments, a target portion comprises a portion of intron 3 of SARM1. In some embodiments, a target portion comprises a portion of intron 4 of SARM1. In some embodiments, a target portion comprises a portion of intron 5 of SARM1. In some embodiments, a target portion comprises a portion of intron 6 of SARM1. In some embodiments, a target portion comprises a portion of intron 7 of SARM1. In some embodiments, a target portion comprises a portion of intron 8 of SARM1. Exons and introns alternate, e.g., intron 1 is between exon 1 and exon 2; intron 2 is between exon 2 and exon 3; etc. In some embodiments, a target portion is within an exon. In some embodiments, a target portion is within an intron. In some embodiments, a target portion comprises a portion of an exon and a portion of an exon. In some embodiments, such a sequence complementary to a target portion is about 10, 11, 12, 13, 14, 15, 16, 17, 18, 19, 20, 21, 22, 23, 24, 25 or more nucleobases in length.

In some embodiments, a target portion is or comprises a characteristic portion of a nucleic acid sequence (e.g., of a SARM1 gene or a transcript thereof) which characteristic portion defines the nucleic acid sequence over others in a relevant organism; for example, the characteristic portion is not in other genomic nucleic acid sequences (e.g., genes) or transcripts thereof in a relevant organism (e.g., for human SARM1, its characteristic portion is not in other human nucleic acid sequences or transcripts thereof). In some embodiments, a characteristic portion of a transcript defines that transcript over other transcripts in a relevant organism; for example, in some embodiments, the characteristic portion is not in transcripts that are transcribed from a different nucleic acid sequence (e.g., a different gene). In some embodiments, transcript variants from a nucleic acid sequence (e.g., mRNA variants of a gene) may share a common characteristic portion that defines them from transcripts of other nucleic acids, e.g., transcripts of other genes. In some embodiments, a characteristic portion in a transcript defines the transcript from other transcript(s) of the same nucleic acid sequence (e.g., a gene) and/or other alleles of the nucleic acid sequence. In some embodiments, a characteristic portion defines a particular allele (and/or transcripts thereof) over other allele(s) (and/or transcripts thereof). In some embodiments, a characteristic portion comprises sequences that are separated in a nucleic acid. In some embodiments, a characteristic portion is a contiguous stretch of nucleobases in a nucleic acid (a “characteristic sequence”). A characteristic portion or sequence may have various numbers of nucleobases. In some embodiments, there are about 10. 11, 12, 13, 14, 15, 16, 17, 18, 19, 20, 21, 22, 23, 24, 25 or more nucleobases in a characteristic portion or sequence; in some embodiments, there are about 10; in some embodiments, there are about 11; in some embodiments, there are about 12; in some embodiments, there are about 13; in some embodiments, there are about 14; in some embodiments, there are about 15; in some embodiments, there are about 16; in some embodiments, there are about 17; in some embodiments, there are about 18; in some embodiments, there are about 19; in some embodiments, there are about 20; in some embodiments, there are about 21; in some embodiments, there are about 22; in some embodiments, there are about 23; in some embodiments, there are about 24; in some embodiments, there are about 25; in some embodiments, there are about 25 or more.

In some embodiments, an oligonucleotide comprises a sequence that is identical or complementary to a characteristic portion of a nucleic acid. In some embodiments, an oligonucleotide comprises a sequence that is identical or complementary to a characteristic portion of a SARM1 transcript. In some embodiments, an oligonucleotide comprises a sequence that is complementary to a characteristic portion of a SARM1 transcript. In some embodiments, the base sequence of an oligonucleotide is identical or complementary to a characteristic portion of a nucleic acid. In some embodiments, the base sequence of an oligonucleotide is identical or complementary to a characteristic portion of a SARM1 transcript. In some embodiments, the base sequence of an oligonucleotide is complementary to a characteristic portion of a SARM1 transcript. In some embodiments, a characteristic portion is a characteristic sequence.

In some embodiments, a characteristic sequence of a SARM1 transcript is or comprises a complementary sequence of the sequence of an oligonucleotide in Table 1. In some embodiments, a characteristic sequence is or comprises UUUGCUCCCAGGGCUAGUGG (SEQ ID NO: 161), wherein each U may be independently replaced with T. In some embodiments, a characteristic sequence is or comprises GCUCUAUGGAUGGAGAUGGC (SEQ ID NO: 162), wherein each U may be independently replaced with T. In some embodiments, a characteristic sequence is or comprises CCCAAGCCCACAGCUCUCCU (SEQ ID NO: 163), wherein each U may be independently replaced with T. In some embodiments, a characteristic sequence is or comprises UCUGCUGGGAGGCAUGGGUG (SEQ ID NO: 164), wherein each U may be independently replaced with T. In some embodiments, a characteristic sequence is or comprises GGAGAGUGAGUACAGCCAGC (SEQ ID NO: 165), wherein each U may be independently replaced with T. In some embodiments, a characteristic sequence is or comprises CAGGACCAAGGACAGAGCAC (SEQ ID NO: 166), wherein each U may be independently replaced with T. In some embodiments, a characteristic sequence is or comprises GUAGGCUGCAUGAGAAUGGG (SEQ ID NO: 167), wherein each U may be independently replaced with T. In some embodiments, a characteristic sequence is or comprises UCAAGGAGCCUAGGUCACAG (SEQ ID NO: 168), wherein each U may be independently replaced with T. In some embodiments, a characteristic sequence is or comprises GACCCACAGCCUCUCAGACC (SEQ ID NO: 169), wherein each U may be independently replaced with T. In some embodiments, a characteristic sequence is or comprises ACCACAGAAGAACUGGGAGC (SEQ ID NO: 170), wherein each U may be independently replaced with T. In some embodiments, a characteristic sequence is or comprises UGUCACCUGUGGAGGACAUC (SEQ ID NO: 171), wherein each U may be independently replaced with T. In some embodiments, a characteristic sequence is or comprises AGGUCAGUAAGGCAGGAAGC (SEQ ID NO: 172), wherein each U may be independently replaced with T. In some embodiments, a characteristic sequence is or comprises UGGUCAGGGACAAAGGAGAG (SEQ ID NO: 173), wherein each U may be independently replaced with T. In some embodiments, a characteristic sequence is or comprises GAGUGAGGAAAAGGCAAGGC (SEQ ID NO: 174), wherein each U may be independently replaced with T. In some embodiments, a characteristic sequence is or comprises GAGAGGGUUAGUGACCAGGC (SEQ ID NO: 175), wherein each U may be independently replaced with T. In some embodiments, a characteristic sequence is or comprises AGGCAAGACCAAGGUGGGUG (SEQ ID NO: 176), wherein each U may be independently replaced with T. In some embodiments, a characteristic sequence is or comprises UGGGACAGGACAUCAGUGUG (SEQ ID NO: 177), wherein each U may be independently replaced with T. In some embodiments, a characteristic sequence is or comprises GGCCAAGACCCAGAGGUGUG (SEQ ID NO: 178), wherein each U may be independently replaced with T. In some embodiments, a characteristic sequence is or comprises AACUGGGAGUGAUGGGCAGC (SEQ ID NO: 179), wherein each U may be independently replaced with T. In some embodiments, a characteristic sequence is or comprises GGGCCAGGGCAGAUGGAGAG (SEQ ID NO: 180), wherein each U may be independently replaced with T. In some embodiments, a characteristic sequence is or comprises AGAGACAAGGAGAGGGACUG (SEQ ID NO: 181), wherein each U may be independently replaced with T. In some embodiments, a characteristic sequence is or comprises CCCAGGAGCAGCAGGUGGAU (SEQ ID NO: 182), wherein each U may be independently replaced with T. In some embodiments, a characteristic sequence is or comprises GCACCCACAAGACACAAGGG (SEQ ID NO: 183), wherein each U may be independently replaced with T. In some embodiments, a characteristic sequence is or comprises CAACAGGAAAAUCCUAGGGC (SEQ ID NO: 184), wherein each U may be independently replaced with T. In some embodiments, a characteristic sequence is or comprises UGGGAGGCAGGAGUUGAGGC (SEQ ID NO: 185), wherein each U may be independently replaced with T. In some embodiments, a characteristic sequence is or comprises AGGCUGAUACCCAGGUGUCU (SEQ ID NO: 186), wherein each U may be independently replaced with T. In some embodiments, a characteristic sequence is or comprises UGGGAAAUAGGGAAGAAGGA (SEQ ID NO: 187), wherein each U may be independently replaced with T. In some embodiments, a characteristic sequence is or comprises UACCUGACAGUGAGUGAUGC (SEQ ID NO: 188), wherein each U may be independently replaced with T. In some embodiments, a characteristic sequence is or comprises GACAAGGCUGUGGCACUGAC (SEQ ID NO: 189), wherein each U may be independently replaced with T. In some embodiments, a characteristic sequence is or comprises GGGUGCAUAAGGUAGGUGCC (SEQ ID NO: 190), wherein each U may be independently replaced with T. In some embodiments, a characteristic sequence is or comprises GGGCUGAGGGAUGCAGUAGU (SEQ ID NO: 191), wherein each U may be independently replaced with T. In some embodiments, a characteristic sequence is or comprises AGAGACAGGAUGAGACAAGC (SEQ ID NO: 192), wherein each U may be independently replaced with T. In some embodiments, a characteristic sequence is or comprises AAGCAGUCAGCUCAGAGACA (SEQ ID NO: 193), wherein each U may be independently replaced with T. In some embodiments, a characteristic sequence is or comprises UGGAGUGUGGAGUCAAGCCC (SEQ ID NO: 194), wherein each U may be independently replaced with T. In some embodiments, a characteristic sequence is or comprises UGAAGCUGAGAUGCCAUGCC (SEQ ID NO: 195), wherein each U may be independently replaced with T. In some embodiments, a characteristic sequence is or comprises ACCAGCUAGGUGAUCCUGAA (SEQ ID NO: 196), wherein each U may be independently replaced with T. In some embodiments, a characteristic sequence is or comprises CCCAGCAGAUGGCAAAGAGG (SEQ ID NO: 197), wherein each U may be independently replaced with T. In some embodiments, a characteristic sequence is or comprises ACCACAAGUGAACUGCACUC (SEQ ID NO: 198), wherein each U may be independently replaced with T. In some embodiments, a characteristic sequence is or comprises UGACAGGCAGAGUGUGGGCA (SEQ ID NO: 199), wherein each U may be independently replaced with T. In some embodiments, a characteristic sequence is or comprises CUGACUAGCAGCUCCCUCUG (SEQ ID NO: 200), wherein each U may be independently replaced with T. In some embodiments, a characteristic sequence is or comprises GCCUGCAUCACCUUUGCCAA (SEQ ID NO: 201), wherein each U may be independently replaced with T. In some embodiments, a characteristic sequence is or comprises GGUCUUCCAACUGGUGGAGG (SEQ ID NO: 202), wherein each U may be independently replaced with T. In some embodiments, a characteristic sequence is or comprises GAUCCUGGUGGCUGAGAACC (SEQ ID NO: 203), wherein each U may be independently replaced with T. In some embodiments, a characteristic sequence is or comprises UGGCAGGCAUCUUGGAGCAC (SEQ ID NO: 204), wherein each U may be independently replaced with T. In some embodiments, a characteristic sequence is or comprises GCCAUCAAGAGCCUGCAAGG (SEQ ID NO: 205), wherein each U may be independently replaced with T. In some embodiments, a characteristic sequence is or comprises UCCUACUCUACCAAUGGCAC (SEQ ID NO: 206), wherein each U may be independently replaced with T. In some embodiments, a characteristic sequence is or comprises AGGUUCUUUAGGGAGCUCAC (SEQ ID NO: 207), wherein each U may be independently replaced with T. In some embodiments, a characteristic sequence is or comprises CCCUGUACUGGUGGCAAACC (SEQ ID NO: 208), wherein each U may be independently replaced with T. In some embodiments, a characteristic sequence is or comprises AUUGAUGUGGAGAAGCUGGA (SEQ ID NO: 209), wherein each U may be independently replaced with T. In some embodiments, a characteristic sequence is or comprises AGCUGGAAGCAGGCAAGUUC (SEQ ID NO: 210), wherein each U may be independently replaced with T. In some embodiments, a characteristic sequence is or comprises GACAAACUCAUCCAGAGUGU (SEQ ID NO: 211), wherein each U may be independently replaced with T. In some embodiments, a characteristic sequence is or comprises CCAGAGUGUCAUGGGUGCCC (SEQ ID NO: 212), wherein each U may be independently replaced with T. In some embodiments, a characteristic sequence is or comprises ACCUGGAGCACUGGACAAGU (SEQ ID NO: 213), wherein each U may be independently replaced with T. In some embodiments, a characteristic sequence is or comprises CCAUGACUGCAAGGAUUGGG (SEQ ID NO: 214), wherein each U may be independently replaced with T. In some embodiments, a characteristic sequence is or comprises GAGGACAUGCAGGCUGUGCU (SEQ ID NO: 215), wherein each U may be independently replaced with T. In some embodiments, a characteristic sequence is or comprises CAGGCUCUGACACCAGUUUG (SEQ ID NO: 216), wherein each U may be independently replaced with T. In some embodiments, a characteristic sequence is or comprises ACCAGUUUGGAGGGUGCUGC (SEQ ID NO: 217), wherein each U may be independently replaced with T. In some embodiments, a characteristic sequence is or comprises CCAUGGGUCCAACCUAACCA (SEQ ID NO: 218), wherein each U may be independently replaced with T. In some embodiments, a characteristic sequence is or comprises UGGGCUGAGACAACCUGGGC (SEQ ID NO: 219), wherein each U may be independently replaced with T. In some embodiments, a characteristic sequence is or comprises CCUCAGUAUCUGGAGAGGGA (SEQ ID NO: 220), wherein each U may be independently replaced with T. In some embodiments, a characteristic sequence is or comprises CCUGCCAUUGGGUUGUCUGU (SEQ ID NO: 221), wherein each U may be independently replaced with T. In some embodiments, a characteristic sequence is or comprises CCCUGCUCAGUUCUGGAGAC (SEQ ID NO: 222), wherein each U may be independently replaced with T. In some embodiments, a characteristic sequence is or comprises GCCUCAGACAGGAAUUAAGG (SEQ ID NO: 223), wherein each U may be independently replaced with T. In some embodiments, a characteristic sequence is or comprises GGCCUGGGCACUGUAUUCUG (SEQ ID NO: 224), wherein each U may be independently replaced with T. In some embodiments, a characteristic sequence is or comprises AUUCUGAGCAAGGGCCUGGG (SEQ ID NO: 225), wherein each U may be independently replaced with T. In some embodiments, a characteristic sequence is or comprises AGCCAGCCAGGGAUGAGUGC (SEQ ID NO: 226), wherein each U may be independently replaced with T. In some embodiments, a characteristic sequence is or comprises UGUGGCCUUGCCCUGUAAUC (SEQ ID NO: 227), wherein each U may be independently replaced with T. In some embodiments, a characteristic sequence is or comprises UCUUUUGCCACAUCCAGGGC (SEQ ID NO: 228), wherein each U may be independently replaced with T. In some embodiments, a characteristic sequence is or comprises GCCCUCCCUCUGACUUCCUU (SEQ ID NO: 229), wherein each U may be independently replaced with T. In some embodiments, a characteristic sequence is or comprises GAGCCUCCUGUUUGGGCCUG (SEQ ID NO: 230), wherein each U may be independently replaced with T. In some embodiments, a characteristic sequence is or comprises UUGGGCCUGGGUCUGGGCAU (SEQ ID NO: 231), wherein each U may be independently replaced with T. In some embodiments, a characteristic sequence is or comprises GCCUUGGUGCUGUGCCUCAG (SEQ ID NO: 232), wherein each U may be independently replaced with T. In some embodiments, a characteristic sequence is or comprises AGGCUCCUUCCUGGUCUGGC (SEQ ID NO: 233), wherein each U may be independently replaced with T. In some embodiments, a characteristic sequence is or comprises GGAAAGAGGCAAAGUCCUGA (SEQ ID NO: 234), wherein each U may be independently replaced with T. In some embodiments, a characteristic sequence is or comprises GGGUAGCAAAUCUCUAAAGC (SEQ ID NO: 235), wherein each U may be independently replaced with T. In some embodiments, a characteristic sequence is or comprises GAAUCAUUCUGAGGCUGGGC (SEQ ID NO: 236), wherein each U may be independently replaced with T. In some embodiments, a characteristic sequence is or comprises UCCUCCACUGGGUUCAGAGG (SEQ ID NO: 237), wherein each U may be independently replaced with T. In some embodiments, a characteristic sequence is or comprises ACCCUCCAAUAAACCCAGGC (SEQ ID NO: 238), wherein each U may be independently replaced with T. In some embodiments, a characteristic sequence is or comprises CCACUCUUGGCUGUGCUGGC (SEQ ID NO: 239), wherein each U may be independently replaced with T. In some embodiments, a characteristic sequence is or comprises GGCUCCUUCCUUCCACUCCC (SEQ ID NO: 240), wherein each U may be independently replaced with T.

In some embodiments, an oligonucleotide can hybridize to a region of a nucleic acid. In some embodiments, oligonucleotides that can specifically hybridize (e.g., through sequence complementarity) to certain region(s) of a nucleic acid can more effectively reduce levels of the nucleic acid than oligonucleotides that specifically hybridize (e.g., through sequence complementarity) to one or more reference regions of the nucleic acid. In some embodiments, a region has a length of about 20-200 (e.g., about 20-150, 20-100, 30-200, 30-150, 40-200, 40-150, 50-100, or about 20, 25, 30, 35, 40, 50, 60, 70, 80, 90, 100, 110, 120, 130, 140, 150, 160, 170, 180, 190 or 200) nucleobases. In some embodiments, a region has a length of about 30 nucleobases. In some embodiments, a region has a length of about 40 nucleobases. In some embodiments, a region has a length of about 50 nucleobases. In some embodiments, a region has a length of about 60 nucleobases. In some embodiments, a region has a length of about 70 nucleobases. In some embodiments, a region has a length of about 80 nucleobases. In some embodiments, a region has a length of about 90 nucleobases. In some embodiments, a region has a length of about 100 nucleobases. In some embodiments, a region has a length of about 120 nucleobases. In some embodiments, a region has a length of about 150 nucleobases. In some embodiments, a region has a length of about 200 nucleobases. In some embodiments, a region comprises a complementary sequence of a base sequence of an oligonucleotide in Table 1, which in some embodiments, is in the middle of a region. For example, In some embodiments, a region is or comprises UUUGCUCCCAGGGCUAGUGG (SEQ ID NO: 241). In some embodiments, a region is or comprises GCUCUAUGGAUGGAGAUGGC (SEQ ID NO: 242). In some embodiments, a region is or comprises CCCAAGCCCACAGCUCUCCU (SEQ ID NO: 243). In some embodiments, a region is or comprises UCUGCUGGGAGGCAUGGGUG (SEQ ID NO: 244). In some embodiments, a region is or comprises GGAGAGUGAGUACAGCCAGC (SEQ ID NO: 245). In some embodiments, a region is or comprises CAGGACCAAGGACAGAGCAC (SEQ ID NO: 246). In some embodiments, a region is or comprises GUAGGCUGCAUGAGAAUGGG (SEQ ID NO: 247). In some embodiments, a region is or comprises UCAAGGAGCCUAGGUCACAG (SEQ ID NO: 248). In some embodiments, a region is or comprises GACCCACAGCCUCUCAGACC (SEQ ID NO: 249). In some embodiments, a region is or comprises ACCACAGAAGAACUGGGAGC (SEQ ID NO: 250). In some embodiments, a region is or comprises UGUCACCUGUGGAGGACAUC (SEQ ID NO: 251). In some embodiments, a region is or comprises AGGUCAGUAAGGCAGGAAGC (SEQ ID NO: 252). In some embodiments, a region is or comprises UGGUCAGGGACAAAGGAGAG (SEQ ID NO: 253). In some embodiments, a region is or comprises GAGUGAGGAAAAGGCAAGGC (SEQ ID NO: 254). In some embodiments, a region is or comprises GAGAGGGUUAGUGACCAGGC (SEQ ID NO: 255). In some embodiments, a region is or comprises AGGCAAGACCAAGGUGGGUG (SEQ ID NO: 256). In some embodiments, a region is or comprises UGGGACAGGACAUCAGUGUG (SEQ ID NO: 257). In some embodiments, a region is or comprises GGCCAAGACCCAGAGGUGUG (SEQ ID NO: 258). In some embodiments, a region is or comprises AACUGGGAGUGAUGGGCAGC (SEQ ID NO: 259). In some embodiments, a region is or comprises GGGCCAGGGCAGAUGGAGAG (SEQ ID NO: 260). In some embodiments, a region is or comprises AGAGACAAGGAGAGGGACUG (SEQ ID NO: 261). In some embodiments, a region is or comprises CCCAGGAGCAGCAGGUGGAU (SEQ ID NO: 262). In some embodiments, a region is or comprises GCACCCACAAGACACAAGGG (SEQ ID NO: 263). In some embodiments, a region is or comprises CAACAGGAAAAUCCUAGGGC (SEQ ID NO: 264). In some embodiments, a region is or comprises UGGGAGGCAGGAGUUGAGGC (SEQ ID NO: 265). In some embodiments, a region is or comprises AGGCUGAUACCCAGGUGUCU (SEQ ID NO: 266). In some embodiments, a region is or comprises UGGGAAAUAGGGAAGAAGGA (SEQ ID NO: 267). In some embodiments, a region is or comprises UACCUGACAGUGAGUGAUGC (SEQ ID NO: 268). In some embodiments, a region is or comprises GACAAGGCUGUGGCACUGAC (SEQ ID NO: 269). In some embodiments, a region is or comprises GGGUGCAUAAGGUAGGUGCC (SEQ ID NO: 270). In some embodiments, a region is or comprises GGGCUGAGGGAUGCAGUAGU (SEQ ID NO: 271). In some embodiments, a region is or comprises AGAGACAGGAUGAGACAAGC (SEQ ID NO: 272). In some embodiments, a region is or comprises AAGCAGUCAGCUCAGAGACA (SEQ ID NO: 273). In some embodiments, a region is or comprises UGGAGUGUGGAGUCAAGCCC (SEQ ID NO: 274). In some embodiments, a region is or comprises UGAAGCUGAGAUGCCAUGCC (SEQ ID NO: 275). In some embodiments, a region is or comprises ACCAGCUAGGUGAUCCUGAA (SEQ ID NO: 276). In some embodiments, a region is or comprises CCCAGCAGAUGGCAAAGAGG (SEQ ID NO: 277). In some embodiments, a region is or comprises ACCACAAGUGAACUGCACUC (SEQ ID NO: 278). In some embodiments, a region is or comprises UGACAGGCAGAGUGUGGGCA (SEQ ID NO: 279). In some embodiments, a region is or comprises CUGACUAGCAGCUCCCUCUG (SEQ ID NO: 280). In some embodiments, a region is or comprises GCCUGCAUCACCUUUGCCAA (SEQ ID NO: 281). In some embodiments, a region is or comprises GGUCUUCCAACUGGUGGAGG (SEQ ID NO: 282). In some embodiments, a region is or comprises GAUCCUGGUGGCUGAGAACC (SEQ ID NO: 283). In some embodiments, a region is or comprises UGGCAGGCAUCUUGGAGCAC (SEQ ID NO: 284). In some embodiments, a region is or comprises GCCAUCAAGAGCCUGCAAGG (SEQ ID NO: 285). In some embodiments, a region is or comprises UCCUACUCUACCAAUGGCAC (SEQ ID NO: 286). In some embodiments, a region is or comprises AGGUUCUUUAGGGAGCUCAC (SEQ ID NO: 287). In some embodiments, a region is or comprises CCCUGUACUGGUGGCAAACC (SEQ ID NO: 288). In some embodiments, a region is or comprises AUUGAUGUGGAGAAGCUGGA (SEQ ID NO: 289). In some embodiments, a region is or comprises AGCUGGAAGCAGGCAAGUUC (SEQ ID NO: 290). In some embodiments, a region is or comprises GACAAACUCAUCCAGAGUGU (SEQ ID NO: 291). In some embodiments, a region is or comprises CCAGAGUGUCAUGGGUGCCC (SEQ ID NO: 292). In some embodiments, a region is or comprises ACCUGGAGCACUGGACAAGU (SEQ ID NO: 293). In some embodiments, a region is or comprises CCAUGACUGCAAGGAUUGGG (SEQ ID NO: 294). In some embodiments, a region is or comprises GAGGACAUGCAGGCUGUGCU (SEQ ID NO: 295). In some embodiments, a region is or comprises CAGGCUCUGACACCAGUUUG (SEQ ID NO: 296). In some embodiments, a region is or comprises ACCAGUUUGGAGGGUGCUGC (SEQ ID NO: 297). In some embodiments, a region is or comprises CCAUGGGUCCAACCUAACCA (SEQ ID NO: 298). In some embodiments, a region is or comprises UGGGCUGAGACAACCUGGGC (SEQ ID NO: 299). In some embodiments, a region is or comprises CCUCAGUAUCUGGAGAGGGA (SEQ ID NO: 300). In some embodiments, a region is or comprises CCUGCCAUUGGGUUGUCUGU (SEQ ID NO: 301). In some embodiments, a region is or comprises CCCUGCUCAGUUCUGGAGAC (SEQ ID NO: 302). In some embodiments, a region is or comprises GCCUCAGACAGGAAUUAAGG (SEQ ID NO: 303). In some embodiments, a region is or comprises GGCCUGGGCACUGUAUUCUG (SEQ ID NO: 304). In some embodiments, a region is or comprises AUUCUGAGCAAGGGCCUGGG (SEQ ID NO: 305). In some embodiments, a region is or comprises AGCCAGCCAGGGAUGAGUGC (SEQ ID NO: 306). In some embodiments, a region is or comprises UGUGGCCUUGCCCUGUAAUC (SEQ ID NO: 307). In some embodiments, a region is or comprises UCUUUUGCCACAUCCAGGGC (SEQ ID NO: 308). In some embodiments, a region is or comprises GCCCUCCCUCUGACUUCCUU (SEQ ID NO: 309). In some embodiments, a region is or comprises GAGCCUCCUGUUUGGGCCUG (SEQ ID NO: 310). In some embodiments, a region is or comprises UUGGGCCUGGGUCUGGGCAU (SEQ ID NO: 311). In some embodiments, a region is or comprises GCCUUGGUGCUGUGCCUCAG (SEQ ID NO: 312). In some embodiments, a region is or comprises AGGCUCCUUCCUGGUCUGGC (SEQ ID NO: 313). In some embodiments, a region is or comprises GGAAAGAGGCAAAGUCCUGA (SEQ ID NO: 314). In some embodiments, a region is or comprises GGGUAGCAAAUCUCUAAAGC (SEQ ID NO: 315). In some embodiments, a region is or comprises GAAUCAUUCUGAGGCUGGGC (SEQ ID NO: 316). In some embodiments, a region is or comprises UCCUCCACUGGGUUCAGAGG (SEQ ID NO: 317). In some embodiments, a region is or comprises ACCCUCCAAUAAACCCAGGC (SEQ ID NO: 318). In some embodiments, a region is or comprises CCACUCUUGGCUGUGCUGGC (SEQ ID NO: 319). In some embodiments, a region is or comprises GGCUCCUUCCUUCCACUCCC (SEQ ID NO: 320).

In some embodiments, base sequences of oligonucleotides comprise or consist of about 10-50 (e.g., about 15-50, 16-50, 17-50, 18-50, 19-50, 20-50, 15-30, 20-30, 15-25 or 20-25, or at least about 10, 11, 12, 13, 14, 15, 16, 17, 18, 19, 20, 21, 22, 23, 24, 25, 30, 35, 40, 45; in some embodiments, at least about 15; in some embodiments, at least about 16; in some embodiments, at least about 17; in some embodiments, at least about 18; in some embodiments, at least about 19; in some embodiments, at least about 20; in some embodiments, at least about 21; in some embodiments, at least about 22; in some embodiments, at least about 23; in some embodiments, at least about 24; in some embodiments, at least about 25) contiguous bases that are identical to or complementary to a base sequence of equal length in a SARM1 gene or a transcript (e.g., mRNA) thereof. In some embodiments, they ae complementary to a base sequence of equal length in a SARM1 transcript.

In certain embodiments, a base sequence of an oligonucleotide is at least about 50%, 60%, 70%, 75%, 80%, 85%, 90%, 91%, 92%, 93%, 94%, 95%, 96%, 97%, 98%, or 99%, or 100% complementary to a target sequence in a SARM1 transcript. In some embodiments, a base sequence of an oligonucleotide is fully complementary to a target sequence in a SARM1 transcript.

In some embodiments, the base sequence of an oligonucleotide has about 80% or more identity with the base sequence of an oligonucleotide disclosed in Table 1, wherein each T can be independently replaced with U and vice versa. In some embodiments, the base sequence of an oligonucleotide has about 85% or more identity with the base sequence of an oligonucleotide disclosed in Table 1, wherein each T can be independently replaced with U and vice versa. In some embodiments, the base sequence of an oligonucleotide has about 90% or more identity with the base sequence of an oligonucleotide disclosed in Table 1, wherein each T can be independently replaced with U and vice versa. In some embodiments, the base sequence of an oligonucleotide has about 95% or more identity with the base sequence of an oligonucleotide disclosed in Table 1, wherein each T can be independently replaced with U and vice versa.

In some embodiments, the base sequence of an oligonucleotide comprises a continuous span of about 15 or more bases of an oligonucleotide disclosed in a Table 1, wherein each T can be independently replaced with U and vice versa. In some embodiments, the base sequence of an oligonucleotide comprises a continuous span of about 16 or more bases of an oligonucleotide disclosed in a Table 1, wherein each T can be independently replaced with U and vice versa. In some embodiments, the base sequence of an oligonucleotide comprises a continuous span of about 17 or more bases of an oligonucleotide disclosed in a Table 1, wherein each T can be independently replaced with U and vice versa. In some embodiments, the base sequence of an oligonucleotide comprises a continuous span of about 18 or more bases of an oligonucleotide disclosed in a Table 1, wherein each T can be independently replaced with U and vice versa. In some embodiments, the base sequence of an oligonucleotide comprises a continuous span of about 19 or more bases of an oligonucleotide disclosed in a Table 1, wherein each T can be independently replaced with U and vice versa. In some embodiments, the base sequence of an oligonucleotide comprises a continuous span of about 20 or more bases of an oligonucleotide disclosed in a Table 1, wherein each T can be independently replaced with U and vice versa.

In some embodiments, the base sequence of an oligonucleotide comprises the base sequence of an oligonucleotide in Table 1, wherein each T may be independently replaced with U and vice versa. In some embodiments, the base sequence of an oligonucleotide is the base sequence of an oligonucleotide in Table 1, wherein each T may be independently replaced with U and vice versa. For example, in some embodiments, the base sequence of an oligonucleotide comprises CCACTAGCCCTGGGAGCAAA (SEQ ID NO: 1), wherein each T may be independently replaced with U. In some embodiments, the base sequence of an oligonucleotide comprises GCCATCTCCATCCATAGAGC (SEQ ID NO: 2), wherein each T may be independently replaced with U. In some embodiments, the base sequence of an oligonucleotide comprises AGGAGAGCTGTGGGCTTGGG (SEQ ID NO: 3), wherein each T may be independently replaced with U. In some embodiments, the base sequence of an oligonucleotide comprises CACCCATGCCTCCCAGCAGA (SEQ ID NO: 4), wherein each T may be independently replaced with U. In some embodiments, the base sequence of an oligonucleotide comprises GCTGGCTGTACTCACTCTCC (SEQ ID NO: 5), wherein each T may be independently replaced with U. In some embodiments, the base sequence of an oligonucleotide comprises GTGCTCTGTCCTTGGTCCTG (SEQ ID NO: 6), wherein each T may be independently replaced with U. In some embodiments, the base sequence of an oligonucleotide comprises CCCATTCTCATGCAGCCTAC (SEQ ID NO: 7), wherein each T may be independently replaced with U. In some embodiments, the base sequence of an oligonucleotide comprises CTGTGACCTAGGCTCCTTGA (SEQ ID NO: 8), wherein each T may be independently replaced with U. In some embodiments, the base sequence of an oligonucleotide comprises GGTCTGAGAGGCTGTGGGTC (SEQ ID NO: 9), wherein each T may be independently replaced with U. In some embodiments, the base sequence of an oligonucleotide comprises GCTCCCAGTTCTTCTGTGGT (SEQ ID NO: 10), wherein each T may be independently replaced with U. In some embodiments, the base sequence of an oligonucleotide comprises GATGTCCTCCACAGGTGACA (SEQ ID NO: 11), wherein each T may be independently replaced with U. In some embodiments, the base sequence of an oligonucleotide comprises GCTTCCTGCCTTACTGACCT (SEQ ID NO: 12), wherein each T may be independently replaced with U. In some embodiments, the base sequence of an oligonucleotide comprises CTCTCCTTTGTCCCTGACCA (SEQ ID NO: 13), wherein each T may be independently replaced with U. In some embodiments, the base sequence of an oligonucleotide comprises GCCTTGCCTTTTCCTCACTC (SEQ ID NO: 14), wherein each T may be independently replaced with U. In some embodiments, the base sequence of an oligonucleotide comprises GCCTGGTCACTAACCCTCTC (SEQ ID NO: 15), wherein each T may be independently replaced with U. In some embodiments, the base sequence of an oligonucleotide comprises CACCCACCTTGGTCTTGCCT (SEQ ID NO: 16), wherein each T may be independently replaced with U. In some embodiments, the base sequence of an oligonucleotide comprises CACACTGATGTCCTGTCCCA (SEQ ID NO: 17), wherein each T may be independently replaced with U. In some embodiments, the base sequence of an oligonucleotide comprises CACACCTCTGGGTCTTGGCC (SEQ ID NO: 18), wherein each T may be independently replaced with U. In some embodiments, the base sequence of an oligonucleotide comprises GCTGCCCATCACTCCCAGTT (SEQ ID NO: 19), wherein each T may be independently replaced with U. In some embodiments, the base sequence of an oligonucleotide comprises CTCTCCATCTGCCCTGGCCC (SEQ ID NO: 20), wherein each T may be independently replaced with U. In some embodiments, the base sequence of an oligonucleotide comprises CAGTCCCTCTCCTTGTCTCT (SEQ ID NO: 21), wherein each T may be independently replaced with U. In some embodiments, the base sequence of an oligonucleotide comprises ATCCACCTGCTGCTCCTGGG (SEQ ID NO: 22), wherein each T may be independently replaced with U. In some embodiments, the base sequence of an oligonucleotide comprises CCCTTGTGTCTTGTGGGTGC (SEQ ID NO: 23), wherein each T may be independently replaced with U. In some embodiments, the base sequence of an oligonucleotide comprises GCCCTAGGATTTTCCTGTTG (SEQ ID NO: 24), wherein each T may be independently replaced with U. In some embodiments, the base sequence of an oligonucleotide comprises GCCTCAACTCCTGCCTCCCA (SEQ ID NO: 25), wherein each T may be independently replaced with U. In some embodiments, the base sequence of an oligonucleotide comprises AGACACCTGGGTATCAGCCT (SEQ ID NO: 26), wherein each T may be independently replaced with U. In some embodiments, the base sequence of an oligonucleotide comprises TCCTTCTTCCCTATTTCCCA (SEQ ID NO: 27), wherein each T may be independently replaced with U. In some embodiments, the base sequence of an oligonucleotide comprises GCATCACTCACTGTCAGGTA (SEQ ID NO: 28), wherein each T may be independently replaced with U. In some embodiments, the base sequence of an oligonucleotide comprises GTCAGTGCCACAGCCTTGTC (SEQ ID NO: 29), wherein each T may be independently replaced with U. In some embodiments, the base sequence of an oligonucleotide comprises GGCACCTACCTTATGCACCC (SEQ ID NO: 30), wherein each T may be independently replaced with U. In some embodiments, the base sequence of an oligonucleotide comprises ACTACTGCATCCCTCAGCCC (SEQ ID NO: 31), wherein each T may be independently replaced with U. In some embodiments, the base sequence of an oligonucleotide comprises GCTTGTCTCATCCTGTCTCT (SEQ ID NO: 32), wherein each T may be independently replaced with U. In some embodiments, the base sequence of an oligonucleotide comprises TGTCTCTGAGCTGACTGCTT (SEQ ID NO: 33), wherein each T may be independently replaced with U. In some embodiments, the base sequence of an oligonucleotide comprises GGGCTTGACTCCACACTCCA (SEQ ID NO: 34), wherein each T may be independently replaced with U. In some embodiments, the base sequence of an oligonucleotide comprises GGCATGGCATCTCAGCTTCA (SEQ ID NO: 35), wherein each T may be independently replaced with U. In some embodiments, the base sequence of an oligonucleotide comprises TTCAGGATCACCTAGCTGGT (SEQ ID NO: 36), wherein each T may be independently replaced with U. In some embodiments, the base sequence of an oligonucleotide comprises CCTCTTTGCCATCTGCTGGG (SEQ ID NO: 37), wherein each T may be independently replaced with U. In some embodiments, the base sequence of an oligonucleotide comprises GAGTGCAGTTCACTTGTGGT (SEQ ID NO: 38), wherein each T may be independently replaced with U. In some embodiments, the base sequence of an oligonucleotide comprises TGCCCACACTCTGCCTGTCA (SEQ ID NO: 39), wherein each T may be independently replaced with U. In some embodiments, the base sequence of an oligonucleotide comprises CAGAGGGAGCTGCTAGTCAG (SEQ ID NO: 40), wherein each T may be independently replaced with U. In some embodiments, the base sequence of an oligonucleotide comprises TTGGCAAAGGTGATGCAGGC (SEQ ID NO: 41), wherein each T may be independently replaced with U. In some embodiments, the base sequence of an oligonucleotide comprises CCTCCACCAGTTGGAAGACC (SEQ ID NO: 42), wherein each T may be independently replaced with U. In some embodiments, the base sequence of an oligonucleotide comprises GGTTCTCAGCCACCAGGATC (SEQ ID NO: 43), wherein each T may be independently replaced with U. In some embodiments, the base sequence of an oligonucleotide comprises GTGCTCCAAGATGCCTGCCA (SEQ ID NO: 44), wherein each T may be independently replaced with U. In some embodiments, the base sequence of an oligonucleotide comprises CCTTGCAGGCTCTTGATGGC (SEQ ID NO: 45), wherein each T may be independently replaced with U. In some embodiments, the base sequence of an oligonucleotide comprises GTGCCATTGGTAGAGTAGGA (SEQ ID NO: 46), wherein each T may be independently replaced with U. In some embodiments, the base sequence of an oligonucleotide comprises GTGAGCTCCCTAAAGAACCT (SEQ ID NO: 47), wherein each T may be independently replaced with U. In some embodiments, the base sequence of an oligonucleotide comprises GGTTTGCCACCAGTACAGGG (SEQ ID NO: 48), wherein each T may be independently replaced with U. In some embodiments, the base sequence of an oligonucleotide comprises TCCAGCTTCTCCACATCAAT (SEQ ID NO: 49), wherein each T may be independently replaced with U. In some embodiments, the base sequence of an oligonucleotide comprises GAACTTGCCTGCTTCCAGCT (SEQ ID NO: 50), wherein each T may be independently replaced with U. In some embodiments, the base sequence of an oligonucleotide comprises ACACTCTGGATGAGTTTGTC (SEQ ID NO: 51), wherein each T may be independently replaced with U. In some embodiments, the base sequence of an oligonucleotide comprises GGGCACCCATGACACTCTGG (SEQ ID NO: 52), wherein each T may be independently replaced with U. In some embodiments, the base sequence of an oligonucleotide comprises ACTTGTCCAGTGCTCCAGGT (SEQ ID NO: 53), wherein each T may be independently replaced with U. In some embodiments, the base sequence of an oligonucleotide comprises CCCAATCCTTGCAGTCATGG (SEQ ID NO: 54), wherein each T may be independently replaced with U. In some embodiments, the base sequence of an oligonucleotide comprises AGCACAGCCTGCATGTCCTC (SEQ ID NO: 55), wherein each T may be independently replaced with U. In some embodiments, the base sequence of an oligonucleotide comprises CAAACTGGTGTCAGAGCCTG (SEQ ID NO: 56), wherein each T may be independently replaced with U. In some embodiments, the base sequence of an oligonucleotide comprises GCAGCACCCTCCAAACTGGT (SEQ ID NO: 57), wherein each T may be independently replaced with U. In some embodiments, the base sequence of an oligonucleotide comprises TGGTTAGGTTGGACCCATGG (SEQ ID NO: 58), wherein each T may be independently replaced with U. In some embodiments, the base sequence of an oligonucleotide comprises GCCCAGGTTGTCTCAGCCCA (SEQ ID NO: 59), wherein each T may be independently replaced with U. In some embodiments, the base sequence of an oligonucleotide comprises TCCCTCTCCAGATACTGAGG (SEQ ID NO: 60), wherein each T may be independently replaced with U. In some embodiments, the base sequence of an oligonucleotide comprises ACAGACAACCCAATGGCAGG (SEQ ID NO: 61), wherein each T may be independently replaced with U. In some embodiments, the base sequence of an oligonucleotide comprises GTCTCCAGAACTGAGCAGGG (SEQ ID NO: 62), wherein each T may be independently replaced with U. In some embodiments, as confirmed in the Examples, such oligonucleotide, e.g., oligonucleotide 62, can provide high and persistent reduction of SARM1 mRNA and polypeptide levels. In some embodiments, the base sequence of an oligonucleotide comprises CCTTAATTCCTGTCTGAGGC (SEQ ID NO: 63), wherein each T may be independently replaced with U. In some embodiments, the base sequence of an oligonucleotide comprises CAGAATACAGTGCCCAGGCC (SEQ ID NO: 64), wherein each T may be independently replaced with U. In some embodiments, the base sequence of an oligonucleotide comprises CCCAGGCCCTTGCTCAGAAT (SEQ ID NO: 65), wherein each T may be independently replaced with U. In some embodiments, the base sequence of an oligonucleotide comprises GCACTCATCCCTGGCTGGCT (SEQ ID NO: 66), wherein each T may be independently replaced with U. In some embodiments, the base sequence of an oligonucleotide comprises GATTACAGGGCAAGGCCACA (SEQ ID NO: 67), wherein each T may be independently replaced with U. In some embodiments, the base sequence of an oligonucleotide comprises GCCCTGGATGTGGCAAAAGA (SEQ ID NO: 68), wherein each T may be independently replaced with U. In some embodiments, the base sequence of an oligonucleotide comprises AAGGAAGTCAGAGGGAGGGC (SEQ ID NO: 69), wherein each T may be independently replaced with U. In some embodiments, the base sequence of an oligonucleotide comprises CAGGCCCAAACAGGAGGCTC (SEQ ID NO: 70), wherein each T may be independently replaced with U. In some embodiments, the base sequence of an oligonucleotide comprises ATGCCCAGACCCAGGCCCAA (SEQ ID NO: 71), wherein each T may be independently replaced with U. In some embodiments, the base sequence of an oligonucleotide comprises CTGAGGCACAGCACCAAGGC (SEQ ID NO: 72), wherein each T may be independently replaced with U. In some embodiments, the base sequence of an oligonucleotide comprises GCCAGACCAGGAAGGAGCCT (SEQ ID NO: 73), wherein each T may be independently replaced with U. In some embodiments, the base sequence of an oligonucleotide comprises TCAGGACTTTGCCTCTTTCC (SEQ ID NO: 74), wherein each T may be independently replaced with U. In some embodiments, the base sequence of an oligonucleotide comprises GCTTTAGAGATTTGCTACCC (SEQ ID NO: 75), wherein each T may be independently replaced with U. In some embodiments, the base sequence of an oligonucleotide comprises GCCCAGCCTCAGAATGATTC (SEQ ID NO: 76), wherein each T may be independently replaced with U. In some embodiments, the base sequence of an oligonucleotide comprises CCTCTGAACCCAGTGGAGGA (SEQ ID NO: 77), wherein each T may be independently replaced with U. In some embodiments, the base sequence of an oligonucleotide comprises GCCTGGGTTTATTGGAGGGT (SEQ ID NO: 78), wherein each T may be independently replaced with U. In some embodiments, the base sequence of an oligonucleotide comprises GCCAGCACAGCCAAGAGTGG (SEQ ID NO: 79), wherein each T may be independently replaced with U. In some embodiments, the base sequence of an oligonucleotide comprises GGGAGTGGAAGGAAGGAGCC (SEQ ID NO: 80), wherein each T may be independently replaced with U.

In some embodiments, the base sequence of an oligonucleotide comprises CCACTAGCCCTGGGAGCAAA (SEQ ID NO: 81). In some embodiments, the base sequence of an oligonucleotide comprises GCCATCTCCATCCATAGAGC (SEQ ID NO: 82). In some embodiments, the base sequence of an oligonucleotide comprises AGGAGAGCTGTGGGCTTGGG (SEQ ID NO: 83). In some embodiments, the base sequence of an oligonucleotide comprises CACCCATGCCTCCCAGCAGA (SEQ ID NO: 84). In some embodiments, the base sequence of an oligonucleotide comprises GCTGGCTGTACTCACTCTCC (SEQ ID NO: 85). In some embodiments, the base sequence of an oligonucleotide comprises GTGCTCTGTCCTTGGTCCTG (SEQ ID NO: 86). In some embodiments, the base sequence of an oligonucleotide comprises CCCATTCTCATGCAGCCTAC (SEQ ID NO: 87). In some embodiments, the base sequence of an oligonucleotide comprises CTGTGACCTAGGCTCCTTGA (SEQ ID NO: 88). In some embodiments, the base sequence of an oligonucleotide comprises GGTCTGAGAGGCTGTGGGTC (SEQ ID NO: 89). In some embodiments, the base sequence of an oligonucleotide comprises GCTCCCAGTTCTTCTGTGGT (SEQ ID NO: 90). In some embodiments, the base sequence of an oligonucleotide comprises GATGTCCTCCACAGGTGACA (SEQ ID NO: 91). In some embodiments, the base sequence of an oligonucleotide comprises GCTTCCTGCCTTACTGACCT (SEQ ID NO: 92). In some embodiments, the base sequence of an oligonucleotide comprises CTCTCCTTTGTCCCTGACCA (SEQ ID NO: 93). In some embodiments, the base sequence of an oligonucleotide comprises GCCTTGCCTTTTCCTCACTC (SEQ ID NO: 94). In some embodiments, the base sequence of an oligonucleotide comprises GCCTGGTCACTAACCCTCTC (SEQ ID NO: 95). In some embodiments, the base sequence of an oligonucleotide comprises CACCCACCTTGGTCTTGCCT (SEQ ID NO: 96). In some embodiments, the base sequence of an oligonucleotide comprises CACACTGATGTCCTGTCCCA (SEQ ID NO: 97). In some embodiments, the base sequence of an oligonucleotide comprises CACACCTCTGGGTCTTGGCC (SEQ ID NO: 98). In some embodiments, the base sequence of an oligonucleotide comprises GCTGCCCATCACTCCCAGTT (SEQ ID NO: 99). In some embodiments, the base sequence of an oligonucleotide comprises CTCTCCATCTGCCCTGGCCC (SEQ ID NO: 100). In some embodiments, the base sequence of an oligonucleotide comprises CAGTCCCTCTCCTTGTCTCT (SEQ ID NO: 101). In some embodiments, the base sequence of an oligonucleotide comprises ATCCACCTGCTGCTCCTGGG (SEQ ID NO: 102). In some embodiments, the base sequence of an oligonucleotide comprises CCCTTGTGTCTTGTGGGTGC (SEQ ID NO: 103). In some embodiments, the base sequence of an oligonucleotide comprises GCCCTAGGATTTTCCTGTTG (SEQ ID NO: 104). In some embodiments, the base sequence of an oligonucleotide comprises GCCTCAACTCCTGCCTCCCA (SEQ ID NO: 105). In some embodiments, the base sequence of an oligonucleotide comprises AGACACCTGGGTATCAGCCT (SEQ ID NO: 106). In some embodiments, the base sequence of an oligonucleotide comprises TCCTTCTTCCCTATTTCCCA (SEQ ID NO: 107). In some embodiments, the base sequence of an oligonucleotide comprises GCATCACTCACTGTCAGGTA (SEQ ID NO: 108). In some embodiments, the base sequence of an oligonucleotide comprises GTCAGTGCCACAGCCTTGTC (SEQ ID NO: 109). In some embodiments, the base sequence of an oligonucleotide comprises GGCACCTACCTTATGCACCC (SEQ ID NO: 110). In some embodiments, the base sequence of an oligonucleotide comprises ACTACTGCATCCCTCAGCCC (SEQ ID NO: 111). In some embodiments, the base sequence of an oligonucleotide comprises GCTTGTCTCATCCTGTCTCT (SEQ ID NO: 112). In some embodiments, the base sequence of an oligonucleotide comprises TGTCTCTGAGCTGACTGCTT (SEQ ID NO: 113). In some embodiments, the base sequence of an oligonucleotide comprises GGGCTTGACTCCACACTCCA (SEQ ID NO: 114). In some embodiments, the base sequence of an oligonucleotide comprises GGCATGGCATCTCAGCTTCA (SEQ ID NO: 115). In some embodiments, the base sequence of an oligonucleotide comprises TTCAGGATCACCTAGCTGGT (SEQ ID NO: 116). In some embodiments, the base sequence of an oligonucleotide comprises CCTCTTTGCCATCTGCTGGG (SEQ ID NO: 117). In some embodiments, the base sequence of an oligonucleotide comprises GAGTGCAGTTCACTTGTGGT (SEQ ID NO: 118). In some embodiments, the base sequence of an oligonucleotide comprises TGCCCACACTCTGCCTGTCA (SEQ ID NO: 119). In some embodiments, the base sequence of an oligonucleotide comprises CAGAGGGAGCTGCTAGTCAG (SEQ ID NO: 120). In some embodiments, the base sequence of an oligonucleotide comprises TTGGCAAAGGTGATGCAGGC (SEQ ID NO: 121). In some embodiments, the base sequence of an oligonucleotide comprises CCTCCACCAGTTGGAAGACC (SEQ ID NO: 122). In some embodiments, the base sequence of an oligonucleotide comprises GGTTCTCAGCCACCAGGATC (SEQ ID NO: 123). In some embodiments, the base sequence of an oligonucleotide comprises GTGCTCCAAGATGCCTGCCA (SEQ ID NO: 124). In some embodiments, the base sequence of an oligonucleotide comprises CCTTGCAGGCTCTTGATGGC (SEQ ID NO: 125). In some embodiments, the base sequence of an oligonucleotide comprises GTGCCATTGGTAGAGTAGGA (SEQ ID NO: 126). In some embodiments, the base sequence of an oligonucleotide comprises GTGAGCTCCCTAAAGAACCT (SEQ ID NO: 127). In some embodiments, the base sequence of an oligonucleotide comprises GGTTTGCCACCAGTACAGGG (SEQ ID NO: 128). In some embodiments, the base sequence of an oligonucleotide comprises TCCAGCTTCTCCACATCAAT (SEQ ID NO: 129). In some embodiments, the base sequence of an oligonucleotide comprises GAACTTGCCTGCTTCCAGCT (SEQ ID NO: 130). In some embodiments, the base sequence of an oligonucleotide comprises ACACTCTGGATGAGTTTGTC (SEQ ID NO: 131). In some embodiments, the base sequence of an oligonucleotide comprises GGGCACCCATGACACTCTGG (SEQ ID NO: 132). In some embodiments, the base sequence of an oligonucleotide comprises ACTTGTCCAGTGCTCCAGGT (SEQ ID NO: 133). In some embodiments, the base sequence of an oligonucleotide comprises CCCAATCCTTGCAGTCATGG (SEQ ID NO: 134). In some embodiments, the base sequence of an oligonucleotide comprises AGCACAGCCTGCATGTCCTC (SEQ ID NO: 135). In some embodiments, the base sequence of an oligonucleotide comprises CAAACTGGTGTCAGAGCCTG (SEQ ID NO: 136). In some embodiments, the base sequence of an oligonucleotide comprises GCAGCACCCTCCAAACTGGT (SEQ ID NO: 137). In some embodiments, the base sequence of an oligonucleotide comprises TGGTTAGGTTGGACCCATGG (SEQ ID NO: 138). In some embodiments, the base sequence of an oligonucleotide comprises GCCCAGGTTGTCTCAGCCCA (SEQ ID NO: 139). In some embodiments, the base sequence of an oligonucleotide comprises TCCCTCTCCAGATACTGAGG (SEQ ID NO: 140). In some embodiments, the base sequence of an oligonucleotide comprises ACAGACAACCCAATGGCAGG (SEQ ID NO: 141). In some embodiments, the base sequence of an oligonucleotide comprises GTCTCCAGAACTGAGCAGGG (SEQ ID NO: 142). In some embodiments, the base sequence of an oligonucleotide comprises CCTTAATTCCTGTCTGAGGC (SEQ ID NO: 143). In some embodiments, the base sequence of an oligonucleotide comprises CAGAATACAGTGCCCAGGCC (SEQ ID NO: 144). In some embodiments, the base sequence of an oligonucleotide comprises CCCAGGCCCTTGCTCAGAAT (SEQ ID NO: 145). In some embodiments, the base sequence of an oligonucleotide comprises GCACTCATCCCTGGCTGGCT (SEQ ID NO: 146). In some embodiments, the base sequence of an oligonucleotide comprises GATTACAGGGCAAGGCCACA (SEQ ID NO: 147). In some embodiments, the base sequence of an oligonucleotide comprises GCCCTGGATGTGGCAAAAGA (SEQ ID NO: 148). In some embodiments, the base sequence of an oligonucleotide comprises AAGGAAGTCAGAGGGAGGGC (SEQ ID NO: 149). In some embodiments, the base sequence of an oligonucleotide comprises CAGGCCCAAACAGGAGGCTC (SEQ ID NO: 150). In some embodiments, the base sequence of an oligonucleotide comprises ATGCCCAGACCCAGGCCCAA (SEQ ID NO: 151). In some embodiments, the base sequence of an oligonucleotide comprises CTGAGGCACAGCACCAAGGC (SEQ ID NO: 152). In some embodiments, the base sequence of an oligonucleotide comprises GCCAGACCAGGAAGGAGCCT (SEQ ID NO: 153). In some embodiments, the base sequence of an oligonucleotide comprises TCAGGACTTTGCCTCTTTCC (SEQ ID NO: 154). In some embodiments, the base sequence of an oligonucleotide comprises GCTTTAGAGATTTGCTACCC (SEQ ID NO: 155). In some embodiments, the base sequence of an oligonucleotide comprises GCCCAGCCTCAGAATGATTC (SEQ ID NO: 156). In some embodiments, the base sequence of an oligonucleotide comprises CCTCTGAACCCAGTGGAGGA (SEQ ID NO: 157). In some embodiments, the base sequence of an oligonucleotide comprises GCCTGGGTTTATTGGAGGGT (SEQ ID NO: 158). In some embodiments, the base sequence of an oligonucleotide comprises GCCAGCACAGCCAAGAGTGG (SEQ ID NO: 159). In some embodiments, the base sequence of an oligonucleotide comprises GGGAGTGGAAGGAAGGAGCC (SEQ ID NO: 160).

### Lengths

As appreciated by those skilled in the art, oligonucleotides can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, provided oligonucleotides are of suitable lengths to hybridize with their targets and reduce levels of their targets and/or a product thereof. In some embodiments, an oligonucleotide is long enough to recognize a target nucleic acid (e.g., a SARM1 mRNA). In some embodiments, an oligonucleotide is sufficiently long to distinguish between a target nucleic acid and other nucleic acids (e.g., a nucleic acid having a base sequence which is not a SARM1 sequence) to reduce off-target effects. In some embodiments, an oligonucleotide is sufficiently short to reduce complexity of manufacture or production and to reduce cost of products.

In some embodiments, the base sequence of an oligonucleotide is about 10-100 nucleobases in length. In some embodiments, a base sequence is about 10-50 nucleobases in length. In some embodiments, a base sequence is about 15-50 nucleobases in length. In some embodiments, a base sequence is about 15-30 nucleobases in length. In some embodiments, a base sequence is about 15-25 nucleobases in length. In some embodiments, a base sequence is about 15-22 nucleobases in length. In some embodiments, a base sequence is about 18-22 nucleobases in length. In some embodiments, a base sequence is about 10, 11, 12, 13, 14, 15, 16, 17, 18, 19, 20, 21, 22, 23, 24, or 25 nucleobases in length. In some embodiments, a base sequence is at least about 12 nucleobases in length. In some embodiments, a base sequence is at least about 13 nucleobases in length. In some embodiments, a base sequence is at least about 14 nucleobases in length. In some embodiments, a base sequence is at least about 15 nucleobases in length. In some embodiments, a base sequence is at least about 16 nucleobases in length. In some embodiments, a base sequence is at least about 17 nucleobases in length. In some embodiments, a base sequence is at least about 18 nucleobases in length. In some embodiments, a base sequence is at least about 19 nucleobases in length. In some embodiments, a base sequence is at least about 20 nucleobases in length. In some embodiments, a base sequence is at least about 21 nucleobases in length. In some embodiments, a base sequence is at least about 22 nucleobases in length. In some embodiments, a base sequence is at least about 23 nucleobases in length. In some embodiments, a base sequence is at least about 24 nucleobases in length. In some embodiments, a base sequence is at least about 25 nucleobases in length. In some embodiments, a base sequence is about 15 nucleobases in length. In some embodiments, a base sequence is about 16 nucleobases in length. In some embodiments, a base sequence is about 17 nucleobases in length. In some embodiments, a base sequence is about 18 nucleobases in length. In some embodiments, a base sequence is about 19 nucleobases in length. In some embodiments, a base sequence is about 20 nucleobases in length. In some embodiments, a base sequence is about 21 nucleobases in length. In some embodiments, a base sequence is about 22 nucleobases in length. In some embodiments, a base sequence is about 23 nucleobases in length. In some embodiments, a base sequence is about 24 nucleobases in length. In some embodiments, a base sequence is about 25 nucleobases in length. In some other embodiments, a base sequence is about at least about 30 nucleobases in length. In some embodiments, each nucleobase independently comprises an optionally substituted monocyclic, bicyclic or polycyclic ring wherein at least one ring atom is nitrogen. In some embodiments, each nucleobase is independently optionally substituted adenine, cytosine, guanosine, thymine, or uracil, or an optionally substituted tautomer of adenine, cytosine, guanosine, thymine, or uracil.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)].

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytosine (5mC), 5-hydroxymethylcytosine, 5-formylcytosine, or 5-carboxylcytosine. In some embodiments, an oligonucleotide comprises one or more 5mC. In some embodiments, each nucleobase in an oligonucleotide is independently selected from optionally substituted A, T, C, G and U, and optionally substituted tautomers of A, T, C, G and U. In some embodiments, each nucleobase in an oligonucleotide is independently optionally protected A, T, C, 5mC, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

In some embodiments, a nucleobase is optionally substituted 2AP or DAP. In some embodiments, a nucleobase is optionally substituted 2AP. In some embodiments, a nucleobase is optionally substituted DAP. In some embodiments, a nucleobase is 2AP. In some embodiments, a nucleobase is DAP.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313.

In some embodiments, a provided oligonucleotide comprises one or more 5-methylcytosine. In some embodiments, the present disclosure provides an oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide—such oligonucleotide comprises a nucleobase modification at the C position (e.g., see various oligonucleotides in Table 1 or A2). In description of oligonucleotides, unless otherwise noted, nucleobases, sugars and internucleotidic linkages are non-modified.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added.

Nucleobases may be protected during oligonucleotide synthesis. Various protection technologies are available and can be utilized in accordance with the present disclosure.

In some embodiments, a modified nucleobase is 5-substituted pyrimidines, 6-azapyrimidines, alkyl or alkynyl substituted pyrimidines, alkyl substituted purines, or N-2, N-6 and 0-6 substituted purines. In certain embodiments, a modified nucleobase is selected form 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, a modified nucleobases is a tricyclic pyrimidine, such as 1,3-diazaphenoxazine-2-one, 1,3-diazaphenothiazine-2-one or 9-(2-aminoethoxy)-1,3-diazaphenoxazine-2-one (G-clamp). In some embodiments, a modified nucleobases is one in which a purine or pyrimidine base is replaced with other heterocycles, for example, 7-deaza-adenine, 7-deazaguanosine, 2-aminopyridine or 2-pyridone.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

In some embodiments, a nucleobase is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobase of each of which is incorporated herein by reference.

### Sugars

Various sugars, including modified sugars, can be utilized in accordance with the present disclosure. In some embodiments, the present disclosure provides sugar modifications and patterns thereof optionally in combination with other structural elements (e.g., nucleobase modifications and patterns thereof, internucleotidic linkage modifications and patterns thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

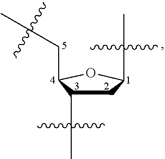

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

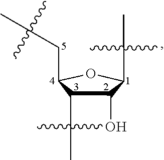

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability and/or affinity. In some embodiments, modified sugars can be utilized to alter and/or optimize one or more hybridization characteristics. In some embodiments, modified sugars can be utilized to alter and/or optimize target recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. As most commonly in natural nucleic acids, an internucleotidic linkage typically connects with one sugar at the 5′ position and another sugar at the 3′ position unless otherwise indicated.

In some embodiments, a sugar is an optionally substituted natural DNA or RNA sugar. In some embodiments, a sugar is optionally substituted

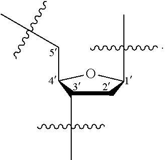

In some embodiments, the 2′ position is optionally substituted. In some embodiments, a sugar is

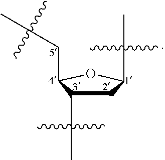

In some embodiments, a sugar has the structure of

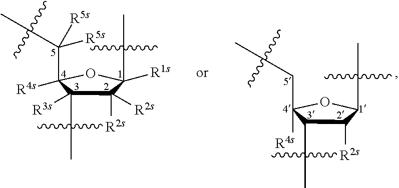

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, descriptions of R^{1s}, R^{2s}, R^{3a}, R^{4s}, and R^{5s}, and modified sugars of each of which are independently incorporated herein by reference). In some embodiments, each of R^{1s}, R^{2s}, R³, R^{4s}, and R^{5s} is independently —F, —Cl, —Br, —I, —CN, —N₃, —NO, —NO₂, -L^{s}-R′, -L^{s}-OR^{s}, -L^{s}-SR′, -L^{s}-N(R′)₂, —O-L^{s}-OR^{s}, —O-L^{s}-SR′, or —O-L^{s}-N(R′)₂, wherein each R′ is independently —H or an optionally substituted group selected from C₁₋₁₀ aliphatic, C₆₋₁₄ aryl, C₁₋₁₀ heteroaliphatic having 1-5 heteroatoms, 5-10 membered heteroaryl having 1-5 heteroatoms and 3-10 membered heterocyclyl having 1-4 heteroatoms, or two or more R′ groups are taken together with their intervening atoms to from an optionally substituted 3-10 membered ring having 0-5 heteroatoms in addition to the intervening atoms. independently as described herein, and L^{s} is a covalent bond or optionally substituted bivalent C₁₋₆ aliphatic or heteroaliphatic having 1-4 heteroatoms. In some embodiments, a sugar has the structure of

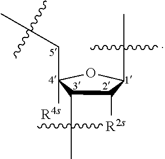

In some embodiments, R^{4s} is —H. In some embodiments, a sugar has the structure of

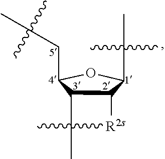

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, R^{2s} is —OCH₂CH₂OMe.

In some embodiments, a sugar has the structure of

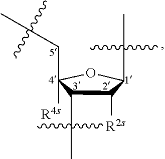

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a modified sugar contains one or more substituents at the 2′ position ((typically one substituent, and often at the axial position or R^{2s}) independently selected from —F; —CF₃, —CN, —N₃, —NO, —NO₂, —OR^{s}, —SR′, or —N(R′)₂, wherein each R′ is independently described in the present disclosure, and in some embodiments, optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂)·OCH₃, —O(CH₂).NH₂, MOE, DMAOE, or DMAEOE, wherein n is from 1 to about 10.

In some embodiments, a modified sugar is a natural RNA sugar whose 2′-OH is replaced with a group selected from —F, —CF₃, —CN, —N₃, —NO, —NO₂, —OR^{s}, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR^{s}. In some embodiments, the 2′-OH is replaced with —OMe. In some embodiments, the 2′-OH is replaced with —OCH₂CH₂OMe.

In some embodiments, a sugar modification is a 2′-modification. In some embodiments, a 2′-modification is a 2′-OR modification. In some embodiments, R^{s} is optionally substituted C₁₋₄ aliphatic. In some embodiments, R^{s} is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F.

In some embodiments, a sugar modification replaces a sugar moiety with another cyclic or acyclic moiety. Examples of such moieties are widely known in the art, e.g., those in morpholino, glycol nucleic acids, PNA, etc., and may be utilized in accordance with the present disclosure.

In some embodiments, one or more sugars of an oligonucleotide are independently modified. In some embodiments, each sugar of an oligonucleotide or a portion thereof (e.g., a wing) is independently modified. In some embodiments, a modified sugar comprises a 2′-modification. In some embodiments, each modified sugar independently comprises a 2′-modification. In some embodiments, a 2′-modification is 2′-OR^{s}, wherein R^{s} is optionally substituted C₁₋₆ aliphatic. In some embodiments, a 2′-modification is a 2′-OMe modification. In some embodiments, a 2′-modification is a 2′-MOE modification. In some embodiments, a 2′-modification is an LNA sugar modification. In some embodiments, a 2′-modification is 2′-F. In some embodiments, each sugar modification is independently a 2′-modification. In some embodiments, each sugar modification is independently a 2′-OR^{s} modification. In some embodiments, each sugar modification is independently 2′-OR^{s}, wherein R^{s} is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

As those skilled in the art will appreciate, modifications of sugars, nucleobases, internucleotidic linkages, etc. can and are often utilized in combination in oligonucleotides, e.g., see various oligonucleotides in Table 1.

In some embodiments, a sugar is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugars of each of which is incorporated herein by reference.

Various additional sugars useful for preparing oligonucleotides or analogs thereof are known in the art and may be utilized in accordance with the present disclosure.

### Internucleotidic Linkages

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S—)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, an internucleotidic linkage is described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the internucleotidic linkages of each of which is incorporated herein by reference. In some embodiments, an internucleotidic linkage is described in U.S. Pat. Nos. 3,687,808, 4,469,863, 4,476,301, 5,177,195, 5,023,243, 5,034,506, 5,166,315, 5,185,444, 5,188,897, 5,214,134, 5,216,141, 5,235,033, 5,264,423, 5,264,564, 5,276,019, 5,278,302, 5,286,717, 5,321,131, 5,399,676, 5,405,938, 5,405,939, 5,434,257, 5,453,496, 5,455,233, 5,466,677, 5,466,677, 5,470,967, 5,476,925, 5,489,677, 5,519,126, 5,536,821, 5,541,307, 5,541,316, 5,550,111, 5,561,225, 5,563,253, 5,571,799, 5,587,361, 5,596,086, 5,602,240, 5,608,046, 5,610,289, 5,618,704, 5,623,070, 5,625,050, 5,633,360, 564,562, 5663312, 5677437, 5677439, 6160109, 6239265, 6028188, 6124445, 6169170, 6172209, 6277603, 6326199, 6346614, 6444423, 6531590, 6534639, 6608035, 6683167, 6858715, 6867294, 6878805, 7015315, 7041816, 7273933, 7321029, or RE39464, the internucleotidic linkages of each of which is incorporated herein by reference.

In some embodiments, an oligonucleotide comprises one or more modified internucleotidic linkages. In some embodiments, each modified internucleotidic linkage is independently a phosphorothioate internucleotidic linkage. In some embodiments, one or more, e.g., about 1-20, 1-15, 1-10, or about 1, 2, 3, 4, 5, 6, 7, 8, 9, 10, 11, 12, 13, 14, 15, 16, 17, 18, 19, 20, or about 10%, 20%, 25%, 30%, 40%, 50%, 60%, 70%, 75%, 80%, 85%, 90%, 95% or more of all internucleotidic linkages in an oligonucleotide, are independently phosphorothioate internucleotidic linkage internucleotidic linkage. In some embodiments, about 10% or more of all internucleotidic linkages are independently phosphorothioate internucleotidic linkages. In some embodiments, about 25% or more of all internucleotidic linkages are independently phosphorothioate internucleotidic linkages. In some embodiments, about 50% or more of all internucleotidic linkages are independently phosphorothioate internucleotidic linkages. In some embodiments, about 60% or more of all internucleotidic linkages are independently phosphorothioate internucleotidic linkages. In some embodiments, about 70% or more of all internucleotidic linkages are independently phosphorothioate internucleotidic linkages. In some embodiments, about 75% or more of all internucleotidic linkages are independently phosphorothioate internucleotidic linkages. In some embodiments, about 80% or more of all internucleotidic linkages are independently phosphorothioate internucleotidic linkages. In some embodiments, about 85% or more of all internucleotidic linkages are independently phosphorothioate internucleotidic linkages. In some embodiments, about 90% or more of all internucleotidic linkages are independently phosphorothioate internucleotidic linkages. In some embodiments, about 95% or more of all internucleotidic linkages are independently phosphorothioate internucleotidic linkages. In some embodiments, each internucleotidic linkage bonded to a natural DNA sugar is independently a phosphorothioate internucleotidic linkage. In some embodiments, each internucleotidic linkage in an oligonucleotide is independently a phosphorothioate internucleotidic linkage.

In some embodiments, an oligonucleotide comprises one or more natural phosphate linkages. In some embodiments, each natural phosphate linkage independently bonds to at least one modified sugar. In some embodiments, each sugar bonded to a natural phosphate linkage is independently a modified sugar. In some embodiments, each sugar bonded to a natural phosphate linkage is independently a 2′-OR modified sugar or a bicyclic sugar (e.g., a LNA sugar). In some embodiments, each sugar bonded to a natural phosphate linkage is independently a 2′-OR^{s} modified sugar. In some embodiments, each sugar bonded to a natural phosphate linkage is independently a 2′-MOE modified sugar.

### Wings and Cores

In some embodiments, an oligonucleotide comprises or consists of a 5′-wing-core-wing-3′ structure.

Wings and cores can independently be of various suitable lengths. In some embodiments, there are about 1, 2, 3, 4, 5, 6, 7, 8, 9, 10, 11, 12, 13, 14, 15, 16, 17, 18, 19, 20, 21, 22, 23, 24, 25 or more nucleobases independently in a wing or core. In some embodiments, each nucleobase independently comprises an optionally substituted monocyclic, bicyclic or polycyclic ring, which ring has at least one nitrogen ring atom; in some embodiments, each nucleobase is independently optionally substituted A, T, C, G or U, or a substituted tautomer of A, T, C, G or U. In some embodiments, the number of nucleobases in a wing is 1, 2, 3, 4, 5, 6, 7, 8, 9, or 10. In some embodiments, the number is 1 for a wing. In some embodiments, the number is 2 for a wing. In some embodiments, the number is 3 for a wing. In some embodiments, the number is 4 for a wing. In some embodiments, the number is 5 for a wing. In some embodiments, the number is 6 for a wing. In some embodiments, the number is 7 for a wing. In some embodiments, the number is 8 for a wing. In some embodiments, the number is 9 for a wing. In some embodiments, the number is 10 for a wing. In some embodiments, in a wing of a wing-core-wing structure, the two wings are of the same length. In some embodiments, the two wings are of different length. In some embodiments, the number is 1, 2, 3, 4, 5, 6, 7, 8, 9, 10, 11, 12, 13, 14, 15 or more for a core. In some embodiments, the number is about 5-15, e.g., about 5, 6, 7, 8, 9, 10, 11, 12, 13, 14, 15, for a core. In some embodiments, the number is 1 for a core. In some embodiments, the number is 2 for a core. In some embodiments, the number is 3 for a core. In some embodiments, the number is 4 for a core. In some embodiments, the number is 5 for a core. In some embodiments, the number is 6 for a core. In some embodiments, the number is 7 for a core. In some embodiments, the number is 8 for a core. In some embodiments, the number is 9 for a core. In some embodiments, the number is 10 for a core. In some embodiments, the number is 11 for a core. In some embodiments, the number is 12 for a core. In some embodiments, the number is 13 for a core. In some embodiments, the number is 14 for a core. In some embodiments, the number is 15 for a core.

In some embodiments, a wing-core-wing is described as “X—Y—Z”, where “X” represents the length of the 5′ wing (as number of nucleobases), “Y” represents the length of the core (as number of nucleobases), and “Z” represents the length of the 3′ wing (as number of nucleobases). Example embodiments of X, Y, and Z include those lengths described as numbers (e.g., above) and exemplified in oligonucleotide species (e.g., in Table 1). In some embodiments, the two wings are of the same or different lengths and/or have the same or different modifications or patterns of modifications. In some embodiments, Y is between 8 and 15. In some embodiments, X, Y or Z can each independently be 1, 2, 3, 4, 5, 6, 7, 8, 9, 10, 11, 12, 13, 14, 15, 16, 17, 18, 19, 20, 25, 30 or more. In some embodiments, each of X, Y and Z is independently 1-30. In some embodiments, X—Z—Z is 5-10-5, 5-10-4, 4-10-4, 4-10-3, 3-10-3, 2-10-2, 5-9-5, 5-9-4, 4-9-5, 5-8-5, 5-8-4, 4-8-5,5-7-5,4-7-5, 5-7-4, or 4-7-4.

In some embodiments, a wing comprises one or more sugar modifications. In some embodiments, each sugar in a wing is independently modified. In some embodiments, each wing sugar in an oligonucleotide is independently modified. In some embodiments, each modified sugar independently comprises a 2′-modification (e.g., a 2′-OR^{s} modified sugar, a LNA sugar, etc.). In some embodiments, each wing sugar is independently a 2′-OR^{s} modified sugar. In some embodiments, each sugar modification in a wing is the same. In some embodiments, a wing comprises different sugar modifications, e.g., different 2′-OR^{s} modifications. In some embodiments, 2′-OR^{s} is 2′-OMe. In some embodiments, 2′-OR^{s} is 2′-MOE. In some embodiments, each sugar in a wing is a 2′-MOE modified sugar. In some embodiments, each sugar in a wing is a 2′-OMe modified sugar. In some embodiments, a wing comprises one or more 2′-OMe modified sugars and one or more 2′-MOE modified sugars.

In some embodiments, the two wings of a wing-core-wing structure comprise different sugar modifications or patterns thereof.

In some embodiments, certain sugar modifications, e.g., 2′-MOE, provide more stability under certain conditions than other sugar modifications, e.g., 2′-OMe or natural DNA or RNA sugars.

In some embodiments, a wing comprises a bicyclic sugar. In some embodiments, a bicyclic sugar is a LNA, a cEt or a BNA sugar.

In some embodiments, one or more internucleotidic linkages bonded to a 5′-wing sugar are each independently a modified internucleotidic linkage. In some embodiments, they are each independently a phosphorothioate internucleotidic linkage. In some embodiments, each internucleotidic linkage bonded to a 5′-wing sugar is independently a modified internucleotidic linkage. In some embodiments, each such internucleotidic linkage is independently a phosphorothioate internucleotidic linkage.

In some embodiments, one or more internucleotidic linkages bonded to a 3′-wing sugar are each independently a modified internucleotidic linkage. In some embodiments, they are each independently a phosphorothioate internucleotidic linkage. In some embodiments, each internucleotidic linkage bonded to a 3′-wing sugar is independently a modified internucleotidic linkage. In some embodiments, each such internucleotidic linkage is independently a phosphorothioate internucleotidic linkage.

In some embodiments, a core comprises one or more, e.g., about 1-20, 5-20, 6-20, 7-20, 8-20, 9-20, 10-20, or 5-15, or about 1, 2, 3, 4, 5, 6, 7, 8, 9, 10, 11, 12, 13, 14, 15, 16, 17, 18, 19, 20 natural DNA sugars. In some embodiments, a core comprises 2 or more natural DNA sugars. In some embodiments, a core comprises 3 or more natural DNA sugars. In some embodiments, a core comprises 4 or more natural DNA sugars. In some embodiments, a core comprises 5 or more natural DNA sugars. In some embodiments, a core comprises 6 or more natural DNA sugars. In some embodiments, a core comprises 7 or more natural DNA sugars. In some embodiments, a core comprises 8 or more natural DNA sugars. In some embodiments, a core comprises 9 or more natural DNA sugars. In some embodiments, a core comprises 10 or more natural DNA sugars. In some embodiments, a core comprises 11 or more natural DNA sugars. In some embodiments, a core comprises 12 or more natural DNA sugars. In some embodiments, a core comprises 13 or more natural DNA sugars. In some embodiments, a core comprises 14 or more natural DNA sugars. In some embodiments, a core comprises 15 or more natural DNA sugars. In some embodiments, such DNA sugars are consecutive. In some embodiments, each sugar in a core is independently a natural DNA sugar.

In some embodiments, one or more internucleotidic linkages bonded to a core sugar are each independently a modified internucleotidic linkage. In some embodiments, they are each independently a phosphorothioate internucleotidic linkage. In some embodiments, each internucleotidic linkage bonded to a core sugar is independently a modified internucleotidic linkage. In some embodiments, each such internucleotidic linkage is independently a phosphorothioate internucleotidic linkage.

In some embodiments, a core is able to hybridize to a target mRNA, forming a duplex structure recognizable by RNase H, such that RNase H is able to cleave the mRNA.

### Oligonucleotides

Among other things, the present disclosure provides various oligonucleotides. As described herein, oligonucleotides may contain various nucleobase modifications, sugar modifications, internucleotidic linkages and patterns thereof. In some embodiments, the present disclosure provides the oligonucleotides in Table 1 as examples.

TABLE 1
Example oligonucleotides and compositions.

| Oligo- | | SEQ | | SEQ |
|---|---|---|---|---|
| nucleo- | Base | ID | | ID |
| tide | Sequence | NO. | Description | NO. |
| 1 | CCACTAGCCCT | 81 | /52MOErC/*/i2MOErC/*/i2MOErA/*/i2MOErC/*/i2MOErT/*A*G*/iMe-dC/*/iMe- | 321 |
| | GGGAGCAAA | | dC/*/iMe- | |
| | | | dC/*T*G*G*G*A*/i2MOErG/*/i2MOErC/*/i2MOErA/*/i2MOErA/*/32MOErA/ | |
| 2 | GCCATCTCCAT | 82 | /52MOErG/*/i2MOErC/*/i2MOErC/*/i2MOErA/*/i2MOErT/*/iMe-dC/*T*/iMe- | 322 |
| | CCATAGAGC | | dC/*/iMe-dC/*A*T*/iMe-dC/*/iMe- | |
| | | | dC/*A*T*/i2MOErA/*/i2MOErG/*/i2MOErA/*/i2MOErG/*/32MOErC/ | |
| 3 | AGGAGAGCTGT | 83 | /52MOErA/*/i2MOErG/*/i2MOErG/*/i2MOErA/*/i2MOErG/*A*G*/iMe- | 323 |
| | GGGCTTGGG | | dC/*T*G*T*G*G*G*/iMe- | |
| | | | dC/*/i2MOErT/*/i2MOErT/*/i2MOErG/*/i2MOErG/*/32MOErG/ | |
| 4 | CACCCATGCCT | 84 | /52MOErC/*/i2MOErA/*/i2MOErC/*/i2MOErC/*/i2MOErC/*A*T*G*/iMe-dC/ | 324 |
| | CCCAGCAGA | | */iMe-dC/*T*/iMe-dC/*/iMe-dC/*/iMe- | |
| | | | dC/*A*/i2MOErG/*/i2MOErC/*/i2MOErA/*/i2MOErG/*/32MOErA/ | |
| 5 | GCTGGCTGTAC | 85 | /52MOErG/*/i2MOErC/*/i2MOErT/*/i2MOErG/*/i2MOErG/*/iMe- | 325 |
| | TCACTCTCC | | dC/*T*G*T*A*/iMe-dC/*T*/iMe-dC/*A*/iMe- | |
| | | | dC/*/i2MOErT/*/i2MOErC/*/i2MOErT/*/i2MOErC/*/32MOErC/ | |
| 6 | GTGCTCTGTCCT | 86 | /52MOErG/*/i2MOErT/*/i2MOErG/*/i2MOErC/*/i2MOErT/*/iMe-dC/*T*G* | 326 |
| | TGGTCCTG | | T*/iMe-dC/*/iMe-dC/*T*T*G*G*/i2MOET/*/i2MOErC/*/i2MOErC/*/ | |
| | | | i2MOErT/*/32MOErG/ | |
| 7 | CCCATTCTCATG | 87 | /52MOErC/*/i2MOErC/*/i2MOErC/*/i2MOErA/*/i2MOErT/*T*/iMe-dC/* | 327 |
| | CAGCCTAC | | T*/iMe-dC/*A*T*G*/iMe- | |
| | | | dC/*A*G*/i2MOErC/*/i2MOErC/*/i2MOErT/*/i2MOErA/*/32MOErC/ | |
| 8 | CTGTGACCTAG | 88 | /52MOErC/*/i2MOErT/*/i2MOErG/*/i2MOErT/*/i2MOErG/*A*/iMe-dC/*/iMe- | 328 |
| | GCTCCTTGA | | dC/*T*A*G*G*/iMe-dC/*T*/iMe- | |
| | | | dC/*/i2MOErC/*/i2MOErT/*/i2MOErT/*/i2MOErG/*/32MOErA/ | |
| 9 | GGTCTGAGAGG | 89 | /52MOErG/*/i2MOErG/*/i2MOErT/*/i2MOErC/*/i2MOErT/*G*A*G*A*G*G*/iMe- | 329 |
| | CTGTGGGTC | | dC/*T*G*T*/i2MOErG/*/i2MOErG/*/i2MOErG/*/i2MOErT/*/32MOErC/ | |
| 10 | GCTCCCAGTTCT | 90 | /52MOErG/*/i2MOErC/*/i2MOErT/*/i2MOErC/*/i2MOErC/*/iMe- | 330 |
| | TCTGTGGT | | dC/*A*G*T*T*/iMe-dC/*T*T*/iMe- | |
| | | | dC/*T*/i2MOErG/*/i2MOErT/*/i2MOErG/*/i2MOErG/*/32MOErT/ | |
| 11 | GATGTCCTCCA | 91 | /52MOErG/*/i2MOErA/*/i2MOErT/*/i2MOErG/*/i2MOErT/*/iMe-dC/*/iMe- | 331 |
| | CAGGTGACA | | dC/*T*/iMe-dC/*/iMe-dC/*A*/iMe- | |
| | | | dC/*A*G*G*/i2MOErT/*/i2MOErG/*/i2MOErA/*/i2MOErC/*/32MOErA/ | |
| 12 | GCTTCCTGCCTT | 92 | /52MOErG/*/i2MOErC/*/i2MOErT/*/i2MOErT/*/i2MOErC/*/iMe-dC/*T*G*/iMe- | 332 |
| | ACTGACCT | | dC/*/iMe-dC/*T*T*A*/iMe- | |
| | | | dC/*T*/i2MOErG/*/i2MOErA/*/i2MOErC/*/i2MOErC/*/32MOErT/ | |
| 13 | CTCTCCTTTGTC | 93 | /52MOErC/*/i2MOET/*/i2MOErC/*/i2MOErT/*/i2MOErC/*/iMe- | 333 |
| | CCTGACCA | | dC/*T*T*T*G*T*/iMe-dC/*/iMe-dC/*/iMe- | |
| | | | dC/*T*/i2MOErG/*/i2MOErA/*/i2MOErC/*/i2MOErC/*/32MOErA/ | |
| 14 | GCCTTGCCTTTT | 94 | /52MOErG/*/i2MOErC/*/i2MOErC/*/i2MOErT/*/i2MOErT/*G*/iMe-dC/*/iMe- | 334 |
| | CCTCACTC | | dC/*T*T*T*T*/iMe-dC/*/iMe- | |
| | | | dC/*T*/i2MOErC/*/i2MOErA/*/i2MOErC/*/i2MOErT/*/32MOErC/ | |
| 15 | GCCTGGTCACT | 95 | /52MOErG/*/i2MOErC/*/i2MOErC/*/i2MOErT/*/i2MOErG/*G*T*/iMe- | 335 |
| | AACCCTCTC | | dC/*A*/iMe-dC/*T*A*A*/iMe-dC/*/iMe- | |
| | | | dC/*/i2MOErC/*/i2MOErT/*/i2MOErC/*/i2MOErT/*/32MOErC/ | |
| 16 | CACCCACCTTG | 96 | /52MOErC/*/i2MOErA/*/i2MOErC/*/i2MOErC/*/i2MOErC/*A*/iMe-dC/*/iMe- | 336 |
| | GTCTTGCCT | | dC/*T*T*G*G*T*/iMe- | |
| | | | dC/*T*/i2MOErT/*/i2MOErG/*/i2MOErC/*/i2MOErC/*/32MOErT/ | |
| 17 | CACACTGATGT | 97 | /52MOErC/*/i2MOErA/*/i2MOErC/*/i2MOErA/*/i2MOErC/*T*G*A*T*G*T*/iMe- | 337 |
| | CCTGTCCCA | | dC/*/iMe-dC/*T*G*/i2MOErT/*/i2MOErC/*/i2MOErC/*/i2MOErC/*/32MOErA/ | |
| 18 | CACACCTCTGG | 98 | /52MOErC/*/i2MOErA/*/i2MOErC/*/i2MOErA/*/i2MOErC/*/iMe-dC/*T*/iMe- | 338 |
| | GTCTTGGCC | | dC/*T*G*G*G*T*/iMe- | |
| | | | dC/*T*/i2MOErT/*/i2MOErG/*/i2MOErG/*/i2MOErC/*/32MOErC/ | |
| 19 | GCTGCCCATCA | 99 | /52MOErG/*/i2MOErC/*/i2MOErT/*/i2MOErG/*/i2MOErC/*/iMe-dC/*/iMe- | 339 |
| | CTCCCAGTT | | dC/*A*T*/iMe-dC/*A*/iMe-dC/*T*/iMe-dC/*/iMe- | |
| | | | dC/*/i2MOErC/*/i2MOErA/*/i2MOErG/*/i2MOErT/*/32MOErT/ | |
| 20 | CTCTCCATCTGC | 100 | /52MOErC/*/i2MOErT/*/i2MOErC/*/i2MOErT/*/i2MOErC/*/iMe-dC/*A*T*/iMe- | 340 |
| | CCTGGCCC | | dC/*T*G*/iMe-dC/*/iMe-dC/*/iMe- | |
| | | | dC/*T*/i2MOErG/*/i2MOErG/*/i2MOErC/*/i2MOErC/*/32MOErC/ | |
| 21 | CAGTCCCTCTCC | 101 | /52MOErC/*/i2MOErA/*/i2MOErG/*/i2MOErT/*/i2MOErC/*/iMe-dC/* | 341 |
| | TTGTCTCT | | /iMe-dC/*T*/iMe-dC/*T*/iMe-dC/*/iMe- | |
| | | | dC/*T*T*G*/i2MOErT/*/i2MOErC/*/i2MOErT/*/i2MOErC/*/32MOErT/ | |
| 22 | ATCCACCTGCT | 102 | /52MOErA/*/i2MOErT/*/i2MOErC/*/i2MOErC/*/i2MOErA/*/iMe-dC/*/iMe- | 342 |
| | GCTCCTGGG | | dC/*T*G*/iMe-dC/*T*G*/iMe-dC/*T*/iMe- | |
| | | | dC/*/i2MOErC/*/i2MOErT/*/i2MOErG/*/i2MOErG/*/32MOErG/ | |
| 23 | CCCTTGTGTCTT | 103 | /52MOErC/*/i2MOErC/*/i2MOErC/*/i2MOErT/*/i2MOErT/*G*T*G*T*/iMe- | 343 |
| | GTGGGTGC | | dC/*T*T*G*T*G*/i2MOErG/*/i2MOErG/*/i2MOErT/*/i2MOErG/*/32MOErC/ | |
| 24 | GCCCTAGGATT | 104 | /52MOErG/*/i2MOErC/*/i2MOErC/*/i2MOErC/*/i2MOErT/*A*G*G*A*T*T*T*T*/i | 344 |
| | TTCCTGTTG | | Me-dC/*/iMe-dC/*/i2MOErT/*/i2MOErG/*/i2MOErT/*/i2MOErT/*/32MOErG/ | |
| 25 | GCCTCAACTCC | 105 | /52MOErG/*/i2MOErC/*/i2MOErC/*/i2MOErT/*/i2MOErC/*A*A*/iMe-dC/ | 345 |
| | TGCCTCCCA | | *T*/iMe-dC/*/iMe-dC/*T*G*/iMe-dC/*/iMe- | |
| | | | dC/*/i2MOErT/*/i2MOErC/*/i2MOErC/*/i2MOErC/*/32MOErA/ | |
| 26 | AGACACCTGGG | 106 | /52MOErA/*/i2MOErG/*/i2MOErA/*/i2MOErC/*/i2MOErA/*/iMe-dC/*/iMe- | 346 |
| | TATCAGCCT | | dC/*T*G*G*G*T*A*T*/iMe- | |
| | | | dC/*/i2MOErA/*/i2MOErG/*/i2MOErC/*/i2MOErC/*/32MOErT/ | |
| 27 | TCCTTCTTCCCT | 107 | /52MOErT/*/i2MOErC/*/i2MOErC/*/i2MOErT/*/i2MOErT/*/iMe-dC/*T*T*/iMe- | 347 |
| | ATTTCCCA | | dC/*/iMe-dC/*/iMe- | |
| | | | dC/*T*A*T*T*/i2MOErT/*/i2MOErC/*/i2MOErC/*/i2MOErC/*/32MOErA/ | |
| 28 | GCATCACTCAC | 108 | /52MOErG/*/i2MOErC/*/i2MOErA/*/i2MOErT/*/i2MOErC/*A*/iMe-dC/*T*/iMe- | 348 |
| | TGTCAGGTA | | dC/*A*/iMe-dC/*T*G*T*/iMe- | |
| | | | dC/*/i2MOErA/*/i2MOErG/*/i2MOErG/*/i2MOErT/*/32MOErA/ | |
| 29 | GTCAGTGCCAC | 109 | /52MOErG/*/i2MOErT/*/i2MOErC/*/i2MOErA/*/i2MOErG/*T*G*/iMe-dC/*/iMe- | 349 |
| | AGCCTTGTC | | dC/*A*/iMe-dC/*A*G*/iMe-dC/*/iMe- | |
| | | | dC/*/i2MOErT/*/i2MOErT/*/i2MOErG/*/i2MOErT/*/32MOErC/ | |
| 30 | GGCACCTACCT | 110 | /52MOErG/*/i2MOErG/*/i2MOErC/*/i2MOErA/*/i2MOErC/*/iMe-dC/*T*A*/iMe- | 350 |
| | TATGCACCC | | dC/*/iMe- | |
| | | | dC/*T*T*A*T*G*/i2MOErC/*/i2MOErA/*/i2MOErC/*/i2MOErC/*/32MOErC/ | |
| 31 | ACTACTGCATC | 111 | /52MOErA/*/i2MOErC/*/i2MOErT/*/i2MOErA/*/i2MOErC/*T*G*/iMe- | 351 |
| | CCTCAGCCC | | dC/*A*T*/iMe-dC/*/iMe-dC/*/iMe-dC/*T*/iMe- | |
| | | | dC/*/i2MOErA/*/i2MOErG/*/i2MOErC/*/i2MOErC/*/32MOErC/ | |
| 32 | GCTTGTCTCATC | 112 | /52MOErG/*/i2MOErC/*/i2MOErT/*/i2MOET/*/i2MOErG/*T*/iMe-dC/*T*/iMe- | 352 |
| | CTGTCTCT | | dC/*A*T*/iMe-dC/*/iMe- | |
| | | | dC/*T*G*/i2MOErT/*/i2MOErC/*/i2MOErT/*/i2MOErC/*/32MOErT/ | |
| 33 | TGTCTCTGAGCT | 113 | /52MOErT/*/i2MOErG/*/i2MOErT/*/i2MOErC/*/i2MOErT/*/iMe- | 353 |
| | GACTGCTT | | dC/*T*G*A*G*/iMe-dC/*T*G*A*/iMe- | |
| | | | dC/*/i2MOErT/*/i2MOErG/*/i2MOErC/*/i2MOErT/*/32MOErT/ | |
| 34 | GGGCTTGACTC | 114 | /52MOErG/*/i2MOErG/*/i2MOErG/*/i2MOErC/*/i2MOErT/*T*G*A*/iMe- | 354 |
| | CACACTCCA | | dC/*T*/iMe-dC/*/iMe-dC/*A*/iMe- | |
| | | | dC/*A*/i2MOErC/*/i2MOErT/*/i2MOErC/*/i2MOErC/*/32MOErA/ | |
| 35 | GGCATGGCATC | 115 | /52MOErG/*/i2MOErG/*/i2MOErC/*/i2MOErA/*/i2MOErT/*G*G*/iMe- | 355 |
| | TCAGCTTCA | | dC/*A*T*/iMe-dC/*T*/iMe- | |
| | | | dC/*A*G*/i2MOErC/*/i2MOErT/*/i2MOErT/*/i2MOErC/*/32MOErA/ | |
| 36 | TTCAGGATCAC | 116 | /52MOErT/*/i2MOErT/*/i2MOErC/*/i2MOErA/*/i2MOErG/*G*A*T*/iMe- | 356 |
| | CTAGCTGGT | | dC/*A*/iMe-dC/*/iMe- | |
| | | | dC/*T*A*G*/i2MOErC/*/i2MOErT/*/i2MOErG/*/i2MOErG/*/32MOErT/ | |
| 37 | CCTCTTTGCCAT | 117 | /52MOErC/*/i2MOErC/*/i2MOErT/*/i2MOErC/*/i2MOErT/*T*T*G*/ | 357 |
| | CTGCTGGG | | iMe-dC/*/iMe-dC/*A*T*/iMe- | |
| | | | dC/*T*G*/i2MOErC/*/i2MOErT/*/i2MOErG/*/i2MOErG/*/32MOErG/ | |
| 38 | GAGTGCAGTTC | 118 | /52MOErG/*/i2MOErA/*/i2MOErG/*/i2MOErT/*/i2MOErG/*/iMe- | 358 |
| | ACTTGTGGT | | dC/*A*G*T*T*/iMe-dC/*A*/iMe- | |
| | | | dC/*T*T*/i2MOErG/*/i2MOErT/*/i2MOErG/*/i2MOErG/*/32MOErT/ | |
| 39 | TGCCCACACTC | 119 | /52MOErT/*/i2MOErG/*/i2MOErC/*/i2MOErC/*/i2MOErC/*A*/iMe-dC/*A*/iMe- | 359 |
| | TGCCTGTCA | | dC/*T*/iMe-dC/*T*G*/iMe-dC/*/iMe- | |
| | | | dC/*/i2MOErT/*/i2MOErG/*/i2MOErT/*/i2MOErC/*/32MOErA/ | |
| 40 | CAGAGGGAGCT | 120 | /52MOErC/*/i2MOErA/*/i2MOErG/*/i2MOErA/*/i2MOErG/*G*G*A*G*/iMe- | 360 |
| | GCTAGTCAG | | dC/*T*G*/iMe- | |
| | | | dC/*T*A*/i2MOErG/*/i2MOErT/*/i2MOErC/*/i2MOErA/*/32MOErG/ | |
| 41 | TTGGCAAAGGT | 121 | /52MOErT/*/i2MOErT/*/i2MOErG/*/i2MOErG/*/i2MOErC/*A*A*A*G*G*T*G*A* | 361 |
| | GATGCAGGC | | T*G*/i2MOErC/*/i2MOErA/*/i2MOErG/*/i2MOErG/*/32MOErC/ | |
| 42 | CCTCCACCAGT | 122 | /52MOErC/*/i2MOErC/*/i2MOErT/*/i2MOErC/*/i2MOErC/*A*/iMe-dC/*/iMe- | 362 |
| | TGGAAGACC | | dC/*A*G*T*T*G*G*A*/i2MOErA/*/i2MOErG/*/i2MOErA/*/i2MOErC/*/32MOErC/ | |
| 43 | GGTTCTCAGCC | 123 | /52MOErG/*/i2MOErG/*/i2MOErT/*/i2MOErT/*/i2MOErC/*T*/iMe-dC/*A*G*/iMe- | 363 |
| | ACCAGGATC | | dC/*/iMe-dC/*A*/iMe-dC/*/iMe- | |
| | | | dC/*A*/i2MOErG/*/i2MOErG/*/i2MOErA/*/i2MOErT/*/32MOErC/ | |
| 44 | GTGCTCCAAGA | 124 | /52MOErG/*/i2MOErT/*/i2MOErG/*/i2MOErC/*/i2MOErT/*/iMe-dC/*/iMe- | 364 |
| | TGCCTGCCA | | dC/*A*A*G*A*T*G*/iMe-dC/*/iMe- | |
| | | | dC/*/i2MOErT/*/i2MOErG/*/i2MOErC/*/i2MOErC/*/32MOErA/ | |
| 45 | CCTTGCAGGCT | 125 | /52MOErC/*/i2MOErC/*/i2MOErT/*/i2MOErT/*/i2MOErG/*/iMe-dC/*A*G*G*/iMe- | 365 |
| | CTTGATGGC | | dC/*T*/iMe- | |
| | | | dC/*T*T*G*/i2MOErA/*/i2MOErT/*/i2MOErG/*/i2MOErG/*/32MOErC/ | |
| 46 | GTGCCATTGGT | 126 | /52MOErG/*/i2MOErT/*/i2MOErG/*/i2MOErC/*/i2MOErC/*A*T*T*G*G*T*A*G*A | 366 |
| | AGAGTAGGA | | *G*/i2MOErT/*/i2MOErA/*/i2MOErG/*/i2MOErG/*/32MOErA/ | |
| 47 | GTGAGCTCCCT | 127 | /52MOErG/*/i2MOErT/*/i2MOErG/*/i2MOErA/*/i2MOErG/*/iMe-dC/*T*/iMe- | 367 |
| | AAAGAACCT | | dC/*/iMe-dC/*/iMe- | |
| | | | dC/*T*A*A*A*G*/i2MOErA/*/i2MOErA/*/i2MOErC/*/i2MOErC/*/32MOErT/ | |
| 48 | GGTTTGCCACC | 128 | /52MOErG/*/i2MOErG/*/i2MOErT/*/i2MOErT/*/i2MOErT/*G*/iMe-dC/*/iMe- | 368 |
| | AGTACAGGG | | dC/*A*/iMe-dC/*/iMe- | |
| | | | dC/*A*G*T*A*/i2MOErC/*/i2MOErA/*/i2MOErG/*/i2MOErG/*/32MOErG/ | |
| 49 | TCCAGCTTCTCC | 129 | /52MOErT/*/i2MOErC/*/i2MOErC/*/i2MOErA/*/i2MOErG/*/iMe-dC/*T*T*/iMe- | 369 |
| | ACATCAAT | | dC/*T*/iMe-dC/*/iMe-dC/*A*/iMe- | |
| | | | dC/*A*/i2MOErT/*/i2MOErC/*/i2MOErA/*/i2MOErA/*/32MOErT/ | |
| 50 | GAACTTGCCTG | 130 | /52MOErG/*/i2MOErA/*/i2MOErA/*/i2MOErC/*/i2MOErT/*T*G*/iMe-dC/*/iMe- | 370 |
| | CTTCCAGCT | | dC/*T*G*/iMe-dC/*T*T*/iMe- | |
| | | | dC/*/i2MOErC/*/i2MOErA/*/i2MOErG/*/i2MOErC/*/32MOErT/ | |
| 51 | ACACTCTGGAT | 131 | /52MOErA/*/i2MOErC/*/i2MOErA/*/i2MOErC/*/i2MOErT/*/iMe- | 371 |
| | GAGTTTGTC | | dC/*T*G*G*A*T*G*A*G*T*/i2MOErT/*/i2MOErT/*/i2MOErG/*/i2MOErT/*/32MO | |
| | | | ErC/ | |
| 52 | GGGCACCCATG | 132 | /52MOErG/*/i2MOErG/*/i2MOErG/*/i2MOErC/*/i2MOErA/*/iMe-dC/*/iMe- | 372 |
| | ACACTCTGG | | dC/*/iMe-dC/*A*T*G*A*/iMe-dC/*A*/iMe- | |
| | | | dC/*/i2MOErT/*/i2MOErC/*/i2MOErT/*/i2MOErG/*/32MOErG/ | |
| 53 | ACTTGTCCAGT | 133 | /52MOErA/*/i2MOErC/*/i2MOErT/*/i2MOErT/*/i2MOErG/*T*/iMe-dC/*/iMe- | 373 |
| | GCTCCAGGT | | dC/*A*G*T*G*/iMe-dC/*T*/iMe- | |
| | | | dC/*/i2MOErC/*/i2MOErA/*/i2MOErG/*/i2MOErG/*/32MOErT/ | |
| 54 | CCCAATCCTTG | 134 | /52MOErC/*/i2MOErC/*/i2MOErC/*/i2MOErA/*/i2MOErA/*T*/iMe-dC/*/iMe- | 374 |
| | CAGTCATGG | | dC/*T*T*G*/iMe- | |
| | | | dC/*A*G*T*/i2MOErC/*/i2MOErA/*/i2MOErT/*/i2MOErG/*/32MOErG/ | |
| 55 | AGCACAGCCTG | 135 | /52MOErA/*/i2MOErG/*/i2MOErC/*/i2MOErA/*/i2MOErC/*A*G*/iMe-dC/*/iMe- | 375 |
| | CATGTCCTC | | dC/*T*G*/iMe- | |
| | | | dC/*A*T*G*/i2MOErT/*/i2MOErC/*/i2MOErC/*/i2MOErT/*/32MOErC/ | |
| 56 | CAAACTGGTGT | 136 | /52MOErC/*/i2MOErA/*/i2MOErA/*/i2MOErA/*/i2MOErC/*T*G*G*T*G*T*/iMe- | 376 |
| | CAGAGCCTG | | dC/*A*G*A*/i2MOErG/*/i2MOErC/*/i2MOErC/*/i2MOErT/*/32MOErG/ | |
| 57 | GCAGCACCCTC | 137 | /52MOErG/*/i2MOErC/*/i2MOErA/*/i2MOErG/*/i2MOErC/*A*/iMe-dC/*/iMe- | 377 |
| | CAAACTGGT | | dC/*/iMe-dC/*T*/iMe-dC/*/iMe- | |
| | | | dC/*A*A*A*/i2MOErC/*/i2MOErT/*/i2MOErG/*/i2MOErG/*/32MOErT/ | |
| 58 | TGGTTAGGTTG | 138 | /52MOErT/*/i2MOErG/*/i2MOErG/*/i2MOErT/*/i2MOErT/*A*G*G*T*T*G*G*A*/i | 378 |
| | GACCCATGG | | Me-dC/*/iMe-dC/*/i2MOErC/*/i2MOErA/*/i2MOErT/*/i2MOErG/*/32MOErG/ | |
| 59 | GCCCAGGTTGT | 139 | /52MOErG/*/i2MOErC/*/i2MOErC/*/i2MOErC/*/i2MOErA/*G*G*T*T*G*T*/iMe- | 379 |
| | CTCAGCCCA | | dC/*T*/iMe-dC/*A*/i2MOErG/*/i2MOErC/*/i2MOErC/*/i2MOErC/*/32MOErA/ | |
| 60 | TCCCTCTCCAG | 140 | /52MOErT/*/i2MOErC/*/i2MOErC/*/i2MOErC/*/i2MOErT/*/iMe-dC/*T*/iMe- | 380 |
| | ATACTGAGG | | dC/*/iMe-dC/*A*G*A*T*A*/iMe- | |
| | | | dC/*/i2MOErT/*/i2MOErG/*/i2MOErA/*/i2MOErG/*/32MOErG/ | |
| 61 | ACAGACAACCC | 141 | /52MOErA/*/i2MOErC/*/i2MOErA/*/i2MOErG/*/i2MOErA/*/iMe-dC/*A*A*/iMe- | 381 |
| | AATGGCAGG | | dC/*/iMe-dC/*/iMe- | |
| | | | dC/*A*A*T*G*/i2MOErG/*/i2MOErC/*/i2MOErA/*/i2MOErG/*/32MOErG/ | |
| 62 | GTCTCCAGAAC | 142 | /52MOErG/*/i2MOErT/*/i2MOErC/*/i2MOErT/*/i2MOErC/*/iMe- | 382 |
| | TGAGCAGGG | | dC/*A*G*A*A*/iMe- | |
| | | | dC/*T*G*A*G*/i2MOErC/*/i2MOErA/*/i2MOErG/*/i2MOErG/*/32MOErG/ | |
| 63 | CCTTAATTCCTG | 143 | /52MOErC/*/i2MOErC/*/i2MOErT/*/i2MOErT/*/i2MOErA/*A*T*T*/ | 383 |
| | TCTGAGGC | | iMe-dC/*/iMe-dC/*T*G*T*/iMe- | |
| | | | dC/*T*/i2MOErG/*/i2MOErA/*/i2MOErG/*/i2MOErG/*/32MOErC/ | |
| 64 | CAGAATACAGT | 144 | /52MOErC/*/i2MOErA/*/i2MOErG/*/i2MOErA/*/i2MOErA/*T*A*/iMe- | 384 |
| | GCCCAGGCC | | dC/*A*G*T*G*/iMe-dC/*/iMe-dC/*/iMe- | |
| | | | dC/*/i2MOErA/*/i2MOErG/*/i2MOErG/*/i2MOErC/*/32MOErC/ | |
| 65 | CCCAGGCCCTT | 145 | /52MOErC/*/i2MOErC/*/i2MOErC/*/i2MOErA/*/i2MOErG/*G*/iMe-dC/*/iMe- | 385 |
| | GCTCAGAAT | | dC/*/iMe-dC/*T*T*G*/iMe-dC/*T*/iMe- | |
| | | | dC/*/i2MOErA/*/i2MOErG/*/i2MOErA/*/i2MOErA/*/32MOErT/ | |
| 66 | GCACTCATCCC | 146 | /52MOErG/*/i2MOErC/*/i2MOErA/*/i2MOErC/*/i2MOErT/*/iMe-dC/*A*T*/iMe- | 386 |
| | TGGCTGGCT | | dC/*/iMe-dC/*/iMe-dC/*T*G*G*/iMe- | |
| | | | dC/*/i2MOErT/*/i2MOErG/*/i2MOErG/*/i2MOErC/*/32MOErT/ | |
| 67 | GATTACAGGGC | 147 | /52MOErG/*/i2MOErA/*/i2MOErT/*/i2MOErT/*/i2MOErA/*/iMe- | 387 |
| | AAGGCCACA | | dC/*A*G*G*G*/iMe- | |
| | | | dC/*A*A*G*G*/i2MOErC/*/i2MOErC/*/i2MOErA/*/i2MOErC/*/32MOErA/ | |
| 68 | GCCCTGGATGT | 148 | /52MOErG/*/i2MOErC/*/i2MOErC/*/i2MOErC/*/i2MOErT/*G*G*A*T*G*T*G*G*/i | 388 |
| | GGCAAAAGA | | Me-dC/*A*/i2MOErA/*/i2MOErA/*/i2MOErA/*/i2MOErG/*/32MOErA/ | |
| 69 | AAGGAAGTCAG | 149 | /52MOErA/*/i2MOErA/*/i2MOErG/*/i2MOErG/*/i2MOErA/*A*G*T*/iMe- | 389 |
| | AGGGAGGGC | | dC/*A*G*A*G*G*G*/i2MOErA/*/i2MOErG/*/i2MOErG/*/i2MOErG/*/32MOErC/ | |
| 70 | CAGGCCCAAAC | 150 | /52MOErC/*/i2MOErA/*/i2MOErG/*/i2MOErG/*/i2MOErC/*/iMe-dC/*/iMe- | 390 |
| | AGGAGGCTC | | dC/*A*A*A*/iMe- | |
| | | | dC/*A*G*G*A*/i2MOErG/*/i2MOErG/*/i2MOErC/*/i2MOErT/*/32MOErC/ | |
| 71 | ATGCCCAGACC | 151 | /52MOErA/*/i2MOErT/*/i2MOErG/*/i2MOErC/*/i2MOErC/*/iMe-dC/* | 391 |
| | CAGGCCCAA | | A*G*A*/iMe-dC/*/iMe-dC/*/iMe- | |
| | | | dC/*A*G*G*/i2MOErC/*/i2MOErC/*/i2MOErC/*/i2MOErA/*/32MOErA/ | |
| 72 | CTGAGGCACAG | 152 | /52MOErC/*/i2MOErT/*/i2MOErG/*/i2MOErA/*/i2MOErG/*G*/iMe-dC/*A*/iMe- | 392 |
| | CACCAAGGC | | dC/*A*G*/iMe-dC/*A*/iMe-dC/*/iMe- | |
| | | | dC/*/i2MOErA/*/i2MOErA/*/i2MOErG/*/i2MOErG/*/32MOErC/ | |
| 73 | GCCAGACCAGG | 153 | /52MOErG/*/i2MOErC/*/i2MOErC/*/i2MOErA/*/i2MOErG/*A*/iMe-dC/*/iMe- | 393 |
| | AAGGAGCCT | | dC/*A*G*G*A*A*G*G*/i2MOErA/*/i2MOErG/*/i2MOErC/*/i2MOErC/*/32MOErT/ | |
| 74 | TCAGGACTTTG | 154 | /52MOErT/*/i2MOErC/*/i2MOErA/*/i2MOErG/*/i2MOErG/*A*/iMe- | 394 |
| | CCTCTTTCC | | dC/*T*T*T*G*/iMe-dC/*/iMe-dC/*T*/iMe- | |
| | | | dC/*/i2MOErT/*/i2MOErT/*/i2MOErT/*/i2MOErC/*/32MOErC/ | |
| 75 | GCTTTAGAGAT | 155 | /52MOErG/*/i2MOErC/*/i2MOET/*/i2MOErT/*/i2MOErT/*A*G*A*G*A*T*T*T*G | 395 |
| | TTGCTACCC | | */iMe-dC/*/i2MOErT/*/i2MOErA/*/i2MOErC/*/i2MOErC/*/32MOErC/ | |
| 76 | GCCCAGCCTCA | 156 | /52MOErG/*/i2MOErC/*/i2MOErC/*/i2MOErC/*/i2MOErA/*G*/iMe-dC/*/iMe- | 396 |
| | GAATGATTC | | dC/*T*/iMe- | |
| | | | dC/*A*G*A*A*T*/i2MOErG/*/i2MOErA/*/i2MOErT/*/i2MOErT/*/32MOErC/ | |
| 77 | CCTCTGAACCC | 157 | /52MOErC/*/i2MOErC/*/i2MOErT/*/i2MOErC/*/i2MOErT/*G*A*A*/iMe-dC/* | 397 |
| | AGTGGAGGA | | /iMe-dC/*/iMe- | |
| | | | dC/*A*G*T*G*/i2MOErG/*/i2MOErA/*/i2MOErG/*/i2MOErG/*/32MOErA/ | |
| 78 | GCCTGGGTTTA | 158 | /52MOErG/*/i2MOErC/*/i2MOErC/*/i2MOErT/*/i2MOErG/*G*G*T*T*T*A*T*T*G | 398 |
| | TTGGAGGGT | | *G*/i2MOErA/*/i2MOErG/*/i2MOErG/*/i2MOErG/*/32MOErT/ | |
| 79 | GCCAGCACAGC | 159 | /52MOErG/*/i2MOErC/*/i2MOErC/*/i2MOErA/*/i2MOErG/*/iMe-dC/*A*/iMe- | 399 |
| | CAAGAGTGG | | dC/*A*G*/iMe-dC/*/iMe- | |
| | | | dC/*A*A*G*/i2MOErA/*/i2MOErG/*/i2MOErT/*/i2MOErG/*/32MOErG/ | |
| 80 | GGGAGTGGAAG | 160 | /52MOErG/*/i2MOErG/*/i2MOErG/*/i2MOErA/*/i2MOErG/*T*G*G*A*A*G*G*A* | 400 |
| | GAAGGAGCC | | A*G*/i2MOErG/*/i2MOErA/*/i2MOErG/*/i2MOErC/*/32MOErC/ | |

Notes: Unless noted otherwise, oligonucleotides are described from 5′ to 3′. As appreciated by those skilled in the art, internucleotidic linkages connect 5′ and 3′ positions of sugars. Unless otherwise noted (e.g., by “*” which is for a phosphorothioate internucleotidic linkage), an internucleotidic linkage is a natural phosphate linkage. Unless otherwise noted, each of A, T, C and G is independently deoxyadenosine, thymidine, deoxycytidine, and deoxyguanosine, respectively (e.g., as typically found in natural DNA). “2MOEr” indicates a 2′-MOE modification to a sugar; “5” indicates a nucleoside has a 5′-OH group (e.g., when at the 5′-end of an oligonucleotide); “3” indicates a nucleoside has a 3′-OH group (e.g., when at the 3′-end of an oligonucleotide); “i” indicates an nucleoside is in the middle of an oligonucleotide and its 5′- and 3′-positions are bonded to internucleotidic linkages as indicated; “Me-dC” indicates a 5-methyl-2′-deoxycytidine nucleoside. As those skilled in the art appreciates, oligonucleotides may exist in various forms including various salt forms.

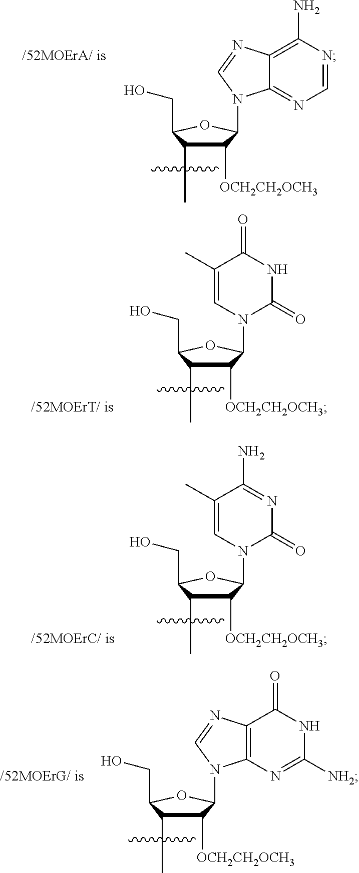

### * is —O—P(O)(SH)—O—;

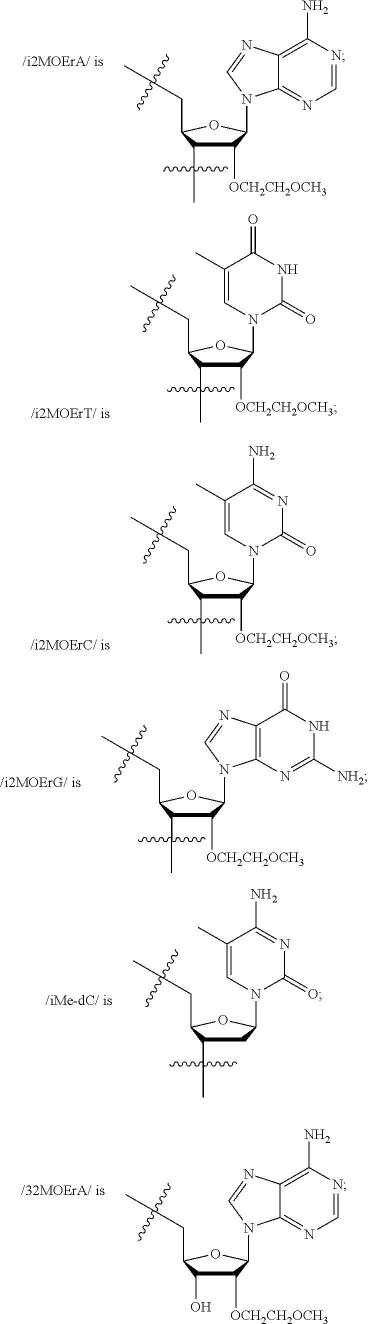

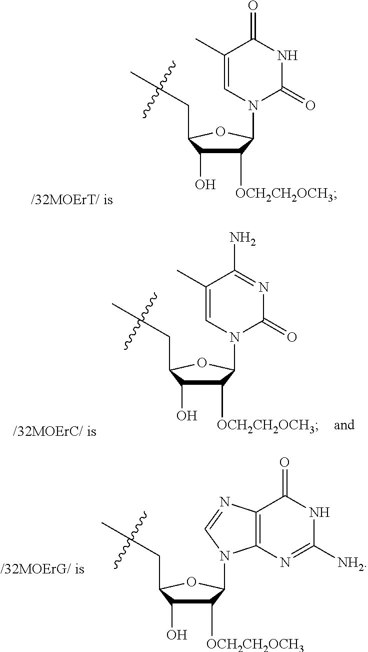

In some embodiments, provided oligonucleotides are capable of hybridizing to a SARM1 transcript. In some embodiments, provided oligonucleotides can reduce levels of SARM1 transcripts or products thereof. In some embodiments, provided oligonucleotide can reduce levels of SARM1 mRNA. In some embodiments, provided oligonucleotide can reduce levels of SARM1 polypeptides. In some embodiments, provided oligonucleotide can reduce activity levels of SARM1 polypeptides observed in a system (e.g., a sample, a subject, etc.). In some embodiments, an oligonucleotide is selected from Table 1. In some embodiments, an oligonucleotide is a pharmaceutically acceptable salt of an oligonucleotide selected from Table 1.

In some embodiments, the present disclosure provides oligonucleotides that are particularly effective in reducing levels of SARM1 transcripts, polypeptides and/or activities.

In some embodiments, an oligonucleotide has the structure of /52MOErC/*/i2MOErC/*/i2MOErA/*/i2MOErC/*/i2MOErT/*A*G*/iMe-dC/*/iMe-dC/*/iMe-dC/*T*G*G*G*A*/i2MOErG/*/i2MOErC/*/i2MOErA/*/i2MOErA/*/32MOErA/ (SEQ ID NO: 321) or a salt thereof.

In some embodiments, an oligonucleotide has the structure of /52MOErG/*/i2MOErC/*/i2MOErC/*/i2MOErA/*/i2MOErT/*/iMe-dC/*T*/iMe-dC/*/iMe-dC/*A*T*/iMe-dC/*/iMe-dC/*A*T*/i2MOErA/*/i2MOErG/*/i2MOErA/*/i2MOErG/*/32MOErC/(SEQ ID NO: 322) or a salt thereof.

In some embodiments, an oligonucleotide has the structure of /52MOErA/*/i2MOErG/*/i2MOErG/*/i2MOErA/*/i2MOErG/*A*G*/iMe-dC/*T*G*T*G*G*G*/iMe-dC/*/i2MOErT/*/i2MOErT/*/i2MOErG/*/i2MOErG/*/32MOErG/ (SEQ ID NO: 323) or a salt thereof.

In some embodiments, an oligonucleotide has the structure of /52MOErC/*/i2MOErA/*/i2MOErC/*/i2MOErC/*/i2MOErC/*A*T*G*/iMe-dC/*/iMe-dC/*T*/iMe-dC/*/iMe-dC/*/iMe-dC/*A*/i2MOErG/*/i2MOErC/*/i2MOErA/*/i2MOErG/*/32MOErA/ (SEQ ID NO: 324) or a salt thereof.

In some embodiments, an oligonucleotide has the structure of /52MOErG/*/i2MOErC/*/i2MOErT/*/i2MOErG/*/i2MOErG/*/iMe-dC/*T*G*T*A*/iMe-dC/*T*/iMe-dC/*A*/iMe-dC/*/i2MOErT/*/i2MOErC/*/i2MOErT/*/i2MOErC/*/32MOErC/ (SEQ ID NO: 325) or a salt thereof.

In some embodiments, an oligonucleotide has the structure of /52MOErG/*/i2MOErT/*/i2MOErG/*/i2MOErC/*/i2MOErT/*/iMe-dC/*T*G*T*/iMe-dC/*/iMe-dC/*T*T*G*G*/i2MOErT/*/i2MOErC/*/i2MOErC/*/i2MOErT/*/32MOErG/ (SEQ ID NO: 326) or a salt thereof.

In some embodiments, an oligonucleotide has the structure of /52MOErC/*/i2MOErC/*/i2MOErC/*/i2MOErA/*/i2MOErT/*T*/iMe-dC/*T*/iMe-dC/*A*T*G*/iMe-dC/*A*G*/i2MOErC/*/i2MOErC/*/i2MOErT/*/i2MOErA/*/32MOErC/ (SEQ ID NO: 327) or a salt thereof.

In some embodiments, an oligonucleotide has the structure of /52MOErC/*/i2MOErT/*/i2MOErG/*/i2MOErT/*/i2MOErG/*A*/iMe-dC/*/iMe-dC/*T*A*G*G*/iMe-dC/*T*/iMe-dC/*/i2MOErC/*/i2MOErT/*/i2MOErT/*/i2MOErG/*/32MOErA/ (SEQ ID NO: 328) or a salt thereof.

In some embodiments, an oligonucleotide has the structure of /52MOErG/*/i2MOErG/*/i2MOErT/*/i2MOErC/*/i2MOErT/*G*A*G*A*G*G*/iMe-dC/*T*G*T*/i2MOErG/*/i2MOErG/*/i2MOErG/*/i2MOErT/*/32MOErC/ (SEQ ID NO: 329) or a salt thereof.

In some embodiments, an oligonucleotide has the structure of /52MOErG/*/i2MOErC/*/i2MOErT/*/i2MOErC/*/i2MOErC/*/iMe-dC/*A*G*T*T*/iMe-dC/*T*T*/iMe-dC/*T*/i2MOErG/*/i2MOErT/*/i2MOErG/*/i2MOErG/*/32MOErT/ (SEQ ID NO: 330) or a salt thereof.

In some embodiments, an oligonucleotide has the structure of /52MOErG/*/i2MOErA/*/i2MOErT/*/i2MOErG/*/i2MOErT/*/iMe-dC/*/iMe-dC/*T*/iMe-dC/*/iMe-dC/*A*/iMe-dC/*A*G*G*/i2MOErT/*/i2MOErG/*/i2MOErA/*/i2MOErC/*/32MOErA/ (SEQ ID NO: 331) or a salt thereof.

In some embodiments, an oligonucleotide has the structure of /52MOErG/*/i2MOErC/*/i2MOErT/*/i2MOErT/*/i2MOErC/*/iMe-dC/*T*G*/iMe-dC/*/iMe-dC/*T*T*A*/iMe-dC/*T*/i2MOErG/*/i2MOErA/*/i2MOErC/*/i2MOErC/*/32MOErT/ (SEQ ID NO: 332) or a salt thereof.

In some embodiments, an oligonucleotide has the structure of /52MOErC/*/i2MOErT/*/i2MOErC/*/i2MOErT/*/i2MOErC/*/iMe-dC/*T*T*T*G*T*/iMe-dC/*/iMe-dC/*/iMe-dC/*T*/i2MOErG/*/i2MOErA/*/i2MOErC/*/i2MOErC/*/32MOErA/ (SEQ ID NO: 333) or a salt thereof.

In some embodiments, an oligonucleotide has the structure of /52MOErG/*/i2MOErC/*/i2MOErC/*/i2MOErT/*/i2MOErT/*G*/iMe-dC/*/iMe-dC/*T*T*T*T*/iMe-dC/*/iMe-dC/*T*/i2MOErC/*/i2MOErA/*/i2MOErC/*/i2MOErT/*/32MOErC/ (SEQ ID NO: 334) or a salt thereof.

In some embodiments, an oligonucleotide has the structure of /52MOErG/*/i2MOErC/*/i2MOErC/*/i2MOErT/*/i2MOErG/*G*T*/iMe-dC/*A*/iMe-dC/*T*A*A*/iMe-dC/*/iMe-dC/*/i2MOErC/*/i2MOErT/*/i2MOErC/*/i2MOErT/*/32MOErC/ (SEQ ID NO: 335) or a salt thereof.

In some embodiments, an oligonucleotide has the structure of /52MOErC/*/i2MOErA/*/i2MOErC/*/i2MOErC/*/i2MOErC/*A*/iMe-dC/*/iMe-dC/*T*T*G*G*T*/iMe-dC/*T*/i2MOErT/*/i2MOErG/*/i2MOErC/*/i2MOErC/*/32MOErT/ (SEQ ID NO: 336) or a salt thereof.

In some embodiments, an oligonucleotide has the structure of /52MOErC/*/i2MOErA/*/i2MOErC/*/i2MOErA/*/i2MOErC/*T*G*A*T*G*T*/iMe-dC/*/iMe-dC/*T*G*/i2MOErT/*/i2MOErC/*/i2MOErC/*/i2MOErC/*/32MOErA/ (SEQ ID NO: 337) or a salt thereof.

In some embodiments, an oligonucleotide has the structure of /52MOErC/*/i2MOErA/*/i2MOErC/*/i2MOErA/*/i2MOErC/*/iMe-dC/*T*/iMe-dC/*T*G*G*G*T*/iMe-dC/*T*/i2MOErT/*/i2MOErG/*/i2MOErG/*/i2MOErC/*/32MOErC/ (SEQ ID NO: 338) or a salt thereof.

In some embodiments, an oligonucleotide has the structure of /52MOErG/*/i2MOErC/*/i2MOErT/*/i2MOErG/*/i2MOErC/*/iMe-dC/*/iMe-dC/*A*T*/iMe-dC/*A*/iMe-dC/*T*/iMe-dC/*/iMe-dC/*/i2MOErC/*/i2MOErA/*/i2MOErG/*/i2MOErT/*/32MOErT/(SEQ ID NO: 339) or a salt thereof.

In some embodiments, an oligonucleotide has the structure of /52MOErC/*/i2MOErT/*/i2MOErC/*/i2MOErT/*/i2MOErC/*/iMe-dC/*A*T*/iMe-dC/*T*G*/iMe-dC/*/iMe-dC/*/iMe-dC/*T*/i2MOErG/*/i2MOErG/*/i2MOErC/*/i2MOErC/*/32MOErC/ (SEQ ID NO: 340) or a salt thereof.

In some embodiments, an oligonucleotide has the structure of /52MOErC/*/i2MOErA/*/i2MOErG/*/i2MOErT/*/i2MOErC/*/iMe-dC/*/iMe-dC/*T*/iMe-dC/*T*/iMe-dC/*/iMe-dC/*T*T*G*/i2MOErT/*/i2MOErC/*/i2MOErT/*/i2MOErC/*/32MOErT/ (SEQ ID NO: 341) or a salt thereof.

In some embodiments, an oligonucleotide has the structure of /52MOErA/*/i2MOErT/*/i2MOErC/*/i2MOErC/*/i2MOErA/*/iMe-dC/*/iMe-dC/*T*G*/iMe-dC/*T*G*/iMe-dC/*T*/iMe-dC/*/i2MOErC/*/i2MOErT/*/i2MOErG/*/i2MOErG/*/32MOErG/ (SEQ ID NO: 342) or a salt thereof.

In some embodiments, an oligonucleotide has the structure of /52MOErC/*/i2MOErC/*/i2MOErC/*/i2MOErT/*/i2MOErT/*G*T*G*T*/iMe-dC/*T*T*G*T*G*/i2MOErG/*/i2MOErG/*/i2MOErT/*/i2MOErG/*/32MOErC/ (SEQ ID NO: 343) or a salt thereof.

In some embodiments, an oligonucleotide has the structure of /52MOErG/*/i2MOErC/*/i2MOErC/*/i2MOErC/*/i2MOErT/*A*G*G*A*T*T*T*T*/iMe-dC/*/iMe-dC/*/i2MOErT/*/i2MOErG/*/i2MOErT/*/i2MOErT/*/32MOErG/ (SEQ ID NO: 344) or a salt thereof.

In some embodiments, an oligonucleotide has the structure of /52MOErG/*/i2MOErC/*/i2MOErC/*/i2MOErT/*/i2MOErC/*A*A*/iMe-dC/*T*/iMe-dC/*/iMe-dC/*T*G*/iMe-dC/*/iMe-dC/*/i2MOErT/*/i2MOErC/*/i2MOErC/*/i2MOErC/*/32MOErA/ (SEQ ID NO: 345) or a salt thereof.

In some embodiments, an oligonucleotide has the structure of /52MOErA/*/i2MOErG/*/i2MOErA/*/i2MOErC/*/i2MOErA/*/iMe-dC/*/iMe-dC/*T*G*G*G*T*A*T*/iMe-dC/*/i2MOErA/*/i2MOErG/*/i2MOErC/*/i2MOErC/*/32MOErT/(SEQ ID NO: 346) or a salt thereof.

In some embodiments, an oligonucleotide has the structure of /52MOErT/*/i2MOErC/*/i2MOErC/*/i2MOErT/*/i2MOErT/*/iMe-dC/*T*T*/iMe-dC/*/iMe-dC/*/iMe-dC/*T*A*T*T*/i2MOErT/*/i2MOErC/*/i2MOErC/*/i2MOErC/*/32MOErA/ (SEQ ID NO: 347) or a salt thereof.

In some embodiments, an oligonucleotide has the structure of /52MOErG/*/i2MOErC/*/i2MOErA/*/i2MOErT/*/i2MOErC/*A*/iMe-dC/*T*/iMe-dC/*A*/iMe-dC/*T*G*T*/iMe-dC/*/i2MOErA/*/i2MOErG/*/i2MOErG/*/i2MOErT/*/32MOErA/ (SEQ ID NO: 348) or a salt thereof.

In some embodiments, an oligonucleotide has the structure of /52MOErG/*/i2MOErT/*/i2MOErC/*/i2MOErA/*/i2MOErG/*T*G*/iMe-dC/*/iMe-dC/*A*/iMe-dC/*A*G*/iMe-dC/*/iMe-dC/*/i2MOErT/*/i2MOErT/*/i2MOErG/*/i2MOErT/*/32MOErC/ (SEQ ID NO: 349) or a salt thereof.

In some embodiments, an oligonucleotide has the structure of /52MOErG/*/i2MOErG/*/i2MOErC/*/i2MOErA/*/i2MOErC/*/iMe-dC/*T*A*/iMe-dC/*/iMe-dC/*T*T*A*T*G*/i2MOErC/*/i2MOErA/*/i2MOErC/*/i2MOErC/*/32MOErC/ (SEQ ID NO: 350) or a salt thereof.

In some embodiments, an oligonucleotide has the structure of /52MOErA/*/i2MOErC/*/i2MOErT/*/i2MOErA/*/i2MOErC/*T*G*/iMe-dC/*A*T*/iMe-dC/*/iMe-dC/*/iMe-dC/*T*/iMe-dC/*/i2MOErA/*/i2MOErG/*/i2MOErC/*/i2MOErC/*/32MOErC/ (SEQ ID NO: 351) or a salt thereof.

In some embodiments, an oligonucleotide has the structure of /52MOErG/*/i2MOErC/*/i2MOErT/*/i2MOErT/*/i2MOErG/*T*/iMe-dC/*T*/iMe-dC/*A*T*/iMe-dC/*/iMe-dC/*T*G*/i2MOErT/*/i2MOErC/*/i2MOErT/*/i2MOErC/*/32MOErT/ (SEQ ID NO: 352) or a salt thereof.

In some embodiments, an oligonucleotide has the structure of /52MOErT/*/i2MOErG/*/i2MOErT/*/i2MOErC/*/i2MOErT/*/iMe-dC/*T*G*A*G*/iMe-dC/*T*G*A*/iMe-dC/*/i2MOErT/*/i2MOErG/*/i2MOErC/*/i2MOErT/*/32MOErT/ (SEQ ID NO: 353) or a salt thereof.

In some embodiments, an oligonucleotide has the structure of /52MOErG/*/i2MOErG/*/i2MOErG/*/i2MOErC/*/i2MOErT/*T*G*A*/iMe-dC/*T*/iMe-dC/*/iMe-dC/*A*/iMe-dC/*A*/i2MOErC/*/i2MOErT/*/i2MOErC/*/i2MOErC/*/32MOErA/ (SEQ ID NO: 354) or a salt thereof.

In some embodiments, an oligonucleotide has the structure of /52MOErG/*/i2MOErG/*/i2MOErC/*/i2MOErA/*/i2MOErT/*G*G*/iMe-dC/*A*T*/iMe-dC/*T*/iMe-dC/*A*G*/i2MOErC/*/i2MOErT/*/i2MOErT/*/i2MOErC/*/32MOErA/ (SEQ ID NO: 355) or a salt thereof.

In some embodiments, an oligonucleotide has the structure of /52MOErT/*/i2MOErT/*/i2MOErC/*/i2MOErA/*/i2MOErG/*G*A*T*/iMe-dC/*A*/iMe-dC/*/iMe-dC/*T*A*G*/i2MOErC/*/i2MOErT/*/i2MOErG/*/i2MOErG/*/32MOErT/ (SEQ ID NO: 356) or a salt thereof.

In some embodiments, an oligonucleotide has the structure of /52MOErC/*/i2MOErC/*/i2MOErT/*/i2MOErC/*/i2MOErT/*T*T*G*/iMe-dC/*/iMe-dC/*A*T*/iMe-dC/*T*G*/i2MOErC/*/i2MOErT/*/i2MOErG/*/i2MOErG/*/32MOErG/ (SEQ ID NO: 357) or a salt thereof.

In some embodiments, an oligonucleotide has the structure of /52MOErG/*/i2MOErA/*/i2MOErG/*/i2MOErT/*/i2MOErG/*/iMe-dC/*A*G*T*T*/iMe-dC/*A*/iMe-dC/*T*T*/i2MOErG/*/i2MOErT/*/i2MOErG/*/i2MOErG/*/32MOErT/ (SEQ ID NO: 358) or a salt thereof.

In some embodiments, an oligonucleotide has the structure of /52MOErT/*/i2MOErG/*/i2MOErC/*/i2MOErC/*/i2MOErC/*A*/iMe-dC/*A*/iMe-dC/*T*/iMe-dC/*T*G*/iMe-dC/*/iMe-dC/*/i2MOErT/*/i2MOErG/*/i2MOErT/*/i2MOErC/*/32MOErA/ (SEQ ID NO: 359) or a salt thereof.

In some embodiments, an oligonucleotide has the structure of /52MOErC/*/i2MOErA/*/i2MOErG/*/i2MOErA/*/i2MOErG/*G*G*A*G*/iMe-dC/*T*G*/iMe-dC/*T*A*/i2MOErG/*/i2MOErT/*/i2MOErC/*/i2MOErA/*/32MOErG/ (SEQ ID NO: 360) or a salt thereof.

In some embodiments, an oligonucleotide has the structure of /52MOErT/*/i2MOErT/*/i2MOErG/*/i2MOErG/*/i2MOErC/*A*A*A*G*G*T*G*A*T*G*/i2MOErC/*/i2MOErA/*/i2MOErG/*/i2MOErG/*/32MOErC/ (SEQ ID NO: 361) or a salt thereof.

In some embodiments, an oligonucleotide has the structure of /52MOErC/*/i2MOErC/*/i2MOErT/*/i2MOErC/*/i2MOErC/*A*/iMe-dC/*/iMe-dC/*A*G*T*T*G*G*A*/i2MOErA/*/i2MOErG/*/i2MOErA/*/i2MOErC/*/32MOErC/ (SEQ ID NO: 362) or a salt thereof.

In some embodiments, an oligonucleotide has the structure of /52MOErG/*/i2MOErG/*/i2MOErT/*/i2MOErT/*/i2MOErC/*T*/iMe-dC/*A*G*/iMe-dC/*/iMe-dC/*A*/iMe-dC/*/iMe-dC/*A*/i2MOErG/*/i2MOErG/*/i2MOErA/*/i2MOErT/*/32MOErC/ (SEQ ID NO: 363) or a salt thereof.

In some embodiments, an oligonucleotide has the structure of /52MOErG/*/i2MOErT/*/i2MOErG/*/i2MOErC/*/i2MOErT/*/iMe-dC/*/iMe-dC/*A*A*G*A*T*G*/iMe-dC/*/iMe-dC/*/i2MOErT/*/i2MOErG/*/i2MOErC/*/i2MOErC/*/32MOErA/ (SEQ ID NO: 364) or a salt thereof.

In some embodiments, an oligonucleotide has the structure of /52MOErC/*/i2MOErC/*/i2MOErT/*/i2MOErT/*/i2MOErG/*/iMe-dC/*A*G*G*/iMe-dC/*T*/iMe-dC/*T*T*G*/i2MOErA/*/i2MOErT/*/i2MOErG/*/i2MOErG/*/32MOErC/ (SEQ ID NO: 365) or a salt thereof.

In some embodiments, an oligonucleotide has the structure of /52MOErG/*/i2MOErT/*/i2MOErG/*/i2MOErC/*/i2MOErC/*A*T*T*G*G*T*A*G*A*G*/i2MOErT/* /i2MOErA/*/i2MOErG/*/i2MOErG/*/32MOErA/ (SEQ ID NO: 366) or a salt thereof.

In some embodiments, an oligonucleotide has the structure of /52MOErG/*/i2MOErT/*/i2MOErG/*/i2MOErA/*/i2MOErG/*/iMe-dC/*T*/iMe-dC/*/iMe-dC/*/iMe-dC/*T*A*A*A*G*/i2MOErA/*/i2MOErA/*/i2MOErC/*/i2MOErC/*/32MOErT/ (SEQ ID NO: 367) or a salt thereof.

In some embodiments, an oligonucleotide has the structure of /52MOErG/*/i2MOErG/*/i2MOErT/*/i2MOErT/*/i2MOErT/*G*/iMe-dC/*/iMe-dC/*A*/iMe-dC/*/iMe-dC/*A*G*T*A*/i2MOErC/*/i2MOErA/*/i2MOErG/*/i2MOErG/*/32MOErG/ (SEQ ID NO: 368) or a salt thereof.

In some embodiments, an oligonucleotide has the structure of /52MOErT/*/i2MOErC/*/i2MOErC/*/i2MOErA/*/i2MOErG/*/iMe-dC/*T*T*/iMe-dC/*T*/iMe-dC/*/iMe-dC/*A*/iMe-dC/*A*/i2MOErT/*/i2MOErC/*/i2MOErA/*/i2MOErA/*/32MOErT/ (SEQ ID NO: 369) or a salt thereof.

In some embodiments, an oligonucleotide has the structure of /52MOErG/*/i2MOErA/*/i2MOErA/*/i2MOErC/*/i2MOErT/*T*G*/iMe-dC/*/iMe-dC/*T*G*/iMe-dC/*T*T*/iMe-dC/*/i2MOErC/*/i2MOErA/*/i2MOErG/*/i2MOErC/*/32MOErT/ (SEQ ID NO: 370) or a salt thereof.

In some embodiments, an oligonucleotide has the structure of /52MOErA/*/i2MOErC/*/i2MOErA/*/i2MOErC/*/i2MOErT/*/iMe-dC/*T*G*G*A*T*G*A*G*T*/i2MOErT/*/i2MOErT/*/i2MOErG/*/i2MOErT/*/32MOErC/ (SEQ ID NO: 371) or a salt thereof.

In some embodiments, an oligonucleotide has the structure of /52MOErG/*/i2MOErG/*/i2MOErG/*/i2MOErC/*/i2MOErA/*/iMe-dC/*/iMe-dC/*/iMe-dC/*A*T*G*A*/iMe-dC/*A*/iMe-dC/*/i2MOErT/*/i2MOErC/*/i2MOErT/*/i2MOErG/*/32MOErG/(SEQ ID NO: 372) or a salt thereof.

In some embodiments, an oligonucleotide has the structure of /52MOErA/*/i2MOErC/*/i2MOErT/*/i2MOErT/*/i2MOErG/*T*/iMe-dC/*/iMe-dC/*A*G*T*G*/iMe-dC/*T*/iMe-dC/*/i2MOErC/*/i2MOErA/*/i2MOErG/*/i2MOErG/*/32MOErT/ (SEQ ID NO: 373) or a salt thereof.

In some embodiments, an oligonucleotide has the structure of /52MOErC/*/i2MOErC/*/i2MOErC/*/i2MOErA/*/i2MOErA/*T*/iMe-dC/*/iMe-dC/*T*T*G*/iMe-dC/*A*G*T*/i2MOErC/*/i2MOErA/*/i2MOErT/*/i2MOErG/*/32MOErG/ (SEQ ID NO: 374) or a salt thereof.

In some embodiments, an oligonucleotide has the structure of /52MOErA/*/i2MOErG/*/i2MOErC/*/i2MOErA/*/i2MOErC/*A*G*/iMe-dC/*/iMe-dC/*T*G*/iMe-dC/*A*T*G*/i2MOErT/*/i2MOErC/*/i2MOErC/*/i2MOErT/*/32MOErC/ (SEQ ID NO: 375) or a salt thereof.

In some embodiments, an oligonucleotide has the structure of /52MOErC/*/i2MOErA/*/i2MOErA/*/i2MOErA/*/i2MOErC/*T*G*G*T*G*T*/iMe-dC/*A*G*A*/i2MOErG/*/i2MOErC/*/i2MOErC/*/i2MOErT/*/32MOErG/ (SEQ ID NO: 376) or a salt thereof.

In some embodiments, an oligonucleotide has the structure of /52MOErG/*/i2MOErC/*/i2MOErA/*/i2MOErG/*/i2MOErC/*A*/iMe-dC/*/iMe-dC/*/iMe-dC/*T*/iMe-dC/*/iMe-dC/*A*A*A*/i2MOErC/*/i2MOErT/*/i2MOErG/*/i2MOErG/*/32MOErT/(SEQ ID NO: 377) or a salt thereof.

In some embodiments, an oligonucleotide has the structure of /52MOErT/*/i2MOErG/*/i2MOErG/*/i2MOErT/*/i2MOErT/*A*G*G*T*T*G*G*A*/iMe-dC/*/iMe-dC/*/i2MOErC/*/i2MOErA/*/i2MOErT/*/i2MOErG/*/32MOErG/ (SEQ ID NO: 378) or a salt thereof.

In some embodiments, an oligonucleotide has the structure of /52MOErG/*/i2MOErC/*/i2MOErC/*/i2MOErC/*/i2MOErA/*G*G*T*T*G*T*/iMe-dC/*T*/iMe-dC/*A*/i2MOErG/*/i2MOErC/*/i2MOErC/*/i2MOErC/*/32MOErA/ (SEQ ID NO: 379) or a salt thereof.

In some embodiments, an oligonucleotide has the structure of /52MOErT/*/i2MOErC/*/i2MOErC/*/i2MOErC/*/i2MOErT/*/iMe-dC/*T*/iMe-dC/*/iMe-dC/*A*G*A*T*A*/iMe-dC/*/i2MOErT/*/i2MOErG/*/i2MOErA/*/i2MOErG/*/32MOErG/ (SEQ ID NO: 380) or a salt thereof.

In some embodiments, an oligonucleotide has the structure of /52MOErA/*/i2MOErC/*/i2MOErA/*/i2MOErG/*/i2MOErA/*/iMe-dC/*A*A*/iMe-dC/*/iMe-dC/*/iMe-dC/*A*A*T*G*/i2MOErG/*/i2MOErC/*/i2MOErA/*/i2MOErG/*/32MOErG/ (SEQ ID NO: 381) or a salt thereof.

In some embodiments, an oligonucleotide has the structure of /52MOErG/*/i2MOErT/*/i2MOErC/*/i2MOErT/*/i2MOErC/*/iMe-dC/*A*G*A*A*/iMe-dC/*T*G*A*G*/i2MOErC/*/i2MOErA/*/i2MOErG/*/i2MOErG/*/32MOErG/ (SEQ ID NO: 382) or a salt thereof.

In some embodiments, an oligonucleotide has the structure of /52MOErC/*/i2MOErC/*/i2MOErT/*/i2MOErT/*/i2MOErA/*A*T*T*/iMe-dC/*/iMe-dC/*T*G*T*/iMe-dC/*T*/i2MOErG/*/i2MOErA/*/i2MOErG/*/i2MOErG/*/32MOErC/ (SEQ ID NO: 383) or a salt thereof.

In some embodiments, an oligonucleotide has the structure of /52MOErC/*/i2MOErA/*/i2MOErG/*/i2MOErA/*/i2MOErA/*T*A*/iMe-dC/*A*G*T*G*/iMe-dC/*/iMe-dC/*/iMe-dC/*/i2MOErA/*/i2MOErG/*/i2MOErG/*/i2MOErC/*/32MOErC/ (SEQ ID NO: 384) or a salt thereof.

In some embodiments, an oligonucleotide has the structure of /52MOErC/*/i2MOErC/*/i2MOErC/*/i2MOErA/*/i2MOErG/*G*/iMe-dC/*/iMe-dC/*/iMe-dC/*T*T*G*/iMe-dC/*T*/iMe-dC/*/i2MOErA/*/i2MOErG/*/i2MOErA/*/i2MOErA/*/32MOErT/(SEQ ID NO: 385) or a salt thereof.

In some embodiments, an oligonucleotide has the structure of /52MOErG/*/i2MOErC/*/i2MOErA/*/i2MOErC/*/i2MOErT/*/iMe-dC/*A*T*/iMe-dC/*/iMe-dC/*/iMe-dC/*T*G*G*/iMe-dC/*/i2MOErT/*/i2MOErG/*/i2MOErG/*/i2MOErC/*/32MOErT/ (SEQ ID NO: 386) or a salt thereof.

In some embodiments, an oligonucleotide has the structure of /52MOErG/*/i2MOErA/*/i2MOErT/*/i2MOErT/*/i2MOErA/*/iMe-dC/*A*G*G*G*/iMe-dC/*A*A*G*G*/i2MOErC/*/i2MOErC/*/i2MOErA/*/i2MOErC/*/32MOErA/ (SEQ ID NO: 387) or a salt thereof.

In some embodiments, an oligonucleotide has the structure of /52MOErG/*/i2MOErC/*/i2MOErC/*/i2MOErC/*/i2MOErT/*G*G*A*T*G*T*G*G*/iMe-dC/*A*/i2MOErA/*/i2MOErA/*/i2MOErA/*/i2MOErG/*/32MOErA/ (SEQ ID NO: 388) or a salt thereof.

In some embodiments, an oligonucleotide has the structure of /52MOErA/*/i2MOErA/*/i2MOErG/*/i2MOErG/*/i2MOErA/*A*G*T*/iMe-dC/*A*G*A*G*G*G*/i2MOErA/*/i2MOErG/*/i2MOErG/*/i2MOErG/*/32MOErC/ (SEQ ID NO: 389) or a salt thereof.

In some embodiments, an oligonucleotide has the structure of /52MOErC/*/i2MOErA/*/i2MOErG/*/i2MOErG/*/i2MOErC/*/iMe-dC/*/iMe-dC/*A*A*A*/iMe-dC/*A*G*G*A*/i2MOErG/*/i2MOErG/*/i2MOErC/*/i2MOErT/*/32MOErC/ (SEQ ID NO: 390) or a salt thereof.

In some embodiments, an oligonucleotide has the structure of /52MOErA/*/i2MOErT/*/i2MOErG/*/i2MOErC/*/i2MOErC/*/iMe-dC/*A*G*A*/iMe-dC/*/iMe-dC/*/iMe-dC/*A*G*G*/i2MOErC/*/i2MOErC/*/i2MOErC/*/i2MOErA/*/32MOErA/ (SEQ ID NO: 391) or a salt thereof.

In some embodiments, an oligonucleotide has the structure of /52MOErC/*/i2MOErT/*/i2MOErG/*/i2MOErA/*/i2MOErG/*G*/iMe-dC/*A*/iMe-dC/*A*G*/iMe-dC/*A*/iMe-dC/*/iMe-dC/*/i2MOErA/*/i2MOErA/*/i2MOErG/*/i2MOErG/*/32MOErC/ (SEQ ID NO: 392) or a salt thereof.

In some embodiments, an oligonucleotide has the structure of /52MOErG/*/i2MOErC/*/i2MOErC/*/i2MOErA/*/i2MOErG/*A*/iMe-dC/*/iMe-dC/*A*G*G*A*A*G*G*/i2MOErA/*/i2MOErG/*/i2MOErC/*/i2MOErC/*/32MOErT/ (SEQ ID NO: 393) or a salt thereof.

In some embodiments, an oligonucleotide has the structure of /52MOErT/*/i2MOErC/*/i2MOErA/*/i2MOErG/*/i2MOErG/*A*/iMe-dC/*T*T*T*G*/iMe-dC/*/iMe-dC/*T*/iMe-dC/*/i2MOErT/*/i2MOErT/*/i2MOErT/*/i2MOErC/*/32MOErC/ (SEQ ID NO: 394) or a salt thereof.

In some embodiments, an oligonucleotide has the structure of /52MOErG/*/i2MOErC/*/i2MOErT/*/i2MOErT/*/i2MOErT/*A*G*A*G*A*T*T*T*G*/iMe-dC/*/i2MOErT/*/i2MOErA/*/i2MOErC/*/i2MOErC/*/32MOErC/ (SEQ ID NO: 395) or a salt thereof.

In some embodiments, an oligonucleotide has the structure of /52MOErG/*/i2MOErC/*/i2MOErC/*/i2MOErC/*/i2MOErA/*G*/iMe-dC/*/iMe-dC/*T*/iMe-dC/*A*G*A*A*T*/i2MOErG/*/i2MOErA/*/i2MOErT/*/i2MOErT/*/32MOErC/ (SEQ ID NO: 396) or a salt thereof.

In some embodiments, an oligonucleotide has the structure of /52MOErC/*/i2MOErC/*/i2MOErT/*/i2MOErC/*/i2MOErT/*G*A*A*/iMe-dC/*/iMe-dC/*/iMe-dC/*A*G*T*G*/i2MOErG/*/i2MOErA/*/i2MOErG/*/i2MOErG/*/32MOErA/ (SEQ ID NO: 397) or a salt thereof.

In some embodiments, an oligonucleotide has the structure of /52MOErG/*/i2MOErC/*/i2MOErC/*/i2MOErT/*/i2MOErG/*G*G*T*T*T*A*T*T*G*G*/i2MOErA/* /i2MOErG/*/i2MOErG/*/i2MOErG/*/32MOErT/ (SEQ ID NO: 398) or a salt thereof.

In some embodiments, an oligonucleotide has the structure of /52MOErG/*/i2MOErC/*/i2MOErC/*/i2MOErA/*/i2MOErG/*/iMe-dC/*A*/iMe-dC/*A*G*/iMe-dC/*/iMe-dC/*A*A*G*/i2MOErA/*/i2MOErG/*/i2MOErT/*/i2MOErG/*/32MOErG/ (SEQ ID NO: 399) or a salt thereof.

In some embodiments, an oligonucleotide has the structure of /52MOErG/*/i2MOErG/*/i2MOErG/*/i2MOErA/*/i2MOErG/*T*G*G*A*A*G*G*A*A*G*/i2MOErG/*/i2MOErA/*/i2MOErG/*/i2MOErC/*/32MOErC/ (SEQ ID NO: 400) or a salt thereof.

In some embodiments, the present disclosure provides compositions comprising provided oligonucleotides. In some embodiments, an oligonucleotide composition comprises a provided oligonucleotide or a salt thereof, and various diastereomers and salt thereof. In some embodiments, an oligonucleotide composition comprises a provided oligonucleotide or a salt thereof, and various diastereomers with respect to chiral linkage phosphorus and salt thereof. In some embodiments, oligonucleotides may exist in one or more forms. In some embodiments, oligonucleotides in a composition exist in a salt form. In some embodiments, oligonucleotides in a composition exist in one or more salt forms. In some embodiments, a salt form is a pharmaceutically acceptable salt form. In some embodiments, a salt form is a metal salt. In some embodiments, a salt form is a alkali metal salt. In some embodiments, a salt form is a sodium salt. In some embodiments, a salt form is a potassium salt. In some embodiments, a salt form is a calcium salt. In some embodiments, a salt form is an ammonium salt form (e.g., of N(R′)₃ wherein R′ is as described herein; in some embodiments, each R′ is independently —H or optionally substituted C₁₋₆ alkyl). In some embodiments, an oligonucleotide composition is a liquid composition, wherein an oligonucleotide is dissolved in a solution. In some embodiments, a solution is a buffer. In some embodiments, a solution is a buffered saline. In some embodiments, in a composition acidic internucleotidic linkages, e.g., natural phosphate linkages, phosphorothioate internucleotidic linkages, e.g., independently exist as anionic forms, and the composition comprises one or more types of cations, e.g., Na⁺, K⁺, etc.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

Certain useful additional chemical moieties are described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the additional chemical moieties of each of which are incorporated herein by reference.

### Manufacturing

Various technologies are available in the art to manufacture provided oligonucleotide and may be utilized in accordance with the present disclosure. For example, in some embodiments, oligonucleotides are manufactured on solid support using phosphoramidites. In some embodiments, oligonucleotides are manufactured in solution. In some embodiments, manufacturing of oligonucleotides comprise multiple cycles, in each of which one or more nucleoside units, typically one, are added. In some embodiments, a cycle comprises coupling of a phosphoramidite, blocking unreacted 5′-OH groups, modification (e.g., sulfurization), and/or de-blocking protected 5′-OH groups in newly coupled nucleosides. In some embodiments, modification may be performed at the end of cycles when certain lengths of oligonucleotides are achieved.

Certain technologies for manufacturing oligonucleotides are described in U.S. Pat. Nos. 3,687,808, 4,469,863, 4,476,301, 5,177,195, 5,023,243, 5,034,506, 5,166,315, 5,185,444, 5,188,897, 5,214,134, 5,216,141, 5,235,033, 5,264,423, 5,264,564, 5,276,019, 5,278,302, 5,286,717, 5,321,131, 5,399,676, 5,405,938, 5,405,939, 5,434,257, 5,453,496, 5,455,233, 5,466,677, 5,466,677, 5,470,967, 5,476,925, 5,489,677, 5,519,126, 5,536,821, 5,541,307, 5,541,316, 5,550,111, 5,561,225, 5,563,253, 5,571,799, 5,587,361, 5,596,086, 5,602,240, 5,608,046, 5,610,289, 5,618,704, 5,623,070, 5,625,050, 5,633,360, 564,562, 5,663,312, 5,677,437, 5,677,439, 6,160,109, 6,239,265, 6,028,188, 6,124,445, 6,169,170, 6,172,209, 6,277,603, 6,326,199, 6,346,614, 6,444,423, 6,531,590, 6,534,639, 6,608,035, 6,683,167, 6,858,715, 6,867,294, 6,878,805, 7,015,315, 7,041,816, 7,273,933, 7,321,029, U.S. RE39464, U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, or WO 2019/032612.

In some embodiments, oligonucleotides and/or compositions are provided as stereorandom compositions with respect to chiral linkage phosphorus. For example, when using traditional phosphoramidites comprising N, N-diisopropylamino and 2-cyanoethyloxy groups for oligonucleotide synthesis, chiral linkages may be formed with no or low stereoselectivity. In some embodiments, oligonucleotides are provided as a mixture of various diastereomers and/or salts thereof. In some embodiments, a composition comprises an oligonucleotide, and/or one or more or all of its diastereomers with respect to chiral linkage phosphorus. In some embodiments, an oligonucleotide and/or its diastereomers are independently in one or more forms. In some embodiments, an oligonucleotide and/or its diastereomers are independently in one or more salt forms, e.g., one or more pharmaceutically acceptable salt forms. In some embodiments, for each chiral linkage phosphorus, both the Rp and the Sp configurations are present in the composition. In some embodiments, for each chiral linkage phosphorus, both the Rp and the Sp configurations have a percentage of at least about 10%, 15%, 20%, 25%, 30% 35%, 40%, or 45%. In some embodiments, for a chiral internucleotidic linkage both the Rp and the Sp configurations have a percentage of about 50%. In some embodiments, for each chiral internucleotidic linkage both the Rp and the Sp configurations have a percentage of about 50%. In some embodiments, for each chiral internucleotidic linkage both the Rp and the Sp configurations have a percentage of about 20-80%. In some embodiments, for each chiral internucleotidic linkage both the Rp and the Sp configurations have a percentage of about 30-70%. In some embodiments, for each chiral internucleotidic linkage both the Rp and the Sp configurations have a percentage of about 40-60%. In some embodiments, for each chiral internucleotidic linkage both the Rp and the Sp configurations have a percentage of about 45-55%. In some embodiments, for each linkage phosphorus the Rp configuration independently has a percentage of about 20-80%, 30-70%, 40-60%, or 45-55%, or about 20%, 30%, 40%, 50%, 60%, 70% or 80%.

Amount, concentration, etc., of provided oligonucleotides may be assessed utilizing various technologies in accordance with the present disclosure, e.g., by UV (e.g., at 260 nm), weight, etc. In some embodiments, amount, concentration, etc., of all oligonucleotides present in a composition is assessed. In some embodiments, amount, concentration, etc., include all oligonucleotides share the same constitution (e.g., diastereomers with respect to chiral linkage phosphorus) including all their forms including pharmaceutically acceptable salt forms.

### SARM1

In some embodiments, SARM1 refers to a gene or a gene product thereof (e.g., a nucleic acid (e.g., DNA, RNA, etc.), a transcript (e.g., a SARM1 mRNA), a protein encoded thereby (e.g., a SARM1 polypeptide), etc.) from a species, which may be known as SARM2, NAD(+) hydrolase SARM1, NADP(+) hydrolase SARM1, NADase SARM1, sterile alpha and Armadillo repeat protein, sterile alpha and TIR motif-containing protein 1, SAMD2, MyD88-5, SAM domain-containing protein 2, sterile alpha motif domain-containing protein 2, HsTIR, etc. Various SARM1 sequences including variants thereof are readily available to those of skill in the art. Various technologies, e.g., assays, cells, animal models, etc., have also been reported and can be utilized for characterization and/or assessment of provided technologies (e.g., oligonucleotides, compositions, methods, etc.) in accordance with the present disclosure.

The SARM1 gene is reported to encode a SARM1 protein, which according to various reports comprises 724 or 690 amino acids, depending on isoform, and primarily localizes to cellular cytoplasm and mitochondria. Some studies have also noted that SARM1 may localize to the axon, dendrites, and/or synapses of a neuronal cell. It has been reported that, in some embodiments, SARM1 comprises multiple domains, including, from N-terminal region to C-terminal region: (i) a mitochondrial targeting region; (ii) an armadillo repeat (ARM) domain; (iii) a first sterile alpha motif (SAM) domain; (iv) a second sterile alpha motif (SAM) domain; and (v) a toll/interleukin-1 (IL-1) receptor (TIR) domain. SARM1 from other species, e.g., monkeys, rats, and mice, have been reported to comprise various conserved domains similar to human SAR1.

SARM1 protein reportedly acts an enzyme which cleaves nicotinamide adenine dinucleotide (NAD⁺) into nicotinamide (NAM) and adenosine diphosphate ribose (ADPR) and cyclic adenosine diphosphate ribose (cADPR) (Essuman et al., Neuron, 2017). Further, homodimerzation of SARM1 has been reported to increase this enzymatic activity (Gerdts et al., J Neurosci., 2013; Summer et al., Proc Natl Acad Sci USA, 2016). Certain studies suggest that neuronal stress or injury may lead to upregulation of SARM1 activity by allowing for this dimerization by disengaging inhibition by the ARM domains (Loring and Thompson, Cell Chem Biol., 2020). SARM1 has been reported to be linked to the presence and functioning of nicotinamide mononucleotide adenylyltransferase 2 (NMNAT2), which has been described as neuroprotective and reportedly acts to maintain a low level of nicotinamide mononucleotide (NMN) and a relatively high level of NAD+(Figley et al., Neuron, 2021).

The purported NADase activity of SARM1 has been suggested to contribute to cell death and/or axonal degeneration by depleting NAD+ and/or triggering signaling cascades by increasing ADPR and cADPR (Loring and Thompson, Cell Chem Biol., 2020). It has been reported that knockdown and/or knockout of SARM1 can be neuroprotective by preventing axonal degeneration induced by depletion or loss of NMNAT2 and/or inhibiting Wallerian degeneration (Gilley et al., Cell Rep., 2015; Gilley et al., Cell Rep., 2017). Some studies have also detailed in vivo prevention of peripheral neuropathy or retinal degeneration by SARM1 knockout, further supporting potential neuroprotective effects of SARM1 downregulation (Geisler et al., Brain, 2016; Finnegan et al., Int J Mol Sci, 2022).

In some embodiments, a SARM1 gene, transcript (e.g., mRNA before or after splicing), or protein variant or isoform comprises a mutation (which in some embodiments may be referred to or reported as SNP). SARM1 mutations have been reported as enriched in subjects with ALS (Bloom et al., Mol Neurodegener, 2022). Among these mutations, some mutations have been reported to result in constitutively active SARM1 protein. In some embodiments, a mutation is rs782325355, rs71373646, rs71373646, rs11652384, rs1555585331, rs781854217, rs782421919, rs782331635, rs1032963037, rs1449836804, rs1555585243, rs376587698, rs369186722, rs373458416, rs782706244, rs1555585662, rs1555585804, rs377210302, rs1555585809, rs782228906, rs782106973, rs571724138, rs539229444, rs782196205, rs782398426, rs782321764, rs782753946, rs1451417529, rs782676389, rs782225125, rs782774927, rs782256561, rs372946020, and/or rs781850558. In some embodiments, a mutation is A226-232, A249-252, V184G, G206S, L223P, R267W, V331E, E340K, C482Y, T385A, T502P, E693D, V112I, A240E, R244S, A250T, A275V, R310H, A341V, R403P, Y429F, E431G, R465T, N478S, D483E, R484C, A488E, V518L, S558N, R569C, R570Q, 1593T, E604K, M612V, R615H, D637Y, A646S, V654M, M672V, S684F, R697C, and/or R702C. In some embodiments, a SARM1 protein, e.g., a mutant SARM1 protein is constitutively active. In some embodiments, an oligonucleotide targets both wild-type and mutant SARM1. In some embodiments, an oligonucleotide can reduce levels of both wild-type and mutant SARM1 transcripts and/or products (e.g. polypeptides) encoded thereby. In some embodiments, the base sequence of an oligonucleotide is complementary to a characteristic portion shared by various forms of SARM1 transcripts, e.g., in some embodiments, all forms of SARM1 transcripts in a subject. In some embodiments, provided technologies can selectively reduce levels of SARM1 transcripts associated with a condition, disorder or disease. In some embodiments, provided technologies can selectively reduce levels of mutant SARM1 transcripts and/or products (e.g. polypeptides) encoded thereby. Among other things, the present disclosure encompasses the recognition that in various conditions, disorders or diseases, overall levels of SARM1 transcripts, e.g. both wild-type and mutant forms, and/or products (e.g. polypeptides) encoded thereby, are to be reduced and provide technologies to reduce overall levels of SARM1 transcripts and/or products (e.g. polypeptides) encoded thereby.

### SARM1-Associated Conditions, Disorders or Diseases

Various conditions, disorders or diseases are reported to be associated with SARM1 and may be prevented or treated with present disclosure. Generally, a disease, disorder, or condition is associated with SARM1 if the presence, level, activity, and/or form of SARM1 and/or products (e.g., transcripts, encoded proteins, etc.) thereof correlates with incidence of and/or susceptibility to the disease, disorder, or condition (e.g., across a relevant population). In some embodiments, a condition, disorder or disease associated with SARM1 may be treated and/or prevented by reducing expression, level and/or activity of SARM1 transcripts and/or proteins.

Among other things, the present disclosure provides technologies for preventing and/or treating various conditions, disorders or diseases. In some embodiments, a condition, disorder, or disease is a neurodegenerative disease. In some embodiments, a condition, disorder or disease is amyotrophic lateral sclerosis (ALS). In some embodiments, a condition, disorder or disease is traumatic brain injury (TBI). In some embodiments, a condition, disorder or disease is Alzheimer's disease (AD). In some embodiments, a condition, disorder or disease is Parkinson's disease (PD). In some embodiments, a condition, disorder or disease is frontotemporal dementia (FTD). In some embodiments, a condition, disorder or disease is progressive supranuclear palsy (PSP). In some embodiments, a condition, disorder or disease is corticobasal degeneration (CBD). In some embodiments, a condition, disorder or disease is Wolfram syndrome (WS). In some embodiments, a condition, disorder or disease is Friedreich's ataxia (FRDA). In some embodiments, a condition, disorder or disease is multiple system atrophy (MSA). In some embodiments, a condition, disorder or disease is spinocerebellar ataxia (SCA). In some embodiments, a condition, disorder or disease is spinal muscular atrophy (SMA). In some embodiments, a condition, disorder or disease is Pick's disease (PD). In some embodiments, a condition, disorder or disease is progressive motor atrophy. In some embodiments, a condition, disorder or disease is concussion. In some embodiments, a condition, disorder or disease is spinal cord injury (SCI). In some embodiments, a condition, disorder or disease is chronic traumatic encephalopathy (CTE). In some embodiments, a condition, disorder or disease is seizure. In some embodiments, a condition, disorder or disease is stroke. In some embodiments, a condition, disorder or disease is intracerebral hemorrhage. In some embodiments, a condition, disorder or disease is a tauopathy. In some embodiments, a condition, disorder or disease is associated with Wallerian degeneration. In some embodiments, a condition, disorder or disease is acute glaucoma. In some embodiments, a condition, disorder or disease is cancer. In some embodiments, a condition, disorder or disease is diabetes. In some embodiments, a condition, disorder or disease is chemotherapy-induced peripheral neuropathy.

### Characterization and Assessment

In some embodiments, properties and/or activities of provided oligonucleotides and compositions thereof can be characterized and/or assessed using various technologies available to those skilled in the art, e.g., biochemical assays (e.g., RNase H assays), cell based assays, animal models, clinical trials, etc. Certain useful technologies are described in the Examples. Those skilled in the art reading the present disclosure will readily appreciate that other technologies, e.g., in vitro models (e.g., cell lines) for various conditions, disorders or diseases, animal models for various conditions, disorders or diseases, clinical trials, etc. may be designed and/or utilized to assess provided technologies (e.g., oligonucleotides, compositions, methods, etc.) in accordance with the present disclosure.

### Biological Applications

As appreciated by those skilled in the art, oligonucleotides are useful for many purposes. In some embodiments, provided technologies (e.g., oligonucleotides, compositions, methods, etc.) are useful for reducing levels and/or activities of various SARM1 transcripts (e.g., RNA) and/or products encoded thereby (e.g., proteins). In some embodiments, provided technologies reduce levels and/or activities SARM1 RNA transcripts. In some embodiments, provided oligonucleotides and compositions provide knockdown of SARM1 mRNA. In some embodiments, provided technologies reduce levels of SARM1 polypeptides. In some embodiments, provided technologies reduce levels of SARM1 activities.

In some embodiments, the present disclosure provides a method for reducing level of SARM1 mRNA in a system, comprising administering or delivering to the system an effective amount of an oligonucleotide or an oligonucleotide composition. In some embodiments, the present disclosure provides a method for reducing level of a SARM1 polypeptide in a system, comprising administering or delivering to the system an effective amount of an oligonucleotide or an oligonucleotide composition. In some embodiments, the present disclosure provides a method for reducing level of a SARM1 activity in a system, comprising administering or delivering to the system an effective amount of an oligonucleotide or an oligonucleotide composition.

In some embodiments, a system comprises SARM1 mRNA. In some embodiments, a system comprises wild-type SARM1 mRNA. In some embodiments, a system comprises SARM1 mRNA comprising a mutation. In some embodiments, a system comprises wild-type mRNA and SARM1 mRNA comprising amutation. In some embodiments, a system expresses SARM1 mRNA. In some embodiments, a system expresses wild-type SARM1 mRNA. In some embodiments, a system expresses SARM1 mRNA comprising a mutation. In some embodiments, a system expresses wild-type SARM1 and SARM1 mRNA comprising a mutation. In some embodiments, a system expresses SARM1 polypeptides. In some embodiments, a system expresses wild-type SARM1 polypeptides. In some embodiments, a system expresses SARM1 polypeptides comprising a mutation. In some embodiments, a system expresses wild-type SARM1 polypeptides and SARM1 polypeptides comprising amutation.

In some embodiments, a system is an in vitro system. In some embodiments, a system is an in vivo system.

In some embodiments, a system comprises a cell. In some embodiments, a system is a cell. In some embodiments, a system comprises a population of cells. In some embodiments, a system is a population of cells. In some embodiments, a cell is a neuronal cell. In some embodiments, a cell is a cell in the neuronal system. In some embodiments, a cell is a cell in CNS. In some embodiments, a cell possesses one or more characteristics, properties and/or activities of a neuronal cell.

In some embodiments, a system is a tissue. In some embodiments, a system comprises a tissue. In some embodiments, a system is an organ. In some embodiments, a system comprises an organ. In some embodiments, a system is a brain or a portion thereof. In some embodiments, a system comprises a brain or a portion thereof. In some embodiments, a system is an organism. In some embodiments, a system comprises an organism. In some embodiments, a system is a subject. In some embodiments, a system is a mammal, e.g., a mouse, rat, monkey, etc. In some embodiments, a system is a human.

In some embodiments, a level is reduced by at least about 10%, 15%, 20%, 25%, 30%, 35%, 40%, 45%, 50%, 60%, 70%, 75%, 80%, 85%, 90%, or 95% compared to absence of a provided oligonucleotide or composition and/or presence of a reference oligonucleotide or composition. In some embodiments, such a reduction is achieved at certain oligonucleotide concentrations (e.g., 1 nM, 5 nM, 10 nM, 100 nM, 500 nM, 1 uM, 5 uM, etc.) or doses. In some embodiments, such a reduction is achieved in a system, e.g., various suitable assay (e.g., in vitro cell-based assays, assays described in the Examples, etc.). In some embodiments, a reference composition comprises no oligonucleotides targeting SARM1. In some embodiments, a reference oligonucleotide targets a different nucleic acid than SARM1. In some embodiments, a level is of mRNA, e.g., SARM1 mRNA. In some embodiments, a level is of a polypeptide, e.g., a SARM1 protein. In some embodiments, a level is reduced by at least about 10%. In some embodiments, a level is reduced by at least about 20%. In some embodiments, a level is reduced by at least about 30%. In some embodiments, a level is reduced by at least about 40%. In some embodiments, a level is reduced by at least about 50%. In some embodiments, a level is reduced by at least about 60%. In some embodiments, a level is reduced by at least about 70%. In some embodiments, a level is reduced by at least about 75%. In some embodiments, a level is reduced by at least about 80%. In some embodiments, a level is reduced by at least about 85%. In some embodiments, a level is reduced by at least about 90%. In some embodiments, a level is reduced by at least about 95%. In some embodiments, level of SARM1 mRNA is reduced about or at least about 10%, 15%, 20%, 25%, 30%, 35%, 40%, 45%, 50%, 55%, 60%, 65%, 70%, 75%, 80%, 85%, 90%, or 95% at about 0.01-50 uM, e.g., about 0.01-30, about 1-30, about 5-30, about 1, about 5, about 10, about 15, about 20, about 25, about 30 uM, etc. oligonucleotides when assessed, e.g., using an assay in the Examples; in some embodiments, it is reduced about or at least about 40%, 45%, 50%, 55%, 60%, 65%, 70%, 75%, 80%, 85%, 90%, or 95% at about 30 uM oligonucleotides; in some embodiments, it is reduced about or at least about 10%, 15%, 20%, 25%, 30%, 35%, 40%, 45%, 50%, 55%, 60%, 65%, 70%, 75%, 80%, or 85% at about 20 uM oligonucleotides; in some embodiments, it is reduced about or at least about 10%, 15%, 20%, 25%, 30%, 35%, 40%, 45%, 50%, 55%, 60%, 65%, 70%, 75%, 80%, or 85% at about 19 uM oligonucleotides; in some embodiments, it is reduced about or at least about 10%, 15%, 20%, 25%, 30%, 35%, 40%, 45%, 50%, 55%, 60%, 65%, 70%, 75%, 80%, or 85% at about 16 uM oligonucleotides; in some embodiments, it is reduced about or at least about 30%, 35%, 40%, 45%, 50%, 55%, 60%, 65%, 70%, 75%, 80%, 85%, 90%, or 95% at about 10 uM oligonucleotides; in some embodiments, it is reduced about or at least about 20%, 25%, 30%, 35%, 40%, 45%, 50%, 55%, 65%, 70%, 75%, 80%, or 85% at about 3.33 uM oligonucleotides; in some embodiments, it is reduced about or at least about 20%, 25%, 30%, 35%, 40%, 45%, 50%, 55%, 60%, 65%, 70%, 75%, 80%, or 85% at about 1.11 uM oligonucleotides; in some embodiments, it is reduced about or at least about 20%, 25%, 30%, 35%, 40%, 45%, 50%, 55%, 60%, 65%, or 70% at about 0.37 uM oligonucleotides; in some embodiments, it is reduced about or at least about 10%, 15%, 20%, 25%, 30%, 35% 40%, 45%, or 50% at about 0.12 uM oligonucleotides; in some embodiments, it is reduced about or at least about 10%, 15%, 20%, 25% 30%, 35%, or 40% at about 0.04 uM oligonucleotides; and in some embodiments, it is reduced about or at least about 10%, 15%, 20%, 25% or 30% at about 0.01 uM oligonucleotides. In some embodiments, a reduction is about or at least about 50%. In some embodiments, a reduction is about or at least about 55%. In some embodiments, a reduction is about or at least about 60%. In some embodiments, a reduction is about or at least about 65%. In some embodiments, a reduction is about or at least about 70%. In some embodiments, a reduction is about or at least about 75%. In some embodiments, a reduction is about or at least about 80%. In some embodiments, a reduction is about or at least about 85%. In some embodiments, a reduction is about or at least about 90%. In some embodiments, a reduction is about or at least about 95%. In some embodiments, a reduction is assessed at or after about or at least about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following administering or delivering a provided oligonucleotide or composition. In some embodiments, a reduction is assessed at or after about or at least about 0, 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following removal or washout of a provided oligonucleotide or composition. In some embodiments, a reduction is assessed at day 0. In some embodiments, a reduction is assessed at about day 3. In some embodiments, a reduction is assessed at about day 10. In some embodiments, a reduction is assessed at about day 14. In some embodiments, a reduction is assessed at about day 21. In some embodiments, reductions of at one or more assessments, e.g., of SARM1 mRNA levels, are independently about or at least about 5%, 10%, 15%, 20%, 25%, 30%, 35% 40%, 45%, 50%, 55% 60%, 65%, 70%, 75%, 80%, 85%, 90%, or 95%; in some embodiments, reductions are independently about or at least about 60%; in some embodiments, reductions are about or at least about 65%; in some embodiments, reductions are about or at least about 70%; in some embodiments, reductions are about or at least about 75%; in some embodiments, reductions are independently about or at least about 80%. In some embodiments, a reduction is assessed at about day 21. In some embodiments, reductions of at one or more assessments, e.g., of a SARM1 protein level or an activity thereof, are independently about or at least about 5%, 10%, 15%, 20%, 25%, 30%, 35% 40%, 45%, 50%, 55%, 60%, 65%, 70%, 75%, 80%, 85%, 90%, or 95%; in some embodiments, reductions are independently about or at least about 10%; in some embodiments, reductions are about or at least about 15%; in some embodiments, reductions are about or at least about 20%; in some embodiments, reductions are about or at least about 25%; in some embodiments, reductions are independently about or at least about 30%; in some embodiments, reductions are independently about or at least about 35%; in some embodiments, reductions are independently about or at least about 40%.; in some embodiments, reductions are independently about or at least about 45%.; in some embodiments, reductions are independently about or at least about 50%; in some embodiments, reductions are independently about or at least about 55%; in some embodiments, reductions are independently about or at least about 60%. In some embodiments, a reduction is about or at least about 40% at day 7 after removal of a provided oligonucleotide or composition. In some embodiments, a reduction is about or at least about 40% at day 10 after removal of a provided oligonucleotide or composition. In some embodiments, a reduction is about or at least about 40% at day 14 after removal of a provided oligonucleotide or composition. In some embodiments, a reduction is about or at least about 40% at day 21 after removal of a provided oligonucleotide or composition. In some embodiments, a reduction is about or at least about 40%-50% at or after about or at least about day 7, 10, 14 and/or 21 after removal of a provided oligonucleotide or composition. Certain results are presented in the Figures and Examples as examples. Those skilled in the art reading the present disclosure appreciate that various technologies can be utilized to assess provided technologies, including reductions of mRNA and/or polypeptide levels by provided oligonucleotides and compositions. Certain useful technologies are described in the Examples. In some embodiments, reductions are relative to presence of reference oligonucleotides or compositions, e.g., scrambled oligonucleotides as described herein. In some embodiments, reductions are relative to absence of oligonucleotides or compositions. In some embodiments, assessments are performed according to those described in the Examples, e.g., in some embodiments, using iPSC-derived motor neurons with oligonucleotide concentrations at about 16, 19, or 20 uM with gymnotic delivery.

In some embodiments, activity of a provided oligonucleotide or oligonucleotide composition may be assessed by IC50 which is the concentration to reduce a level, e.g., of SARM1 mRNA, polypeptide, activity, etc., by 50% in a suitable condition, e.g., cell-based in vitro assays, assays described in the Examples, etc. In some embodiments, provided oligonucleotides or compositions have an IC50 of about or no more than about 0.001, 0.01, 0.1, 0.5, 1, 2, 5, 10, 50, 100, 200, 500, 1000, 2000, 5000 or 10000 nM. In some embodiments, an oligonucleotide has an IC50 of about or no more than about 10000 nM. In some embodiments, an oligonucleotide has an IC50 of about or no more than about 5000 nM. In some embodiments, an oligonucleotide has an IC50 of about or no more than about 2000 nM. In some embodiments, an oligonucleotide has an IC50 of about or no more than about 1000 nM. In some embodiments, an IC50 is about or no more than about 500 nM. In some embodiments, an IC50 is about or no more than about 200 nM. In some embodiments, an IC50 is about or no more than about 100 nM. In some embodiments, an IC50 is about or no more than about 50 nM. In some embodiments, an IC50 is about or no more than about 20 nM. In some embodiments, an IC50 is about or no more than about 10 nM. In some embodiments, an IC50 is about or no more than about 5 nM. In some embodiments, an IC50 is about or no more than about 2 nM. In some embodiments, an IC50 is about or no more than about 1 nM.

In some embodiments, provided oligonucleotides and compositions are useful for treating various conditions, disorders or diseases, by reducing levels and/or activities of SARM1 transcripts and/or products encoded thereby that are associated with the conditions, disorders or diseases.

In some embodiments, the present disclosure provides method for preventing a condition, disorder or disease, comprising administering or delivering to a subject susceptible thereto an effective amount of an oligonucleotide or composition of the present disclosure. In some embodiments, the present disclosure provides method for treating a condition, disorder or disease, comprising administering or delivering to a subject suffering therefrom an effective amount of an oligonucleotide or composition of the present disclosure.

Various conditions, disorders or diseases associated with SARM1 may be prevented or treated with provided technologies. In some embodiments, a subject benefits from reduction of levels of SARM1 transcripts, polypeptides and/or activities in certain cells, tissues and/or organs.

In some embodiments, a condition, disorder or disease is a neurodegenerative condition, disorder or disease. In some embodiments, a condition, disorder or disease is or comprises Wallerian degeneration. In some embodiments, a condition, disorder or disease is associated with Wallerian degeneration. In some embodiments, a condition, disorder or disease is amyotrophic lateral sclerosis (ALS). In some embodiments, a condition, disorder or disease is neuropathy. In some embodiments, a condition, disorder or disease is peripheral neuropathy. In some embodiments, a condition, disorder or disease is peripheral neuropathy induced by chemotherapy. In some embodiments, a condition, disorder or disease is Parkinson's disease. In some embodiments, a condition, disorder or disease is Huntington's disease. In some embodiments, a condition, disorder or disease is Alzheimer's disease. In some embodiments, a condition, disorder or disease is frontotemporal dementia. In some embodiments, a condition, disorder or disease is brain injury. In some embodiments, a condition, disorder or disease is traumatic brain injury. In some embodiments, a condition, disorder or disease is progressive supranuclear palsy. In some embodiments, a condition, disorder or disease is corticobasal degeneration. In some embodiments, a condition, disorder or disease is Wolfram Syndrome. In some embodiments, a condition, disorder or disease is Friedreich's Ataxia. In some embodiments, a condition, disorder or disease is Multiple System Atrophy. In some embodiments, a condition, disorder or disease is Spinal Cerebellar Ataxia. In some embodiments, a condition, disorder or disease is Spinal Muscular Atrophy (SMA). In some embodiments, a condition, disorder or disease is Pick's Disease. In some embodiments, a condition, disorder or disease is progressive motor atrophy. In some embodiments, a condition, disorder or disease is associated with neuronal damage. In some embodiments, a condition, disorder or disease is associated with neuronal cell damage. In some embodiments, a condition, disorder or disease is associated with neuronal cell death.

In some embodiments, a condition, disorder or disease that can be prevented and/or treated in accordance with the present disclosure is described in WO 2022/031736, WO 2021/108602, or WO 2022/125377.

Various technologies may be utilized to administer or deliver provided oligonucleotides or compositions. In some embodiments, an oligonucleotide or a composition is administered or delivered orally. In some embodiments, an oligonucleotide or a composition is administered or delivered via parenteral routes. In some embodiments, parenteral routes include intravenous, intrarterial, intramuscular, intradermal, subcutaneous, intranasal, and intraperitoneal routes. In some embodiments, an oligonucleotide or a composition is administered or delivered via intraocular, intraorbital, subconjuctival, intravitreal, subretinal, transscleral or intracochlear route. In some embodiments, an oligonucleotide or a composition is administered or delivered parenterally. In some embodiments, an oligonucleotide or a composition is administered or delivered intrathecally. In some embodiments, an oligonucleotide or a composition is administered or delivered intravenously. In some embodiments, oligonucleotides are administered or delivered as a liquid composition. In some embodiments, oligonucleotides are dissolved in a liquid, e.g., a buffered saline such as aCSF, for administration or delivery.

In some embodiments, an oligonucleotide or composition may be utilized in combination with another therapy, e.g., another therapeutic agent.

In some embodiments, provided technologies, e.g., oligonucleotides, compositions, methods, etc., delay or prevent onset of one or more symptoms and/or hallmarks of a condition, disorder or disease. In some embodiments, provided technologies delay, slow down, or prevent progression of a condition, disorder or disease. In some embodiments, provided technologies alleviate, ameliorate, relieve, inhibit, prevent, delay onset of, reduce severity of, and/or reduce incidence of one or more symptoms or features of a disease, disorder, and/or condition. In some embodiments, provided technologies improve performance of a subject in one or more assessments. In some embodiments, provided technologies improve performance of a subject in one or more clinical assessments. In some embodiments, provided technologies independently improve one or more clinical assessment results of a subject.

### Pharmaceutical Compositions

In some embodiments, the present disclosure provides pharmaceutical compositions comprising a provided compound, e.g., an oligonucleotide, or a pharmaceutically acceptable salt thereof, and a pharmaceutical carrier. In some embodiments, for example, for therapeutic and clinical purposes, oligonucleotides of the present disclosure are provided as pharmaceutical compositions.

As appreciated by those skilled in the art, oligonucleotides can be provided in various forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, provided oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, oligonucleotides of the present disclosure can exist in acid, base and/or salt forms. In some embodiments, a composition comprises one or more forms of an oligonucleotide. In some embodiments, a composition comprises one or more salt forms of an oligonucleotide. In some embodiments, a composition comprises one or more pharmaceutically acceptable salt forms of an oligonucleotide.

When used as therapeutics, a provided oligonucleotide or composition is typically administered as a pharmaceutical composition. In some embodiments, a pharmaceutical composition is suitable for administration or delivery of an oligonucleotide to an area or portion of a body affected by a condition, disorder or disease. In some embodiments, a pharmaceutical composition comprises a therapeutically effective amount of a provided oligonucleotide or a pharmaceutically acceptable salt thereof, and a pharmaceutically acceptable carrier. In some embodiments, a pharmaceutical composition comprises a therapeutically effective amount of oligonucleotides which are diastereomers of each other, wherein the oligonucleotides exist in one or more forms. In some embodiments, a pharmaceutical composition comprises a therapeutically effective amount of oligonucleotides which are diastereomers of each other with respect to chiral linkage phosphorus, wherein the oligonucleotides exist in one or more forms.

In some embodiments, a pharmaceutically acceptable carrier is a buffer. In some embodiments, a pharmaceutically acceptable carrier is a buffered saline. In some embodiments, a pharmaceutically acceptable carrier is artificial cerebrospinal fluid. In some embodiments, a composition is a liquid composition comprising dissolved oligonucleotides.

In some embodiments, a pharmaceutical composition is formulated for intravenous injection, oral administration, buccal administration, inhalation, nasal administration, topical administration, ophthalmic administration or otic administration. In some embodiments, a pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop. In some embodiments, a pharmaceutical composition is formulated for intrathecal administration.

As those skilled in the art will appreciate, oligonucleotides may exist in various salt forms. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R′)₄+). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺ or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage (acid form —O—P(O)(SH)—O—), if any, exists as its sodium salt form (—O—P(O)(SNa)—O—).

In some embodiments, an oligonucleotide or a composition, e.g., a pharmaceutical composition, is provided as solid. In some embodiments, an oligonucleotide or a composition, e.g., a pharmaceutical composition, is lyophilized.

In some embodiments, an oligonucleotide or a composition, e.g., a pharmaceutical composition, is stored at a temperature lower than an ambient temperature, e.g., at about or no more than about −78, −20, 0, 4, or 10° C.

Various technologies for delivering nucleic acids and/or oligonucleotides are known in the art and can be utilized in accordance with the present disclosure. For example, a variety of supramolecular nanocarriers can be used to deliver nucleic acids. Example nanocarriers include liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers may help to release contents from endosomes. Other approaches may include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In some embodiments, oligonucleotides are administered or delivered via gymnotic uptake.

In some embodiments, oligonucleotides or compositions are formulated for a variety of modes of administration, including systemic and topical or localized administration. Techniques and formulations generally may be found in Remington, The Science and Practice of Pharmacy (20th ed. 2000).

In certain embodiments, oligonucleotides or compositions are delivered to CNS. In certain embodiments, oligonucleotides and compositions are delivered to cerebrospinal fluid. In certain embodiments, oligonucleotides and compositions are administered to brain parenchyma. In certain embodiments, oligonucleotides and compositions are delivered to an animal/subject by intrathecal administration, or intracerebroventricular administration. Broad distribution of oligonucleotides and compositions may be achieved with methods of administration described herein and/or known in the art.

In certain embodiments, parenteral administration is by injection, by, e.g., a syringe, a pump, etc. In certain embodiments, an injection is a bolus injection. In certain embodiments, an injection is administered directly to a tissue or location, such as cerebrospinal fluid, striatum, caudate, cortex, hippocampus and/or cerebellum.

Provided oligonucleotides and compositions thereof are effective over a wide dose range. In some embodiments, a dose is from about 0.01 to about 1000 mg, from about 0.5 to about 100 mg, from about 1 to about 50 mg, or from about 5 to about 100 mg. Exact doses may depend upon routes of administration, forms in which oligonucleotides are administered, subjects (e.g., body weight, age, body surface area, etc.), conditions, disorders or diseases, and/or preferences and experiences of physicians. In some embodiments, a fixed dose is administered. In some embodiments, a provided oligonucleotide or composition is administered or delivered, e.g., by injection or infusion, once every week, every two weeks, every month, every two months, every 90 days, every 3 months, every 6 months, every 9 months, or once a year. In some embodiments, two or more doses are about the same amount. In some embodiments, one or more doses are independently more than one or more other doses. For example, in some embodiments, one or more loading doses each independently of a higher amount are administered before one or more maintenance doses each independently of a lower amount. In some embodiments, two or more or all loading doses are about the same amount. In some embodiments, a loading dose is of a higher amount than another loading dose. In some embodiments, two or more or all maintenance doses are about the same amount. In some embodiments, a maintenance dose is of a higher amount than another maintenance dose.

### Example Embodiments

Among other things, the present disclosure provides the following Example Embodiments:
1. An oligonucleotide, wherein:
   the base sequence of the oligonucleotide comprises 10 or more contiguous nucleobases of, GTCTCCAGAACTGAGCAGGG (SEQ ID NO: 62), wherein each T is optionally and independently replaced with U; and
   the oligonucleotide comprises a modified nucleobase, a modified sugar or a modified internucleotidic linkage.
2. The oligonucleotide of Embodiment 1, wherein the base sequence of the oligonucleotide is GTCTCCAGAACTGAGCAGGG (SEQ ID NO: 142).
3. An oligonucleotide, wherein:
   the base sequence of the oligonucleotide comprises 10 or more contiguous nucleobases of, CCTTGCAGGCTCTTGATGGC (SEQ ID NO: 45), wherein each T is optionally and independently replaced with U; and
   the oligonucleotide comprises a modified nucleobase, a modified sugar or a modified internucleotidic linkage.
4. The oligonucleotide of Embodiment 3, wherein the base sequence of the oligonucleotide is CCTTGCAGGCTCTTGATGGC (SEQ ID NO: 125).
5. An oligonucleotide, wherein:
   the base sequence of the oligonucleotide comprises 10 or more contiguous nucleobases of GCTGGCTGTACTCACTCTCC (SEQ ID NO: 5), wherein each T is optionally and independently replaced with U; and
   the oligonucleotide comprises a modified nucleobase, a modified sugar or a modified internucleotidic linkage.
6. The oligonucleotide of Embodiment 5, wherein the base sequence of the oligonucleotide is GCTGGCTGTACTCACTCTCC (SEQ ID NO: 85).
7. An oligonucleotide, wherein:
   the base sequence of the oligonucleotide comprises 10 or more contiguous nucleobases of TCAGGACTTTGCCTCTTTCC (SEQ ID NO: 74), wherein each T is optionally and independently replaced with U; and
   the oligonucleotide comprises a modified nucleobase, a modified sugar or a modified internucleotidic linkage.
8. The oligonucleotide of Embodiment 7, wherein the base sequence of the oligonucleotide is TCAGGACTTTGCCTCTTTCC (SEQ ID NO: 154).
9. An oligonucleotide, wherein:
   the base sequence of the oligonucleotide comprises 10 or more contiguous nucleobases of GGGAGTGGAAGGAAGGAGCC (SEQ ID NO: 80), wherein each T is optionally and independently replaced with U; and
   the oligonucleotide comprises a modified nucleobase, a modified sugar or a modified internucleotidic linkage.
10. The oligonucleotide of Embodiment 9, wherein the base sequence of the oligonucleotide is GGGAGTGGAAGGAAGGAGCC (SEQ ID NO: 160).
11. An oligonucleotide, wherein:
   the base sequence of the oligonucleotide comprises 10 or more contiguous nucleobases of GCTTTAGAGATTTGCTACCC (SEQ ID NO: 75), wherein each T is optionally and independently replaced with U; and
   the oligonucleotide comprises a modified nucleobase, a modified sugar or a modified internucleotidic linkage.
12. The oligonucleotide of Embodiment 11, wherein the base sequence of the oligonucleotide is GCTTTAGAGATTTGCTACCC (SEQ ID NO: 155).
13. An oligonucleotide, wherein:
   the base sequence of the oligonucleotide comprises 10 or more contiguous nucleobases of GCATCACTCACTGTCAGGTA (SEQ ID NO: 28), wherein each T is optionally and independently replaced with U; and
   the oligonucleotide comprises a modified nucleobase, a modified sugar or a modified internucleotidic linkage.
14. The oligonucleotide of Embodiment 13, wherein the base sequence of the oligonucleotide is GCATCACTCACTGTCAGGTA (SEQ ID NO: 108).
15. An oligonucleotide, wherein:
   the base sequence of the oligonucleotide comprises 10 or more contiguous nucleobases of GCCCTAGGATTTTCCTGTTG (SEQ ID NO: 24), wherein each T is optionally and independently replaced with U; and
   the oligonucleotide comprises a modified nucleobase, a modified sugar or a modified internucleotidic linkage.
16. The oligonucleotide of Embodiment 15, wherein the base sequence of the oligonucleotide is GCCCTAGGATTTTCCTGTTG (SEQ ID NO: 104).
17. An oligonucleotide, wherein:
   the base sequence of the oligonucleotide comprises 10 or more contiguous nucleobases of GTGCCATTGGTAGAGTAGGA (SEQ ID NO: 46), wherein each T is optionally and independently replaced with U; and
   the oligonucleotide comprises a modified nucleobase, a modified sugar or a modified internucleotidic linkage.
18. The oligonucleotide of Embodiment 17, wherein the base sequence of the oligonucleotide is GTGCCATTGGTAGAGTAGGA (SEQ ID NO: 126).
19. An oligonucleotide, wherein:
   the base sequence of the oligonucleotide comprises 10 or more contiguous nucleobases of CTGTGACCTAGGCTCCTTGA (SEQ ID NO: 8), wherein each T is optionally and independently replaced with U; and
   the oligonucleotide comprises a modified nucleobase, a modified sugar or a modified internucleotidic linkage.
20. The oligonucleotide of Embodiment 19, wherein the base sequence of the oligonucleotide is CTGTGACCTAGGCTCCTTGA (SEQ ID NO: 88).
21. An oligonucleotide, wherein:
   the base sequence of the oligonucleotide comprises 10 or more contiguous nucleobases of AGACACCTGGGTATCAGCCT (SEQ ID NO: 26), wherein each T is optionally and independently replaced with U; and
   the oligonucleotide comprises a modified nucleobase, a modified sugar or a modified internucleotidic linkage.
22. The oligonucleotide of Embodiment 21, wherein the base sequence of the oligonucleotide is AGACACCTGGGTATCAGCCT (SEQ ID NO: 106).
23. An oligonucleotide, wherein:
   the base sequence of the oligonucleotide comprises 10 or more contiguous nucleobases of GCCCAGGTTGTCTCAGCCCA (SEQ ID NO: 59), wherein each T is optionally and independently replaced with U; and
   the oligonucleotide comprises a modified nucleobase, a modified sugar or a modified internucleotidic linkage.
24. The oligonucleotide of Embodiment 23, wherein the base sequence of the oligonucleotide is GCCCAGGTTGTCTCAGCCCA (SEQ ID NO: 139).
25. An oligonucleotide, wherein:
   the base sequence of the oligonucleotide comprises 10 or more contiguous nucleobases of GCTTGTCTCATCCTGTCTCT (SEQ ID NO: 32), wherein each T is optionally and independently replaced with U; and
   the oligonucleotide comprises a modified nucleobase, a modified sugar or a modified internucleotidic linkage.
26. The oligonucleotide of Embodiment 25, wherein the base sequence of the oligonucleotide is GCTTGTCTCATCCTGTCTCT (SEQ ID NO: 112).
27. An oligonucleotide, wherein:
   the base sequence of the oligonucleotide comprises 10 or more contiguous nucleobases of GGTTCTCAGCCACCAGGATC (SEQ ID NO: 43), wherein each T is optionally and independently replaced with U; and
   the oligonucleotide comprises a modified nucleobase, a modified sugar or a modified internucleotidic linkage.
28. The oligonucleotide of Embodiment 27, wherein the base sequence of the oligonucleotide is GGTTCTCAGCCACCAGGATC (SEQ ID NO: 123).
29. An oligonucleotide, wherein:
   the base sequence of the oligonucleotide comprises 10 or more contiguous nucleobases of CAAACTGGTGTCAGAGCCTG (SEQ ID NO: 56), wherein each T is optionally and independently replaced with U; and
   the oligonucleotide comprises a modified nucleobase, a modified sugar or a modified internucleotidic linkage.
30. The oligonucleotide of Embodiment 29, wherein the base sequence of the oligonucleotide is CAAACTGGTGTCAGAGCCTG (SEQ ID NO: 136).
31. An oligonucleotide, wherein:
   the base sequence of the oligonucleotide comprises 10 or more contiguous nucleobases of GCAGCACCCTCCAAACTGGT (SEQ ID NO: 57), wherein each T is optionally and independently replaced with U; and
   the oligonucleotide comprises a modified nucleobase, a modified sugar or a modified internucleotidic linkage.
32. The oligonucleotide of Embodiment 31, wherein the base sequence of the oligonucleotide is GCAGCACCCTCCAAACTGGT (SEQ ID NO: 137).
33. An oligonucleotide, wherein:
   the base sequence of the oligonucleotide comprises 10 or more contiguous nucleobases of TGTCTCTGAGCTGACTGCTT (SEQ ID NO: 33), wherein each T is optionally and independently replaced with U; and
   the oligonucleotide comprises a modified nucleobase, a modified sugar or a modified internucleotidic linkage.
34. The oligonucleotide of Embodiment 33, wherein the base sequence of the oligonucleotide is TGTCTCTGAGCTGACTGCTT (SEQ ID NO: 113).
35. An oligonucleotide, wherein:
   the base sequence of the oligonucleotide comprises 10 or more contiguous nucleobases of CCACTAGCCCTGGGAGCAAA (SEQ ID NO: 1), wherein each T is optionally and independently replaced with U; and
   the oligonucleotide comprises a modified nucleobase, a modified sugar or a modified internucleotidic linkage.
36. The oligonucleotide of Embodiment 35, wherein the base sequence of the oligonucleotide is CCACTAGCCCTGGGAGCAAA (SEQ ID NO: 81).
37. An oligonucleotide, wherein:
   the base sequence of the oligonucleotide comprises 10 or more contiguous nucleobases of GCCATCTCCATCCATAGAGC (SEQ ID NO: 2), wherein each T is optionally and independently replaced with U; and
   the oligonucleotide comprises a modified nucleobase, a modified sugar or a modified internucleotidic linkage.
38. The oligonucleotide of Embodiment 37, wherein the base sequence of the oligonucleotide is GCCATCTCCATCCATAGAGC (SEQ ID NO: 82).
39. An oligonucleotide, wherein:
   the base sequence of the oligonucleotide comprises 10 or more contiguous nucleobases of AGGAGAGCTGTGGGCTTGGG (SEQ ID NO: 3), wherein each T is optionally and independently replaced with U; and
   the oligonucleotide comprises a modified nucleobase, a modified sugar or a modified internucleotidic linkage.
40. The oligonucleotide of Embodiment 39, wherein the base sequence of the oligonucleotide is AGGAGAGCTGTGGGCTTGGG (SEQ ID NO: 83).
41. An oligonucleotide, wherein:
   the base sequence of the oligonucleotide comprises 10 or more contiguous nucleobases of CACCCATGCCTCCCAGCAGA (SEQ ID NO: 4), wherein each T is optionally and independently replaced with U; and
   the oligonucleotide comprises a modified nucleobase, a modified sugar or a modified internucleotidic linkage.
42. The oligonucleotide of Embodiment 41, wherein the base sequence of the oligonucleotide is CACCCATGCCTCCCAGCAGA (SEQ ID NO: 84).
43. An oligonucleotide, wherein:
   the base sequence of the oligonucleotide comprises 10 or more contiguous nucleobases of GTGCTCTGTCCTTGGTCCTG (SEQ ID NO: 6), wherein each T is optionally and independently replaced with U; and
   the oligonucleotide comprises a modified nucleobase, a modified sugar or a modified internucleotidic linkage.
44. The oligonucleotide of Embodiment 43, wherein the base sequence of the oligonucleotide is GTGCTCTGTCCTTGGTCCTG (SEQ ID NO: 86).
45. An oligonucleotide, wherein:
   the base sequence of the oligonucleotide comprises 10 or more contiguous nucleobases of CCCATTCTCATGCAGCCTAC (SEQ ID NO: 7), wherein each T is optionally and independently replaced with U; and
   the oligonucleotide comprises a modified nucleobase, a modified sugar or a modified internucleotidic linkage.
46. The oligonucleotide of Embodiment 45, wherein the base sequence of the oligonucleotide is CCCATTCTCATGCAGCCTAC (SEQ ID NO: 87).
47. An oligonucleotide, wherein:
   the base sequence of the oligonucleotide comprises 10 or more contiguous nucleobases of GGTCTGAGAGGCTGTGGGTC (SEQ ID NO: 9), wherein each T is optionally and independently replaced with U; and
   the oligonucleotide comprises a modified nucleobase, a modified sugar or a modified internucleotidic linkage.
48. The oligonucleotide of Embodiment 47, wherein the base sequence of the oligonucleotide is GGTCTGAGAGGCTGTGGGTC (SEQ ID NO: 89).
49. An oligonucleotide, wherein:
   the base sequence of the oligonucleotide comprises 10 or more contiguous nucleobases of GCTCCCAGTTCTTCTGTGGT (SEQ ID NO: 10), wherein each T is optionally and independently replaced with U; and
   the oligonucleotide comprises a modified nucleobase, a modified sugar or a modified internucleotidic linkage.
50. The oligonucleotide of Embodiment 49, wherein the base sequence of the oligonucleotide is GCTCCCAGTTCTTCTGTGGT (SEQ ID NO: 90).
51. An oligonucleotide, wherein:
   the base sequence of the oligonucleotide comprises 10 or more contiguous nucleobases of GATGTCCTCCACAGGTGACA (SEQ ID NO: 11), wherein each T is optionally and independently replaced with U; and
   the oligonucleotide comprises a modified nucleobase, a modified sugar or a modified internucleotidic linkage.
52. The oligonucleotide of Embodiment 51, wherein the base sequence of the oligonucleotide is GATGTCCTCCACAGGTGACA (SEQ ID NO: 91).
53. An oligonucleotide, wherein:
   the base sequence of the oligonucleotide comprises 10 or more contiguous nucleobases of GCTTCCTGCCTTACTGACCT (SEQ ID NO: 12), wherein each T is optionally and independently replaced with U; and
   the oligonucleotide comprises a modified nucleobase, a modified sugar or a modified internucleotidic linkage.
54. The oligonucleotide of Embodiment 53, wherein the base sequence of the oligonucleotide is GCTTCCTGCCTTACTGACCT (SEQ ID NO: 92).
55. An oligonucleotide, wherein:
   the base sequence of the oligonucleotide comprises 10 or more contiguous nucleobases of CTCTCCTTTGTCCCTGACCA (SEQ ID NO: 13), wherein each T is optionally and independently replaced with U; and
   the oligonucleotide comprises a modified nucleobase, a modified sugar or a modified internucleotidic linkage.
56. The oligonucleotide of Embodiment 55, wherein the base sequence of the oligonucleotide is CTCTCCTTTGTCCCTGACCA (SEQ ID NO: 93).
57. An oligonucleotide, wherein:
   the base sequence of the oligonucleotide comprises 10 or more contiguous nucleobases of GCCTTGCCTTTTCCTCACTC (SEQ ID NO: 14), wherein each T is optionally and independently replaced with U; and
   the oligonucleotide comprises a modified nucleobase, a modified sugar or a modified internucleotidic linkage.
58. The oligonucleotide of Embodiment 57, wherein the base sequence of the oligonucleotide is GCCTTGCCTTTTCCTCACTC (SEQ ID NO: 94).
59. An oligonucleotide, wherein:
   the base sequence of the oligonucleotide comprises 10 or more contiguous nucleobases of GCCTGGTCACTAACCCTCTC (SEQ ID NO: 15), wherein each T is optionally and independently replaced with U; and
   the oligonucleotide comprises a modified nucleobase, a modified sugar or a modified internucleotidic linkage.
60. The oligonucleotide of Embodiment 59, wherein the base sequence of the oligonucleotide is GCCTGGTCACTAACCCTCTC (SEQ ID NO: 95).
61. An oligonucleotide, wherein:
   the base sequence of the oligonucleotide comprises 10 or more contiguous nucleobases of CACCCACCTTGGTCTTGCCT (SEQ ID NO: 16), wherein each T is optionally and independently replaced with U; and
   the oligonucleotide comprises a modified nucleobase, a modified sugar or a modified internucleotidic linkage.
62. The oligonucleotide of Embodiment 61, wherein the base sequence of the oligonucleotide is CACCCACCTTGGTCTTGCCT (SEQ ID NO: 96).
63. An oligonucleotide, wherein:
   the base sequence of the oligonucleotide comprises 10 or more contiguous nucleobases of CACACTGATGTCCTGTCCCA (SEQ ID NO: 17), wherein each T is optionally and independently replaced with U; and
   the oligonucleotide comprises a modified nucleobase, a modified sugar or a modified internucleotidic linkage.
64. The oligonucleotide of Embodiment 63, wherein the base sequence of the oligonucleotide is CACACTGATGTCCTGTCCCA (SEQ ID NO: 97).
65. An oligonucleotide, wherein:
   the base sequence of the oligonucleotide comprises 10 or more contiguous nucleobases of CACACCTCTGGGTCTTGGCC (SEQ ID NO: 18), wherein each T is optionally and independently replaced with U; and
   the oligonucleotide comprises a modified nucleobase, a modified sugar or a modified internucleotidic linkage.
66. The oligonucleotide of Embodiment 65, wherein the base sequence of the oligonucleotide is CACACCTCTGGGTCTTGGCC (SEQ ID NO: 98).
67. An oligonucleotide, wherein:
   the base sequence of the oligonucleotide comprises 10 or more contiguous nucleobases of GCTGCCCATCACTCCCAGTT (SEQ ID NO: 19), wherein each T is optionally and independently replaced with U; and
   the oligonucleotide comprises a modified nucleobase, a modified sugar or a modified internucleotidic linkage.
68. The oligonucleotide of Embodiment 67, wherein the base sequence of the oligonucleotide is GCTGCCCATCACTCCCAGTT (SEQ ID NO: 99).
69. An oligonucleotide, wherein:
   the base sequence of the oligonucleotide comprises 10 or more contiguous nucleobases of CTCTCCATCTGCCCTGGCCC (SEQ ID NO: 20), wherein each T is optionally and independently replaced with U; and
   the oligonucleotide comprises a modified nucleobase, a modified sugar or a modified internucleotidic linkage.
70. The oligonucleotide of Embodiment 69, wherein the base sequence of the oligonucleotide is CTCTCCATCTGCCCTGGCCC (SEQ ID NO: 100).
71. An oligonucleotide, wherein:
   the base sequence of the oligonucleotide comprises 10 or more contiguous nucleobases of CAGTCCCTCTCCTTGTCTCT (SEQ ID NO: 21), wherein each T is optionally and independently replaced with U; and
   the oligonucleotide comprises a modified nucleobase, a modified sugar or a modified internucleotidic linkage.
72. The oligonucleotide of Embodiment 71, wherein the base sequence of the oligonucleotide is CAGTCCCTCTCCTTGTCTCT (SEQ ID NO: 101).
73. An oligonucleotide, wherein:
   the base sequence of the oligonucleotide comprises 10 or more contiguous nucleobases of ATCCACCTGCTGCTCCTGGG (SEQ ID NO: 22), wherein each T is optionally and independently replaced with U; and
   the oligonucleotide comprises a modified nucleobase, a modified sugar or a modified internucleotidic linkage.
74. The oligonucleotide of Embodiment 73, wherein the base sequence of the oligonucleotide is ATCCACCTGCTGCTCCTGGG (SEQ ID NO: 102).
75. An oligonucleotide, wherein:
   the base sequence of the oligonucleotide comprises 10 or more contiguous nucleobases of CCCTTGTGTCTTGTGGGTGC (SEQ ID NO: 23), wherein each T is optionally and independently replaced with U; and
   the oligonucleotide comprises a modified nucleobase, a modified sugar or a modified internucleotidic linkage.
76. The oligonucleotide of Embodiment 75, wherein the base sequence of the oligonucleotide is CCCTTGTGTCTTGTGGGTGC (SEQ ID NO: 103).
77. An oligonucleotide, wherein:
   the base sequence of the oligonucleotide comprises 10 or more contiguous nucleobases of GCCTCAACTCCTGCCTCCCA (SEQ ID NO: 25), wherein each T is optionally and independently replaced with U; and
   the oligonucleotide comprises a modified nucleobase, a modified sugar or a modified internucleotidic linkage.
78. The oligonucleotide of Embodiment 77, wherein the base sequence of the oligonucleotide is GCCTCAACTCCTGCCTCCCA (SEQ ID NO: 105).
79. An oligonucleotide, wherein:
   the base sequence of the oligonucleotide comprises 10 or more contiguous nucleobases of TCCTTCTTCCCTATTTCCCA (SEQ ID NO: 27), wherein each T is optionally and independently replaced with U; and
   the oligonucleotide comprises a modified nucleobase, a modified sugar or a modified internucleotidic linkage.
80. The oligonucleotide of Embodiment 79, wherein the base sequence of the oligonucleotide is TCCTTCTTCCCTATTTCCCA (SEQ ID NO: 107).
81. An oligonucleotide, wherein:
   the base sequence of the oligonucleotide comprises 10 or more contiguous nucleobases of GTCAGTGCCACAGCCTTGTC (SEQ ID NO: 29), wherein each T is optionally and independently replaced with U; and
   the oligonucleotide comprises a modified nucleobase, a modified sugar or a modified internucleotidic linkage.
82. The oligonucleotide of Embodiment 81, wherein the base sequence of the oligonucleotide is GTCAGTGCCACAGCCTTGTC (SEQ ID NO: 109).
83. An oligonucleotide, wherein:
   the base sequence of the oligonucleotide comprises 10 or more contiguous nucleobases of GGCACCTACCTTATGCACCC (SEQ ID NO: 30), wherein each T is optionally and independently replaced with U; and
   the oligonucleotide comprises a modified nucleobase, a modified sugar or a modified internucleotidic linkage.
84. The oligonucleotide of Embodiment 83, wherein the base sequence of the oligonucleotide is GGCACCTACCTTATGCACCC (SEQ ID NO: 110).
85. An oligonucleotide, wherein:
   the base sequence of the oligonucleotide comprises 10 or more contiguous nucleobases of ACTACTGCATCCCTCAGCCC (SEQ ID NO: 31), wherein each T is optionally and independently replaced with U; and
   the oligonucleotide comprises a modified nucleobase, a modified sugar or a modified internucleotidic linkage.
86. The oligonucleotide of Embodiment 85, wherein the base sequence of the oligonucleotide is ACTACTGCATCCCTCAGCCC (SEQ ID NO: 111).
87. An oligonucleotide, wherein:
   the base sequence of the oligonucleotide comprises 10 or more contiguous nucleobases of GGGCTTGACTCCACACTCCA (SEQ ID NO: 34), wherein each T is optionally and independently replaced with U; and
   the oligonucleotide comprises a modified nucleobase, a modified sugar or a modified internucleotidic linkage.
88. The oligonucleotide of Embodiment 87, wherein the base sequence of the oligonucleotide is GGGCTTGACTCCACACTCCA (SEQ ID NO: 114).
89. An oligonucleotide, wherein:
   the base sequence of the oligonucleotide comprises 10 or more contiguous nucleobases of GGCATGGCATCTCAGCTTCA (SEQ ID NO: 35), wherein each T is optionally and independently replaced with U; and
   the oligonucleotide comprises a modified nucleobase, a modified sugar or a modified internucleotidic linkage.
90. The oligonucleotide of Embodiment 89, wherein the base sequence of the oligonucleotide is GGCATGGCATCTCAGCTTCA (SEQ ID NO: 115).
91. An oligonucleotide, wherein:
   the base sequence of the oligonucleotide comprises 10 or more contiguous nucleobases of TTCAGGATCACCTAGCTGGT (SEQ ID NO: 36), wherein each T is optionally and independently replaced with U; and
   the oligonucleotide comprises a modified nucleobase, a modified sugar or a modified internucleotidic linkage.
92. The oligonucleotide of Embodiment 91, wherein the base sequence of the oligonucleotide is TTCAGGATCACCTAGCTGGT (SEQ ID NO: 116).
93. An oligonucleotide, wherein:
   the base sequence of the oligonucleotide comprises 10 or more contiguous nucleobases of CCTCTTTGCCATCTGCTGGG (SEQ ID NO: 37), wherein each T is optionally and independently replaced with U; and
   the oligonucleotide comprises a modified nucleobase, a modified sugar or a modified internucleotidic linkage.
94. The oligonucleotide of Embodiment 93, wherein the base sequence of the oligonucleotide is CCTCTTTGCCATCTGCTGGG (SEQ ID NO: 117).
95. An oligonucleotide, wherein:
   the base sequence of the oligonucleotide comprises 10 or more contiguous nucleobases of GAGTGCAGTTCACTTGTGGT (SEQ ID NO: 38), wherein each T is optionally and independently replaced with U; and
   the oligonucleotide comprises a modified nucleobase, a modified sugar or a modified internucleotidic linkage.
96. The oligonucleotide of Embodiment 95, wherein the base sequence of the oligonucleotide is GAGTGCAGTTCACTTGTGGT (SEQ ID NO: 118).
97. An oligonucleotide, wherein:
   the base sequence of the oligonucleotide comprises 10 or more contiguous nucleobases of TGCCCACACTCTGCCTGTCA (SEQ ID NO: 39), wherein each T is optionally and independently replaced with U; and
   the oligonucleotide comprises a modified nucleobase, a modified sugar or a modified internucleotidic linkage.
98. The oligonucleotide of Embodiment 97, wherein the base sequence of the oligonucleotide is TGCCCACACTCTGCCTGTCA (SEQ ID NO: 119).
99. An oligonucleotide, wherein:
   the base sequence of the oligonucleotide comprises 10 or more contiguous nucleobases of CAGAGGGAGCTGCTAGTCAG (SEQ ID NO: 40), wherein each T is optionally and independently replaced with U; and
   the oligonucleotide comprises a modified nucleobase, a modified sugar or a modified internucleotidic linkage.
100. The oligonucleotide of Embodiment 99, wherein the base sequence of the oligonucleotide is CAGAGGGAGCTGCTAGTCAG (SEQ ID NO: 120).
101. An oligonucleotide, wherein:
   the base sequence of the oligonucleotide comprises 10 or more contiguous nucleobases of TTGGCAAAGGTGATGCAGGC (SEQ ID NO: 41), wherein each T is optionally and independently replaced with U; and
   the oligonucleotide comprises a modified nucleobase, a modified sugar or a modified internucleotidic linkage.
102. The oligonucleotide of Embodiment 101, wherein the base sequence of the oligonucleotide is TTGGCAAAGGTGATGCAGGC (SEQ ID NO: 121).
103. An oligonucleotide, wherein:
   the base sequence of the oligonucleotide comprises 10 or more contiguous nucleobases of CCTCCACCAGTTGGAAGACC (SEQ ID NO: 42), wherein each T is optionally and independently replaced with U; and
   the oligonucleotide comprises a modified nucleobase, a modified sugar or a modified internucleotidic linkage.
104. The oligonucleotide of Embodiment 103, wherein the base sequence of the oligonucleotide is CCTCCACCAGTTGGAAGACC (SEQ ID NO: 122).
105. An oligonucleotide, wherein:
   the base sequence of the oligonucleotide comprises 10 or more contiguous nucleobases of GTGCTCCAAGATGCCTGCCA (SEQ ID NO: 44), wherein each T is optionally and independently replaced with U; and
   the oligonucleotide comprises a modified nucleobase, a modified sugar or a modified internucleotidic linkage.
106. The oligonucleotide of Embodiment 105, wherein the base sequence of the oligonucleotide is GTGCTCCAAGATGCCTGCCA (SEQ ID NO: 124).
107. An oligonucleotide, wherein:
   the base sequence of the oligonucleotide comprises 10 or more contiguous nucleobases of GTGAGCTCCCTAAAGAACCT (SEQ ID NO: 47), wherein each T is optionally and independently replaced with U; and
   the oligonucleotide comprises a modified nucleobase, a modified sugar or a modified internucleotidic linkage.
108. The oligonucleotide of Embodiment 107, wherein the base sequence of the oligonucleotide is GTGAGCTCCCTAAAGAACCT (SEQ ID NO: 127).
109. An oligonucleotide, wherein:
   the base sequence of the oligonucleotide comprises 10 or more contiguous nucleobases of GGTTTGCCACCAGTACAGGG (SEQ ID NO: 48), wherein each T is optionally and independently replaced with U; and
   the oligonucleotide comprises a modified nucleobase, a modified sugar or a modified internucleotidic linkage.
110. The oligonucleotide of Embodiment 109, wherein the base sequence of the oligonucleotide is GGTTTGCCACCAGTACAGGG (SEQ ID NO: 128).
111. An oligonucleotide, wherein:
   the base sequence of the oligonucleotide comprises 10 or more contiguous nucleobases of TCCAGCTTCTCCACATCAAT (SEQ ID NO: 49), wherein each T is optionally and independently replaced with U; and
   the oligonucleotide comprises a modified nucleobase, a modified sugar or a modified internucleotidic linkage.
112. The oligonucleotide of Embodiment 111, wherein the base sequence of the oligonucleotide is TCCAGCTTCTCCACATCAAT (SEQ ID NO: 129).
113. An oligonucleotide, wherein:
   the base sequence of the oligonucleotide comprises 10 or more contiguous nucleobases of GAACTTGCCTGCTTCCAGCT (SEQ ID NO: 50), wherein each T is optionally and independently replaced with U; and
   the oligonucleotide comprises a modified nucleobase, a modified sugar or a modified internucleotidic linkage.
114. The oligonucleotide of Embodiment 113, wherein the base sequence of the oligonucleotide is GAACTTGCCTGCTTCCAGCT (SEQ ID NO: 130).
115. An oligonucleotide, wherein:
   the base sequence of the oligonucleotide comprises 10 or more contiguous nucleobases of ACACTCTGGATGAGTTTGTC (SEQ ID NO: 51), wherein each T is optionally and independently replaced with U; and
   the oligonucleotide comprises a modified nucleobase, a modified sugar or a modified internucleotidic linkage.
116. The oligonucleotide of Embodiment 115, wherein the base sequence of the oligonucleotide is ACACTCTGGATGAGTTTGTC (SEQ ID NO: 131).
117. An oligonucleotide, wherein:
   the base sequence of the oligonucleotide comprises 10 or more contiguous nucleobases of GGGCACCCATGACACTCTGG (SEQ ID NO: 52), wherein each T is optionally and independently replaced with U; and
   the oligonucleotide comprises a modified nucleobase, a modified sugar or a modified internucleotidic linkage.
118. The oligonucleotide of Embodiment 117, wherein the base sequence of the oligonucleotide is GGGCACCCATGACACTCTGG (SEQ ID NO: 132).
119. An oligonucleotide, wherein:
   the base sequence of the oligonucleotide comprises 10 or more contiguous nucleobases of ACTTGTCCAGTGCTCCAGGT (SEQ ID NO: 53), wherein each T is optionally and independently replaced with U; and
   the oligonucleotide comprises a modified nucleobase, a modified sugar or a modified internucleotidic linkage.
120. The oligonucleotide of Embodiment 119, wherein the base sequence of the oligonucleotide is ACTTGTCCAGTGCTCCAGGT (SEQ ID NO: 133).
121. An oligonucleotide, wherein:
   the base sequence of the oligonucleotide comprises 10 or more contiguous nucleobases of CCCAATCCTTGCAGTCATGG (SEQ ID NO: 54), wherein each T is optionally and independently replaced with U; and
   the oligonucleotide comprises a modified nucleobase, a modified sugar or a modified internucleotidic linkage.
122. The oligonucleotide of Embodiment 121, wherein the base sequence of the oligonucleotide is CCCAATCCTTGCAGTCATGG (SEQ ID NO: 134).
123. An oligonucleotide, wherein:
   the base sequence of the oligonucleotide comprises 10 or more contiguous nucleobases of AGCACAGCCTGCATGTCCTC (SEQ ID NO: 55), wherein each T is optionally and independently replaced with U; and
   the oligonucleotide comprises a modified nucleobase, a modified sugar or a modified internucleotidic linkage.
124. The oligonucleotide of Embodiment 123, wherein the base sequence of the oligonucleotide is AGCACAGCCTGCATGTCCTC (SEQ ID NO: 135).
125. An oligonucleotide, wherein:
   the base sequence of the oligonucleotide comprises 10 or more contiguous nucleobases of TGGTTAGGTTGGACCCATGG (SEQ ID NO: 58), wherein each T is optionally and independently replaced with U; and
   the oligonucleotide comprises a modified nucleobase, a modified sugar or a modified internucleotidic linkage.
126. The oligonucleotide of Embodiment 125, wherein the base sequence of the oligonucleotide is TGGTTAGGTTGGACCCATGG (SEQ ID NO: 138).
127. An oligonucleotide, wherein:
   the base sequence of the oligonucleotide comprises 10 or more contiguous nucleobases of TCCCTCTCCAGATACTGAGG (SEQ ID NO: 60), wherein each T is optionally and independently replaced with U; and
   the oligonucleotide comprises a modified nucleobase, a modified sugar or a modified internucleotidic linkage.
128. The oligonucleotide of Embodiment 127, wherein the base sequence of the oligonucleotide is TCCCTCTCCAGATACTGAGG (SEQ ID NO: 140).
129. An oligonucleotide, wherein:
   the base sequence of the oligonucleotide comprises 10 or more contiguous nucleobases of ACAGACAACCCAATGGCAGG (SEQ ID NO: 61), wherein each T is optionally and independently replaced with U; and
   the oligonucleotide comprises a modified nucleobase, a modified sugar or a modified internucleotidic linkage.
130. The oligonucleotide of Embodiment 129, wherein the base sequence of the oligonucleotide is ACAGACAACCCAATGGCAGG (SEQ ID NO: 141).
131. An oligonucleotide, wherein:
   the base sequence of the oligonucleotide comprises 10 or more contiguous nucleobases of CCTTAATTCCTGTCTGAGGC (SEQ ID NO: 63), wherein each T is optionally and independently replaced with U; and
   the oligonucleotide comprises a modified nucleobase, a modified sugar or a modified internucleotidic linkage.
132. The oligonucleotide of Embodiment 131, wherein the base sequence of the oligonucleotide is CCTTAATTCCTGTCTGAGGC (SEQ ID NO: 143).
133. An oligonucleotide, wherein:
   the base sequence of the oligonucleotide comprises 10 or more contiguous nucleobases of CAGAATACAGTGCCCAGGCC (SEQ ID NO: 64), wherein each T is optionally and independently replaced with U; and
   the oligonucleotide comprises a modified nucleobase, a modified sugar or a modified internucleotidic linkage.
134. The oligonucleotide of Embodiment 133, wherein the base sequence of the oligonucleotide is CAGAATACAGTGCCCAGGCC (SEQ ID NO: 144).
135. An oligonucleotide, wherein:
   the base sequence of the oligonucleotide comprises 10 or more contiguous nucleobases of CCCAGGCCCTTGCTCAGAAT (SEQ ID NO: 65), wherein each T is optionally and independently replaced with U; and
   the oligonucleotide comprises a modified nucleobase, a modified sugar or a modified internucleotidic linkage.
136. The oligonucleotide of Embodiment 135, wherein the base sequence of the oligonucleotide is CCCAGGCCCTTGCTCAGAAT (SEQ ID NO: 145).
137. An oligonucleotide, wherein:
   the base sequence of the oligonucleotide comprises 10 or more contiguous nucleobases of GCACTCATCCCTGGCTGGCT (SEQ ID NO: 66), wherein each T is optionally and independently replaced with U; and
   the oligonucleotide comprises a modified nucleobase, a modified sugar or a modified internucleotidic linkage.
138. The oligonucleotide of Embodiment 137, wherein the base sequence of the oligonucleotide is GCACTCATCCCTGGCTGGCT (SEQ ID NO: 146).
139. An oligonucleotide, wherein:
   the base sequence of the oligonucleotide comprises 10 or more contiguous nucleobases of GATTACAGGGCAAGGCCACA (SEQ ID NO: 67), wherein each T is optionally and independently replaced with U; and
   the oligonucleotide comprises a modified nucleobase, a modified sugar or a modified internucleotidic linkage.
140. The oligonucleotide of Embodiment 139, wherein the base sequence of the oligonucleotide is GATTACAGGGCAAGGCCACA (SEQ ID NO: 147).
141. An oligonucleotide, wherein:
   the base sequence of the oligonucleotide comprises 10 or more contiguous nucleobases of GCCCTGGATGTGGCAAAAGA (SEQ ID NO: 68), wherein each T is optionally and independently replaced with U; and
   the oligonucleotide comprises a modified nucleobase, a modified sugar or a modified internucleotidic linkage.
142. The oligonucleotide of Embodiment 141, wherein the base sequence of the oligonucleotide is GCCCTGGATGTGGCAAAAGA (SEQ ID NO: 148).
143. An oligonucleotide, wherein:
   the base sequence of the oligonucleotide comprises 10 or more contiguous nucleobases of AAGGAAGTCAGAGGGAGGGC (SEQ ID NO: 69), wherein each T is optionally and independently replaced with U; and
   the oligonucleotide comprises a modified nucleobase, a modified sugar or a modified internucleotidic linkage.
144. The oligonucleotide of Embodiment 143, wherein the base sequence of the oligonucleotide is AAGGAAGTCAGAGGGAGGGC (SEQ ID NO: 149).
145. An oligonucleotide, wherein:
   the base sequence of the oligonucleotide comprises 10 or more contiguous nucleobases of CAGGCCCAAACAGGAGGCTC (SEQ ID NO: 70), wherein each T is optionally and independently replaced with U; and
   the oligonucleotide comprises a modified nucleobase, a modified sugar or a modified internucleotidic linkage.
146. The oligonucleotide of Embodiment 145, wherein the base sequence of the oligonucleotide is CAGGCCCAAACAGGAGGCTC (SEQ ID NO: 150).
147. An oligonucleotide, wherein:
   the base sequence of the oligonucleotide comprises 10 or more contiguous nucleobases of ATGCCCAGACCCAGGCCCAA (SEQ ID NO: 71), wherein each T is optionally and independently replaced with U; and
   the oligonucleotide comprises a modified nucleobase, a modified sugar or a modified internucleotidic linkage.
148. The oligonucleotide of Embodiment 147, wherein the base sequence of the oligonucleotide is ATGCCCAGACCCAGGCCCAA (SEQ ID NO: 151).
149. An oligonucleotide, wherein:
   the base sequence of the oligonucleotide comprises 10 or more contiguous nucleobases of CTGAGGCACAGCACCAAGGC (SEQ ID NO: 72), wherein each T is optionally and independently replaced with U; and
   the oligonucleotide comprises a modified nucleobase, a modified sugar or a modified internucleotidic linkage.
150. The oligonucleotide of Embodiment 149, wherein the base sequence of the oligonucleotide is CTGAGGCACAGCACCAAGGC (SEQ ID NO: 152).
151. An oligonucleotide, wherein:
   the base sequence of the oligonucleotide comprises 10 or more contiguous nucleobases of GCCAGACCAGGAAGGAGCCT (SEQ ID NO: 73), wherein each T is optionally and independently replaced with U; and
   the oligonucleotide comprises a modified nucleobase, a modified sugar or a modified internucleotidic linkage.
152. The oligonucleotide of Embodiment 151, wherein the base sequence of the oligonucleotide is GCCAGACCAGGAAGGAGCCT (SEQ ID NO: 153).
153. An oligonucleotide, wherein:
   the base sequence of the oligonucleotide comprises 10 or more contiguous nucleobases of GCCCAGCCTCAGAATGATTC (SEQ ID NO: 76), wherein each T is optionally and independently replaced with U; and
   the oligonucleotide comprises a modified nucleobase, a modified sugar or a modified internucleotidic linkage.
154. The oligonucleotide of Embodiment 153, wherein the base sequence of the oligonucleotide is GCCCAGCCTCAGAATGATTC (SEQ ID NO: 156).
155. An oligonucleotide, wherein:
   the base sequence of the oligonucleotide comprises 10 or more contiguous nucleobases of CCTCTGAACCCAGTGGAGGA (SEQ ID NO: 77), wherein each T is optionally and independently replaced with U; and
   the oligonucleotide comprises a modified nucleobase, a modified sugar or a modified internucleotidic linkage.
156. The oligonucleotide of Embodiment 155, wherein the base sequence of the oligonucleotide is CCTCTGAACCCAGTGGAGGA (SEQ ID NO: 157).
157. An oligonucleotide, wherein:
   the base sequence of the oligonucleotide comprises 10 or more contiguous nucleobases of GCCTGGGTTTATTGGAGGGT (SEQ ID NO: 78), wherein each T is optionally and independently replaced with U; and
   the oligonucleotide comprises a modified nucleobase, a modified sugar or a modified internucleotidic linkage.
158. The oligonucleotide of Embodiment 157, wherein the base sequence of the oligonucleotide is GCCTGGGTTTATTGGAGGGT (SEQ ID NO: 158).
159. An oligonucleotide, wherein:
   the base sequence of the oligonucleotide comprises 10 or more contiguous nucleobases of GCCAGCACAGCCAAGAGTGG (SEQ ID NO: 79), wherein each T is optionally and independently replaced with U; and
   the oligonucleotide comprises a modified nucleobase, a modified sugar or a modified internucleotidic linkage.
160. The oligonucleotide of Embodiment 159, wherein the base sequence of the oligonucleotide is GCCAGCACAGCCAAGAGTGG (SEQ ID NO: 159).
161. The oligonucleotide of any one of the preceding Embodiments, wherein the oligonucleotide comprises a 5′-wing-core-wing-3′ structure.
162. The oligonucleotide of any one of the preceding Embodiments, wherein there are about 3-10 nucleosides in the 5′-wing.
163. The oligonucleotide of any one of the preceding Embodiments, wherein there are 5 nucleosides in the 5′-wing.
164. The oligonucleotide of any one of the preceding Embodiments, wherein each sugar in the 5′-wing is independently a modified sugar.
165. The oligonucleotide of any one of the preceding Embodiments, wherein a sugar in a 5′-wing is a 2′-OR^{s} modified sugar wherein R^{s} is C₁₋₆ aliphatic.
166. The oligonucleotide of any one of the preceding Embodiments, wherein a sugar in a 5′-wing is a 2′-MOE modified sugar.
167. The oligonucleotide of any one of the preceding Embodiments, wherein a sugar in a 5′-wing is a 2′-OMe modified sugar.
168. The oligonucleotide of any one of the preceding Embodiments, wherein a sugar in a 5′-wing is a bicyclic sugar.
169. The oligonucleotide of Embodiment 168, wherein the bicyclic sugar is a LNA sugar.
170. The oligonucleotide of Embodiment 168, wherein the bicyclic sugar is a cEt sugar.
171. The oligonucleotide of any one of Embodiments 1-165, wherein each sugar in a 5′-wing is independently a 2′-OR modified sugar wherein R^{s} is C₁₋₆ aliphatic.
172. The oligonucleotide of any one of Embodiments 1-165, wherein each sugar in a 5′-wing is independently a 2′-MOE modified sugar.
173. The oligonucleotide of any one of the preceding Embodiments, wherein there are about 8-15 nucleosides in the core.
174. The oligonucleotide of any one of the preceding Embodiments, wherein there are 10 nucleosides in the core.
175. The oligonucleotide of any one of the preceding Embodiments, wherein each sugar in the core is independently a natural DNA sugar.
176. The oligonucleotide of any one of the preceding Embodiments, wherein the core contains no cytosine.
177. The oligonucleotide of any one of the preceding Embodiments, wherein the core comprises one or more 5-methylcytosine.
178. The oligonucleotide of any one of the preceding Embodiments, wherein there are about 3-10 nucleosides in the 3′-wing.
179. The oligonucleotide of any one of the preceding Embodiments, wherein there are 5 nucleosides in the 3′-wing.
180. The oligonucleotide of any one of the preceding Embodiments, wherein each sugar in the 3′-wing is independently a modified sugar.
181. The oligonucleotide of any one of the preceding Embodiments, wherein a sugar in a 3′-wing is a 2′-OR modified sugar wherein R^{s} is C₁₋₆ aliphatic.
182. The oligonucleotide of any one of the preceding Embodiments, wherein a sugar in a 3′-wing is a 2′-MOE modified sugar.
183. The oligonucleotide of any one of the preceding Embodiments, wherein a sugar in a 3′-wing is a 2′-OMe modified sugar.
184. The oligonucleotide of any one of the preceding Embodiments, wherein a sugar in a 3′-wing is a bicyclic sugar.
185. The oligonucleotide of Embodiment 184, wherein the bicyclic sugar is a LNA sugar.
186. The oligonucleotide of Embodiment 184, wherein the bicyclic sugar is a cEt sugar.
187. The oligonucleotide of any one of Embodiments 1-181, wherein each sugar in a 3′-wing is independently a 2′-OR modified sugar wherein R^{s} is C₁₋₆ aliphatic.
188. The oligonucleotide of any one of Embodiments 1-181, wherein each sugar in a 3′-wing is independently a 2′-MOE modified sugar.
189. The oligonucleotide of any one of the preceding Embodiments, wherein the oligonucleotide comprises a modified internucleotidic linkage.
190. The oligonucleotide of Embodiment 189, wherein the modified internucleotidic linkage is a phosphorothioate internucleotidic linkage.
191. The oligonucleotide of any one of the preceding Embodiments, wherein each internucleotidic linkage is independently a modified internucleotidic linkage.
192. The oligonucleotide of any one of the preceding Embodiments, wherein each internucleotidic linkage is independently a phosphorothioate internucleotidic linkage.
193. An oligonucleotide having the structure selected from:

(SEQ ID NO: 382)
/52MOErG/*/i2MOErT/*/i2MOErC/*/i2MOErT/*/i2MO
ErC/*/iMe-dC/*A*G*A*A*/iMe-dC/*T*G*A*G*/i2MOE
rC/*/i2MOErA/*/i2MOErG/*/i2MOErG/*/32MOErG/,
(SEQ ID NO: 365)
/52MOErC/*/i2MOErC/*/i2MOErT/*/i2MOErT/*/i2MO
ErG/*/iMe-dC/*A*G*G*/iMe-dC/*T*/iMe-dC/*T*T*G
*/i2MOErA/*/i2MOErT/*/i2MOErG/*/i2MOErG/*/32M
OErC/,
(SEQ ID NO: 321)
/52MOErC/*/i2MOErC/*/i2MOErA/*/i2MOErC/*/i2MO
ErT/*A*G*/iMe-dC/*/iMe-dC/*/iMe-dC/*T*G*G*G*A
*/i2MOErG/*/i2MOErC/*/i2MOErA/*/i2MOErA/*/32M
OErA/,
(SEQ ID NO: 322)
/52MOErG/*/i2MOErC/*/i2MOErC/*/i2MOErA/*/i2MO
ErT/*/iMe-dC/*T*/iMe-dC/*/iMe-dC/*A*T*/iMe-dC/
*/iMe-dC/*A*T*/i2MOErA/*/i2MO
ErG/*/i2MOErA/*/i2MOErG/*/32MOErC/,
(SEQ ID NO: 323)
/52MOErA/*/i2MOErG/*/i2MOErG/*/i2MOErA/*/i2MO
ErG/*A*G*/iMe-dC/*T*G*T*G*G*G*/iMe-dC/*/i2MOE
rT/*/i2MOErT/*/i2MOErG/*/i2MOErG/*/32MOErG/,
(SEQ ID NO: 324)
/52MOErC/*/i2MOErA/*/i2MOErC/*/i2MOErC/*/i2MO
ErC/*A*T*G*/iMe-dC/*/iMe-dC/*T*/iMe-dC/*/iMe-
dC/*/iMe-dC/*A*/i2MOErG/*/i2MOErC/*/i2MOErA/*
/i2MOErG/*/32MOErA/,
(SEQ ID NO: 325)
/52MOErG/*/i2MOErC/*/i2MOErT/*/i2MOErG/*/i2MO
ErG/*/iMe-dC/*T*G*T*A*/iMe-dC/*T*/iMe-dC/*A*/
iMe-dC/*/i2MOErT/*/i2MOErC/*/i2MOErT/*/i2MOEr
C/*/32MOErC/,
(SEQ ID NO: 326)
/52MOErG/*/i2MOErT/*/i2MOErG/*/i2MOErC/*/i2MO
ErT/*/iMe-dC/*T*G*T*/iMe-dC/*/iMe-dC/*T*T*G*G
*/i2MOErT/*/i2MOErC/*/i2MOErC/*/i2MOErT/*/32M
OErG/,
(SEQ ID NO: 327)
/52MOErC/*/i2MOErC/*/i2MOErC/*/i2MOErA/*/i2MO
ErT/*T*/iMe-dC/*T*/iMe-dC/*A*T*G*/iMe-dC/*A*G
*/i2MOErC/*/i2MOErC/*/i2MOErT/*/i2MOErA/*/32M
OErC/,
(SEQ ID NO: 328)
/52MOErC/*/i2MOErT/*/i2MOErG/*/i2MOErT/*/i2MO
ErG/*A*/iMe-dC/*/iMe-dC/*T*A*G*G*/iMe-dC/*T*/
iMe-dC/*/i2MOErC/*/i2MOErT/*/i2MOErT/*/i2MOEr
G/*/32MOErA/,
(SEQ ID NO: 329)
/52MOErG/*/i2MOErG/*/i2MOErT/*/i2MOErC/*/i2MO
ErT/*G*A*G*A*G*G*/iMe-dC/*T*G*T*/i2MOErG/*/i2
MOErG/*/i2MOErG/*/i2MOErT/*/32MOErC/,
(SEQ ID NO: 330)
/52MOErG/*/i2MOErC/*/i2MOErT/*/i2MOErC/*/i2MO
ErC/*/iMe-dC/*A*G*T*T*/iMe-dC/*T*T*/iMe-dC/*T
*/i2MOErG/*/i2MOErT/*/i2MOErG/*/i2MOErG/*/32M
OErT/,
(SEQ ID NO: 331)
/52MOErG/*/i2MOErA/*/i2MOErT/*/i2MOErG/*/i2MO
ErT/*/iMe-dC/*/iMe-dC/*T*/iMe-dC/*/iMe-dC/*A*
/iMe-dC/*A*G*G*/i2MOErT/*/i2MOErG/*/i2MOErA/*
/i2MOErC/*/32MOErA/,
(SEQ ID NO: 332)
/52MOErG/*/i2MOErC/*/i2MOErT/*/i2MOErT/*/i2MO
ErC/*/iMe-dC/*T*G*/iMe-dC/*/iMe-dC/*T*T*A*/iM
e-dC/*T*/i2MOErG/*/i2MOErA/*/i2MOErC/*/i2MOEr
C/*/32MOErT/,
(SEQ ID NO: 333)
/52MOErC/*/i2MOErT/*/i2MOErC/*/i2MOErT/*/i2MO
ErC/*/iMe-dC/*T*T*T*G*T*/iMe-dC/*/iMe-dC/*/iM
e-dC/*T*/i2MOErG/*/i2MOErA/*/i2MOErC/*/i2MOEr
C/*/32MOErA/,
(SEQ ID NO: 334)
/52MOErG/*/i2MOErC/*/i2MOErC/*/i2MOErT/*/i2MO
ErT/*G*/iMe-dC/*/iMe-dC/*T*T*T*T*/iMe-dC/*/iM
e-dC/*T*/i2MOErC/*/i2MOErA/*/i2MOErC/*/i2MOEr
T/*/32MOErC/,
(SEQ ID NO: 335)
/52MOErG/*/i2MOErC/*/i2MOErC/*/i2MOErT/*/i2MO
ErG/*G*T*/iMe-dC/*A*/iMe-dC/*T*A*A*/iMe-dC/*/
iMe-dC/*/i2MOErC/*/i2MOErT/*/i2MOErC/*/i2MOEr
T/*/32MOErC/,
(SEQ ID NO: 336)
/52MOErC/*/i2MOErA/*/i2MOErC/*/i2MOErC/*/i2MO
ErC/*A*/iMe-dC/*/iMe-dC/*T*T*G*G*T*/iMe-dC/*T
*/i2MOErT/*/i2MOErG/*/i2MOErC/*/i2MOErC/*/32M
OErT/,
(SEQ ID NO: 337)
/52MOErC/*/i2MOErA/*/i2MOErC/*/i2MOErA/*/i2MO
ErC/*T*G*A*T*G*T*/iMe-dC/*/iMe-dC/*T*G*/i2MOE
rT/*/i2MOErC/*/i2MOErC/*/i2MOErC/*/32MOErA/,
(SEQ ID NO: 338)
/52MOErC/*/i2MOErA/*/i2MOErC/*/i2MOErA/*/i2MO
ErC/*/iMe-dC/*T*/iMe-dC/*T*G*G*G*T*/iMe-dC/*T
*/i2MOErT/*/i2MOErG/*/i2MOErG/*/i2MOErC/*/32M
OErC/,
(SEQ ID NO: 339)
/52MOErG/*/i2MOErC/*/i2MOErT/*/i2MOErG/*/i2MO
ErC/*/iMe-dC/*/iMe-dC/*A*T*/iMe-dC/*A*/iMe-dC
/*T*/iMe-dC/*/iMe-dC/*/i2MOErC/*/i2MOErA/*/i2
MOErG/*/i2MOErT/*/32MOErT/,
(SEQ ID NO: 340)
/52MOErC/*/i2MOErT/*/i2MOErC/*/i2MOErT/*/i2MO
ErC/*/iMe-dC/*A*T*/iMe-dC/*T*G*/iMe-dC/*/iMe-
dC/*/iMe-dC/*T*/i2MOErG/*/i2MOErG/*/i2MOErC/*
/i2MOErC/*/32MOErC/,
(SEQ ID NO: 341)
/52MOErC/*/i2MOErA/*/i2MOErG/*/i2MOErT/*/i2MO
ErC/*/iMe-dC/*/iMe-dC/*T*/iMe-dC/*T*/iMe-dC/*
/iMe-dC/*T*T*G*/i2MOErT/*/i2MOErC/*/i2MOErT/*
/i2MOErC/*/32MOErT/,
(SEQ ID NO: 342)
/52MOErA/*/i2MOErT/*/i2MOErC/*/i2MOErC/*/i2MO
ErA/*/iMe-dC/*/iMe-dC/*T*G*/iMe-dC/*T*G*/iMe-
dC/*T*/iMe-dC/*/i2MOErC/*/i2MOErT/*/i2MOErG/*
/i2MOErG/*/32MOErG/,
(SEQ ID NO: 343)
/52MOErC/*/i2MOErC/*/i2MOErC/*/i2MOErT/*/i2MO
ErT/*G*T*G*T*/iMe-dC/*T*T*G*T*G*/i2MOErG/*/i2
MOErG/*/i2MOErT/*/i2MOErG/*/32MOErC/,
(SEQ ID NO: 344)
/52MOErG/*/i2MOErC/*/i2MOErC/*/i2MOErC/*/i2MO
ErT/*A*G*G*A*T*T*T*T*/iMe-dC/*/iMe-dC/*/i2MOE
rT/*/i2MOErG/*/i2MOErT/*/i2MOErT/*/32MOErG/,
(SEQ ID NO: 345)
/52MOErG/*/i2MOErC/*/i2MOErC/*/i2MOErT/*/i2MO
ErC/*A*A*/iMe-dC/*T*/iMe-dC/*/iMe-dC/*T*G*/iM
e-dC/*/iMe-dC/*/i2MOErT/*/i2MOErC/*/i2MOErC/*
/i2MOErC/*/32MOErA/,
(SEQ ID NO: 346)
/52MOErA/*/i2MOErG/*/i2MOErA/*/i2MOErC/*/i2MO
ErA/*/iMe-dC/*/iMe-dC/*T*G*G*G*T*A*T*/iMe-dC/
*/i2MOErA/*/i2MOErG/*/i2MOErC/*/i2MOErC/*/32M
OErT/,
(SEQ ID NO: 347)
/52MOErT/*/i2MOErC/*/i2MOErC/*/i2MOErT/*/i2MO
ErT/*/iMe-dC/*T*T*/iMe-dC/*/iMe-dC/*/iMe-dC/*
T*A*T*T*/i2MOErT/*/i2MOErC/*/i2MOErC/*/i2MOEr
C/*/32MOErA/,
(SEQ ID NO: 348)
/52MOErG/*/i2MOErC/*/i2MOErA/*/i2MOErT/*/i2MO
ErC/*A*/iMe-dC/*T*/iMe-dC/*A*/iMe-dC/*T*G*T*/
iMe-dC/*/i2MOErA/*/i2MOErG/*/i2MOErG/*/i2MOEr
T/*/32MOErA/,
(SEQ ID NO: 349)
/52MOErG/*/i2MOErT/*/i2MOErC/*/i2MOErA/*/i2MO
ErG/*T*G*/iMe-dC/*/iMe-dC/*A*/iMe-dC/*A*G*/iM
e-dC/*/iMe-dC/*/i2MOErT/*/i2MOErT/*/i2MOErG/*
/i2MOErT/*/32MOErC/,
(SEQ ID NO: 350)
/52MOErG/*/i2MOErG/*/i2MOErC/*/i2MOErA/*/i2MO
ErC/*/iMe-dC/*T*A*/iMe-dC/*/iMe-dC/*T*T*A*T*G
*/i2MOErC/*/i2MOErA/*/i2MOErC/*/i2MOErC/*/32M
OErC/,
(SEQ ID NO: 351)
/52MOErA/*/i2MOErC/*/i2MOErT/*/i2MOErA/*/i2MO
ErC/*T*G*/iMe-dC/*A*T*/iMe-dC/*/iMe-dC/*/iMe-
dC/*T*/iMe-dC/*/i2MOErA/*/i2MOErG/*/i2MOErC/*
/i2MOErC/*/32MOErC/,
(SEQ ID NO: 352)
/52MOErG/*/i2MOErC/*/i2MOErT/*/i2MOErT/*/i2MO
ErG/*T*/iMe-dC/*T*/iMe-dC/*A*T*/iMe-dC/*/iMe-
dC/*T*G*/i2MOErT/*/i2MOErC/*/i2MOErT/*/i2MOEr
C/*/32MOErT/,
(SEQ ID NO: 353)
/52MOErT/*/i2MOErG/*/i2MOErT/*/i2MOErC/*/i2MO
ErT/*/iMe-dC/*T*G*A*G*/iMe-dC/*T*G*A*/iMe-dC/
*/i2MOErT/*/i2MOErG/*/i2MOErC/*/i2MOErT/*/32M
OErT/,
(SEQ ID NO: 354)
/52MOErG/*/i2MOErG/*/i2MOErG/*/i2MOErC/*/i2MO
ErT/*T*G*A*/iMe-dC/*T*/iMe-dC/*/iMe-dC/*A*/iM
e-dC/*A*/i2MOErC/*/i2MOErT/*/i2MOErC/*/i2MOEr
C/*/32MOErA/,
(SEQ ID NO: 355)
/52MOErG/*/i2MOErG/*/i2MOErC/*/i2MOErA/*/i2MO
ErT/*G*G*/iMe-dC/*A*T*/iMe-dC/*T*/iMe-dC/*A*G
*/i2MOErC/*/i2MOErT/*/i2MOErT/*/i2MOErC/*/32M
OErA/,
(SEQ ID NO: 356)
/52MOErT/*/i2MOErT/*/i2MOErC/*/i2MOErA/*/i2MO
ErG/*G*A*T*/iMe-dC/*A*/iMe-dC/*/iMe-dC/*T*A*G
*/i2MOErC/*/i2MOErT/*/i2MOErG/*/i2MOErG/*/32M
OErT/,
(SEQ ID NO: 357)
/52MOErC/*/i2MOErC/*/i2MOErT/*/i2MOErC/*/i2MO
ErT/*T*T*G*/iMe-dC/*/iMe-dC/*A*T*/iMe-dC/*T*G
*/i2MOErC/*/i2MOErT/*/i2MOErG/*/i2MOErG/*/32M
OErG/,
(SEQ ID NO: 358)
/52MOErG/*/i2MOErA/*/i2MOErG/*/i2MOErT/*/i2MO
ErG/*/iMe-dC/*A*G*T*T*/iMe-dC/*A*/iMe-dC/*T*T
*/i2MOErG/*/i2MOErT/*/i2MOErG/*/i2MOErG/*/32M
OErT/,
(SEQ ID NO: 359)
/52MOErT/*/i2MOErG/*/i2MOErC/*/i2MOErC/*/i2MO
ErC/*A*/iMe-dC/*A*/iMe-dC/*T*/iMe-dC/*T*G*/iM
e-dC/*/iMe-dC/*/i2MOErT/*/i2MOErG/*/i2MOErT/*
/i2MOErC/*/32MOErA/,
(SEQ ID NO: 360)
/52MOErC/*/i2MOErA/*/i2MOErG/*/i2MOErA/*/i2MO
ErG/*G*G*A*G*/iMe-dC/*T*G*/iMe-dC/*T*A*/i2MOE
rG/*/i2MOErT/*/i2MOErC/*/i2MOErA/*/32MOErG/,
(SEQ ID NO: 361)
/52MOErT/*/i2MOErT/*/i2MOErG/*/i2MOErG/*/i2MO
ErC/*A*A*A*G*G*T*G*A*T*G*/i2MOErC/*/i2MOErA/*
/i2MOErG/*/i2MOErG/*/32MOErC/,
(SEQ ID NO: 362)
/52MOErC/*/i2MOErC/*/i2MOErT/*/i2MOErC/*/i2MO
ErC/*A*/iMe-dC/*/iMe-dC/*A*G*T*T*G*G*A*/i2MOE
rA/*/i2MOErG/*/i2MOErA/*/i2MOErC/*/32MOErC/,
(SEQ ID NO: 363)
/52MOErG/*/i2MOErG/*/i2MOErT/*/i2MOErT/*/i2MO
ErC/*T*/iMe-dC/*A*G*/iMe-dC/*/iMe-dC/*A*/iMe-
dC/*/iMe-dC/*A*/i2MOErG/*/i2MOErG/*/i2MOErA/*
/i2MOErT/*/32MOErC/,
(SEQ ID NO: 364)
/52MOErG/*/i2MOErT/*/i2MOErG/*/i2MOErC/*/i2MO
ErT/*/iMe-dC/*/iMe-dC/*A*A*G*A*T*G*/iMe-dC/*/
iMe-dC/*/i2MOErT/*/i2MOErG/*/i2MOErC/*/i
2MOErC/*/32MOErA/,
(SEQ ID NO: 366)
/52MOErG/*/i2MOErT/*/i2MOErG/*/i2MOErC/*/i2MO
ErC/*A*T*T*G*G*T*A*G*A*G*/i2MOErT/*/i2MOErA/*
/i2MOErG/*/i2MOErG/*/32MOErA/,
(SEQ ID NO: 367)
/52MOErG/*/i2MOErT/*/i2MOErG/*/i2MOErA/*/i2MO
ErG/*/iMe-dC/*T*/iMe-dC/*/iMe-dC/*/iMe-dC/*T*
A*A*A*G*/i2MOErA/*/i2MOErA/*/i2MOErC/*/
i2MOErC/*/32MOErT/,
(SEQ ID NO: 368)
/52MOErG/*/i2MOErG/*/i2MOErT/*/i2MOErT/*/i2MO
ErT/*G*/iMe-dC/*/iMe-dC/*A*/iMe-dC/*/iMe-dC/*
A*G*T*A*/i2MOErC/*/i2MOErA/*/i2MOErG/*/i2MOEr
G/*/32MOErG/,
(SEQ ID NO: 369)
/52MOErT/*/i2MOErC/*/i2MOErC/*/i2MOErA/*/i2MO
ErG/*/iMe-dC/*T*T*/iMe-dC/*T*/iMe-dC/*/iMe-dC
/*A*/iMe-dC/*A*/i2MOErT/*/i2MOErC/*/i2MOErA/*
/i2MOErA/*/32MOErT/,
(SEQ ID NO: 370)
/52MOErG/*/i2MOErA/*/i2MOErA/*/i2MOErC/*/i2MO
ErT/*T*G*/iMe-dC/*/iMe-dC/*T*G*/iMe-dC/*T*T*/
iMe-dC/*/i2MOErC/*/i2MOErA/*/i2MOErG/*/i2MOEr
C/*/32MOErT/,
(SEQ ID NO: 371)
/52MOErA/*/i2MOErC/*/i2MOErA/*/i2MOErC/*/i2MO
ErT/*/iMe-dC/*T*G*G*A*T*G*A*G*T*/i2MOErT/*/i2
MOErT/*/i2MOErG/*/i2MOErT/*/32MOErC/,
(SEQ ID NO: 372)
/52MOErG/*/i2MOErG/*/i2MOErG/*/i2MOErC/*/i2MO
ErA/*/iMe-dC/*/iMe-dC/*/iMe-dC/*A*T*G*A*/iMe-
dC/*A*/iMe-dC/*/i2MOErT/*/i2MOErC/*/i2MOErT/*
/i2MOErG/*/32MOErG/,
(SEQ ID NO: 373)
/52MOErA/*/i2MOErC/*/i2MOErT/*/i2MOErT/*/i2MO
ErG/*T*/iMe-dC/*/iMe-dC/*A*G*T*G*/iMe-dC/*T*/
iMe-dC/*/i2MOErC/*/i2MOErA/*/i2MOErG/*/i2MOEr
G/*/32MOErT/,
(SEQ ID NO: 374)
/52MOErC/*/i2MOErC/*/i2MOErC/*/i2MOErA/*/i2MO
ErA/*T*/iMe-dC/*/iMe-dC/*T*T*G*/iMe-dC/*A*G*T
*/i2MOErC/*/i2MOErA/*/i2MOErT/*/i2MOErG/*/32M
OErG/,
(SEQ ID NO: 375)
/52MOErA/*/i2MOErG/*/i2MOErC/*/i2MOErA/*/i2MO
ErC/*A*G*/iMe-dC/*/iMe-dC/*T*G*/iMe-dC/*A*T*G
*/i2MOErT/*/i2MOErC/*/i2MOErC/*/i2MOErT/*/32M
OErC/,
(SEQ ID NO: 376)
/52MOErC/*/i2MOErA/*/i2MOErA/*/i2MOErA/*/i2MO
ErC/*T*G*G*T*G*T*/iMe-dC/*A*G*A*/i2MOErG/*/i2
MOErC/*/i2MOErC/*/i2MOErT/*/32MOErG/,
(SEQ ID NO: 377)
/52MOErG/*/i2MOErC/*/i2MOErA/*/i2MOErG/*/i2MO
ErC/*A*/iMe-dC/*/iMe-dC/*/iMe-dC/*T*/iMe-dC/*
/iMe-dC/*A*A*A*/i2MOErC/*/i2MOErT/*/i2MOErG/*
/i2MOErG/*/32MOErT/,
(SEQ ID NO: 378)
/52MOErT/*/i2MOErG/*/i2MOErG/*/i2MOErT/*/i2MO
ErT/*A*G*G*T*T*G*G*A*/iMe-dC/*/iMe-dC/*/i2MOE
rC/*/i2MOErA/*/i2MOErT/*/i2MOErG/*/32MOErG/,
(SEQ ID NO: 379)
/52MOErG/*/i2MOErC/*/i2MOErC/*/i2MOErC/*/i2MO
ErA/*G*G*T*T*G*T*/iMe-dC/*T*/iMe-dC/*A*/i2MOE
rG/*/i2MOErC/*/i2MOErC/*/i2MOErC/*/32MOErA/,
(SEQ ID NO: 380)
/52MOErT/*/i2MOErC/*/i2MOErC/*/i2MOErC/*/i2MO
ErT/*/iMe-dC/*T*/iMe-dC/*/iMe-dC/*A*G*A*T*A*/
iMe-dC/*/i2MOErT/*/i2MOErG/*/i2MOErA/*/i2MOEr
G/*/32MOErG/,
(SEQ ID NO: 381)
/52MOErA/*/i2MOErC/*/i2MOErA/*/i2MOErG/*/i2MO
ErA/*/iMe-dC/*A*A*/iMe-dC/*/iMe-dC/*/iMe-dC/*
A*A*T*G*/i2MOErG/*/i2MOErC/*/i2MOErA/*/i2MOEr
G/*/32MOErG/,
(SEQ ID NO: 383)
/52MOErC/*/i2MOErC/*/i2MOErT/*/i2MOErT/*/i2MO
ErA/*A*T*T*/iMe-dC/*/iMe-dC/*T*G*T*/iMe-dC/*T
*/i2MOErG/*/i2MOErA/*/i2MOErG/*/i2MOErG/*/32M
OErC/,
(SEQ ID NO: 384)
/52MOErC/*/i2MOErA/*/i2MOErG/*/i2MOErA/*/i2MO
ErA/*T*A*/iMe-dC/*A*G*T*G*/iMe-dC/*/iMe-dC/*/
iMe-dC/*/i2MOErA/*/i2MOErG/*/i2MOErG/*/i2MOEr
C/*/32MOErC/,
(SEQ ID NO: 385)
/52MOErC/*/i2MOErC/*/i2MOErC/*/i2MOErA/*/i2MO
ErG/*G*/iMe-dC/*/iMe-dC/*/iMe-dC/*T*T*G*/iMe-
dC/*T*/iMe-dC/*/i2MOErA/*/i2MOErG/*/i2MOErA/*
/i2MOErA/*/32MOErT/,
(SEQ ID NO: 386)
/52MOErG/*/i2MOErC/*/i2MOErA/*/i2MOErC/*/i2MO
ErT/*/iMe-dC/*A*T*/iMe-dC/*/iMe-dC/*/iMe-dC/*
T*G*G*/iMe-dC/*/i2MOErT/*/i2MOErG/*/i2MOErG/*
/i2MOErC/*/32MOErT/,
(SEQ ID NO: 387)
/52MOErG/*/i2MOErA/*/i2MOErT/*/i2MOErT/*/i2MO
ErA/*/iMe-dC/*A*G*G*G*/iMe-dC/*A*A*G*G*/i2MOE
rC/*/i2MOErC/*/i2MOErA/*/i2MOErC/*/32MOErA/,
(SEQ ID NO: 388)
/52MOErG/*/i2MOErC/*/i2MOErC/*/i2MOErC/*/i2MO
ErT/*G*G*A*T*G**G*G*/iMe-dC/*A*/i2MOErA/*/i2M
OErA/*/i2MOErA/*/i2MOErG/*/32MOErA/,
(SEQ ID NO: 389)
/52MOErA/*/i2MOErA/*/i2MOErG/*/i2MOErG/*/i2MO
ErA/*A*G*T*/iMe-dC/*A*G*A*G*G*G*/i2MOErA/*/i2
MOErG/*/i2MOErG/*/i2MOErG/*/32MOErC/,
(SEQ ID NO: 390)
/52MOErC/*/i2MOErA/*/i2MOErG/*/i2MOErG/*/i2MO
ErC/*/iMe-dC/*/iMe-dC/*A*A*A*/iMe-dC/*A*G*G*A
*/i2MOErG/*/i2MOErG/*/i2MOErC/*/i2MOErT/*/32M
OErC/,
(SEQ ID NO: 391)
/52MOErA/*/i2MOErT/*/i2MOErG/*/i2MOErC/*/i2MO
ErC/*/iMe-dC/*A*G*A*/iMe-dC/*/iMe-dC/*/iMe-dC
/*A*G*G*/i2MOErC/*/i2MOErC/*/i2MOErC/*/i2MOEr
A/*/32MOErA/,
(SEQ ID NO: 392)
/52MOErC/*/i2MOErT/*/i2MOErG/*/i2MOErA/*/i2MO
ErG/*G*/iMe-dC/*A*/iMe-dC/*A*G*/iMe-dC/*A*/iM
e-dC/*/iMe-dC/*/i2MOErA/*/i2MOErA/*/i2MOErG/*
/i2MOErG/*/32MOErC/,
(SEQ ID NO: 393)
/52MOErG/*/i2MOErC/*/i2MOErC/*/i2MOErA/*/i2MO
ErG/*A*/iMe-dC/*/iMe-dC/*A*G*G*A*A*G*G*/i2MOE
rA/*/i2MOErG/*/i2MOErC/*/i2MOErC/*/32MOErT/,
(SEQ ID NO: 394)
/52MOErT/*/i2MOErC/*/i2MOErA/*/i2MOErG/*/i2MO
ErG/*A*/iMe-dC/*T*T*T*G*/iMe-dC/*/iMe-dC/*T*/
iMe-dC/*/i2MOErT/*/i2MOErT/*/i2MOErT/*/i2MOEr
C/*/32MOErC/,
(SEQ ID NO: 395)
/52MOErG/*/i2MOErC/*/i2MOErT/*/i2MOErT/*/i2MO
ErT/*A*G*A*G*A*T*T*T*G*/iMe-dC/*/i2MOErT/*/i2
MOErA/*/i2MOErC/*/i2MOErC/*/32MOErC/,
(SEQ ID NO: 396)
/52MOErG/*/i2MOErC/*/i2MOErC/*/i2MOErC/*/i2MO
ErA/*G*/iMe-dC/*/iMe-dC/*T*/iMe-dC/*A*G*A*A*T
*/i2MOErG/*/i2MOErA/*/i2MOErT/*/i2MOErT/*/32M
OErC/,
(SEQ ID NO: 397)
/52MOErC/*/i2MOErC/*/i2MOErT/*/i2MOErC/*/i2MO
ErT/*G*A*A*/iMe-dC/*/iMe-dC/*/iMe-dC/*A*G*T*G
*/i2MOErG/*/i2MOErA/*/i2MOErG/*/i2MOErG/*/32M
OErA/,
(SEQ ID NO: 398)
/52MOErG/*/i2MOErC/*/i2MOErC/*/i2MOErT/*/i2MO
ErG/*G*G*T*T*T*A*T*T*G*G*/i2MOErA/*/i2MOErG/*
/i2MOErG/*/i2MOErG/*/32MOErT/,
(SEQ ID NO: 399)
/52MOErG/*/i2MOErC/*/i2MOErC/*/i2MOErA/*/i2MO
ErG/*/iMe-dC/*A*/iMe-dC/*A*G*/iMe-dC/*/iMe-
dC/*A*A*G*/i2MOErA/*/i2MOErG/*/i2MOE
rT/*/i2MOErG/*/32MOErG/,
or
(SEQ ID NO: 400)
/52MOErG/*/i2MOErG/*/i2MOErG/*/i2MOErA/*/i2MO
ErG/*T*G*G*A*A*G*G*A*A*G*/i2MOErG/*/i2MOErA/*/
i2MOErG/*/i2MOErC/*/32MOErC/,

or a salt thereof,
wherein:
   * is —O—P(O)(SH)—O—;

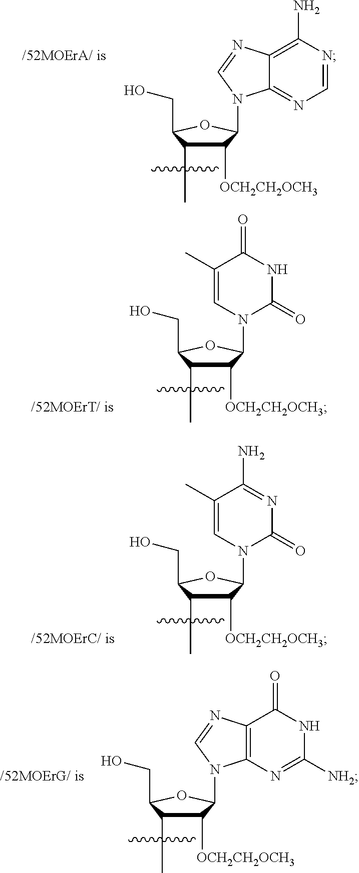

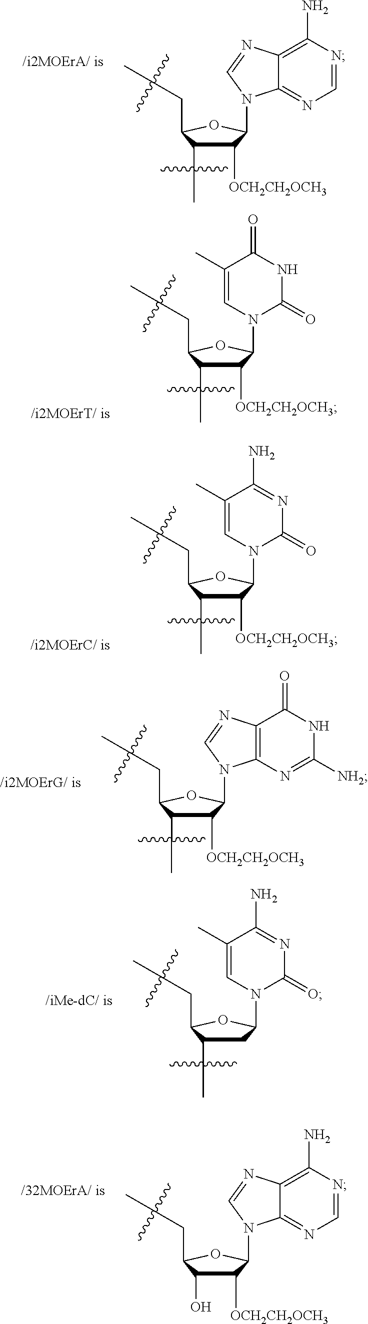

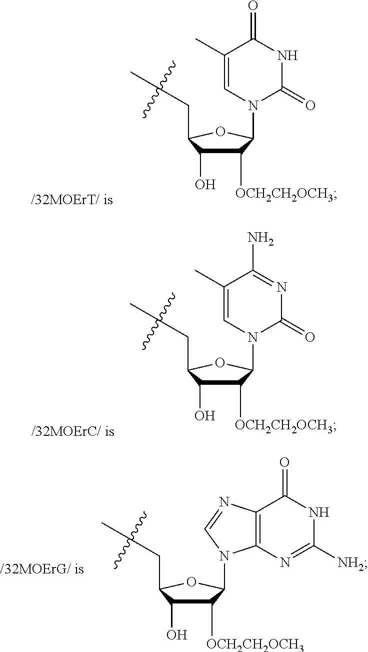

and
each of A, T and G is independently deoxyadenosine, thymidine, and deoxyguanosine, respectively.
194. The oligonucleotide of Embodiment 193, wherein the oligonucleotide is /52MOErG/*/i2MOErT/*/i2MOErC/*/i2MOErT/*/i2MOErC/*/iMe-dC/*A*G*A*A*/iMe-dC/*T*G*A*G*/i2MOErC/*/i2MOErA/*/i2MOErG/*/i2MOErG/*/32MOErG/ (SEQ ID NO: 382) or a salt thereof.
195. The oligonucleotide of Embodiment 193, wherein the oligonucleotide is /52MOErC/*/i2MOErC/*/i2MOErT/*/i2MOErT/*/i2MOErG/*/iMe-dC/*A*G*G*/iMe-dC/*T*/iMe-dC/*T*T*G*/i2MOErA/*/i2MOErT/*/i2MOErG/*/i2MOErG/*/32MOErC/ (SEQ ID NO: 365) or a salt thereof.
196. The oligonucleotide of Embodiment 193, wherein the oligonucleotide is /52MOErG/*/i2MOErC/*/i2MOErT/*/i2MOErG/*/i2MOErG/*/iMe-dC/*T*G*T*A*/iMe-dC/*T*/iMe-dC/*A*/iMe-dC/*/i2MOErT/*/i2MOErC/*/i2MOErT/*/i2MOErC/*/32MOErC/ (SEQ ID NO: 325) or a salt thereof.
197. The oligonucleotide of Embodiment 193, wherein the oligonucleotide is /52MOErT/*/i2MOErC/*/i2MOErA/*/i2MOErG/*/i2MOErG/*A*/iMe-dC/*T*T*T*G*/iMe-dC/*/iMe-dC/*T*/iMe-dC/*/i2MOErT/*/i2MOErT/*/i2MOErT/*/i2MOErC/*/32MOErC/ (SEQ ID NO: 394) or a salt thereof.
198. The oligonucleotide of Embodiment 193, wherein the oligonucleotide is /52MOErG/*/i2MOErG/*/i2MOErG/*/i2MOErA/*/i2MOErG/*T*G*G*A*A*G*G*A*A*G*/i2MOErG/*/i2MOErA/*/i2MOErG/*/i2MOErC/*/32MOErC/ (SEQ ID NO: 400) or a salt thereof.
199. The oligonucleotide of Embodiment 193, wherein the oligonucleotide is /52MOErG/*/i2MOErC/*/i2MOErT/*/i2MOErT/*/i2MOErT/*A*G*A*G*A*T*T*T*G*/iMe-dC/*/i2MOErT/*/i2MOErA/*/i2MOErC/*/i2MOErC/*/32MOErC/ (SEQ ID NO: 395) or a salt thereof.
200. The oligonucleotide of Embodiment 193, wherein the oligonucleotide is /52MOErG/*/i2MOErC/*/i2MOErA/*/i2MOErT/*/i2MOErC/*A*/iMe-dC/*T*/iMe-dC/*A*/iMe-dC/*T*G*T*/iMe-dC/*/i2MOErA/*/i2MOErG/*/i2MOErG/*/i2MOErT/*/32MOErA/ (SEQ ID NO: 348) or a salt thereof.
201. The oligonucleotide of Embodiment 193, wherein the oligonucleotide is /52MOErG/*/i2MOErC/*/i2MOErC/*/i2MOErC/*/i2MOErT/*A*G*G*A*T*T*T*T*/iMe-dC/*/iMe-dC/*/i2MOErT/*/i2MOErG/*/i2MOErT/*/i2MOErT/*/32MOErG/ (SEQ ID NO: 344) or a salt thereof.
202. The oligonucleotide of Embodiment 193, wherein the oligonucleotide is /52MOErG/*/i2MOErT/*/i2MOErG/*/i2MOErC/*/i2MOErC/*A*T*T*G*G*T*A*G*A*G*/i2MOErT/* /i2MOErA/*/i2MOErG/*/i2MOErG/*/32MOErA/ (SEQ ID NO: 366) or a salt thereof.
203. The oligonucleotide of Embodiment 193, wherein the oligonucleotide is /52MOErC/*/i2MOErT/*/i2MOErG/*/i2MOErT/*/i2MOErG/*A*/iMe-dC/*/iMe-dC/*T*A*G*G*/iMe-dC/*T*/iMe-dC/*/i2MOErC/*/i2MOErT/*/i2MOErT/*/i2MOErG/*/32MOErA/ (SEQ ID NO: 328) or a salt thereof.
204. The oligonucleotide of Embodiment 193, wherein the oligonucleotide is /52MOErA/*/i2MOErG/*/i2MOErA/*/i2MOErC/*/i2MOErA/*/iMe-dC/*/iMe-dC/*T*G*G*G*T*A*T*/iMe-dC/*/i2MOErA/*/i2MOErG/*/i2MOErC/*/i2MOErC/*/32MOErT/(SEQ ID NO: 346) or a salt thereof.
205. The oligonucleotide of Embodiment 193, wherein the oligonucleotide is /52MOErG/*/i2MOErC/*/i2MOErC/*/i2MOErC/*/i2MOErA/*G*G*T*T*G*T*/iMe-dC/*T*/iMe-dC/*A*/i2MOErG/*/i2MOErC/*/i2MOErC/*/i2MOErC/*/32MOErA/ (SEQ ID NO: 379) or a salt thereof.
206. The oligonucleotide of Embodiment 193, wherein the oligonucleotide is /52MOErG/*/i2MOErC/*/i2MOErT/*/i2MOErT/*/i2MOErG/*T*/iMe-dC/*T*/iMe-dC/*A*T*/iMe-dC/*/iMe-dC/*T*G*/i2MOErT/*/i2MOErC/*/i2MOErT/*/i2MOErC/*/32MOErT/ (SEQ ID NO: 352) or a salt thereof.
207. The oligonucleotide of Embodiment 193, wherein the oligonucleotide is /52MOErG/*/i2MOErG/*/i2MOErT/*/i2MOErT/*/i2MOErC/*T*/iMe-dC/*A*G*/iMe-dC/*/iMe-dC/*A*/iMe-dC/*/iMe-dC/*A*/i2MOErG/*/i2MOErG/*/i2MOErA/*/i2MOErT/*/32MOErC/ (SEQ ID NO: 363) or a salt thereof.
208. The oligonucleotide of Embodiment 193, wherein the oligonucleotide is /52MOErC/*/i2MOErA/*/i2MOErA/*/i2MOErA/*/i2MOErC/*T*G*G*T*G*T*/iMe-dC/*A*G*A*/i2MOErG/*/i2MOErC/*/i2MOErC/*/i2MOErT/*/32MOErG/ (SEQ ID NO: 376) or a salt thereof.
209. The oligonucleotide of Embodiment 193, wherein the oligonucleotide is /52MOErG/*/i2MOErC/*/i2MOErA/*/i2MOErG/*/i2MOErC/*A*/iMe-dC/*/iMe-dC/*/iMe-dC/*T*/iMe-dC/*/iMe-dC/*A*A*A*/i2MOErC/*/i2MOErT/*/i2MOErG/*/i2MOErG/*/32MOErT/(SEQ ID NO: 377) or a salt thereof.
210. The oligonucleotide of Embodiment 193, wherein the oligonucleotide is /52MOErT/*/i2MOErG/*/i2MOErT/*/i2MOErC/*/i2MOErT/*/iMe-dC/*T*G*A*G*/iMe-dC/*T*G*A*/iMe-dC/*/i2MOErT/*/i2MOErG/*/i2MOErC/*/i2MOErT/*/32MOErT/ (SEQ ID NO: 353) or a salt thereof.
211. The oligonucleotide of Embodiment 193, wherein the oligonucleotide is /52MOErC/*/i2MOErC/*/i2MOErA/*/i2MOErC/*/i2MOErT/*A*G*/iMe-dC/*/iMe-dC/*/iMe-dC/*T*G*G*G*A*/i2MOErG/*/i2MOErC/*/i2MOErA/*/i2MOErA/*/32MOErA/ (SEQ ID NO: 321) or a salt thereof.
212. The oligonucleotide of Embodiment 193, wherein the oligonucleotide is /52MOErG/*/i2MOErC/*/i2MOErC/*/i2MOErA/*/i2MOErT/*/iMe-dC/*T*/iMe-dC/*/iMe-dC/*A*T*/iMe-dC/*/iMe-dC/*A*T*/i2MOErA/*/i2MOErG/*/i2MOErA/*/i2MOErG/*/32MOErC/(SEQ ID NO: 322) or a salt thereof.
213. The oligonucleotide of Embodiment 193, wherein the oligonucleotide is /52MOErA/*/i2MOErG/*/i2MOErG/*/i2MOErA/*/i2MOErG/*A*G*/iMe-dC/*T*G*T*G*G*G*/iMe-dC/*/i2MOErT/*/i2MOErT/*/i2MOErG/*/i2MOErG/*/32MOErG/ (SEQ ID NO: 323) or a salt thereof.
214. The oligonucleotide of Embodiment 193, wherein the oligonucleotide is /52MOErC/*/i2MOErA/*/i2MOErC/*/i2MOErC/*/i2MOErC/*A*T*G*/iMe-dC/*/iMe-dC/*T*/iMe-dC/*/iMe-dC/*/iMe-dC/*A*/i2MOErG/*/i2MOErC/*/i2MOErA/*/i2MOErG/*/32MOErA/ (SEQ ID NO: 324) or a salt thereof.
215. The oligonucleotide of Embodiment 193, wherein the oligonucleotide is /52MOErG/*/i2MOErT/*/i2MOErG/*/i2MOErC/*/i2MOErT/*/iMe-dC/*T*G*T*/iMe-dC/*/iMe-dC/*T*T*G*G*/i2MOErT/*/i2MOErC/*/i2MOErC/*/i2MOErT/*/32MOErG/ (SEQ ID NO: 326) or a salt thereof.
216. The oligonucleotide of Embodiment 193, wherein the oligonucleotide is /52MOErC/*/i2MOErC/*/i2MOErC/*/i2MOErA/*/i2MOErT/*T*/iMe-dC/*T*/iMe-dC/*A*T*G*/iMe-dC/*A*G*/i2MOErC/*/i2MOErC/*/i2MOErT/*/i2MOErA/*/32MOErC/ (SEQ ID NO: 327) or a salt thereof.
217. The oligonucleotide of Embodiment 193, wherein the oligonucleotide is /52MOErG/*/i2MOErG/*/i2MOErT/*/i2MOErC/*/i2MOErT/*G*A*G*A*G*G*/iMe-dC/*T*G*T*/i2MOErG/*/i2MOErG/*/i2MOErG/*/i2MOErT/*/32MOErC/ (SEQ ID NO: 329) or a salt thereof.
218. The oligonucleotide of Embodiment 193, wherein the oligonucleotide is /52MOErG/*/i2MOErC/*/i2MOErT/*/i2MOErC/*/i2MOErC/*/iMe-dC/*A*G*T*T*/iMe-dC/*T*T*/iMe-dC/*T*/i2MOErG/*/i2MOErT/*/i2MOErG/*/i2MOErG/*/32MOErT/ (SEQ ID NO: 330) or a salt thereof.
219. The oligonucleotide of Embodiment 193, wherein the oligonucleotide is /52MOErG/*/i2MOErA/*/i2MOErT/*/i2MOErG/*/i2MOErT/*/iMe-dC/*/iMe-dC/*T*/iMe-dC/*/iMe-dC/*A*/iMe-dC/*A*G*G*/i2MOErT/*/i2MOErG/*/i2MOErA/*/i2MOErC/*/32MOErA/ (SEQ ID NO: 331) or a salt thereof
220. The oligonucleotide of Embodiment 193, wherein the oligonucleotide is /52MOErG/*/i2MOErC/*/i2MOErT/*/i2MOErT/*/i2MOErC/*/iMe-dC/*T*G*/iMe-dC/*/iMe-dC/*T*T*A*/iMe-dC/*T*/i2MOErG/*/i2MOErA/*/i2MOErC/*/i2MOErC/*/32MOErT/ (SEQ ID NO: 332) or a salt thereof.
221. The oligonucleotide of Embodiment 193, wherein the oligonucleotide is /52MOErC/*/i2MOErT/*/i2MOErC/*/i2MOErT/*/i2MOErC/*/iMe-dC/*T*T*T*G*T*/iMe-dC/*/iMe-dC/*/iMe-dC/*T*/i2MOErG/*/i2MOErA/*/i2MOErC/*/i2MOErC/*/32MOErA/ (SEQ ID NO: 333) or a salt thereof.
222. The oligonucleotide of Embodiment 193, wherein the oligonucleotide is /52MOErG/*/i2MOErC/*/i2MOErC/*/i2MOErT/*/i2MOErT/*G*/iMe-dC/*/iMe-dC/*T*T*T*T*/iMe-dC/*/iMe-dC/*T*/i2MOErC/*/i2MOErA/*/i2MOErC/*/i2MOErT/*/32MOErC/ (SEQ ID NO: 334) or a salt thereof.
223. The oligonucleotide of Embodiment 193, wherein the oligonucleotide is /52MOErG/*/i2MOErC/*/i2MOErC/*/i2MOErT/*/i2MOErG/*G*T*/iMe-dC/*A*/iMe-dC/*T*A*A*/iMe-dC/*/iMe-dC/*/i2MOErC/*/i2MOErT/*/i2MOErC/*/i2MOErT/*/32MOErC/ (SEQ ID NO: 335) or a salt thereof.
224. The oligonucleotide of Embodiment 193, wherein the oligonucleotide is /52MOErC/*/i2MOErA/*/i2MOErC/*/i2MOErC/*/i2MOErC/*A*/iMe-dC/*/iMe-dC/*T*T*G*G*T*/iMe-dC/*T*/i2MOErT/*/i2MOErG/*/i2MOErC/*/i2MOErC/*/32MOErT/ (SEQ ID NO: 336) or a salt thereof.
225. The oligonucleotide of Embodiment 193, wherein the oligonucleotide is /52MOErC/*/i2MOErA/*/i2MOErC/*/i2MOErA/*/i2MOErC/*T*G*A*T*G*T*/iMe-dC/*/iMe-dC/*T*G*/i2MOErT/*/i2MOErC/*/i2MOErC/*/i2MOErC/*/32MOErA/ (SEQ ID NO: 337) or a salt thereof.
226. The oligonucleotide of Embodiment 193, wherein the oligonucleotide is /52MOErC/*/i2MOErA/*/i2MOErC/*/i2MOErA/*/i2MOErC/*/iMe-dC/*T*/iMe-dC/*T*G*G*G*T*/iMe-dC/*T*/i2MOErT/*/i2MOErG/*/i2MOErG/*/i2MOErC/*/32MOErC/ (SEQ ID NO: 338) or a salt thereof.
227. The oligonucleotide of Embodiment 193, wherein the oligonucleotide is /52MOErG/*/i2MOErC/*/i2MOErT/*/i2MOErG/*/i2MOErC/*/iMe-dC/*/iMe-dC/*A*T*/iMe-dC/*A*/iMe-dC/*T*/iMe-dC/*/iMe-dC/*/i2MOErC/*/i2MOErA/*/i2MOErG/*/i2MOErT/*/32MOErT/(SEQ ID NO: 339) or a salt thereof.
228. The oligonucleotide of Embodiment 193, wherein the oligonucleotide is /52MOErC/*/i2MOErT/*/i2MOErC/*/i2MOErT/*/i2MOErC/*/iMe-dC/*A*T*/iMe-dC/*T*G*/iMe-dC/*/iMe-dC/*/iMe-dC/*T*/i2MOErG/*/i2MOErG/*/i2MOErC/*/i2MOErC/*/32MOErC/ (SEQ ID NO: 340) or a salt thereof.
229. The oligonucleotide of Embodiment 193, wherein the oligonucleotide is /52MOErC/*/i2MOErA/*/i2MOErG/*/i2MOErT/*/i2MOErC/*/iMe-dC/*/iMe-dC/*T*/iMe-dC/*T*/iMe-dC/*/iMe-dC/*T*T*G*/i2MOErT/*/i2MOErC/*/i2MOErT/*/i2MOErC/*/32MOErT/ (SEQ ID NO: 341) or a salt thereof.
230. The oligonucleotide of Embodiment 193, wherein the oligonucleotide is /52MOErA/*/i2MOErT/*/i2MOErC/*/i2MOErC/*/i2MOErA/*/iMe-dC/*/iMe-dC/*T*G*/iMe-dC/*T*G*/iMe-dC/*T*/iMe-dC/*/i2MOErC/*/i2MOErT/*/i2MOErG/*/i2MOErG/*/32MOErG/(SEQ ID NO: 342) or a salt thereof.
231. The oligonucleotide of Embodiment 193, wherein the oligonucleotide is /52MOErC/*/i2MOErC/*/i2MOErC/*/i2MOErT/*/i2MOErT/*G*T*G*T*/iMe-dC/*T*T*G*T*G*/i2MOErG/*/i2MOErG/*/i2MOErT/*/i2MOErG/*/32MOErC/ (SEQ ID NO: 343) or a salt thereof.
232. The oligonucleotide of Embodiment 193, wherein the oligonucleotide is /52MOErG/*/i2MOErC/*/i2MOErC/*/i2MOErT/*/i2MOErC/*A*A*/iMe-dC/*T*/iMe-dC/*/iMe-dC/*T*G*/iMe-dC/*/iMe-dC/*/i2MOErT/*/i2MOErC/*/i2MOErC/*/i2MOErC/*/32MOErA/ (SEQ ID NO: 345) or a salt thereof.
233. The oligonucleotide of Embodiment 193, wherein the oligonucleotide is /52MOErT/*/i2MOErC/*/i2MOErC/*/i2MOErT/*/i2MOErT/*/iMe-dC/*T*T*/iMe-dC/*/iMe-dC/*/iMe-dC/*T*A*T*T*/i2MOErT/*/i2MOErC/*/i2MOErC/*/i2MOErC/*/32MOErA/ (SEQ ID NO: 347) or a salt thereof.
234. The oligonucleotide of Embodiment 193, wherein the oligonucleotide is /52MOErG/*/i2MOErT/*/i2MOErC/*/i2MOErA/*/i2MOErG/*T*G*/iMe-dC/*/iMe-dC/*A*/iMe-dC/*A*G*/iMe-dC/*/iMe-dC/*/i2MOErT/*/i2MOErT/*/i2MOErG/*/i2MOErT/*/32MOErC/ (SEQ ID NO: 349) or a salt thereof.
235. The oligonucleotide of Embodiment 193, wherein the oligonucleotide is /52MOErG/*/i2MOErG/*/i2MOErC/*/i2MOErA/*/i2MOErC/*/iMe-dC/*T*A*/iMe-dC/*/iMe-dC/*T*T*A*T*G*/i2MOErC/*/i2MOErA/*/i2MOErC/*/i2MOErC/*/32MOErC/ (SEQ ID NO: 350) or a salt thereof.
236. The oligonucleotide of Embodiment 193, wherein the oligonucleotide is /52MOErA/*/i2MOErC/*/i2MOErT/*/i2MOErA/*/i2MOErC/*T*G*/iMe-dC/*A*T*/iMe-dC/*/iMe-dC/*/iMe-dC/*T*/iMe-dC/*/i2MOErA/*/i2MOErG/*/i2MOErC/*/i2MOErC/*/32MOErC/ (SEQ ID NO: 351) or a salt thereof 237. The oligonucleotide of Embodiment 193, wherein the oligonucleotide is /52MOErG/*/i2MOErG/*/i2MOErG/*/i2MOErC/*/i2MOErT/*T*G*A*/iMe-dC/*T*/iMe-dC/*/iMe-dC/*A*/iMe-dC/*A*/i2MOErC/*/i2MOErT/*/i2MOErC/*/i2MOErC/*/32MOErA/ (SEQ ID NO: 354) or a salt thereof.
238. The oligonucleotide of Embodiment 193, wherein the oligonucleotide is /52MOErG/*/i2MOErG/*/i2MOErC/*/i2MOErA/*/i2MOErT/*G*G*/iMe-dC/*A*T*/iMe-dC/*T*/iMe-dC/*A*G*/i2MOErC/*/i2MOErT/*/i2MOErT/*/i2MOErC/*/32MOErA/ (SEQ ID NO: 355) or a salt thereof.
239. The oligonucleotide of Embodiment 193, wherein the oligonucleotide is /52MOErT/*/i2MOErT/*/i2MOErC/*/i2MOErA/*/i2MOErG/*G*A*T*/iMe-dC/*A*/iMe-dC/*/iMe-dC/*T*A*G*/i2MOErC/*/i2MOErT/*/i2MOErG/*/i2MOErG/*/32MOErT/ (SEQ ID NO: 356) or a salt thereof.
240. The oligonucleotide of Embodiment 193, wherein the oligonucleotide is /52MOErC/*/i2MOErC/*/i2MOErT/*/i2MOErC/*/i2MOErT/*T*T*G*/iMe-dC/*/iMe-dC/*A*T*/iMe-dC/*T*G*/i2MOErC/*/i2MOErT/*/i2MOErG/*/i2MOErG/*/32MOErG/ (SEQ ID NO: 357) or a salt thereof.
241. The oligonucleotide of Embodiment 193, wherein the oligonucleotide is /52MOErG/*/i2MOErA/*/i2MOErG/*/i2MOErT/*/i2MOErG/*/iMe-dC/*A*G*T*T*/iMe-dC/*A*/iMe-dC/*T*T*/i2MOErG/*/i2MOErT/*/i2MOErG/*/i2MOErG/*/32MOErT/ (SEQ ID NO: 358) or a salt thereof.
242. The oligonucleotide of Embodiment 193, wherein the oligonucleotide is /52MOErT/*/i2MOErG/*/i2MOErC/*/i2MOErC/*/i2MOErC/*A*/iMe-dC/*A*/iMe-dC/*T*/iMe-dC/*T*G*/iMe-dC/*/iMe-dC/*/i2MOErT/*/i2MOErG/*/i2MOErT/*/i2MOErC/*/32MOErA/ (SEQ ID NO: 359) or a salt thereof.
243. The oligonucleotide of Embodiment 193, wherein the oligonucleotide is /52MOErC/*/i2MOErA/*/i2MOErG/*/i2MOErA/*/i2MOErG/*G*G*A*G*/iMe-dC/*T*G*/iMe-dC/*T*A*/i2MOErG/*/i2MOErT/*/i2MOErC/*/i2MOErA/*/32MOErG/ (SEQ ID NO: 360) or a salt thereof.
244. The oligonucleotide of Embodiment 193, wherein the oligonucleotide is /52MOErT/*/i2MOErT/*/i2MOErG/*/i2MOErG/*/i2MOErC/*A*A*A*G*G*T*G*A*T*G*/i2MOErC/*/i2MOErA/*/i2MOErG/*/i2MOErG/*/32MOErC/ (SEQ ID NO: 361) or a salt thereof.
245. The oligonucleotide of Embodiment 193, wherein the oligonucleotide is /52MOErC/*/i2MOErC/*/i2MOErT/*/i2MOErC/*/i2MOErC/*A*/iMe-dC/*/iMe-dC/*A*G*T*T*G*G*A*/i2MOErA/*/i2MOErG/*/i2MOErA/*/i2MOErC/*/32MOErC/ (SEQ ID NO: 362) or a salt thereof.
246. The oligonucleotide of Embodiment 193, wherein the oligonucleotide is /52MOErG/*/i2MOErT/*/i2MOErG/*/i2MOErC/*/i2MOErT/*/iMe-dC/*/iMe-dC/*A*A*G*A*T*G*/iMe-dC/*/iMe-dC/*/i2MOErT/*/i2MOErG/*/i2MOErC/*/i2MOErC/*/32MOErA/ (SEQ ID NO: 364) or a salt thereof.
247. The oligonucleotide of Embodiment 193, wherein the oligonucleotide is /52MOErG/*/i2MOErT/*/i2MOErG/*/i2MOErA/*/i2MOErG/*/iMe-dC/*T*/iMe-dC/*/iMe-dC/*/iMe-dC/*T*A*A*A*G*/i2MOErA/*/i2MOErA/*/i2MOErC/*/i2MOErC/*/32MOErT/ (SEQ ID NO: 367) or a salt thereof.
248. The oligonucleotide of Embodiment 193, wherein the oligonucleotide is /52MOErG/*/i2MOErG/*/i2MOErT/*/i2MOErT/*/i2MOErT/*G*/iMe-dC/*/iMe-dC/*A*/iMe-dC/*/iMe-dC/*A*G*T*A*/i2MOErC/*/i2MOErA/*/i2MOErG/*/i2MOErG/*/32MOErG/ (SEQ ID NO: 368) or a salt thereof.
249. The oligonucleotide of Embodiment 193, wherein the oligonucleotide is /52MOErT/*/i2MOErC/*/i2MOErC/*/i2MOErA/*/i2MOErG/*/iMe-dC/*T*T*/iMe-dC/*T*/iMe-dC/*/iMe-dC/*A*/iMe-dC/*A*/i2MOErT/*/i2MOErC/*/i2MOErA/*/i2MOErA/*/32MOErT/ (SEQ ID NO: 369) or a salt thereof.
250. The oligonucleotide of Embodiment 193, wherein the oligonucleotide is /52MOErG/*/i2MOErA/*/i2MOErA/*/i2MOErC/*/i2MOErT/*T*G*/iMe-dC/*/iMe-dC/*T*G*/iMe-dC/*T*T*/iMe-dC/*/i2MOErC/*/i2MOErA/*/i2MOErG/*/i2MOErC/*/32MOErT/ (SEQ ID NO: 370) or a salt thereof.
251. The oligonucleotide of Embodiment 193, wherein the oligonucleotide is /52MOErA/*/i2MOErC/*/i2MOErA/*/i2MOErC/*/i2MOErT/*/iMe-dC/*T*G*G*A*T*G*A*G*T*/i2MOErT/*/i2MOErT/*/i2MOErG/*/i2MOErT/*/32MOErC/ (SEQ ID NO: 371) or a salt thereof.
252. The oligonucleotide of Embodiment 193, wherein the oligonucleotide is /52MOErG/*/i2MOErG/*/i2MOErG/*/i2MOErC/*/i2MOErA/*/iMe-dC/*/iMe-dC/*/iMe-dC/*A*T*G*A*/iMe-dC/*A*/iMe-dC/*/i2MOErT/*/i2MOErC/*/i2MOErT/*/i2MOErG/*/32MOErG/(SEQ ID NO: 372) or a salt thereof.
253. The oligonucleotide of Embodiment 193, wherein the oligonucleotide is /52MOErA/*/i2MOErC/*/i2MOErT/*/i2MOErT/*/i2MOErG/*T*/iMe-dC/*/iMe-dC/*A*G*T*G*/iMe-dC/*T*/iMe-dC/*/i2MOErC/*/i2MOErA/*/i2MOErG/*/i2MOErG/*/32MOErT/ (SEQ ID NO: 373) or a salt thereof.
254. The oligonucleotide of Embodiment 193, wherein the oligonucleotide is /52MOErC/*/i2MOErC/*/i2MOErC/*/i2MOErA/*/i2MOErA/*T*/iMe-dC/*/iMe-dC/*T*T*G*/iMe-dC/*A*G*T*/i2MOErC/*/i2MOErA/*/i2MOErT/*/i2MOErG/*/32MOErG/ (SEQ ID NO: 374) or a salt thereof.
255. The oligonucleotide of Embodiment 193, wherein the oligonucleotide is /52MOErA/*/i2MOErG/*/i2MOErC/*/i2MOErA/*/i2MOErC/*A*G*/iMe-dC/*/iMe-dC/*T*G*/iMe-dC/*A*T*G*/i2MOErT/*/i2MOErC/*/i2MOErC/*/i2MOErT/*/32MOErC/ (SEQ ID NO: 375) or a salt thereof.
256. The oligonucleotide of Embodiment 193, wherein the oligonucleotide is /52MOErT/*/i2MOErG/*/i2MOErG/*/i2MOErT/*/i2MOErT/*A*G*G*T*T*G*G*A*/iMe-dC/*/iMe-dC/*/i2MOErC/*/i2MOErA/*/i2MOErT/*/i2MOErG/*/32MOErG/ (SEQ ID NO: 378) or a salt thereof.
257. The oligonucleotide of Embodiment 193, wherein the oligonucleotide is /52MOErT/*/i2MOErC/*/i2MOErC/*/i2MOErC/*/i2MOErT/*/iMe-dC/*T*/iMe-dC/*/iMe-dC/*A*G*A*T*A*/iMe-dC/*/i2MOErT/*/i2MOErG/*/i2MOErA/*/i2MOErG/*/32MOErG/ (SEQ ID NO: 380) or a salt thereof.
258. The oligonucleotide of Embodiment 193, wherein the oligonucleotide is /52MOErA/*/i2MOErC/*/i2MOErA/*/i2MOErG/*/i2MOErA/*/iMe-dC/*A*A*/iMe-dC/*/iMe-dC/*/iMe-dC/*A*A*T*G*/i2MOErG/*/i2MOErC/*/i2MOErA/*/i2MOErG/*/32MOErG/ (SEQ ID NO: 381) or a salt thereof.
259. The oligonucleotide of Embodiment 193, wherein the oligonucleotide is /52MOErC/*/i2MOErC/*/i2MOErT/*/i2MOErT/*/i2MOErA/*A*T*T*/iMe-dC/*/iMe-dC/*T*G*T*/iMe-dC/*T*/i2MOErG/*/i2MOErA/*/i2MOErG/*/i2MOErG/*/32MOErC/ (SEQ ID NO: 383) or a salt thereof.
260. The oligonucleotide of Embodiment 193, wherein the oligonucleotide is /52MOErC/*/i2MOErA/*/i2MOErG/*/i2MOErA/*/i2MOErA/*T*A*/iMe-dC/*A*G*T*G*/iMe-dC/*/iMe-dC/*/iMe-dC/*/i2MOErA/*/i2MOErG/*/i2MOErG/*/i2MOErC/*/32MOErC/ (SEQ ID NO: 384) or a salt thereof.
261. The oligonucleotide of Embodiment 193, wherein the oligonucleotide is /52MOErC/*/i2MOErC/*/i2MOErC/*/i2MOErA/*/i2MOErG/*G*/iMe-dC/*/iMe-dC/*/iMe-dC/*T*T*G*/iMe-dC/*T*/iMe-dC/*/i2MOErA/*/i2MOErG/*/i2MOErA/*/i2MOErA/*/32MOErT/(SEQ ID NO: 385) or a salt thereof.
262. The oligonucleotide of Embodiment 193, wherein the oligonucleotide is /52MOErG/*/i2MOErC/*/i2MOErA/*/i2MOErC/*/i2MOErT/*/iMe-dC/*A*T*/iMe-dC/*/iMe-dC/*/iMe-dC/*T*G*G*/iMe-dC/*/i2MOErT/*/i2MOErG/*/i2MOErG/*/i2MOErC/*/32MOErT/(SEQ ID NO: 386) or a salt thereof.
263. The oligonucleotide of Embodiment 193, wherein the oligonucleotide is /52MOErG/*/i2MOErA/*/i2MOErT/*/i2MOErT/*/i2MOErA/*/iMe-dC/*A*G*G*G*/iMe-dC/*A*A*G*G*/i2MOErC/*/i2MOErC/*/i2MOErA/*/i2MOErC/*/32MOErA/ (SEQ ID NO: 387) or a salt thereof.
264. The oligonucleotide of Embodiment 193, wherein the oligonucleotide is /52MOErG/*/i2MOErC/*/i2MOErC/*/i2MOErC/*/i2MOErT/*G*G*A*T*G*T*G*G*/iMe-dC/*A*/i2MOErA/*/i2MOErA/*/i2MOErA/*/i2MOErG/*/32MOErA/ (SEQ ID NO: 388) or a salt thereof.
265. The oligonucleotide of Embodiment 193, wherein the oligonucleotide is /52MOErA/*/i2MOErA/*/i2MOErG/*/i2MOErG/*/i2MOErA/*A*G*T*/iMe-dC/*A*G*A*G*G*G*/i2MOErA/*/i2MOErG/*/i2MOErG/*/i2MOErG/*/32MOErC/ (SEQ ID NO: 389) or a salt thereof.
266. The oligonucleotide of Embodiment 193, wherein the oligonucleotide is /52MOErC/*/i2MOErA/*/i2MOErG/*/i2MOErG/*/i2MOErC/*/iMe-dC/*/iMe-dC/*A*A*A*/iMe-dC/*A*G*G*A*/i2MOErG/*/i2MOErG/*/i2MOErC/*/i2MOErT/*/32MOErC/ (SEQ ID NO: 390) or a salt thereof.
267. The oligonucleotide of Embodiment 193, wherein the oligonucleotide is /52MOErA/*/i2MOErT/*/i2MOErG/*/i2MOErC/*/i2MOErC/*/iMe-dC/*A*G*A*/iMe-dC/*/iMe-dC/*/iMe-dC/*A*G*G*/i2MOErC/*/i2MOErC/*/i2MOErC/*/i2MOErA/*/32MOErA/ (SEQ ID NO: 391) or a salt thereof.
268. The oligonucleotide of Embodiment 193, wherein the oligonucleotide is /52MOErC/*/i2MOErT/*/i2MOErG/*/i2MOErA/*/i2MOErG/*G*/iMe-dC/*A*/iMe-dC/*A*G*/iMe-dC/*A*/iMe-dC/*/iMe-dC/*/i2MOErA/*/i2MOErA/*/i2MOErG/*/i2MOErG/*/32MOErC/ (SEQ ID NO: 392) or a salt thereof.
269. The oligonucleotide of Embodiment 193, wherein the oligonucleotide is /52MOErG/*/i2MOErC/*/i2MOErC/*/i2MOErA/*/i2MOErG/*A*/iMe-dC/*/iMe-dC/*A*G*G*A*A*G*G*/i2MOErA/*/i2MOErG/*/i2MOErC/*/i2MOErC/*/32MOErT/ (SEQ ID NO: 393) or a salt thereof.
270. The oligonucleotide of Embodiment 193, wherein the oligonucleotide is /52MOErG/*/i2MOErC/*/i2MOErC/*/i2MOErC/*/i2MOErA/*G*/iMe-dC/*/iMe-dC/*T*/iMe-dC/*A*G*A*A*T*/i2MOErG/*/i2MOErA/*/i2MOErT/*/i2MOErT/*/32MOErC/ (SEQ ID NO: 396) or a salt thereof.
271. The oligonucleotide of Embodiment 193, wherein the oligonucleotide is /52MOErC/*/i2MOErC/*/i2MOErT/*/i2MOErC/*/i2MOErT/*G*A*A*/iMe-dC/*/iMe-dC/*/iMe-dC/*A*G*T*G*/i2MOErG/*/i2MOErA/*/i2MOErG/*/i2MOErG/*/32MOErA/ (SEQ ID NO: 397) or a salt thereof.
272. The oligonucleotide of Embodiment 193, wherein the oligonucleotide is /52MOErG/*/i2MOErC/*/i2MOErC/*/i2MOErT/*/i2MOErG/*G*G*T*T*T*A*T*T*G*G*/i2MOErA/* /i2MOErG/*/i2MOErG/*/i2MOErG/*/32MOErT/ (SEQ ID NO: 398) or a salt thereof.
273. The oligonucleotide of Embodiment 193, wherein the oligonucleotide is /52MOErG/*/i2MOErC/*/i2MOErC/*/i2MOErA/*/i2MOErG/*/iMe-dC/*A*/iMe-dC/*A*G*/iMe-dC/*/iMe-dC/*A*A*G*/i2MOErA/*/i2MOErG/*/i2MOErT/*/i2MOErG/*/32MOErG/ (SEQ ID NO: 399) or a salt thereof.
274. The oligonucleotide of any one of the preceding Embodiments, wherein the oligonucleotide is a pharmaceutically acceptable salt.
275. The oligonucleotide of any one of the preceding Embodiments, wherein the oligonucleotide is a sodium salt.
276. A composition comprising an oligonucleotide of any one of the preceding Embodiments and one or more diastereomers of the oligonucleotide with respect to chiral linkage phosphorus.
277. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 382)
/52MOErG/*/i2MOErT/*/i2MOErC/*/i2MOErT/*/i2MOErC/
*/iMe-dC/*A*G*A*A*/iMe-dC/*T*G*A*G*/i2MOErC/*/
i2MOErA/*/i2MOErG/*/i2MOErG/*/32MOErG/.

278. A composition comprising:
   an oligonucleotide or a salt thereof, and
   one or more diastereomers of the oligonucleotide with respect to chiral linkage phosphorus, or one or more salts of the diastereomers,
   wherein the oligonucleotide is

(SEQ ID NO: 365)
/52MOErC/*/i2MOErC/*/i2MOErT/*/i2MOErT/*/i2MOErG/*
/iMe-dC/*A*G*G*/iMe-dC/*T*/iMe-dC/*T*T*G*/i2MOErA/
*/i2MOErT/*/i2MOErG/*/i2MOErG/*/32MOErC/.

279. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 325)
/52MOErG/*/i2MOErC/*/i2MOErT/*/i2MOErG/*/i2MOErG/
*/iMe-dC/*T*G*T*A*/iMe-dC/*T*/iMe-dC/*A*/iMe-dC/*
/i2MOErT/*/i2MOErC/*/i2MOErT/*/i2MOErC/*/32MOErC/.

280. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 394)
/52MOErT/*/i2MOErC/*/i2MOErA/*/i2MOErG/*/i2MOErG/
*A*/iMe-dC/*T*T*T*G*/iMe-dC/*/iMe-dC/*T*/iMe-dC/*
/i2MOErT/*/i2MOErT/*/i2MOErT/*/i2MOErC/*/32MOErC/.

281. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 400)
/52MOErG/*/i2MOErG/*/i2MOErG/*/i2MOErA/*/i2MOErG/
*T*G*G*A*A*G*G*A*A*G*/i2MOErG/*/i2MOErA/*/i2MOErG/
*/i2MOErC/*/32MOErC/.

282. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 395)
/52MOErG/*/i2MOErC/*/i2MOErT/*/i2MOErT/*/i2MOET/*A*G*A*G*A*T*T*T*G*/iMe-
dC/*/i2MOErT/*/i2MOErA/*/i2MOErC/*/i2MOErC/*/32MOErC/.

283. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 348)
/52MOErG/*/i2MOErC/*/i2MOErA/*/i2MOErT/*/i2MOErC/*A*/iMe-dC/*T*/iMe-
dC/*A*/iMe-dC/*T*G*T*/iMe-
dC/*/i2MOErA/*/i2MOErG/*/i2MOErG/*/i2MOErT/*/32MOErA/.

284. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 344)
/52MOErG/*/i2MOErC/*/i2MOErC/*/i2MOErC/*/i2MOErT/*A*G*G*A*T*T*T*T*/iMe-
dC/*/iMe-dC/*/i2MOErT/*/i2MOErG/*/i2MOErT/*/i2MOErT/*/32MOErG/.

285. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 366)
/52MOErG/*/i2MOErT/*/i2MOErG/*/i2MOErC/*/i2MOErC/*A*T*T*G*G*T*A*G*A*G*/
i2MOErT/*/i2MOErA/*/i2MOErG/*/i2MOErG/*/32MOErA/.

286. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 328)
/52MOErC/*/i2MOErT/*/i2MOErG/*/i2MOErT/*/i2MOErG/*A*/iMe-dC/*/iMe-
dC/*T*A*G*G*/iMe-dC/*T*/iMe-
dC/*/i2MOErC/*/i2MOErT/*/i2MOErT/*/i2MOErG/*/32MOErA/.

287. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 346)
/52MOErA/*/i2MOErG/*/i2MOErA/*/i2MOErC/*/i2MOErA/*/iMe-dC/*/iMe-
dC/*T*G*G*G*T*A*T*/iMe-
dC/*/i2MOErA/*/i2MOErG/*/i2MOErC/*/i2MOErC/*/32MOErT/.

288. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 379)
/52MOErG/*/i2MOErC/*/i2MOErC/*/i2MOErC/*/i2MOErA/*G*G*T*T*G*T*/iMe-
dC/*T*/iMe-dC/*A*/i2MOErG/*/i2MOErC/*/i2MOErC/*/i2MOErC/*/32MOErA/.

289. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 352)
/52MOErG/*/i2MOErC/*/i2MOErT/*/i2MOErT/*/i2MOErG/*T*/iMe-dC/*T*/iMe-
dC/*A*T*/iMe-dC/*/iMe-
dC/*T*G*/i2MOErT/*/i2MOErC/*/i2MOErT/*/i2MOErC/*/32MOErT/.

290. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 363)
/52MOErG/*/i2MOErG/*/i2MOErT/*/i2MOErT/*/i2MOErC/*T*/iMe-dC/*A*G*/iMe-
dC/*/iMe-dC/*A*/iMe-dC/*/iMe-
dC/*A*/i2MOErG/*/i2MOErG/*/i2MOErA/*/i2MOErT/*/32MOErC/.

291. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 376)
/52MOErC/*/i2MOErA/*/i2MOErA/*/i2MOErA/*/i2MOErC/*T*G*G*T*G*T*/iMe-
dC/*A*G*A*/i2MOErG/*/i2MOErC/*/i2MOErC/*/i2MOErT/*/32MOErG/.

292. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 377)
/52MOErG/*/i2MOErC/*/i2MOErA/*/i2MOErG/*/i2MOErC/*A*/iMe-dC/*/iMe-dC/*/iMe-
dC/*T*/iMe-dC/*/iMe-
dC/*A*A*A*/i2MOErC/*/i2MOErT/*/i2MOErG/*/i2MOErG/*/32MOErT/.

293. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 353)
/52MOErT/*/i2MOErG/*/i2MOErT/*/i2MOErC/*/i2MOErT/*/iMe-dC/*T*G*A*G*/iMe-
dC/*T*G*A*/iMe-dC/*/i2MOErT/*/i2MOErG/*/i2MOErC/*/i2MOErT/*/32MOErT/.

294. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 321)
/52MOErC/*/i2MOErC/*/i2MOErA/*/i2MOErC/*/i2MOErT/*A*G*/iMe-dC/*/iMe-dC/*/iMe-
dC/*T*G*G*G*A*/i2MOErG/*/i2MOErC/*/i2MOErA/*/i2MOErA/*/32MOErA/.

295. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 322)
/52MOErG/*/i2MOErC/*/i2MOErC/*/i2MOErA/*/i2MOErT/*/iMe-dC/*T*/iMe-dC/*/iMe-
dC/*A*T*/iMe-dC/*/iMe-
dC/*A*T*/i2MOErA/*/i2MOErG/*/i2MOErA/*/i2MOErG/*/32MOErC/.

296. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 323)
/52MOErA/*/i2MOErG/*/i2MOErG/*/i2MOErA/*/i2MOErG/*A*G*/iMe-
dC/*T*G*T*G*G*G*/iMe-dC/*/i2MOErT/*/i2MOErT/*/i2MOErG/*/i2MOErG/*/32MOErG/.

297. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 324)
/52MOErC/*/i2MOErA/*/i2MOErC/*/i2MOErC/*/i2MOErC/*A*T*G*/iMe-dC/*/iMe-
dC/*T*/iMe-dC/*/iMe-dC/*/iMe-
dC/*A*/i2MOErG/*/i2MOErC/*/i2MOErA/*/i2MOErG/*/32MOErA/.

298. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 326)
/52MOErG/*/i2MOErT/*/i2MOErG/*/i2MOErC/*/i2MOErT/*/iMe-dC/*T*G*T*/iMe-
dC/*/iMe-dC/*T*T*G*G*/i2MOErT/*/i2MOErC/*/i2MOErC/*/i2MOErT/*/32MOErG/.

299. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 327)
/52MOErC/*/i2MOErC/*/i2MOErC/*/i2MOErA/*/i2MOErT/*T*/iMe-dC/*T*/iMe-
dC/*A*T*G*/iMe-dC/*A*G*/i2MOErC/*/i2MOErC/*/i2MOErT/*/i2MOErA/*/32MOErC/.

300. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 329)
/52MOErG/*/i2MOErG/*/i2MOErT/*/i2MOErC/*/i2MOErT/*G*A*G*A*G*G*/iMe-
dC/*T*G*T*/i2MOErG/*/i2MOErG/*/i2MOErG/*/i2MOErT/*/32MOErC/.

301. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 330)
/52MOErG/*/i2MOErC/*/i2MOErT/*/i2MOErC/*/i2MOErC/*/iMe-dC/*A*G*T*T*/iMe-
dC/*T*T*/iMe-dC/*T*/i2MOErG/*/i2MOErT/*/i2MOErG/*/i2MOErG/*/32MOErT/.

302. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 331)
/52MOErG/*/i2MOErA/*/i2MOErT/*/i2MOErG/*/i2MOErT/*/iMe-dC/*/iMe-dC/*T*/iMe-
dC/*/iMe-dC/*A*/iMe-
dC/*A*G*G*/i2MOErT/*/i2MOErG/*/i2MOErA/*/i2MOErC/*/32MOErA/.

303. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 332)
/52MOErG/*/i2MOErC/*/i2MOErT/*/i2MOErT/*/i2MOErC/*/iMe-dC/*T*G*/iMe-dC/*/iMe-
dC/*T*T*A*/iMe-dC/*T*/i2MOErG/*/i2MOErA/*/i2MOErC/*/i2MOErC/*/32MOErT/.

304. A composition comprising:
   an oligonucleotide or a salt thereof, and
   one or more diastereomers of the oligonucleotide with respect to chiral linkage phosphorus, or
   one or more salts of the diastereomers,
      wherein the oligonucleotide is

(SEQ ID NO: 333)
/52MOErC/*/i2MOErT/*/i2MOErC/*/i2MOErT/*/i2MOErC/*/iMe-dC/*T*T*T*G*T*/iMe-
dC/*/iMe-dC/*/iMe-dC/*T*/i2MOErG/*/i2MOErA/*/i2MOErC/*/i2MOErC/*/32MOErA/.

305. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 334)
/52MOErG/*/i2MOErC/*/i2MOErC/*/i2MOErT/*/i2MOErT/*G*/iMe-dC/*/iMe-
dC/*T*T*T*T*/iMe-dC/*/iMe-
dC/*T*/i2MOErC/*/i2MOErA/*/i2MOErC/*/i2MOErT/*/32MOErC/.

306. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 335)
/52MOErG/*/i2MOErC/*/i2MOErC/*/i2MOErT/*/i2MOErG/*G*T*/iMe-dC/*A*/iMe-
dC/*T*A*A*/iMe-dC/*/iMe-dC/*/i2MOErC/*/i2MOErT/*/i2MOErC/*/i2MOErT/*/32MOErC/.

307. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 336)
/52MOErC/*/i2MOErA/*/i2MOErC/*/i2MOErC/*/i2MOErC/*A*/iMe-dC/*/iMe-
dC/*T*T*G*G*T*/iMe-dC/*T*/i2MOErT/*/i2MOErG/*/i2MOErC/*/i2MOErC/*/32MOErT/.

308. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 337)
/52MOErC/*/i2MOErA/*/i2MOErC/*/i2MOErA/*/i2MOErC/*T*G*A*T*G*T*/iMe-dC/*/iMe-
dC/*T*G*/i2MOErT/*/i2MOErC/*/i2MOErC/*/i2MOErC/*/32MOErA/.

309. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 338)
/52MOErC/*/i2MOErA/*/i2MOErC/*/i2MOErA/*/i2MOErC/*/iMe-dC/*T*/iMe-
dC/*T*G*G*G*T*/iMe-dC/*T*/i2MOErT/*/i2MOErG/*/i2MOErG/*/i2MOErC/*/32MOErC/.

310. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 339)
/52MOErG/*/i2MOErC/*/i2MOErT/*/i2MOErG/*/i2MOErC/*/iMe-dC/*/iMe-dC/*A*T*/iMe-
dC/*A*/iMe-dC/*T*/iMe-dC/*/iMe-
dC/*/i2MOErC/*/i2MOErA/*/i2MOErG/*/i2MOErT/*/32MOErT/.

311. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 340)
/52MOErC/*/i2MOErT/*/i2MOErC/*/i2MOErT/*/i2MOErC/*/iMe-dC/*A*T*/iMe-
dC/*T*G*/iMe-dC/*/iMe-dC/*/iMe-
dC/*T*/i2MOErG/*/i2MOErG/*/i2MOErC/*/i2MOErC/*/32MOErC/.

312. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 341)
/52MOErC/*/i2MOErA/*/i2MOErG/*/i2MOErT/*/i2MOErC/*/iMe-dC/*/iMe-dC/*T*/iMe-
dC/*T*/iMe-dC/*/iMe-
dC/*T*T*G*/i2MOErT/*/i2MOErC/*/i2MOErT/*/i2MOErC/*/32MOErT/.

313. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 342)
/52MOErA/*/i2MOErT/*/i2MOErC/*/i2MOErC/*/i2MOErA/*/iMe-dC/*/iMe-dC/*T*G*/iMe-
dC/*T*G*/iMe-dC/*T*/iMe-dC/*/i2MOErC/*/i2MOErT/*/i2MOErG/*/i2MOErG/*/32MOErG/.

314. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 343)
/52MOErC/*/i2MOErC/*/i2MOErC/*/i2MOErT/*/i2MOErT/*G*T*G*T*/iMe-
dC/*T*T*G*T*G*/i2MOErG/*/i2MOErG/*/i2MOErT/*/i2MOErG/*/32MOErC/.

315. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 345)
/52MOErG/*/i2MOErC/*/i2MOErC/*/i2MOErT/*/i2MOErC/*A*A*/iMe-dC/*T*/iMe-
dC/*/iMe-dC/*T*G*/iMe-dC/*/iMe-
dC/*/i2MOErT/*/i2MOErC/*/i2MOErC/*/i2MOErC/*/32MOErA/.

316. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 347)
/52MOErT/*/i2MOErC/*/i2MOErC/*/i2MOErT/*/i2MOErT/*/iMe-dC/*T*T*/iMe-dC/*/iMe-
dC/*/iMe-dC/*T*A*T*T*/i2MOErT/*/i2MOErC/*/i2MOErC/*/i2MOErC/*/32MOErA/.

317. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 349)
/52MOErG/*/i2MOErT/*/i2MOErC/*/i2MOErA/*/i2MOErG/*T*G*/iMe-dC/*/iMe-
dC/*A*/iMe-dC/*A*G*/iMe-dC/*/iMe-
dC/*/i2MOErT/*/i2MOErT/*/i2MOErG/*/i2MOErT/*/32MOErC/.

318. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 350)
/52MOErG/*/i2MOErG/*/i2MOErC/*/i2MOErA/*/i2MOErC/*/iMe-dC/*T*A*/iMe-dC/*/iMe-
dC/*T*T*A*T*G*/i2MOErC/*/i2MOErA/*/i2MOErC/*/i2MOErC/*/32MOErC/.

319. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 351)
/52MOErA/*/i2MOErC/*/i2MOErT/*/i2MOErA/*/i2MOErC/*T*G*/iMe-dC/*A*T*/iMe-
dC/*/iMe-dC/*/iMe-dC/*T*/iMe-
dC/*/i2MOErA/*/i2MOErG/*/i2MOErC/*/i2MOErC/*/32MOErC/.

320. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 354)
/52MOErG/*/i2MOErG/*/i2MOErG/*/i2MOErC/*/i2MOErT/*T*G*A*/iMe-dC/*T*/iMe-
dC/*/iMe-dC/*A*/iMe-dC/*A*/i2MOErC/*/i2MOErT/*/i2MOErC/*/i2MOErC/*/32MOErA/.

321. A composition comprising:
   an oligonucleotide or a salt thereof, and
   one or more diastereomers of the oligonucleotide with respect to chiral linkage phosphorus, or
   one or more salts of the diastereomers,

(SEQ ID NO: 355)
/52MOErG/*/i2MOErG/*/i2MOErC/*/i2MOErA/*/i2MOErT/*G*G*/iMe-dC/*A*T*/iMe-
dC/*T*/iMe-dC/*A*G*/i2MOErC/*/i2MOErT/*/i2MOErT/*/i2MOErC/*/32MOErA/.

wherein the oligonucleotide is
322. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 356)
/52MOErT/*/i2MOErT/*/i2MOErC/*/i2MOErA/*/i2MOErG/*G*A*T*/iMe-dC/*A*/iMe-
dC/*/iMe-dC/*T*A*G*/i2MOErC/*/i2MOErT/*/i2MOErG/*/i2MOErG/*/32MOErT/.

323. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 357)
/52MOErC/*/i2MOErC/*/i2MOErT/*/i2MOErC/*/i2MOErT/*T*T*G*/iMe-dC/*/iMe-
dC/*A*T*/iMe-dC/*T*G*/i2MOErC/*/i2MOErT/*/i2MOErG/*/i2MOErG/*/32MOErG/.

324. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 358)
/52MOErG/*/i2MOErA/*/i2MOErG/*/i2MOErT/*/i2MOErG/*/iMe-dC/*A*G*T*T*/iMe-
dC/*A*/iMe-dC/*T*T*/i2MOErG/*/i2MOErT/*/i2MOErG/*/i2MOErG/*/32MOErT/.

325. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 359)
/52MOErT/*/i2MOErG/*/i2MOErC/*/i2MOErC/*/i2MOErC/*A*/iMe-dC/*A*/iMe-
dC/*T*/iMe-dC/*T*G*/iMe-dC/*/iMe-
dC/*/i2MOErT/*/i2MOErG/*/i2MOErT/*/i2MOErC/*/32MOErA/.

326. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 360)
/52MOErC/*/i2MOErA/*/i2MOErG/*/i2MOErA/*/i2MOErG/*G*G*A*G*/iMe-
dC/*T*G*/iMe-dC/*T*A*/i2MOErG/*/i2MOErT/*/i2MOErC/*/i2MOErA/*/32MOErG/.

327. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 361)
/52MOErT/*/i2MOErT/*/i2MOErG/*/i2MOErG/*/i2MOErC/
*A*A*A*G*G*T*G*A*T*G*/i2MOErC/*/i2MOErA/*/i2MOErG/
*/i2MOErG/*/32MOErC/.

328. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 362)
/52MOErC/*/i2MOErC/*/i2MOErT/*/i2MOErC/*/i2MOErC/
*A*/iMe-dC/*/iMe-dC/*A*G*T*T*G*G*A*/i2MOErA/*/
i2MOErG/*/i2MOErA/*/i2MOErC/*/32MOErC/.

329. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 364)
/52MOErG/*/i2MOErT/*/i2MOErG/*/i2MOErC/*/i2MOErT/*
/iMe-dC/*/iMe-dC/*A*A*G*A*T*G*/iMe-dC/*/iMe-dC/*/
i2MOErT/*/i2MOErG/*/i2MOErC/*/i2MOErC/*/32MOErA/.

330. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 367)
/52MOErG/*/i2MOErT/*/i2MOErG/*/i2MOErA/*/i2MOErG/*
/iMe-dC/*T*/iMe-dC/*/iMe-dC/*/iMe-dC/*T*A*A*A*G*/
i2MOErA/*/i2MOErA/*/i2MOErC/*/i2MOErC/*/32MOErT/.

331. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 368)
/52MOErG/*/i2MOErG/*/i2MOErT/*/i2MOErT/*/i2MOErT/
*G*/iMe-dC/*/iMe-dC/*A*/iMe-dC/*/iMe-dC/*A*G*T*A*/
i2MOErC/*/i2MOErA/*/i2MOErG/*/i2MOErG/*/32MOErG/.

332. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 369)
/52MOErT/*/i2MOErC/*/i2MOErC/*/i2MOErA/*/i2MOErG/*
/iMe-dC/*T*T*/iMe-dC/*T*/iMe-dC/*/iMe-dC/*A*/iMe-
dC/*A*/i2MOErT/*/i2MOErC/*/i2MOErA/*/i2MOErA/*/
32MOErT/.

333. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 370)
/52MOErG/*/i2MOErA/*/i2MOErA/*/i2MOErC/*/i2MOErT/
*T*G*/iMe-dC/*/iMe-dC/*T*G*/iMe-dC/*T*T*/iMe-dC/*/
i2MOErC/*/i2MOErA/*/i2MOErG/*/i2MOErC/*/32MOErT/.

334. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 371)
/52MOErA/*/i2MOErC/*/i2MOErA/*/i2MOErC/*/i2MOErT/
*/iMe-dC/*T*G*G*A*T*G*A*G*T*/i2MOErT/*/i2MOErT/*/
i2MOErG/*/i2MOErT/*/32MOErC/.

335. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 372)
/52MOErG/*/i2MOErG/*/i2MOErG/*/i2MOErC/*/i2MOErA/
*/iMe-dC/*/iMe-dC/*/iMe-dC/*A*T*G*A*/iMe-dC/*A*/
iMe-dC/*/i2MOErT/*/i2MOErC/*/i2MOErT/*/i2MOErG/*/
32MOErG/.

336. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 373)
/52MOErA/*/i2MOErC/*/i2MOErT/*/i2MOErT/*/i2MOErG/
*T*/iMe-dC/*/iMe-dC/*A*G*T*G*/iMe-dC/*T*/iMe-dC/*/
i2MOErC/*/i2MOErA/*/i2MOErG/*/i2MOErG/*/32MOErT/.

337. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 374)
/52MOErC/*/i2MOErC/*/i2MOErC/*/i2MOErA/*/i2MOErA/
*T*/iMe-dC/*/iMe-dC/*T*T*G*/iMe-dC/*A*G*T*/i2MOErC
/*/i2MOErA/*/i2MOErT/*/i2MOErG/*/32MOErG/.

338. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 375)
/52MOErA/*/i2MOErG/*/i2MOErC/*/i2MOErA/*/i2MOErC/
*A*G*/iMe-dC/*/iMe-dC/*T*G*/iMe-dC/*A*T*G*/
i2MOErT/*/i2MOErC/*/i2MOErC/*/i2MOErT/*/32MOErC/.

339. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 378)
/52MOErT/*/i2MOErG/*/i2MOErG/*/i2MOErT/*/i2MOErT/
*A*G*G*T*T*G*G*A*/iMe-dC/*/iMe-dC/*/i2MOErC/*/
i2MOErA/*/i2MOErT/*/i2MOErG/*/32MOErG/.

340. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 380)
/52MOErT/*/i2MOErC/*/i2MOErC/*/i2MOErC/*/i2MOErT/
*/iMe-dC/*T*/iMe-dC/*/iMe-dC/*A*G*A*T*A*/iMe-dC/*/
i2MOErT/*/i2MOErG/*/i2MOErA/*/i2MOErG/*/32MOErG/.

341. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 381)
/52MOErA/*/i2MOErC/*/i2MOErA/*/i2MOErG/*/i2MOErA/
*/iMe-dC/*A*A*/iMe-dC/*/iMe-dC/*/iMe-dC/*A*A*T*G*/
i2MOErG/*/i2MOErC/*/i2MOErA/*/i2MOErG/*/32MOErG/.

342. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 383)
/52MOErC/*/i2MOErC/*/i2MOErT/*/i2MOErT/*
/i2MOErA/*A*T*T*/iMe-dC/*/iMe-dC/*T*G*T*
/iMe-dC/*T*/i2MOErG/*/i2MOErA/*/i2MOErG/
*/i2MOErG/*/32MOErC/.

343. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 384)
/52MOErC/*/i2MOErA/*/i2MOErG/*/i2MOErA/*
/i2MOErA/*T*A*/iMe-dC/*A*G*T*G*/iMe-dC/*
/iMe-dC/*/iMe-dC/*/i2MOErA/*/i2MOErG/*/i
2MOErG/*/i2MOErC/*/32MOErC/.

344. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 385)
/52MOErC/*/i2MOErC/*/i2MOErC/*/i2MOErA/*
/i2MOErG/*G*/iMe-dC/*/iMe-dC/*/iMe-dC/*T
*T*G*/iMe-dC/*T*/iMe-dC/*/i2MOErA/*/i2MO
ErG/*/i2MOErA/*/i2MOErA/*/32MOErT/.

345. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 386)
/52MOErG/*/i2MOErC/*/i2MOErA/*/i2MOErC/*
/i2MOErT/*/iMe-dC/*A*T*/iMe-dC/*/iMe-dC/
*/iMe-dC/*T*G*G*/iMe-dC/*/i2MOErT/*/i2MO
ErG/*/i2MOErG/*/i2MOErC/*/32MOErT/.

346. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 387)
/52MOErG/*/i2MOErA/*/i2MOErT/*/i2MOErT/*
/i2MOErA/*/iMe-dC/*A*G*G*G*/iMe-dC/*A*A*
G*G*/i2MOErC/*/i2MOErC/*/i2MOErA/*/i2MOE
rC/*/32MOErA/.

347. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 388)
/52MOErG/*/i2MOErC/*/i2MOErC/*/i2MOErC/*
/i2MOErT/*G*G*A*T*G*T*G*G*/iMe-dC/*A*/i2
MOErA/*/i2MOErA/*/i2MOErA/*/i2MOErG/*/32
MOErA/.

348. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 389)
/52MOErA/*/i2MOErA/*/i2MOErG/*/i2MOErG/*
/i2MOErA/*A*G*T*/iMe-dC/*A*G*A*G*G*G*/i2
MOErA/*/i2MOErG/*/i2MOErG/*/i2MOErG/*/32
MOErC/.

349. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 390)
/52MOErC/*/i2MOErA/*/i2MOErG/*/i2MOErG/*
/i2MOErC/*/iMe-dC/*/iMe-dC/*A*A*A*/iMe-d
C/*A*G*G*A*/i2MOErG/*/i2MOErG/*/i2MOErC/
*/i2MOErT/*/32MOErC/.

350. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 391)
/52MOErA/*/i2MOErT/*/i2MOErG/*/i2MOErC/*
/i2MOErC/*/iMe-dC/*A*G*A*/iMe-dC/*/iMe-d
C/*/iMe-dC/*A*G*G*/i2MOErC/*/i2MOErC/*/i
2MOErC/*/i2MOErA/*/32MOErA/.

351. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 392)
/52MOErC/*/i2MOErT/*/i2MOErG/*/i2MOErA/*
/i2MOErG/*G*/iMe-dC/*A*/iMe-dC/*A*G*/iMe
-dC/*A*/iMe-dC/*/iMe-dC/*/i2MOErA/*/i2MO
ErA/*/i2MOErG/*/i2MOErG/*/32MOErC/.

352. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 393)
/52MOErG/*/i2MOErC/*/i2MOErC/*/i2MOErA/*
/i2MOErG/*A*/iMe-dC/*/iMe-dC/*A*G*G*A*A*
G*G*/i2MOErA/*/i2MOErG/*/i2MOErC/*/i2MOE
rC/*/32MOErT/.

353. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 396)
/52MOErG/*/i2MOErC/*/i2MOErC/*/i2MOErC/*
/i2MOErA/*G*/iMe-dC/*/iMe-dC/*T*/iMe-dC/
*A*G*A*A*T*/i2MOErG/*/i2MOErA/*/i2MOErT/
*/i2MOErT/*/32MOErC/.

354. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 397)
/52MOErC/*/i2MOErC/*/i2MOErT/*/i2MOErC/*
/i2MOErT/*G*A*A*/iMe-dC/*/iMe-dC/*/iMe-d
C/*A*G*T*G*/i2MOErG/*/i2MOErA/*/i2MOErG/
*/i2MOErG/*/32MOErA/.

355. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 398)
/52MOErG/*/i2MOErC/*/i2MOErC/*/i2MOErT/*
/i2MOErG/*G*G*T*T*T*A*T*T*G*G*/i2MOErA/*
/i2MOErG/*/i2MOErG/*/i2MOErG/*/32MOErT/.

356. A composition comprising:
   an oligonucleotide or a salt thereof, and
   one or more diastereomers of the oligonucleotide with respect to chiral linkage phosphorus, or
   one or more salts of the diastereomers,
   wherein the oligonucleotide is

(SEQ ID NO: 399)
/52MOErG/*/i2MOErC/*/i2MOErC/*/i2MOErA/*
/i2MOErG/*/iMe-dC/*A*/iMe-dC/*A*G*/iMe-d
C/*/iMe-dC/*A*A*G*/i2MOErA/*/i2MOErG/*/i
2MOErT/*/i2MOErG/*/32MOErG/.

357. The composition of any one of Embodiments 276-356, wherein for each chiral linkage phosphorus, the percentage of the Rp configuration is independently about 20%-80%.
358. The composition of any one of Embodiments 276-356, wherein for each chiral linkage phosphorus, the percentage of the Rp configuration is independently about 30%-70%.
359. The composition of any one of Embodiments 276-356, wherein for each chiral linkage phosphorus, the percentage of the Rp configuration is independently about 40%-60%.
360. The composition of any one of Embodiments 276-356, wherein for each chiral linkage phosphorus, the percentage of the Rp configuration is independently about 45%-55%.
361. The composition of any one of Embodiments 276-356, wherein for each chiral linkage phosphorus, the percentage of the Rp configuration is independently about 50%.
362. The composition of any one of Embodiments 276-361, wherein the composition comprises a salt of the oligonucleotide, and one or more salts of one or more diastereomers.
363. The composition of any one of Embodiments 276-362, wherein the composition is a pharmaceutical composition, and further comprising a pharmaceutically acceptable carrier.
364. The composition of any one of Embodiments 276-363, wherein the composition comprises a pharmaceutically acceptable salt of the oligonucleotide, one or more pharmaceutically acceptable salts of one or more diastereomers, and a pharmaceutically acceptable carrier.
365. A pharmaceutical composition comprising an oligonucleotide of any one of the preceding Embodiments and a pharmaceutically acceptable carrier.
366. The composition of Embodiment 365, wherein the composition comprises one or more pharmaceutically acceptable salts of an oligonucleotide.
367. The composition of any one of Embodiments 276-366, wherein the composition is a liquid composition.
368. The composition of any one of Embodiments 363-367, wherein a pharmaceutically acceptable carrier is a buffer.
369. The composition of any one of Embodiments 363-367, wherein a pharmaceutically acceptable carrier is a buffered saline.
370. The composition of any one of Embodiments 363-367, wherein a pharmaceutically acceptable carrier is artificial cerebrospinal fluid.
371. A method for reducing level of SARM1 mRNA in a system, comprising administering or delivering to the system an effective amount of an oligonucleotide or composition of any one of the preceding Embodiments.
372. A method for reducing level of SARM1 polypeptide in a system, comprising administering or delivering to the system an effective amount of an oligonucleotide or composition of any one of the preceding Embodiments.
373. A method for reducing level of SARM1 activity in a system, comprising administering or delivering to the system an effective amount of an oligonucleotide or composition of any one of the preceding Embodiments.
374. The method of any one of Embodiments 371-373, wherein the system expresses SARM1 mRNA.
375. The method of any one of Embodiments 371-374, wherein the system is or comprises a cell.
376. The method of any one of Embodiments 371-374, wherein the system is or comprises a population of neuronal cells.
377. The method of any one of Embodiments 371-374, wherein the system is or comprises a tissue.
378. The method of any one of Embodiments 371-374, wherein the system is or comprises an organ.
379. The method of any one of Embodiments 371-374, wherein the system is or comprises brain or a portion thereof.
380. The method of any one of Embodiments 371-374, wherein the system is or comprises an organism.
381. The method of any one of Embodiments 371-374, wherein the system is a subject.
382. The method of any one of Embodiments 371-374, wherein the system is a human.
383. The method of any one of Embodiments 371-382, wherein level of SARM1 mRNA in the system is reduced by about or at least about 10%, 15%, 20%, 25%, 30%, 35%, 40%, 45%, 55%, 60%, 65%, 70%, 75%, 80%, 85%, or 90% compared to absence of the oligonucleotide or composition.
384. The method of any one of Embodiments 371-382, wherein level of SARM1 mRNA in the system is reduced by about or at least about 10% compared to absence of the oligonucleotide or composition.
385. The method of any one of Embodiments 371-382, wherein level of SARM1 mRNA in the system is reduced by about or at least about 15% compared to absence of the oligonucleotide or composition.
386. The method of any one of Embodiments 371-382, wherein level of SARM1 mRNA in the system is reduced by about or at least about 20% compared to absence of the oligonucleotide or composition.
387. The method of any one of Embodiments 371-382, wherein level of SARM1 mRNA in the system is reduced by about or at least about 25% compared to absence of the oligonucleotide or composition.
388. The method of any one of Embodiments 371-382, wherein level of SARM1 mRNA in the system is reduced by about or at least about 30% compared to absence of the oligonucleotide or composition.
389. The method of any one of Embodiments 371-382, wherein level of SARM1 mRNA in the system is reduced by about or at least about 35% compared to absence of the oligonucleotide or composition.
390. The method of any one of Embodiments 371-382, wherein level of SARM1 mRNA in the system is reduced by about or at least about 40% compared to absence of the oligonucleotide or composition.
391. The method of any one of Embodiments 371-382, wherein level of SARM1 mRNA in the system is reduced by about or at least about 45% compared to absence of the oligonucleotide or composition.
392. The method of any one of Embodiments 371-382, wherein level of SARM1 mRNA in the system is reduced by about or at least about 50% compared to absence of the oligonucleotide or composition.
393. The method of any one of Embodiments 371-382, wherein level of SARM1 mRNA in the system is reduced by about or at least about 55% compared to absence of the oligonucleotide or composition.
394. The method of any one of Embodiments 371-382, wherein level of SARM1 mRNA in the system is reduced by about or at least about 60% compared to absence of the oligonucleotide or composition.
395. The method of any one of Embodiments 371-382, wherein level of SARM1 mRNA in the system is reduced by about or at least about 65% compared to absence of the oligonucleotide or composition.
396. The method of any one of Embodiments 371-382, wherein level of SARM1 mRNA in the system is reduced by about or at least about 70% compared to absence of the oligonucleotide or composition.
397. The method of any one of Embodiments 371-382, wherein level of SARM1 mRNA in the system is reduced by about or at least about 75% compared to absence of the oligonucleotide or composition.
398. The method of any one of Embodiments 371-382, wherein level of SARM1 mRNA in the system is reduced by about or at least about 80% compared to absence of the oligonucleotide or composition.
399. The method of any one of Embodiments 371-382, wherein level of SARM1 mRNA in the system is reduced by about or at least about 85% compared to absence of the oligonucleotide or composition.
400. The method of any one of Embodiments 371-382, wherein level of SARM1 mRNA in the system is reduced by about or at least about 90% compared to absence of the oligonucleotide or composition.
401. The method of any one of Embodiments 371-400, wherein level of SARM1 polypeptide in the system is reduced by about or at least about 10%, 15%, 20%, 25%, 30%, 35%, 40%, 45%, 50%, 55%, or 60% compared to absence of the oligonucleotide or composition.
402. The method of any one of Embodiments 371-400, wherein level of SARM1 polypeptide in the system is reduced by about or at least about 10% compared to absence of the oligonucleotide or composition.
403. The method of any one of Embodiments 371-400, wherein level of SARM1 polypeptide in the system is reduced by about or at least about 15% compared to absence of the oligonucleotide or composition.
404. The method of any one of Embodiments 371-400, wherein level of SARM1 polypeptide in the system is reduced by about or at least about 20% compared to absence of the oligonucleotide or composition.
405. The method of any one of Embodiments 371-400, wherein level of SARM1 polypeptide in the system is reduced by about or at least about 25% compared to absence of the oligonucleotide or composition.
406. The method of any one of Embodiments 371-400, wherein level of SARM1 polypeptide in the system is reduced by about or at least about 30% compared to absence of the oligonucleotide or composition.
407. The method of any one of Embodiments 371-400, wherein level of SARM1 polypeptide in the system is reduced by about or at least about 35% compared to absence of the oligonucleotide or composition.
408. The method of any one of Embodiments 371-400, wherein level of SARM1 polypeptide in the system is reduced by about or at least about 40% compared to absence of the oligonucleotide or composition.
409. The method of any one of Embodiments 371-400, wherein level of SARM1 polypeptide in the system is reduced by about or at least about 45% compared to absence of the oligonucleotide or composition.
410. The method of any one of Embodiments 371-400, wherein level of SARM1 polypeptide in the system is reduced by about or at least about 50% compared to absence of the oligonucleotide or composition.
411. The method of any one of Embodiments 371-400, wherein level of SARM1 polypeptide in the system is reduced by about or at least about 55% compared to absence of the oligonucleotide or composition.
412. The method of any one of Embodiments 371-400, wherein level of SARM1 polypeptide in the system is reduced by about or at least about 60% compared to absence of the oligonucleotide or composition.
413. The method of any one of Embodiments 371-412, wherein level of SARM1 activity in the system is reduced by about or at least about 10%, 15%, 20%, 25%, 30%, 35%, 40%, 45%, 50%, 55%, 60%, 65%, 70%, 75%, or 80% compared to absence of the oligonucleotide or composition.
414. The method of any one of Embodiments 371-412, wherein level of SARM1 activity in the system is reduced by about or at least about 10% compared to absence of the oligonucleotide or composition.
415. The method of any one of Embodiments 371-412, wherein level of SARM1 activity in the system is reduced by about or at least about 20% compared to absence of the oligonucleotide or composition.
416. The method of any one of Embodiments 371-412, wherein level of SARM1 activity in the system is reduced by about or at least about 30% compared to absence of the oligonucleotide or composition.
417. The method of any one of Embodiments 371-412, wherein level of SARM1 activity in the system is reduced by about or at least about 40% compared to absence of the oligonucleotide or composition.
418. The method of any one of Embodiments 371-412, wherein level of SARM1 activity in the system is reduced by about or at least about 50% compared to absence of the oligonucleotide or composition.
419. The method of any one of Embodiments 371-412, wherein level of SARM1 activity in the system is reduced by about or at least about 60% compared to absence of the oligonucleotide or composition.
420. The method of any one of Embodiments 371-412, wherein level of SARM1 activity in the system is reduced by about or at least about 70% compared to absence of the oligonucleotide or composition.
421. The method of any one of Embodiments 371-412, wherein level of SARM1 activity in the system is reduced by about or at least about 80% compared to absence of the oligonucleotide or composition.
422. The method of any one of Embodiments 371-421, wherein level of SARM1 mRNA in the system is reduced by about or at least about 10%, 15%, 20%, 25%, 30%, 35%, 40%, 45%, 55%, 60%, 65%, 70%, 75%, 80%, 85%, or 90% compared to administering or delivering a reference oligonucleotide or composition.
423. The method of any one of Embodiments 371-421, wherein level of SARM1 mRNA in the system is reduced by about or at least about 10% compared to administering or delivering a reference oligonucleotide or composition.
424. The method of any one of Embodiments 371-421, wherein level of SARM1 mRNA in the system is reduced by about or at least about 15% compared to administering or delivering a reference oligonucleotide or composition.
425. The method of any one of Embodiments 371-421, wherein level of SARM1 mRNA in the system is reduced by about or at least about 20% compared to administering or delivering a reference oligonucleotide or composition.
426. The method of any one of Embodiments 371-421, wherein level of SARM1 mRNA in the system is reduced by about or at least about 25% compared to administering or delivering a reference oligonucleotide or composition.
427. The method of any one of Embodiments 371-421, wherein level of SARM1 mRNA in the system is reduced by about or at least about 30% compared to administering or delivering a reference oligonucleotide or composition.
428. The method of any one of Embodiments 371-421, wherein level of SARM1 mRNA in the system is reduced by about or at least about 35% compared to administering or delivering a reference oligonucleotide or composition.
429. The method of any one of Embodiments 371-421, wherein level of SARM1 mRNA in the system is reduced by about or at least about 40% compared to administering or delivering a reference oligonucleotide or composition.
430. The method of any one of Embodiments 371-421, wherein level of SARM1 mRNA in the system is reduced by about or at least about 45% compared to administering or delivering a reference oligonucleotide or composition.
431. The method of any one of Embodiments 371-421, wherein level of SARM1 mRNA in the system is reduced by about or at least about 50% compared to administering or delivering a reference oligonucleotide or composition.
432. The method of any one of Embodiments 371-421, wherein level of SARM1 mRNA in the system is reduced by about or at least about 55% compared to administering or delivering a reference oligonucleotide or composition.
433. The method of any one of Embodiments 371-421, wherein level of SARM1 mRNA in the system is reduced by about or at least about 60% compared to administering or delivering a reference oligonucleotide or composition.
434. The method of any one of Embodiments 371-421, wherein level of SARM1 mRNA in the system is reduced by about or at least about 65% compared to administering or delivering a reference oligonucleotide or composition.
435. The method of any one of Embodiments 371-421, wherein level of SARM1 mRNA in the system is reduced by about or at least about 70% compared to administering or delivering a reference oligonucleotide or composition.
436. The method of any one of Embodiments 371-421, wherein level of SARM1 mRNA in the system is reduced by about or at least about 75% compared to administering or delivering a reference oligonucleotide or composition.
437. The method of any one of Embodiments 371-421, wherein level of SARM1 mRNA in the system is reduced by about or at least about 80% compared to administering or delivering a reference oligonucleotide or composition.
438. The method of any one of Embodiments 371-421, wherein level of SARM1 mRNA in the system is reduced by about or at least about 85% compared to administering or delivering a reference oligonucleotide or composition.
439. The method of any one of Embodiments 371-421, wherein level of SARM1 mRNA in the system is reduced by about or at least about 90% compared to administering or delivering a reference oligonucleotide or composition.
440. The method of any one of Embodiments 371-439, wherein level of SARM1 polypeptide in the system is reduced by about or at least about 10%, 15%, 20%, 25%, 30%, 35%, 40%, 45%, 50%, 55%, or 60% compared to administering or delivering a reference oligonucleotide or composition.
441. The method of any one of Embodiments 371-439, wherein level of SARM1 polypeptide in the system is reduced by about or at least about 10% compared to administering or delivering a reference oligonucleotide or composition.
442. The method of any one of Embodiments 371-439, wherein level of SARM1 polypeptide in the system is reduced by about or at least about 15% compared to administering or delivering a reference oligonucleotide or composition.
443. The method of any one of Embodiments 371-439, wherein level of SARM1 polypeptide in the system is reduced by about or at least about 20% compared to administering or delivering a reference oligonucleotide or composition.
444. The method of any one of Embodiments 371-439, wherein level of SARM1 polypeptide in the system is reduced by about or at least about 25% compared to administering or delivering a reference oligonucleotide or composition.
445. The method of any one of Embodiments 371-439, wherein level of SARM1 polypeptide in the system is reduced by about or at least about 30% compared to administering or delivering a reference oligonucleotide or composition.
446. The method of any one of Embodiments 371-439, wherein level of SARM1 polypeptide in the system is reduced by about or at least about 35% compared to administering or delivering a reference oligonucleotide or composition.
447. The method of any one of Embodiments 371-439, wherein level of SARM1 polypeptide in the system is reduced by about or at least about 40% compared to administering or delivering a reference oligonucleotide or composition.
448. The method of any one of Embodiments 371-439, wherein level of SARM1 polypeptide in the system is reduced by about or at least about 45% compared to administering or delivering a reference oligonucleotide or composition.
449. The method of any one of Embodiments 371-439, wherein level of SARM1 polypeptide in the system is reduced by about or at least about 50% compared to administering or delivering a reference oligonucleotide or composition.
450. The method of any one of Embodiments 371-439, wherein level of SARM1 polypeptide in the system is reduced by about or at least about 55% compared to administering or delivering a reference oligonucleotide or composition.
451. The method of any one of Embodiments 371-439, wherein level of SARM1 polypeptide in the system is reduced by about or at least about 60% compared to administering or delivering a reference oligonucleotide or composition.
452. The method of any one of Embodiments 371-451, wherein level of SARM1 activity in the system is reduced by about or at least about 10%, 15%, 20%, 25%, 30%, 35%, 40%, 45%, 50%, 55%, 60%, 65%, 70%, 75%, or 80% compared to administering or delivering a reference oligonucleotide or composition.
453. The method of any one of Embodiments 371-451, wherein level of SARM1 activity in the system is reduced by about or at least about 10% compared to administering or delivering a reference oligonucleotide or composition.
454. The method of any one of Embodiments 371-451, wherein level of SARM1 activity in the system is reduced by about or at least about 20% compared to administering or delivering a reference oligonucleotide or composition.
455. The method of any one of Embodiments 371-451, wherein level of SARM1 activity in the system is reduced by about or at least about 30% compared to administering or delivering a reference oligonucleotide or composition.
456. The method of any one of Embodiments 371-451, wherein level of SARM1 activity in the system is reduced by about or at least about 40% compared to administering or delivering a reference oligonucleotide or composition.
457. The method of any one of Embodiments 371-451, wherein level of SARM1 activity in the system is reduced by about or at least about 50% compared to administering or delivering a reference oligonucleotide or composition.
458. The method of any one of Embodiments 371-451, wherein level of SARM1 activity in the system is reduced by about or at least about 60% compared to administering or delivering a reference oligonucleotide or composition.
459. The method of any one of Embodiments 371-451, wherein level of SARM1 activity in the system is reduced by about or at least about 70% compared to administering or delivering a reference oligonucleotide or composition.
460. The method of any one of Embodiments 371-451, wherein level of SARM1 activity in the system is reduced by about or at least about 80% compared to administering or delivering a reference oligonucleotide or composition.
461. The method of any one of Embodiments 422-460, wherein the reference oligonucleotide does not target SARM1 or the reference composition comprises no oligonucleotides targeting SARM1.
462. The method of any one of Embodiments 422-460, wherein the reference oligonucleotide is or comprises a scrambled oligonucleotide or the reference composition comprises scrambled oligonucleotides.
463. The method of any one of Embodiments 371-462, wherein the reduction is assessed at or after about or at least about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following administering or delivering the oligonucleotide or composition.
464. The method of any one of Embodiments 371-462, wherein the reduction is assessed at or after about 3 days following administering or delivering the oligonucleotide or composition.
465. The method of any one of Embodiments 371-462, wherein the reduction is assessed at or after about 7 days following administering or delivering the oligonucleotide or composition.
466. The method of any one of Embodiments 371-462, wherein the reduction is assessed at or after about 10 days following administering or delivering the oligonucleotide or composition.
467. The method of any one of Embodiments 371-462, wherein the reduction is assessed at or after about 14 days following administering or delivering the oligonucleotide or composition.
468. The method of any one of Embodiments 371-462, wherein the reduction is assessed at or after about 21 days following administering or delivering the oligonucleotide or composition.
469. The method of any one of Embodiments 371-462, wherein the reduction is assessed at or after about or at least about 1, 2, 3, 4, 5, 6, 7, 8, 9, 10, 11, 12, 13, 14, 15, 16, 17, 18, 19, 20, 21, 22, 23, 24 or 25 days, or about 1, 2, 3 or 4 weeks following removal or washout of the oligonucleotide or composition.
470. The method of any one of Embodiments 371-462, wherein the reduction is assessed at or after about 3 days following removal or washout of the oligonucleotide or composition.
471. The method of any one of Embodiments 371-462, wherein the reduction is assessed at or after about 7 days following removal or washout of the oligonucleotide or composition.
472. The method of any one of Embodiments 371-462, wherein the reduction is assessed at or after about 10 days following removal or washout of the oligonucleotide or composition.
473. The method of any one of Embodiments 371-462, wherein the reduction is assessed at or after about 14 days following removal or washout of the oligonucleotide or composition.
474. The method of any one of Embodiments 371-462, wherein the reduction is assessed at or after about 21 days following removal or washout of the oligonucleotide or composition.
475. The method of any one of Embodiments 371-462, wherein the reduction is about or at least about 40%-50% and wherein the reduction is assessed at or after about 7, 10, 14, or 21 days following removal or washout of the oligonucleotide or composition.
476. The method of any one of Embodiments 371-462, wherein the reduction is about or at least about 40% and wherein the reduction is assessed at or after about 7 days following removal or washout of the oligonucleotide or composition.
477. The method of any one of Embodiments 371-462, wherein the reduction is about or at least about 40% and wherein the reduction is assessed at or after about 10 days following removal or washout of the oligonucleotide or composition.
478. The method of any one of Embodiments 371-462, wherein the reduction is about or at least about 40% and wherein the reduction is assessed at or after about 14 days following removal or washout of the oligonucleotide or composition.
479. The method of any one of Embodiments 371-462, wherein the reduction is about or at least about 40% and wherein the reduction is assessed at or after about 21 days following removal or washout of the oligonucleotide or composition.
480. The method of any one of Embodiments 371-479, wherein the reduction is assessed in iPSC-derived motor neurons with oligonucleotide concentration at about 20 uM with gymnotic delivery.
481. The method of any one of Embodiments 371-479, wherein the reduction is assessed as described in Example 4.
482. A method for preventing or treating a condition, disorder or disease, comprising administering or delivering to a subject susceptible thereto an effective amount of an oligonucleotide or composition of any one of Embodiments 1-370.
483. The method of Embodiment 482, wherein onset of the condition, disorder or disease is delayed.
484. The method of Embodiment 482, wherein onset of the condition, disorder or disease is prevented.
485. A method for treating a condition, disorder or disease, comprising administering or delivering to a subject suffering therefrom an effective amount of an oligonucleotide or composition of any one of Embodiments 1-370.
486. The method of Embodiment 485, wherein severity of a symptom of the condition, disorder or disease is reduced.
487. The method of any one of Embodiments 485-486, wherein one or more clinical assessment results of the subject are independently improved.
488. The method of any one of Embodiments 482-487, wherein a condition, disorder or disease is Wallerian degeneration.
489. The method of any one of Embodiments 482-487, wherein a condition, disorder or disease is a neurodegenerative condition, disorder or disease.
490. The method of any one of Embodiments 482-487, wherein a condition, disorder or disease is amyotrophic lateral sclerosis.
491. The method of any one of Embodiments 482-487, wherein a condition, disorder or disease is peripheral neuropathy.
492. The method of any one of Embodiments 482-487, wherein a condition, disorder or disease is peripheral neuropathy included by chemotherapy.
493. The method of any one of Embodiments 482-487, wherein a condition, disorder or disease is Parkinson's disease.
494. The method of any one of Embodiments 482-487, wherein a condition, disorder or disease is Huntington's disease.
495. The method of any one of Embodiments 482-487, wherein a condition, disorder or disease is Alzheimer's disease.
496. The method of any one of Embodiments 482-487, wherein a condition, disorder or disease is frontotemporal dementia.
497. The method of any one of Embodiments 482-487, wherein a condition, disorder or disease is traumatic brain injury.
498. The method of any one of Embodiments 482-487, wherein a condition, disorder or disease is progressive supranuclear palsy.
499. The method of any one of Embodiments 482-487, wherein a condition, disorder or disease is corticobasal degeneration.
500. The method of any one of Embodiments 482-487, wherein a condition, disorder or disease is Wolfram Syndrome.
501. The method of any one of Embodiments 482-487, wherein a condition, disorder or disease is Friedreich's Ataxia.
502. The method of any one of Embodiments 482-487, wherein a condition, disorder or disease is multiple system atrophy.
503. The method of any one of Embodiments 482-487, wherein a condition, disorder or disease is spinal cerebellar ataxia.
504. The method of any one of Embodiments 482-487, wherein a condition, disorder or disease is spinal muscular atrophy (SMA).
505. The method of any one of Embodiments 482-487, wherein a condition, disorder or disease is Pick's Disease.
506. The method of any one of Embodiments 482-487, wherein a condition, disorder or disease is progressive motor atrophy.
507. The method of any one of Embodiments 482-487, wherein a condition, disorder or disease is stroke.
508. The method of any one of Embodiments 482-487, wherein a condition, disorder or disease is concussion.
509. The method of any one of Embodiments 482-487, wherein a condition, disorder or disease is intracerebral hemorrhage.
510. The method of any one of Embodiments 482-487, wherein a condition, disorder or disease is acute glaucoma.
511. The method of any one of Embodiments 482-487, wherein a condition, disorder or disease is seizure.
512. The method of any one of Embodiments 482-487, wherein a condition, disorder or disease is spinal cord injury.
513. The method of any one of Embodiments 482-512, wherein the oligonucleotide or composition is administered or delivered intrathecally.
514. The method of any one of Embodiments 482-512, wherein the oligonucleotide or composition is administered or delivered intravenously.
515. An oligonucleotide or composition of any one of Embodiments 1-370, for use in a method of any one of Embodiments 371-514.
516. An oligonucleotide or composition of any one of Embodiments 1-370, for use in manufacturing a medicament for use in a method of any one of Embodiments 371-514.
517. A method for manufacturing an oligonucleotide or composition of any one of Embodiments 1-370 according to a method described in the specification.

### Examples

Certain examples of provided technologies (compounds (oligonucleotides, reagents, etc.), compositions, methods (methods of preparation, use, assessment, etc.), etc.) were presented herein. Those skilled in the art appreciate that many technologies can be utilized to prepare and/or assess properties and/or activities of provided technologies in accordance with the present disclosure.

As appreciated by those skilled in the art, various technologies can be utilized to prepare oligonucleotides and compositions thereof in accordance with the present disclosure, including solid-phase processes using phosphoramidite chemistry. In some embodiments, preparation of oligonucleotides or compositions thereof comprises multiple cycles, each of which may independently comprise several steps. For example, in some embodiments, for incorporating a natural phosphate linkage, a cycle comprises detritylation, coupling, capping and oxidation; in some embodiments, for incorporating a phosphorothioate linkage, a cycle comprises detritylation, coupling, sulfurization and capping. Certain useful technologies are described in the “Manufacturing” section. See also, e.g., J. Org. Chem. 2018, 83, 19, 11577-11585.

### Example 1. Various Oligonucleotides and Compositions can Reduce SARM1 Expression

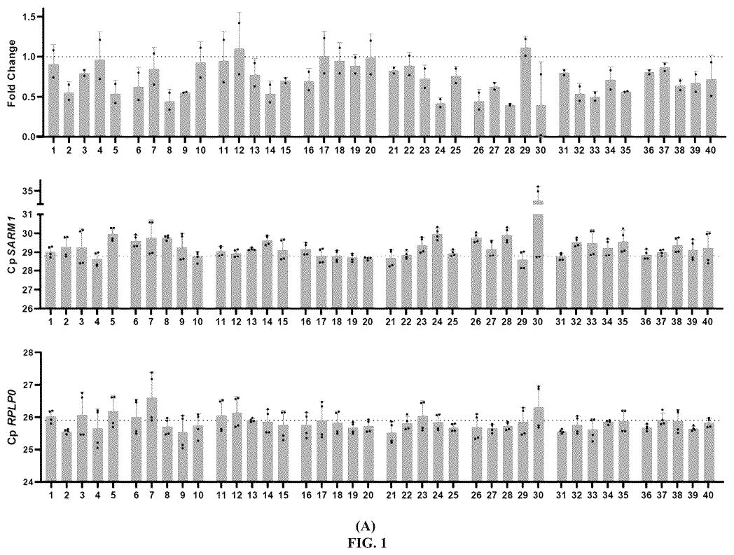

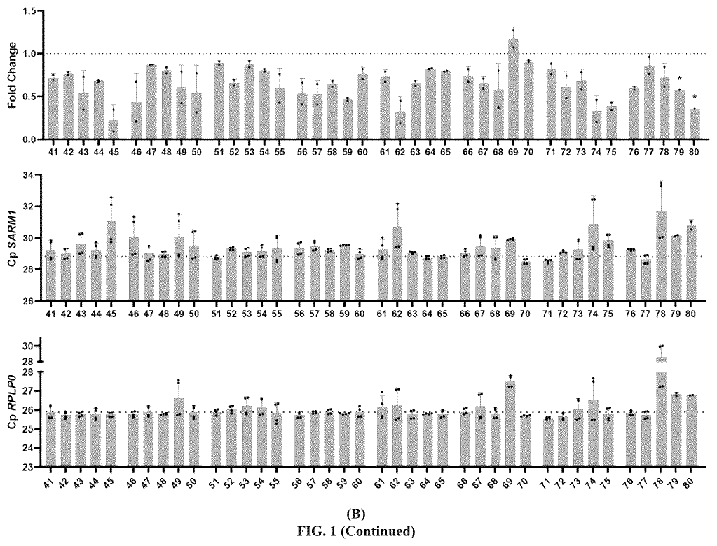

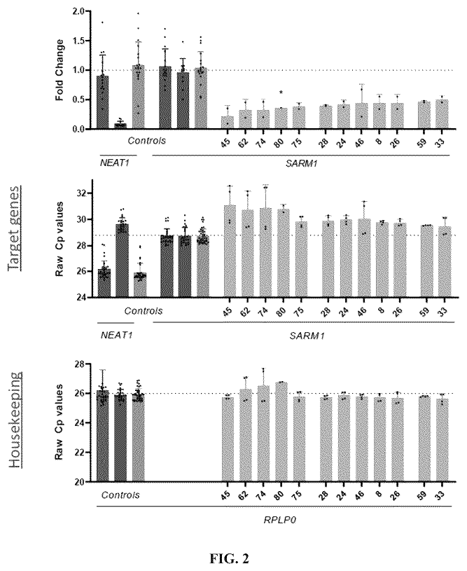

Oligonucleotides and compositions as described herein can provide in vitro knockdown of SARM1 transcript. In an assessment, human induced pluripotent stem cell (iPSC)-derived glutamatergic neurons (bit.bio ioGlutamatergic Neurons, Cat. No.: io1001) were seeded at 20,000 cells/well in a 96-well format. Following culturing of the cells for 4 days, various oligonucleotide compositions were added to the cells at a concentration of 10 μM in 10% TE buffer. Cells were incubated with the oligonucleotides for 72 hours to allow for gymnotic uptake and knockdown of SARM1 expression. Following this incubation period, cells were lysed and RNA was collected (Thermo Fisher cat. #A25603). RNA was used in real time RT-qPCR to quantify fold-change of SARM1 expression. In some embodiments, as shown in FIG. 1 and the tables below, various oligonucleotides provided in vitro knockdown of SARM1 expression. As shown in FIG. 1 and the table below, various oligonucleotides provided substantial in vitro knockdown of SARM1 expression as compared to reference conditions. See also data in FIG. 2 (positive control is <T*G*C*A*A*>G*T*C*T*G*A*C*G*C*C*<C*A*T*C*T> (SEQ ID NO: 401), negative control is <A*C*C*A*G*>T*G*C*A*T*T*C*A*T*T*<C*G*A*G*T> (SEQ ID NO: 402); all nucleosides are DNA nucleosides except those in < > which are 2′-MOE modified, and each * independently represents a phosphorothioate internucleotidic linkage).

TABLE 2
Reduction of SARM1 mRNA by various
oligonucleotide compositions.

| | Avg. % KD of |
|---|---|
| Oligonucleotide | SARM1 mRNA |
| 1 | 8.8 |
| 2 | 44.7 |
| 3 | 20.5 |
| 4 | 3.9 |
| 5 | 46.2 |
| 6 | 37.1 |
| 7 | 15.7 |
| 8 | 55.6 |
| 9 | 44.5 |
| 10 | 7.6 |
| 11 | 5.5 |
| 12 | −10.0 |
| 13 | 22.7 |
| 14 | 45.9 |
| 15 | 30.0 |
| 16 | 30.8 |
| 17 | −0.7 |
| 18 | 5.0 |
| 19 | 10.8 |
| 20 | 1.1 |
| 21 | 17.7 |
| 22 | 10.8 |
| 23 | 27.2 |
| 24 | 58.3 |
| 25 | 24.0 |
| 26 | 55.4 |
| 27 | 36.9 |
| 28 | 60.1 |
| 29 | −11.6 |
| 30 | 59.8 |
| 31 | 20.3 |
| 32 | 46.0 |
| 33 | 49.9 |
| 34 | 29.0 |
| 35 | 43.7 |
| 36 | 19.6 |
| 37 | 13.2 |
| 38 | 36.2 |
| 39 | 32.9 |
| 40 | 27.9 |
| 41 | 27.6 |
| 42 | 23.6 |
| 43 | 46.2 |
| 44 | 31.8 |
| 45 | 78.0 |
| 46 | 56.1 |
| 47 | 13.3 |
| 48 | 19.1 |
| 49 | 39.8 |
| 50 | 45.6 |
| 51 | 11.4 |
| 52 | 34.3 |
| 53 | 12.3 |
| 54 | 19.2 |
| 55 | 40.5 |
| 56 | 46.4 |
| 57 | 47.5 |
| 58 | 35.9 |
| 59 | 53.5 |
| 60 | 24.1 |
| 61 | 27.0 |
| 62 | 68.0 |
| 63 | 35.4 |
| 64 | 17.6 |
| 65 | 20.7 |
| 66 | 25.3 |
| 67 | 35.0 |
| 68 | 41.4 |
| 69 | −17.1 |
| 70 | 9.2 |
| 71 | 17.8 |
| 72 | 39.1 |
| 73 | 31.8 |
| 74 | 66.9 |
| 75 | 61.9 |
| 76 | 40.3 |
| 77 | 13.8 |
| 78 | 27.4 |
| 79 | 41.6 * |
| 80 | 63.9 * |

* indicates data from only 1 technical replicate displayed.

### Example 2. Various Oligonucleotides and Compositions do not Display Obvious Cytotoxicity

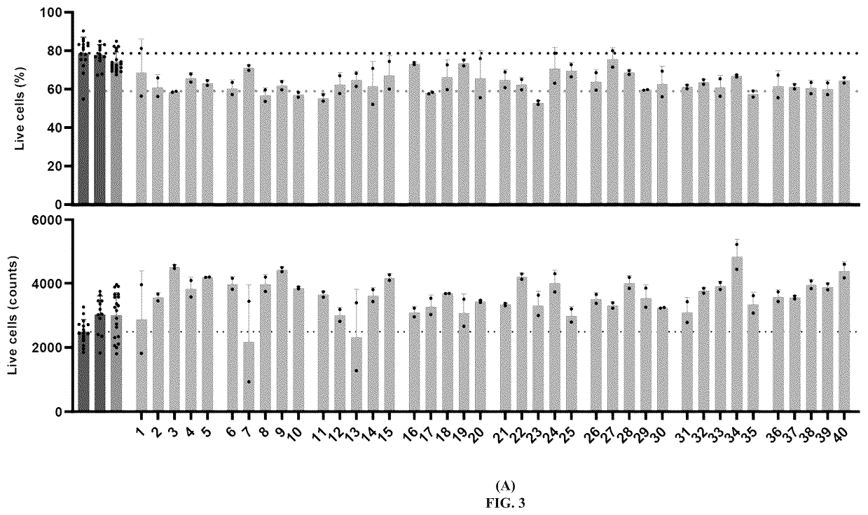

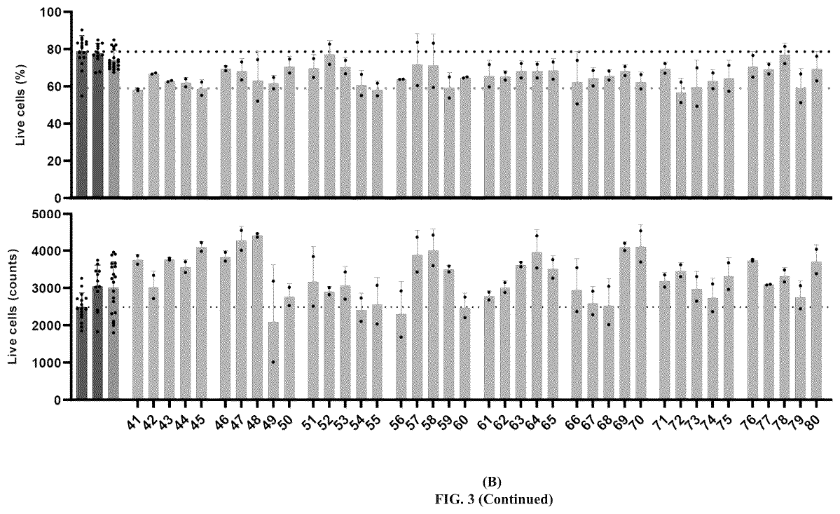

Among other things, the present disclosure provides oligonucleotides and compositions having low or no cytotoxicity. In an assessment, human iPSC-derived glutamatergic neurons (bit.bio ioGlutamatergic Neurons, Cat. No.: IWIO) were seeded at 20,000 cells/well in a 96-well format. Following culturing of the cells for 4 days, various oligonucleotides were added to the cells at a concentration of 10 μM in 10% TE buffer. Cells were incubated with the oligonucleotides for 72 hours. Following this incubation period, cells underwent Hoechst staining (5 μg/mL). The number of live, dead, and total (live+dead) cells were counted and the percentage of live cells was calculated. As shown in FIG. 3, no significant change in the percentage of live cells were observed following treatment with various oligonucleotides as compared to vehicle treatment (TE buffer). Positive control is <T*G*C*A*A*>G*T*C*T*G*A*C*G*C*C*<C*A*T*C*T> (SEQ ID NO: 401) (targeting NEAT1), negative control is <A*C*C*A*G*>T*G*C*A*T*T*C*A*T*T*<C*G*A*G*T> (SEQ ID NO: 402); all nucleosides are DNA nucleosides except those in < > which are 2′-MOE modified, and each * independently represents a phosphorothioate internucleotidic linkage. Staurosporine was utilized to induce toxicity in a separate well.

### Example 3. Various Oligonucleotides and Compositions can Provide Dose-Dependent Reduction of SARM1 Expression

Among other things, the present disclosure demonstrate that various provided oligonucleotides and compositions can provide dose-dependent reduction of SARM1 mRNA levels. Results from an assessment are presented below as an example.

Cells: Human Bit.bio ioGlutamatergic neurons were seeded at 20,000 cells/well in 96-well format. Thawing, seeding and culturing were according to supplier's instructions (e.g., coating: 0.01% PLO and 26 ng/cm2 laminin).

Oligonucleotide treatment: Oligonucleotide compositions 5, 8, 24, 26, 28, 32, 33, 43, 45, 46, 56, 57, 59, 62, 74, 75, and 80 and control (e.g., TUG1 as a negative control) were assessed at multiple concentrations. Concentrations included 30 μM, 10 μM, 3.3 μM, 1.1 μM, 0.37 μM, 0.12 μM, 0.04 μM, and 0.01 μM. Vehicle used was 10% TE buffer for all concentrations except for 30 μM, which used 30% TE buffer. A positive control oligonucleotide composition targeting NEAT1 (10 μM) and a negative control oligonucleotide composition targeting TUG1 (10 μM) were also tested. Vehicle alone (10% TE buffer without oligonucleotides) was also assessed. Oligonucleotides were added to cells 4 days after seeding the cells (by gymnotic uptake, 72 hours incubation). Oligonucleotide treatments were in duplicates on separate plates. The positive control was <T*G*C*A*A*>G*T*C*T*G*A*C*G*C*C*<C*A*T*C*T> (SEQ ID NO: 401) (targeting NEAT1), and the negative control was <A*C*C*A*G*>T*G*C*A*T*T*C*A*T*T*<C*G*A*G*T> (SEQ ID NO: 402) (all nucleosides are DNA nucleosides except those in < > which are 2′-MOE modified, and each * independently represents a phosphorothioate internucleotidic linkage).

Harvesting: Various technologies can be utilized to harvest cells and mRNA. Cells-to-Ct 1-Step TaqMan kit (Life technologies), according to supplier's instructions (cell lysis without separate RNA purification and one-step reverse transcription+real time PCR (real time RT-PCR), was utilized. For each well in the 96 well plate, 30 μL lysis buffer was utilized 3 days after oligonucleotide treatment.

Toxicity Check: Various technologies can be utilized to assess toxicity. Hoechst (5 μg/mL) staining and imaging were performed at 3 days after oligonucleotide treatment. A high content algorithm was used to quantify the number of Hoechst nuclei as a read-out for potential oligonucleotide-induced toxicity.

RT-qPCR: Various technologies can be utilized to assess transcript levels. 10% of Cells-to-Ct lysate samples (or water for non-template control; NTC) were assessed. Assessment was performed as single-plex reactions, in 384-well format, in technical duplicates. TaqMan Gene Expression Assay using LightCycler 480 equipment was utilized. (NEAT1 Tagman assay was utilized for control oligonucleotide samples only, and SARM1 and RPLP0 Tagman assays are utilized for control and test oligonucleotide samples). The following probe sets were utilized.

| Primer-probe Set | Supplier | Assay ID |
|---|---|---|
| Human NEAT1 FAM (target; control | IDT | |
| samples) | | |
| Human SARM1 FAM (target) | Thermo Fisher | Hs00248344_m1 |
| Human RPLP0VIC_PL | Thermo Fisher | Hs00420895_gH |
| (housekeeping) | | |

Data analysis: Various technologies can be utilized to analyze data in accordance with the present disclosure. Cp values were determined using the second derivative method (Lightcycler software). Fold-change gene expression was determined by ΔΔCp method (normalized to negative control oligonucleotide: TUG1 at 10 μM). For NEAT1 expression: ΔCp=Cp_{NEAT1}-Cp_{RPLP0}; ΔΔCp=ΔCp_{NEAT1}-ΔCp_{TUG1}; Fold change of NEAT1 expression=2^{-ΔΔCp}. For SARM1 expression: ΔCp=Cp_{SAPM1}-Cp_{RPLP0}; ΔΔCp=ΔCp_{NEAT1/SARM1}-ΔCp_{TUG1}; fold change of SARM1 expression=2^{-ΔCp}.

A strong separation of positive and negative control oligonucleotide compositions was observed. Treatment with 10 μM NEAT1 oligonucleotide resulted in 90% or more NEAT1 knockdown on plates tested. Some limited marked intra- or inter-plate variability between negative controls, and some plates showed higher variability among the negative controls compared to others. No plate positional effects were observed based on control sample performance (vehicle, positive and negative controls), and no clear plate drift was observed between plates tested. No effects of control treatment on SARM1 and/or RPLP0 expression were observed when compared to vehicle treatment (fold changes and/or Cp values). There was some variability of certain plates in comparison to other plates. There was moderate concordance between biological replicates.

As demonstrated in Table 3 below, concentration-dependent increase in SARM1 knockdown was observed for various provided oligonucleotide compositions. The negative control targeting TUG1 did not show a significant concentration-dependent effect on SARM1 expression.

TABLE 3
Concentration-dependent knockdown of SARM1 mRNA
by various oligonucleotide compositions.

% Knockdown of SARM1 mRNA

| | | | | | | |
|---|---|---|---|---|---|---|
| Conc. | 5 | 8 | 24 | 26 | 28 | 32 |

| | | | | | | | |
|---|---|---|---|---|---|---|---|
| 30 | μM | 76.1 | 84.1 | 81.2 | 81.9 | 86.2 | 56.7 |
| 10 | μM | 57.6 | 63.7 | 60.9 | 50.2 | 51.6 | 31.0 |
| 3.33 | μM | 39.0 | 51.0 | 49.8 | 39.7 | 39.8 | 12.9 |
| 1.11 | μM | 22.8 | 38.3 | 40.3 | 30.2 | 24.0 | 11.1 |
| 0.37 | μM | 13.6 | 24.6 | 22.9 | 24.6 | 24.9 | 2.0 |
| 0.12 | μM | 7.1 | 27.0 | 26.5 | 16.4 | 29.3 | 6.8 |
| 0.04 | μM | 16.6 | 30.2 | 24.2 | 15.2 | 19.5 | 28.1 |
| 0.01 | μM | 11.2 | 26.3 | 25.0 | 13.0 | 15.1 | 15.5 |

% Knockdown of SARM1 mRNA

| | | | | | | |
|---|---|---|---|---|---|---|
| Conc. | 33 | 43 | 45 | 46 | 56 | 57 |

| | | | | | | | |
|---|---|---|---|---|---|---|---|
| 30 | μM | 61.2 | 55.8 | 88.7 | 76.1 | 61.8 | 46.4 |
| 10 | μM | 33.0 | 31.4 | 79.9 | 42.5 | 25.6 | 4.0 |
| 3.33 | μM | 25.1 | 10.8 | 67.1 | 3.3 | 18.5 | −7.5 |
| 1.11 | μM | 0.8 | −20.9 | 52.3 | −15.3 | 3.8 | −11.6 |
| 0.37 | μM | 13.7 | −0.3 | 59.9 | 2.6 | 9.2 | −11.0 |
| 0.12 | μM | −6.8 | −15.8 | 37.4 | −9.3 | −4.6 | −15.3 |
| 0.04 | μM | −5.0 | −12.2 | 21.5 | −7.5 | 5.2 | −12.4 |
| 0.01 | μM | −11.0 | 8.9 | 21.2 | −8.4 | −9.2 | −4.3 |

| | % Knockdown of SARM1 mRNA | | | | |
|---|---|---|---|---|---|
| Conc. | 59 | 62 | 74 | 75 | 80 |

| | | | | | | |
|---|---|---|---|---|---|---|
| 30 | μM | 67.5 | 80.7 | 78.2 | 77.9 | 73.1 |
| 10 | μM | 45.5 | 52.9 | 38.0 | 39.4 | 16.7 |
| 3.33 | μM | 43.9 | 51.7 | 23.9 | 27.9 | −0.6 |
| 1.11 | μM | −0.6 | 1.1 | −4.7 | −8.1 | −23.3 |
| 0.37 | μM | 32.2 | 37.3 | 14.3 | 17.4 | −9.9 |
| 0.12 | μM | 6.8 | −2.3 | 3.0 | −11.2 | −25.7 |
| 0.04 | μM | 6.0 | −5.8 | −6.5 | −15.2 | −18.6 |
| 0.01 | μM | N/A | N/A | N/A | N/A | N/A |

No apparent cell toxicity was observed for all tested oligonucleotide compositions even when tested at high concentrations except for oligonucleotide composition 80, for which at the highest oligonucleotide concentration assessed (30 μM), lower live cells count and lower 00 viability was observed (at 10 μM, higher live cells count and slightly lower 00 viability was observed) compared to the negative control or vehicle. Further, imaging revealed no morphological changes upon treatment with all oligonucleotide compositions assessed except for oligonucleotide composition 80, for which in high-concentration instances fewer neurites were observed when compared to the negative control.

### Example 4. Provided Technologies can Provide Reduction of SARM1 mRNA and Protein Levels

Among other things, the present disclosure demonstrate that provided oligonucleotides and compositions can provide reduction of SARM1 mRNA and protein levels, in some instances, for days, weeks, or longer after administration, delivery, or removal of the oligonucleotides and compositions. Results from certain assessments are presented below as an example. Those skilled in the art appreciate that other technologies can also be utilized to assess provided technologies in accordance with the present disclosure to confirm technical effects, benefits, advantages, etc. of provided technologies.

Cells: iCell Motor Neurons, seeded at 32,000 cells/well in 96-well format. Thawing, seeding and culturing can be performed according to supplier's instructions.

Oligonucleotide treatment: Oligonucleotide compositions 45, 62, and scrambled (negative control). Concentration: 19.0 μM for oligonucleotide composition 45, 16.2 μM for oligonucleotide composition 62, and 20 μM for negative control scrambled oligonucleotide. Vehicle: H₂O without oligonucleotides. Oligonucleotides were added to cells on day 7 after seeding the cells (by gymnotic uptake) and removed by medium refreshment on day 9 (48 hours total incubation). Cells were collected and lysed on day 9 after seeding the cells (baseline) and 3, 7, 10, 14, and 21 days after removal of oligonucleotide compositions. For mRNA collection, treatments were conducted in triplicate for each condition, with a separate plate for each collection time point. For protein collection, treatments were conducted in three triplicates for each condition with a separate plate for each collection time point.

mRNA Harvesting: Cells were harvested using Cells-to-Ct 1-Step TaqMan kit (Thermo Fisher), according to supplier's instructions (25 μL/well lysis buffer) on day 7 after seeding the cells (baseline) and at 3, 7, 10, 14, and 21 days after removal of oligonucleotide compositions.

RT-PCR: 15% of Cells-to-Ct lysate samples and the primer-probe sets in the table below, as single-plex reactions, in 384-well format, in technical duplicates. TaqMan Gene Expression Assay using LightCycler 480 equipment (Roche). (SARM1 and RPLP0 Taqman assays for control and test oligonucleotide samples.)

| Primer-probe Set | Supplier | Assay ID |
|---|---|---|
| Human SAR1 FAM (target) | Thermo Fisher | Hs00248344_m1 |
| Human RPLP0 VIC_PL | Thermo Fisher | Hs00420895 |
| (housekeeping) | | |

mRNA Data Analysis: Cp values were determined using the second derivative method (Lightcycler software). % knockdown of gene expression was determined by Pfaffl method to account for primer efficiency mismatch: RQ=(E_{target})^{ΔCt(target)}/(E_{reference})^{ΔCt(reference)} (normalized to negative control: scrambled), wherein ΔCt(target)=Ct(target gene in calibrator)−Ct(target gene in tests) and ΔCt(reference)=Ct(reference gene in calibrator)−Ct(reference gene in tests).

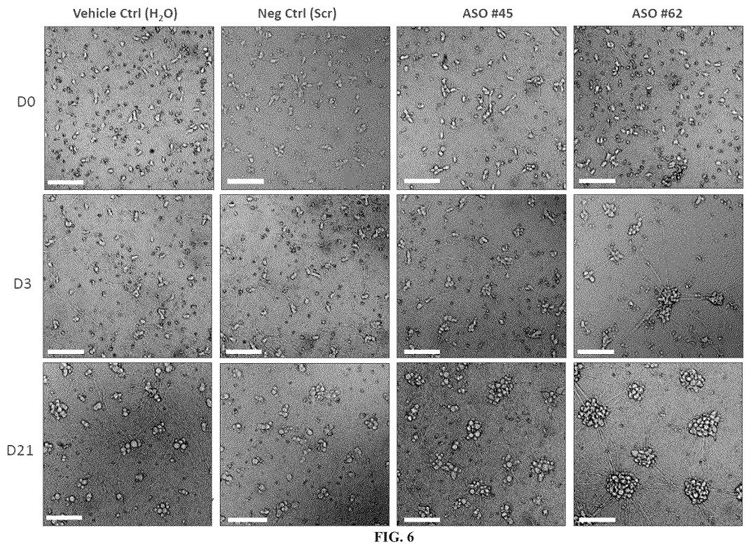

Treatment of cells with vehicle (H₂O) or scrambled negative control oligonucleotide resulted in only minor changes in housekeeping (RPLP0) and target (SARM1) gene Cp values. Small variability in Cp values was observed within replicate samples for SARM1 and RPLP0 upon treatment with vehicle (H₂O) or scrambled negative control oligonucleotide, without the intention to be limited by theory, likely due to well-to-well variability in cell viability. Cell viability was assessed by Hoescht staining (allowing for counting of cell nuclei) and brightfield imaging. No major cytotoxicity was observed. Some variation in cell viability was observed between individual wells, but increased cell counts were observed at day 0 for all wells treated with oligonucleotide composition 62. Additionally, as displayed in FIG. 6, no significant effects on cell morphology were observed upon treatment with vehicle (H₂O) or scrambled negative control oligonucleotide. In an assessment, some cell clustering was observed upon treatment with oligonucleotide composition 45, e.g., at day 21, and some cell clustering was observed upon treatment with oligonucleotide composition 62, e.g., at day 21. Further, neuronal network formation was observed in all treatment conditions.

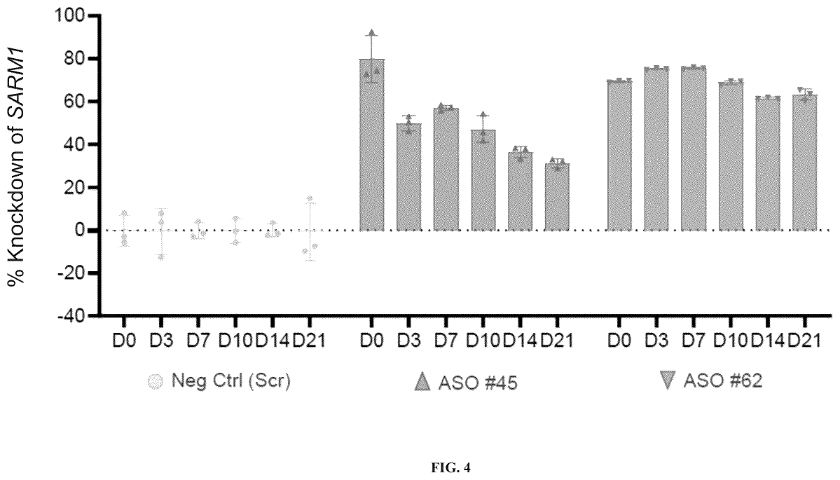

As shown in FIG. 4 and the table below, SARM1 mRNA knockdown was confirmed. Both oligonucleotide 45 and oligonucleotide 62 show initial significant knockdown of SARM1 mRNA on day 0. Oligonucleotide 45 can provide about 79% knockdown of SARM1 mRNA at 19.0 μM on day 0 post-removal of oligonucleotide, about 50% on day 3 post-removal of oligonucleotide, and about 31% knockdown of SARM1 mRNA at 19.0 μM on day 21 post-removal of oligonucleotide. Oligonucleotide 62 can provide about 70% knockdown of SARM1 mRNA at 16.2 μM on day 0 post-removal of oligonucleotide, about 75% knockdown on days 3 and 7 post-removal of oligonucleotide, and about 63% knockdown of SARM1 mRNA at 16.2 μM on day 21 post-removal of oligonucleotide. Among other things, provided technologies, e.g., oligonucleotide 62, demonstrated persistent knockdown of SARM1 mRNA from day 0 post-removal of oligonucleotide to at least day 21 post-removal of oligonucleotide. The scrambled negative control oligonucleotide did not display knockdown of SARM mRNA levels. Among other things, data of the present Example confirm that provided technologies can provide persistent knockdown of SARM1 mRNA, e.g., following removal or washout of the oligonucleotide or composition.

Protein Harvesting: Cell lysates prepared using RIPA lysis buffer (30 μL/well), including Halt protease inhibitor cocktail (Thermo Fisher). Three wells per replicate were pooled to ensure obtainment of sufficient amounts for protein detection. Three replicates were harvested per condition per time point. Protein yield was assessed by BCA assay.

Protein Detection: SARM1 protein was detected using western blot via Jess equipment (ProteinSimple) according to supplier's instructions. Two replicates for each condition and time point were examined. SARM1 antibody (ab226930; Abcam) was used at a 1:20 dilution.

Protein Data Analysis: Chemiluminescence-based detection of target immunoreactivity. Signal analysis for area under curve (AUC), detected molecular (MW), and signal-to-noise ratios using Compass software. Quantification of % target protein knockdown, normalized for total protein.

SARM1 protein levels in an assessment were slightly affected by treatment with vehicle (H₂O) or scrambled negative control oligonucleotide as compared to untreated cells at 48 hours post-treatment. Vehicle (H₂O) treatment appeared to lead to modestly lower SARM1 protein levels. Scrambled negative control oligonucleotide displayed notable variation in an assessment in SARM1 protein levels, which, without the intention to be limited by theory, may have resulted from variation in cell viability and/or cell confluency. In an assessment, significant replicate variability was observed in SARM1 protein levels upon treatment with vehicle (H₂O) or scrambled negative control oligonucleotide at various time points; however, scrambled negative control oligonucleotide did not result in increased replicate variability as compared to vehicle (H₂O). For the scrambled negative control oligonucleotide, variation in SARM1 protein levels was particularly notable at some time points (e.g., day 7) in an assessment and, without the intention to be limited by theory, may be the result of a number of factors, such as well-to-well variability in cell viability, non-specific background staining, variations in protein loading, etc. Cell viability was assessed by Hoescht staining (allowing for counting of cell nuclei) and brightfield imaging. No major cytotoxicity was observed. Some variation in cell viability was observed between individual wells, but increased cell counts were observed at day 0 for all wells treated with oligonucleotide composition 62. Again, as displayed in FIG. 6, no significant effects on cell morphology were observed upon treatment with vehicle (H₂O) or scrambled negative control oligonucleotide. In an assessment, some cell clustering was observed upon treatment with oligonucleotide composition 45, e.g., at day 21, and some cell clustering was observed upon treatment with oligonucleotide composition 62, e.g., at day 21. Further, neuronal network formation was observed in all treatment conditions.

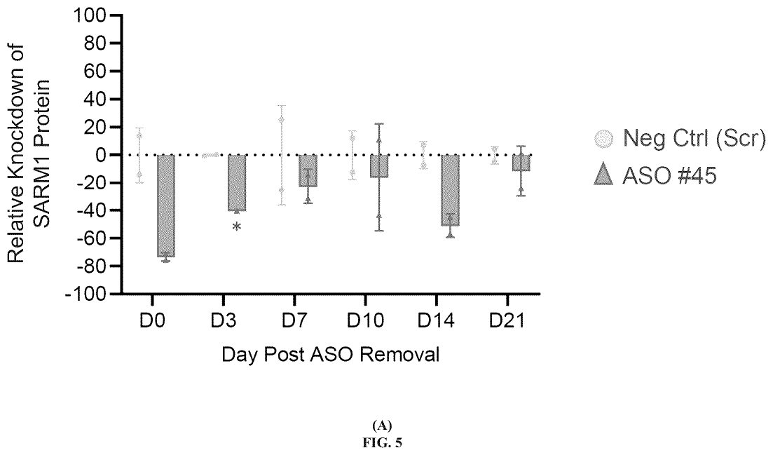

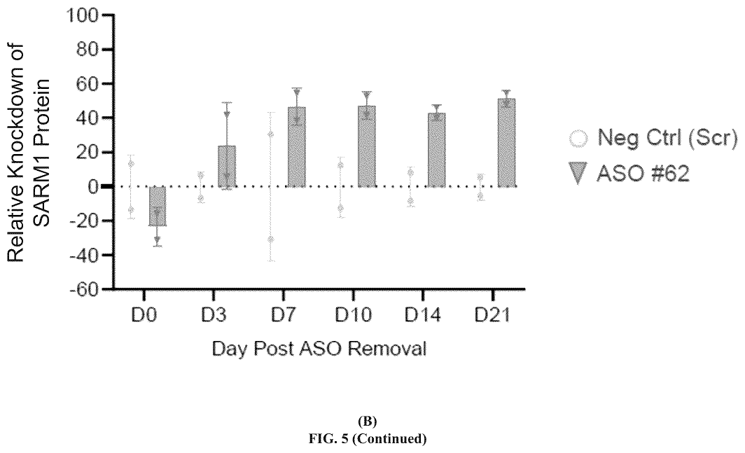

As shown in FIG. 5 and the table below, SARM1 protein knockdown was confirmed. Oligonucleotide 62 provides about 51% knockdown of SARM1 protein at 16.2 μM at day 21 post-removal of oligonucleotide, as shown in FIG. 5B. Further, oligonucleotide 62 demonstrated persistent SARM1 protein knockdown from at least day 7 post-removal of oligonucleotide and maintained persistent SARM1 protein knockdown to at least day 21 post-removal of oligonucleotide. Oligonucleotide 45 did not display knockdown of SARM1 protein in this experiment when assessed at time points as shown in FIG. 5. For treatment with oligonucleotide 45, variation in SARM1 protein levels was higher at some time points (e.g., day 10) and, without the intention to be limited by theory, may be the result of a number of factors, such as well-to-well variability in cell viability, non-specific background staining, variations in protein loading, some SARM1 knockdown levels elicited by the negative control, etc. The scrambled negative control oligonucleotide did not display knockdown of SARM1 protein levels. Among other things, data of the present Example confirm that provided technologies such as oligonucleotide 62 can provide significant and persistent knockdown of SARM1 protein, e.g., following removal or washout of the oligonucleotide or composition.

TABLE 4
Knockdown (KD) of SARM1 mRNA and protein levels
by provided oligonucleotide compositions.

| Oligonucleotide 45 | | | Oligonucleotide 62 | | |
|---|---|---|---|---|---|
| | mRNA | Protein | | mRNA | Protein |
| Day | % KD | % KD | Day | % KD | % KD |
| 0 | 79.9 | −73.5 | 0 | 69.5 | −23.41 |
| 3 | 49.9 | −40.3 * | 3 | 75.3 | 23.9 |
| 7 | 57.1 | −22.8 | 7 | 75.6 | 46.6 |
| 10 | 47.2 | −16.2 | 10 | 69.0 | 47.3 |
| 14 | 36.4 | −51.0 | 14 | 61.6 | 43.2 |
| 21 | 31.3 | −11.6 | 21 | 63.1 | 51.4 |

* indicates data from only 1 technical replicate displayed.

Various other technologies are available for assessing properties and/or activities of provided technologies in accordance with the present disclosure. For example, reduction of levels of SARM1 polypeptides can be assessed through western blot or immunostaining. In some embodiments, provided oligonucleotides and compositions are assessed for reduction of SARM1 activity. In some embodiments, provided oligonucleotides and compositions are assessed for reduction of neuronal degeneration, e.g., through using one or more cell models. In some embodiments, provided oligonucleotides and compositions are assessed for enhanced recovery from axotomy.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. An oligonucleotide having the structure:
/52MOErG/*/i2MOErT/*/i2MOErC/*/i2MOErT/*/i2MOErC/*/iMe-dC/*A*G*A*A*/iMe-dC/*T*G*A*G*/i2MOErC/*/i2MOErA/*/i2MOErG/*/i2MOErG/*/32MOErG/ (SEQ ID NO: 382) or a salt thereof, wherein: 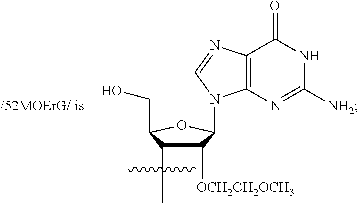 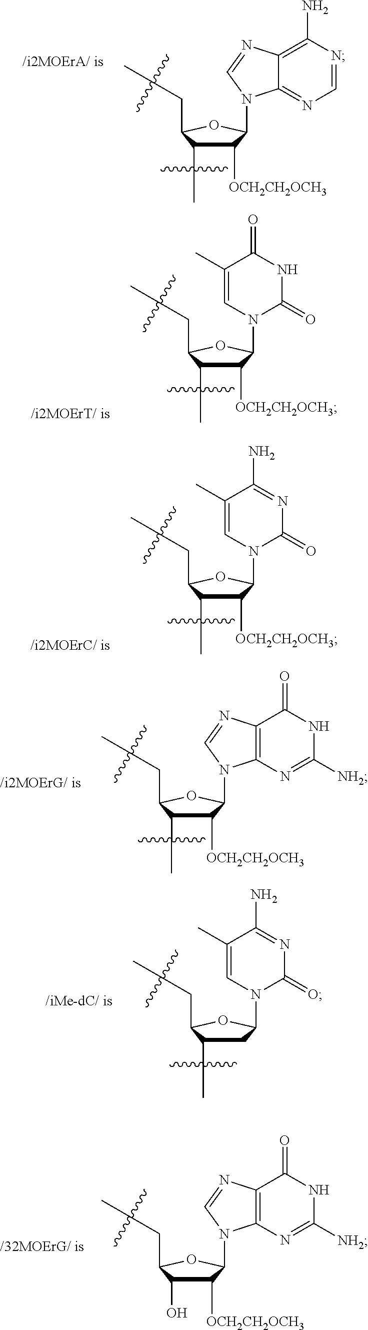
* is —O—P(O)(SH)—O—;
each of A, T and G is independently deoxyadenosine, thymidine, and deoxyguanosine, respectively.

2. An oligonucleotide having the structure:
/52MOErC/*/i2MOErC/*/i2MOErT/*/i2MOErT/*/i2MOErG/*/iMe-dC/*A*G*G*/iMe-dC/*T*/iMe-dC/*T*T*G*/i2MOErA/*/i2MOErT/*/i2MOErG/*/i2MOErG/*/32MOErC/(SEQ ID NO: 365) or a salt thereof, wherein:
* is —O—P(O)(SH)—O—; 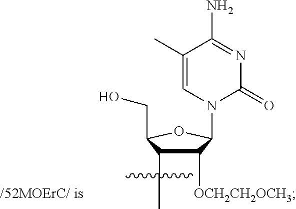 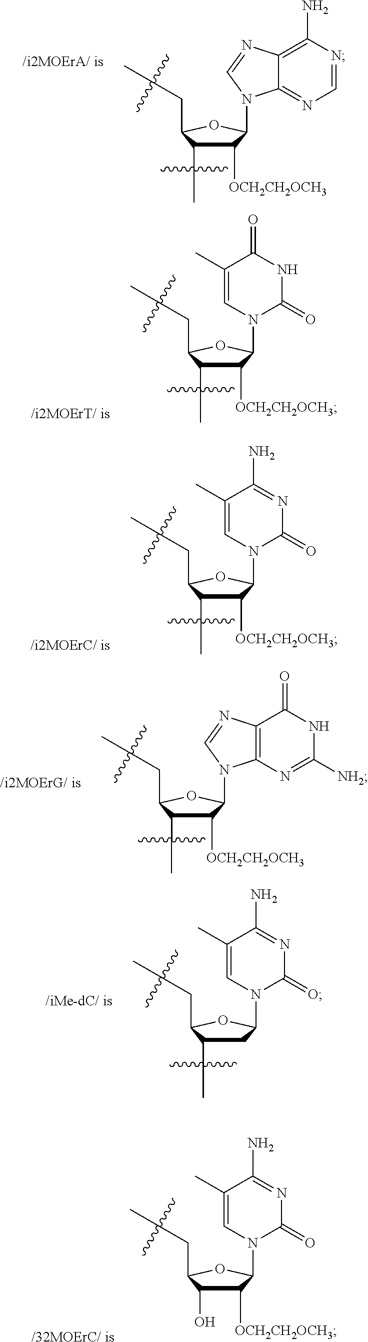
and
each of A, T and G is independently deoxyadenosine, thymidine, and deoxyguanosine, respectively.

3.-25. (canceled)

26. A composition comprising:
an oligonucleotide or a salt thereof, and
one or more diastereomers of the oligonucleotide with respect to chiral linkage phosphorus, or one or more salts of the diastereomers,
wherein the oligonucleotide is /52MOErG/*/i2MOErT/*/i2MOErC/*/i2MOErT/*/i2MOErC/*/iMe-dC/*A*G*A*A*/iMe-dC/*T*G*A*G*/i2MOErC/*/i2MOErA/*/i2MOErG/*/i2MOErG/*/32MOErG/ (SEQ ID NO: 382), wherein:
* is —O—P(O)(SH)—O—; 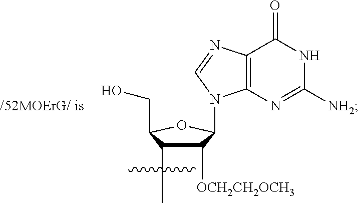 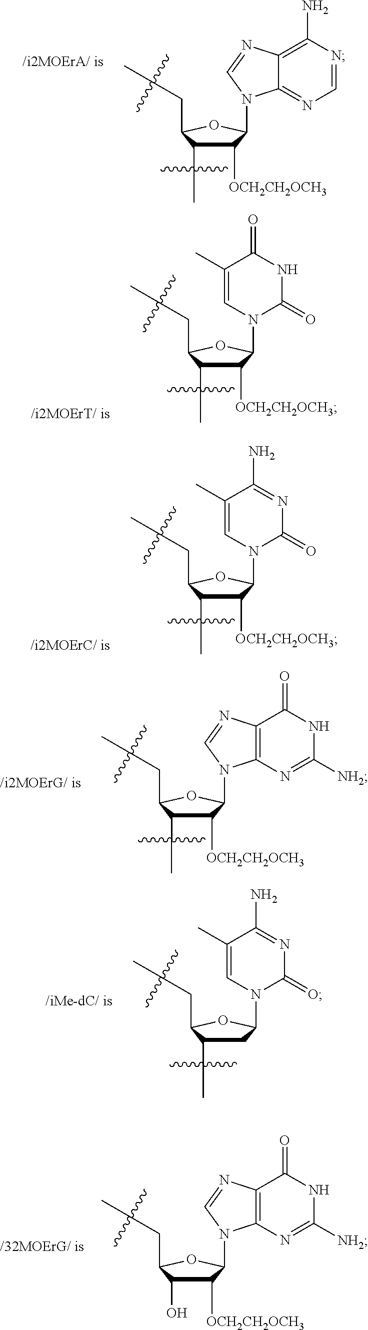
and
each of A, T and G is independently deoxyadenosine, thymidine, and deoxyguanosine, respectively.

27. A composition comprising:
an oligonucleotide or a salt thereof, and
one or more diastereomers of the oligonucleotide with respect to chiral linkage phosphorus, or one or more salts of the diastereomers,
wherein the oligonucleotide is
/52MOErC/*/i2MOErC/*/i2MOErT/*/i2MOErT/*/i2MOErG/*/iMe-dC/*A*G*G*/iMe-dC/*T*/iMe-dC/*T*T*G*/i2MOErA/*/i2MOErT/*/i2MOErG/*/i2MOErG/*/32MOErC/(SEQ ID NO: 365), wherein:
* is —O—P(O)(SH)—O—; 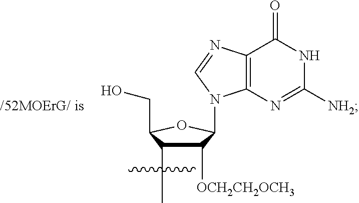 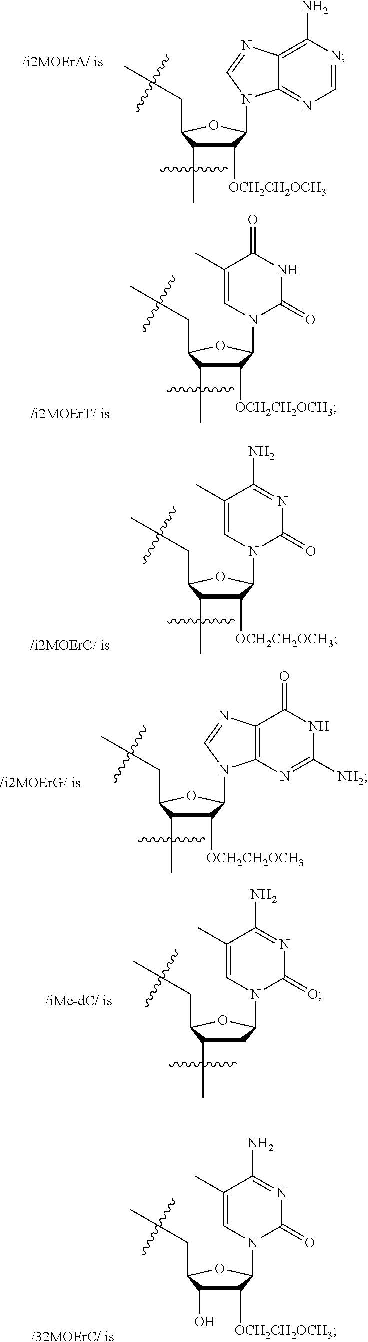
each of A, T and G is independently deoxyadenosine, thymidine, and deoxyguanosine, respectively.

28. (canceled)

29. The composition of claim 26, wherein for each chiral linkage phosphorus, the percentage of the Rp configuration is independently about 20%-80%.

30. The composition of claim 26, wherein the composition comprises a salt of the oligonucleotide, and one or more salts of one or more diastereomers of the oligonucleotide.

31. (canceled)

32. The composition of claim 26, wherein the composition comprises a pharmaceutically acceptable salt of the oligonucleotide, one or more pharmaceutically acceptable salts of one or more diastereomers of the oligonucleotide, and a pharmaceutically acceptable carrier.

33.-36. (canceled)

37. A method for reducing level of SARM1 mRNA in a system, comprising administering or delivering to the system an effective amount of the oligonucleotide of claim 1.

38. A method for reducing level of SARM1 polypeptide in a system, comprising administering or delivering to the system an effective amount of the oligonucleotide of claim 1.

39. A method for reducing level of SARM1 mRNA in a system, comprising administering or delivering to the system an effective amount of the composition of claim 26.

40. A method for reducing level of SARM1 polypeptide in a system, comprising administering or delivering to the system an effective amount the composition of claim 26.

41.-52. (canceled)

53. A method for preventing or treating a condition, disorder or disease, comprising administering or delivering to a subject susceptible thereto an effective amount of the composition of claim 26.

54.-61. (canceled)

62. The composition of claim 27, wherein for each chiral linkage phosphorus, the percentage of the Rp configuration is independently about 20%-80%.

63. The composition of claim 27, wherein the composition comprises a salt of the oligonucleotide, and one or more salts of one or more diastereomers of the oligonucleotide.

64. The composition of claim 27, wherein the composition comprises a pharmaceutically acceptable salt of the oligonucleotide, one or more pharmaceutically acceptable salts of one or more diastereomers of the oligonucleotide, and a pharmaceutically acceptable carrier.

65. A method for reducing level of SARM1 mRNA in a system, comprising administering or delivering to the system an effective amount of the oligonucleotide of claim 2.

66. A method for reducing level of SARM1 polypeptide in a system, comprising administering or delivering to the system an effective amount of the oligonucleotide of claim 2.

67. A method for reducing level of SARM1 mRNA in a system, comprising administering or delivering to the system an effective amount of the composition of claim 27.

68. A method for reducing level of SARM1 polypeptide in a system, comprising administering or delivering to the system an effective amount of the composition of claim 27.

69. A method for preventing or treating a condition, disorder or disease, comprising administering or delivering to a subject susceptible thereto an effective amount of the composition of claim 27.


SEQUENCE LISTING (402 sequences)

SEQ ID NO: 1 - DNA, 20 bp, synthetic construct
  source 1..20: mol_type = other DNA
  misc_difference 5: note = May be uridine
  misc_difference 11: note = May be uridine
ccactagccctgggagcaaa

SEQ ID NO: 2 - DNA, 20 bp, synthetic construct
  source 1..20: mol_type = other DNA
  misc_difference 5: note = May be uridine
  misc_difference 7: note = May be uridine
  misc_difference 11: note = May be uridine
  misc_difference 15: note = May be uridine
gccatctccatccatagagc

SEQ ID NO: 3 - DNA, 20 bp, synthetic construct
  source 1..20: mol_type = other DNA
  misc_difference 9: note = May be uridine
  misc_difference 11: note = May be uridine
  misc_difference 16: note = May be uridine
  misc_difference 17: note = May be uridine
aggagagctgtgggcttggg

SEQ ID NO: 4 - DNA, 20 bp, synthetic construct
  source 1..20: mol_type = other DNA
  misc_difference 7: note = May be uridine
  misc_difference 11: note = May be uridine
cacccatgcctcccagcaga

SEQ ID NO: 5 - DNA, 20 bp, synthetic construct
  source 1..20: mol_type = other DNA
  misc_difference 3: note = May be uridine
  misc_difference 7: note = May be uridine
  misc_difference 9: note = May be uridine
  misc_difference 12: note = May be uridine
  misc_difference 16: note = May be uridine
  misc_difference 18: note = May be uridine
gctggctgtactcactctcc

SEQ ID NO: 6 - DNA, 20 bp, synthetic construct
  source 1..20: mol_type = other DNA
  misc_difference 2: note = May be uridine
  misc_difference 5: note = May be uridine
  misc_difference 7: note = May be uridine
  misc_difference 9: note = May be uridine
  misc_difference 12: note = May be uridine
  misc_difference 13: note = May be uridine
  misc_difference 16: note = May be uridine
  misc_difference 19: note = May be uridine
gtgctctgtccttggtcctg

SEQ ID NO: 7 - DNA, 20 bp, synthetic construct
  source 1..20: mol_type = other DNA
  misc_difference 5: note = May be uridine
  misc_difference 6: note = May be uridine
  misc_difference 8: note = May be uridine
  misc_difference 11: note = May be uridine
  misc_difference 18: note = May be uridine
cccattctcatgcagcctac

SEQ ID NO: 8 - DNA, 20 bp, synthetic construct
  source 1..20: mol_type = other DNA
  misc_difference 2: note = May be uridine
  misc_difference 4: note = May be uridine
  misc_difference 9: note = May be uridine
  misc_difference 14: note = May be uridine
  misc_difference 17: note = May be uridine
  misc_difference 18: note = May be uridine
ctgtgacctaggctccttga

SEQ ID NO: 9 - DNA, 20 bp, synthetic construct
  source 1..20: mol_type = other DNA
  misc_difference 3: note = May be uridine
  misc_difference 5: note = May be uridine
  misc_difference 13: note = May be uridine
  misc_difference 15: note = May be uridine
  misc_difference 19: note = May be uridine
ggtctgagaggctgtgggtc

SEQ ID NO: 10 - DNA, 20 bp, synthetic construct
  source 1..20: mol_type = other DNA
  misc_difference 3: note = May be uridine
  misc_difference 9: note = May be uridine
  misc_difference 10: note = May be uridine
  misc_difference 12: note = May be uridine
  misc_difference 13: note = May be uridine
  misc_difference 15: note = May be uridine
  misc_difference 17: note = May be uridine
  misc_difference 20: note = May be uridine
gctcccagttcttctgtggt

SEQ ID NO: 11 - DNA, 20 bp, synthetic construct
  source 1..20: mol_type = other DNA
  misc_difference 3: note = May be uridine
  misc_difference 5: note = May be uridine
  misc_difference 8: note = May be uridine
  misc_difference 16: note = May be uridine
gatgtcctccacaggtgaca

SEQ ID NO: 12 - DNA, 20 bp, synthetic construct
  source 1..20: mol_type = other DNA
  misc_difference 3: note = May be uridine
  misc_difference 4: note = May be uridine
  misc_difference 7: note = May be uridine
  misc_difference 11: note = May be uridine
  misc_difference 12: note = May be uridine
  misc_difference 15: note = May be uridine
  misc_difference 20: note = May be uridine
gcttcctgccttactgacct

SEQ ID NO: 13 - DNA, 20 bp, synthetic construct
  source 1..20: mol_type = other DNA
  misc_difference 2: note = May be uridine
  misc_difference 4: note = May be uridine
  misc_difference 7: note = May be uridine
  misc_difference 8: note = May be uridine
  misc_difference 9: note = May be uridine
  misc_difference 11: note = May be uridine
  misc_difference 15: note = May be uridine
ctctcctttgtccctgacca

SEQ ID NO: 14 - DNA, 20 bp, synthetic construct
  source 1..20: mol_type = other DNA
  misc_difference 4: note = May be uridine
  misc_difference 5: note = May be uridine
  misc_difference 9: note = May be uridine
  misc_difference 10: note = May be uridine
  misc_difference 11: note = May be uridine
  misc_difference 12: note = May be uridine
  misc_difference 15: note = May be uridine
  misc_difference 19: note = May be uridine
gccttgccttttcctcactc

SEQ ID NO: 15 - DNA, 20 bp, synthetic construct
  source 1..20: mol_type = other DNA
  misc_difference 4: note = May be uridine
  misc_difference 7: note = May be uridine
  misc_difference 11: note = May be uridine
  misc_difference 17: note = May be uridine
  misc_difference 19: note = May be uridine
gcctggtcactaaccctctc

SEQ ID NO: 16 - DNA, 20 bp, synthetic construct
  source 1..20: mol_type = other DNA
  misc_difference 9: note = May be uridine
  misc_difference 10: note = May be uridine
  misc_difference 13: note = May be uridine
  misc_difference 15: note = May be uridine
  misc_difference 16: note = May be uridine
  misc_difference 20: note = May be uridine
cacccaccttggtcttgcct

SEQ ID NO: 17 - DNA, 20 bp, synthetic construct
  source 1..20: mol_type = other DNA
  misc_difference 6: note = May be uridine
  misc_difference 9: note = May be uridine
  misc_difference 11: note = May be uridine
  misc_difference 14: note = May be uridine
  misc_difference 16: note = May be uridine
cacactgatgtcctgtccca

SEQ ID NO: 18 - DNA, 20 bp, synthetic construct
  source 1..20: mol_type = other DNA
  misc_difference 7: note = May be uridine
  misc_difference 9: note = May be uridine
  misc_difference 13: note = May be uridine
  misc_difference 15: note = May be uridine
  misc_difference 16: note = May be uridine
cacacctctgggtcttggcc

SEQ ID NO: 19 - DNA, 20 bp, synthetic construct
  source 1..20: mol_type = other DNA
  misc_difference 3: note = May be uridine
  misc_difference 9: note = May be uridine
  misc_difference 13: note = May be uridine
  misc_difference 19: note = May be uridine
  misc_difference 20: note = May be uridine
gctgcccatcactcccagtt

SEQ ID NO: 20 - DNA, 20 bp, synthetic construct
  source 1..20: mol_type = other DNA
  misc_difference 2: note = May be uridine
  misc_difference 4: note = May be uridine
  misc_difference 8: note = May be uridine
  misc_difference 10: note = May be uridine
  misc_difference 15: note = May be uridine
ctctccatctgccctggccc

SEQ ID NO: 21 - DNA, 20 bp, synthetic construct
  source 1..20: mol_type = other DNA
  misc_difference 4: note = May be uridine
  misc_difference 8: note = May be uridine
  misc_difference 10: note = May be uridine
  misc_difference 13: note = May be uridine
  misc_difference 14: note = May be uridine
  misc_difference 16: note = May be uridine
  misc_difference 18: note = May be uridine
  misc_difference 20: note = May be uridine
cagtccctctccttgtctct

SEQ ID NO: 22 - DNA, 20 bp, synthetic construct
  source 1..20: mol_type = other DNA
  misc_difference 2: note = May be uridine
  misc_difference 8: note = May be uridine
  misc_difference 11: note = May be uridine
  misc_difference 14: note = May be uridine
  misc_difference 17: note = May be uridine
atccacctgctgctcctggg

SEQ ID NO: 23 - DNA, 20 bp, synthetic construct
  source 1..20: mol_type = other DNA
  misc_difference 4: note = May be uridine
  misc_difference 5: note = May be uridine
  misc_difference 7: note = May be uridine
  misc_difference 9: note = May be uridine
  misc_difference 11: note = May be uridine
  misc_difference 12: note = May be uridine
  misc_difference 14: note = May be uridine
  misc_difference 18: note = May be uridine
cccttgtgtcttgtgggtgc

SEQ ID NO: 24 - DNA, 20 bp, synthetic construct
  source 1..20: mol_type = other DNA
  misc_difference 5: note = May be uridine
  misc_difference 10: note = May be uridine
  misc_difference 11: note = May be uridine
  misc_difference 12: note = May be uridine
  misc_difference 13: note = May be uridine
  misc_difference 16: note = May be uridine
  misc_difference 18: note = May be uridine
  misc_difference 19: note = May be uridine
gccctaggattttcctgttg

SEQ ID NO: 25 - DNA, 20 bp, synthetic construct
  source 1..20: mol_type = other DNA
  misc_difference 4: note = May be uridine
  misc_difference 9: note = May be uridine
  misc_difference 12: note = May be uridine
  misc_difference 16: note = May be uridine
gcctcaactcctgcctccca

SEQ ID NO: 26 - DNA, 20 bp, synthetic construct
  source 1..20: mol_type = other DNA
  misc_difference 8: note = May be uridine
  misc_difference 12: note = May be uridine
  misc_difference 14: note = May be uridine
  misc_difference 20: note = May be uridine
agacacctgggtatcagcct

SEQ ID NO: 27 - DNA, 20 bp, synthetic construct
  source 1..20: mol_type = other DNA
  misc_difference 1: note = May be uridine
  misc_difference 4: note = May be uridine
  misc_difference 5: note = May be uridine
  misc_difference 7: note = May be uridine
  misc_difference 8: note = May be uridine
  misc_difference 12: note = May be uridine
  misc_difference 14: note = May be uridine
  misc_difference 15: note = May be uridine
  misc_difference 16: note = May be uridine
tccttcttccctatttccca

SEQ ID NO: 28 - DNA, 20 bp, synthetic construct
  source 1..20: mol_type = other DNA
  misc_difference 4: note = May be uridine
  misc_difference 8: note = May be uridine
  misc_difference 12: note = May be uridine
  misc_difference 14: note = May be uridine
  misc_difference 19: note = May be uridine
gcatcactcactgtcaggta

SEQ ID NO: 29 - DNA, 20 bp, synthetic construct
  source 1..20: mol_type = other DNA
  misc_difference 2: note = May be uridine
  misc_difference 6: note = May be uridine
  misc_difference 16: note = May be uridine
  misc_difference 17: note = May be uridine
  misc_difference 19: note = May be uridine
gtcagtgccacagccttgtc

SEQ ID NO: 30 - DNA, 20 bp, synthetic construct
  source 1..20: mol_type = other DNA
  misc_difference 7: note = May be uridine
  misc_difference 11: note = May be uridine
  misc_difference 12: note = May be uridine
  misc_difference 14: note = May be uridine
ggcacctaccttatgcaccc

SEQ ID NO: 31 - DNA, 20 bp, synthetic construct
  source 1..20: mol_type = other DNA
  misc_difference 3: note = May be uridine
  misc_difference 6: note = May be uridine
  misc_difference 10: note = May be uridine
  misc_difference 14: note = May be uridine
actactgcatccctcagccc

SEQ ID NO: 32 - DNA, 20 bp, synthetic construct
  source 1..20: mol_type = other DNA
  misc_difference 3: note = May be uridine
  misc_difference 4: note = May be uridine
  misc_difference 6: note = May be uridine
  misc_difference 8: note = May be uridine
  misc_difference 11: note = May be uridine
  misc_difference 14: note = May be uridine
  misc_difference 16: note = May be uridine
  misc_difference 18: note = May be uridine
  misc_difference 20: note = May be uridine
gcttgtctcatcctgtctct

SEQ ID NO: 33 - DNA, 20 bp, synthetic construct
  source 1..20: mol_type = other DNA
  misc_difference 1: note = May be uridine
  misc_difference 3: note = May be uridine
  misc_difference 5: note = May be uridine
  misc_difference 7: note = May be uridine
  misc_difference 12: note = May be uridine
  misc_difference 16: note = May be uridine
  misc_difference 19: note = May be uridine
  misc_difference 20: note = May be uridine
tgtctctgagctgactgctt

SEQ ID NO: 34 - DNA, 20 bp, synthetic construct
  source 1..20: mol_type = other DNA
  misc_difference 5: note = May be uridine
  misc_difference 6: note = May be uridine
  misc_difference 10: note = May be uridine
  misc_difference 17: note = May be uridine
gggcttgactccacactcca

SEQ ID NO: 35 - DNA, 20 bp, synthetic construct
  source 1..20: mol_type = other DNA
  misc_difference 5: note = May be uridine
  misc_difference 10: note = May be uridine
  misc_difference 12: note = May be uridine
  misc_difference 17: note = May be uridine
  misc_difference 18: note = May be uridine
ggcatggcatctcagcttca

SEQ ID NO: 36 - DNA, 20 bp, synthetic construct
  source 1..20: mol_type = other DNA
  misc_difference 1: note = May be uridine
  misc_difference 2: note = May be uridine
  misc_difference 8: note = May be uridine
  misc_difference 13: note = May be uridine
  misc_difference 17: note = May be uridine
  misc_difference 20: note = May be uridine
ttcaggatcacctagctggt

SEQ ID NO: 37 - DNA, 20 bp, synthetic construct
  source 1..20: mol_type = other DNA
  misc_difference 3: note = May be uridine
  misc_difference 5: note = May be uridine
  misc_difference 6: note = May be uridine
  misc_difference 7: note = May be uridine
  misc_difference 12: note = May be uridine
  misc_difference 14: note = May be uridine
  misc_difference 17: note = May be uridine
cctctttgccatctgctggg

SEQ ID NO: 38 - DNA, 20 bp, synthetic construct
  source 1..20: mol_type = other DNA
  misc_difference 4: note = May be uridine
  misc_difference 9: note = May be uridine
  misc_difference 10: note = May be uridine
  misc_difference 14: note = May be uridine
  misc_difference 15: note = May be uridine
  misc_difference 17: note = May be uridine
  misc_difference 20: note = May be uridine
gagtgcagttcacttgtggt

SEQ ID NO: 39 - DNA, 20 bp, synthetic construct
  source 1..20: mol_type = other DNA
  misc_difference 1: note = May be uridine
  misc_difference 10: note = May be uridine
  misc_difference 12: note = May be uridine
  misc_difference 16: note = May be uridine
  misc_difference 18: note = May be uridine
tgcccacactctgcctgtca

SEQ ID NO: 40 - DNA, 20 bp, synthetic construct
  source 1..20: mol_type = other DNA
  misc_difference 11: note = May be uridine
  misc_difference 14: note = May be uridine
  misc_difference 17: note = May be uridine
cagagggagctgctagtcag

SEQ ID NO: 41 - DNA, 20 bp, synthetic construct
  source 1..20: mol_type = other DNA
  misc_difference 1: note = May be uridine
  misc_difference 2: note = May be uridine
  misc_difference 11: note = May be uridine
  misc_difference 14: note = May be uridine
ttggcaaaggtgatgcaggc

SEQ ID NO: 42 - DNA, 20 bp, synthetic construct
  source 1..20: mol_type = other DNA
  misc_difference 3: note = May be uridine
  misc_difference 11: note = May be uridine
  misc_difference 12: note = May be uridine
cctccaccagttggaagacc

SEQ ID NO: 43 - DNA, 20 bp, synthetic construct
  source 1..20: mol_type = other DNA
  misc_difference 3: note = May be uridine
  misc_difference 4: note = May be uridine
  misc_difference 6: note = May be uridine
  misc_difference 19: note = May be uridine
ggttctcagccaccaggatc

SEQ ID NO: 44 - DNA, 20 bp, synthetic construct
  source 1..20: mol_type = other DNA
  misc_difference 2: note = May be uridine
  misc_difference 5: note = May be uridine
  misc_difference 12: note = May be uridine
  misc_difference 16: note = May be uridine
gtgctccaagatgcctgcca

SEQ ID NO: 45 - DNA, 20 bp, synthetic construct
  source 1..20: mol_type = other DNA
  misc_difference 3: note = May be uridine
  misc_difference 4: note = May be uridine
  misc_difference 11: note = May be uridine
  misc_difference 13: note = May be uridine
  misc_difference 14: note = May be uridine
  misc_difference 17: note = May be uridine
ccttgcaggctcttgatggc

SEQ ID NO: 46 - DNA, 20 bp, synthetic construct
  source 1..20: mol_type = other DNA
  misc_difference 2: note = May be uridine
  misc_difference 7: note = May be uridine
  misc_difference 8: note = May be uridine
  misc_difference 11: note = May be uridine
  misc_difference 16: note = May be uridine
gtgccattggtagagtagga

SEQ ID NO: 47 - DNA, 20 bp, synthetic construct
  source 1..20: mol_type = other DNA
  misc_difference 2: note = May be uridine
  misc_difference 7: note = May be uridine
  misc_difference 11: note = May be uridine
  misc_difference 20: note = May be uridine
gtgagctccctaaagaacct

SEQ ID NO: 48 - DNA, 20 bp, synthetic construct
  source 1..20: mol_type = other DNA
  misc_difference 3: note = May be uridine
  misc_difference 4: note = May be uridine
  misc_difference 5: note = May be uridine
  misc_difference 14: note = May be uridine
ggtttgccaccagtacaggg

SEQ ID NO: 49 - DNA, 20 bp, synthetic construct
  source 1..20: mol_type = other DNA
  misc_difference 1: note = May be uridine
  misc_difference 7: note = May be uridine
  misc_difference 8: note = May be uridine
  misc_difference 10: note = May be uridine
  misc_difference 16: note = May be uridine
  misc_difference 20: note = May be uridine
tccagcttctccacatcaat

SEQ ID NO: 50 - DNA, 20 bp, synthetic construct
  source 1..20: mol_type = other DNA
  misc_difference 5: note = May be uridine
  misc_difference 6: note = May be uridine
  misc_difference 10: note = May be uridine
  misc_difference 13: note = May be uridine
  misc_difference 14: note = May be uridine
  misc_difference 20: note = May be uridine
gaacttgcctgcttccagct

SEQ ID NO: 51 - DNA, 20 bp, synthetic construct
  source 1..20: mol_type = other DNA
  misc_difference 5: note = May be uridine
  misc_difference 7: note = May be uridine
  misc_difference 11: note = May be uridine
  misc_difference 15: note = May be uridine
  misc_difference 16: note = May be uridine
  misc_difference 17: note = May be uridine
  misc_difference 19: note = May be uridine
acactctggatgagtttgtc

SEQ ID NO: 52 - DNA, 20 bp, synthetic construct
  source 1..20: mol_type = other DNA
  misc_difference 10: note = May be uridine
  misc_difference 16: note = May be uridine
  misc_difference 18: note = May be uridine
gggcacccatgacactctgg

SEQ ID NO: 53 - DNA, 20 bp, synthetic construct
  source 1..20: mol_type = other DNA
  misc_difference 3: note = May be uridine
  misc_difference 4: note = May be uridine
  misc_difference 6: note = May be uridine
  misc_difference 11: note = May be uridine
  misc_difference 14: note = May be uridine
  misc_difference 20: note = May be uridine
acttgtccagtgctccaggt

SEQ ID NO: 54 - DNA, 20 bp, synthetic construct
  source 1..20: mol_type = other DNA
  misc_difference 6: note = May be uridine
  misc_difference 9: note = May be uridine
  misc_difference 10: note = May be uridine
  misc_difference 15: note = May be uridine
  misc_difference 18: note = May be uridine
cccaatccttgcagtcatgg

SEQ ID NO: 55 - DNA, 20 bp, synthetic construct
  source 1..20: mol_type = other DNA
  misc_difference 10: note = May be uridine
  misc_difference 14: note = May be uridine
  misc_difference 16: note = May be uridine
  misc_difference 19: note = May be uridine
agcacagcctgcatgtcctc

SEQ ID NO: 56 - DNA, 20 bp, synthetic construct
  source 1..20: mol_type = other DNA
  misc_difference 6: note = May be uridine
  misc_difference 9: note = May be uridine
  misc_difference 11: note = May be uridine
  misc_difference 19: note = May be uridine
caaactggtgtcagagcctg

SEQ ID NO: 57 - DNA, 20 bp, synthetic construct
  source 1..20: mol_type = other DNA
  misc_difference 10: note = May be uridine
  misc_difference 17: note = May be uridine
  misc_difference 20: note = May be uridine
gcagcaccctccaaactggt

SEQ ID NO: 58 - DNA, 20 bp, synthetic construct
  source 1..20: mol_type = other DNA
  misc_difference 1: note = May be uridine
  misc_difference 4: note = May be uridine
  misc_difference 5: note = May be uridine
  misc_difference 9: note = May be uridine
  misc_difference 10: note = May be uridine
  misc_difference 18: note = May be uridine
tggttaggttggacccatgg

SEQ ID NO: 59 - DNA, 20 bp, synthetic construct
  source 1..20: mol_type = other DNA
  misc_difference 8: note = May be uridine
  misc_difference 9: note = May be uridine
  misc_difference 11: note = May be uridine
  misc_difference 13: note = May be uridine
gcccaggttgtctcagccca

SEQ ID NO: 60 - DNA, 20 bp, synthetic construct
  source 1..20: mol_type = other DNA
  misc_difference 1: note = May be uridine
  misc_difference 5: note = May be uridine
  misc_difference 7: note = May be uridine
  misc_difference 13: note = May be uridine
  misc_difference 16: note = May be uridine
tccctctccagatactgagg

SEQ ID NO: 61 - DNA, 20 bp, synthetic construct
  source 1..20: mol_type = other DNA
  misc_difference 14: note = May be uridine
acagacaacccaatggcagg

SEQ ID NO: 62 - DNA, 20 bp, synthetic construct
  source 1..20: mol_type = other DNA
  misc_difference 2: note = May be uridine
  misc_difference 4: note = May be uridine
  misc_difference 12: note = May be uridine
gtctccagaactgagcaggg

SEQ ID NO: 63 - DNA, 20 bp, synthetic construct
  source 1..20: mol_type = other DNA
  misc_difference 3: note = May be uridine
  misc_difference 4: note = May be uridine
  misc_difference 7: note = May be uridine
  misc_difference 8: note = May be uridine
  misc_difference 11: note = May be uridine
  misc_difference 13: note = May be uridine
  misc_difference 15: note = May be uridine
ccttaattcctgtctgaggc

SEQ ID NO: 64 - DNA, 20 bp, synthetic construct
  source 1..20: mol_type = other DNA
  misc_difference 6: note = May be uridine
  misc_difference 11: note = May be uridine
cagaatacagtgcccaggcc

SEQ ID NO: 65 - DNA, 20 bp, synthetic construct
  source 1..20: mol_type = other DNA
  misc_difference 10: note = May be uridine
  misc_difference 11: note = May be uridine
  misc_difference 14: note = May be uridine
  misc_difference 20: note = May be uridine
cccaggcccttgctcagaat

SEQ ID NO: 66 - DNA, 20 bp, synthetic construct
  source 1..20: mol_type = other DNA
  misc_difference 5: note = May be uridine
  misc_difference 8: note = May be uridine
  misc_difference 12: note = May be uridine
  misc_difference 16: note = May be uridine
  misc_difference 20: note = May be uridine
gcactcatccctggctggct

SEQ ID NO: 67 - DNA, 20 bp, synthetic construct
  source 1..20: mol_type = other DNA
  misc_difference 3: note = May be uridine
  misc_difference 4: note = May be uridine
gattacagggcaaggccaca

SEQ ID NO: 68 - DNA, 20 bp, synthetic construct
  source 1..20: mol_type = other DNA
  misc_difference 5: note = May be uridine
  misc_difference 9: note = May be uridine
  misc_difference 11: note = May be uridine
gccctggatgtggcaaaaga

SEQ ID NO: 69 - DNA, 20 bp, synthetic construct
  source 1..20: mol_type = other DNA
  misc_difference 8: note = May be uridine
aaggaagtcagagggagggc

SEQ ID NO: 70 - DNA, 20 bp, synthetic construct
  source 1..20: mol_type = other DNA
  misc_difference 19: note = May be uridine
caggcccaaacaggaggctc

SEQ ID NO: 71 - DNA, 20 bp, synthetic construct
  source 1..20: mol_type = other DNA
  misc_difference 2: note = May be uridine
atgcccagacccaggcccaa

SEQ ID NO: 72 - DNA, 20 bp, synthetic construct
  source 1..20: mol_type = other DNA
  misc_difference 2: note = May be uridine
ctgaggcacagcaccaaggc

SEQ ID NO: 73 - DNA, 20 bp, synthetic construct
  source 1..20: mol_type = other DNA
  misc_difference 20: note = May be uridine
gccagaccaggaaggagcct

SEQ ID NO: 74 - DNA, 20 bp, synthetic construct
  source 1..20: mol_type = other DNA
  misc_difference 1: note = May be uridine
  misc_difference 8: note = May be uridine
  misc_difference 9: note = May be uridine
  misc_difference 10: note = May be uridine
  misc_difference 14: note = May be uridine
  misc_difference 16: note = May be uridine
  misc_difference 17: note = May be uridine
  misc_difference 18: note = May be uridine
tcaggactttgcctctttcc

SEQ ID NO: 75 - DNA, 20 bp, synthetic construct
  source 1..20: mol_type = other DNA
  misc_difference 3: note = May be uridine
  misc_difference 4: note = May be uridine
  misc_difference 5: note = May be uridine
  misc_difference 11: note = May be uridine
  misc_difference 12: note = May be uridine
  misc_difference 13: note = May be uridine
  misc_difference 16: note = May be uridine
gctttagagatttgctaccc

SEQ ID NO: 76 - DNA, 20 bp, synthetic construct
  source 1..20: mol_type = other DNA
  misc_difference 9: note = May be uridine
  misc_difference 15: note = May be uridine
  misc_difference 18: note = May be uridine
  misc_difference 19: note = May be uridine
gcccagcctcagaatgattc

SEQ ID NO: 77 - DNA, 20 bp, synthetic construct
  source 1..20: mol_type = other DNA
  misc_difference 3: note = May be uridine
  misc_difference 5: note = May be uridine
  misc_difference 14: note = May be uridine
cctctgaacccagtggagga

SEQ ID NO: 78 - DNA, 20 bp, synthetic construct
  source 1..20: mol_type = other DNA
  misc_difference 4: note = May be uridine
  misc_difference 8: note = May be uridine
  misc_difference 9: note = May be uridine
  misc_difference 10: note = May be uridine
  misc_difference 12: note = May be uridine
  misc_difference 13: note = May be uridine
  misc_difference 20: note = May be uridine
gcctgggtttattggagggt

SEQ ID NO: 79 - DNA, 20 bp, synthetic construct
  source 1..20: mol_type = other DNA
  misc_difference 18: note = May be uridine
gccagcacagccaagagtgg

SEQ ID NO: 80 - DNA, 20 bp, synthetic construct
  source 1..20: mol_type = other DNA
  misc_difference 6: note = May be uridine
gggagtggaaggaaggagcc

SEQ ID NO: 81 - DNA, 20 bp, synthetic construct
  source 1..20: mol_type = other DNA
ccactagccctgggagcaaa

SEQ ID NO: 82 - DNA, 20 bp, synthetic construct
  source 1..20: mol_type = other DNA
gccatctccatccatagagc

SEQ ID NO: 83 - DNA, 20 bp, synthetic construct
  source 1..20: mol_type = other DNA
aggagagctgtgggcttggg

SEQ ID NO: 84 - DNA, 20 bp, synthetic construct
  source 1..20: mol_type = other DNA
cacccatgcctcccagcaga

SEQ ID NO: 85 - DNA, 20 bp, synthetic construct
  source 1..20: mol_type = other DNA
gctggctgtactcactctcc

SEQ ID NO: 86 - DNA, 20 bp, synthetic construct
  source 1..20: mol_type = other DNA
gtgctctgtccttggtcctg

SEQ ID NO: 87 - DNA, 20 bp, synthetic construct
  source 1..20: mol_type = other DNA
cccattctcatgcagcctac

SEQ ID NO: 88 - DNA, 20 bp, synthetic construct
  source 1..20: mol_type = other DNA
ctgtgacctaggctccttga

SEQ ID NO: 89 - DNA, 20 bp, synthetic construct
  source 1..20: mol_type = other DNA
ggtctgagaggctgtgggtc

SEQ ID NO: 90 - DNA, 20 bp, synthetic construct
  source 1..20: mol_type = other DNA
gctcccagttcttctgtggt

SEQ ID NO: 91 - DNA, 20 bp, synthetic construct
  source 1..20: mol_type = other DNA
gatgtcctccacaggtgaca

SEQ ID NO: 92 - DNA, 20 bp, synthetic construct
  source 1..20: mol_type = other DNA
gcttcctgccttactgacct

SEQ ID NO: 93 - DNA, 20 bp, synthetic construct
  source 1..20: mol_type = other DNA
ctctcctttgtccctgacca

SEQ ID NO: 94 - DNA, 20 bp, synthetic construct
  source 1..20: mol_type = other DNA
gccttgccttttcctcactc

SEQ ID NO: 95 - DNA, 20 bp, synthetic construct
  source 1..20: mol_type = other DNA
gcctggtcactaaccctctc

SEQ ID NO: 96 - DNA, 20 bp, synthetic construct
  source 1..20: mol_type = other DNA
cacccaccttggtcttgcct

SEQ ID NO: 97 - DNA, 20 bp, synthetic construct
  source 1..20: mol_type = other DNA
cacactgatgtcctgtccca

SEQ ID NO: 98 - DNA, 20 bp, synthetic construct
  source 1..20: mol_type = other DNA
cacacctctgggtcttggcc

SEQ ID NO: 99 - DNA, 20 bp, synthetic construct
  source 1..20: mol_type = other DNA
gctgcccatcactcccagtt

SEQ ID NO: 100 - DNA, 20 bp, synthetic construct
  source 1..20: mol_type = other DNA
ctctccatctgccctggccc

SEQ ID NO: 101 - DNA, 20 bp, synthetic construct
  source 1..20: mol_type = other DNA
cagtccctctccttgtctct

SEQ ID NO: 102 - DNA, 20 bp, synthetic construct
  source 1..20: mol_type = other DNA
atccacctgctgctcctggg

SEQ ID NO: 103 - DNA, 20 bp, synthetic construct
  source 1..20: mol_type = other DNA
cccttgtgtcttgtgggtgc

SEQ ID NO: 104 - DNA, 20 bp, synthetic construct
  source 1..20: mol_type = other DNA
gccctaggattttcctgttg

SEQ ID NO: 105 - DNA, 20 bp, synthetic construct
  source 1..20: mol_type = other DNA
gcctcaactcctgcctccca

SEQ ID NO: 106 - DNA, 20 bp, synthetic construct
  source 1..20: mol_type = other DNA
agacacctgggtatcagcct

SEQ ID NO: 107 - DNA, 20 bp, synthetic construct
  source 1..20: mol_type = other DNA
tccttcttccctatttccca

SEQ ID NO: 108 - DNA, 20 bp, synthetic construct
  source 1..20: mol_type = other DNA
gcatcactcactgtcaggta

SEQ ID NO: 109 - DNA, 20 bp, synthetic construct
  source 1..20: mol_type = other DNA
gtcagtgccacagccttgtc

SEQ ID NO: 110 - DNA, 20 bp, synthetic construct
  source 1..20: mol_type = other DNA
ggcacctaccttatgcaccc

SEQ ID NO: 111 - DNA, 20 bp, synthetic construct
  source 1..20: mol_type = other DNA
actactgcatccctcagccc

SEQ ID NO: 112 - DNA, 20 bp, synthetic construct
  source 1..20: mol_type = other DNA
gcttgtctcatcctgtctct

SEQ ID NO: 113 - DNA, 20 bp, synthetic construct
  source 1..20: mol_type = other DNA
tgtctctgagctgactgctt

SEQ ID NO: 114 - DNA, 20 bp, synthetic construct
  source 1..20: mol_type = other DNA
gggcttgactccacactcca

SEQ ID NO: 115 - DNA, 20 bp, synthetic construct
  source 1..20: mol_type = other DNA
ggcatggcatctcagcttca

SEQ ID NO: 116 - DNA, 20 bp, synthetic construct
  source 1..20: mol_type = other DNA
ttcaggatcacctagctggt

SEQ ID NO: 117 - DNA, 20 bp, synthetic construct
  source 1..20: mol_type = other DNA
cctctttgccatctgctggg

SEQ ID NO: 118 - DNA, 20 bp, synthetic construct
  source 1..20: mol_type = other DNA
gagtgcagttcacttgtggt

SEQ ID NO: 119 - DNA, 20 bp, synthetic construct
  source 1..20: mol_type = other DNA
tgcccacactctgcctgtca

SEQ ID NO: 120 - DNA, 20 bp, synthetic construct
  source 1..20: mol_type = other DNA
cagagggagctgctagtcag

SEQ ID NO: 121 - DNA, 20 bp, synthetic construct
  source 1..20: mol_type = other DNA
ttggcaaaggtgatgcaggc

SEQ ID NO: 122 - DNA, 20 bp, synthetic construct
  source 1..20: mol_type = other DNA
cctccaccagttggaagacc

SEQ ID NO: 123 - DNA, 20 bp, synthetic construct
  source 1..20: mol_type = other DNA
ggttctcagccaccaggatc

SEQ ID NO: 124 - DNA, 20 bp, synthetic construct
  source 1..20: mol_type = other DNA
gtgctccaagatgcctgcca

SEQ ID NO: 125 - DNA, 20 bp, synthetic construct
  source 1..20: mol_type = other DNA
ccttgcaggctcttgatggc

SEQ ID NO: 126 - DNA, 20 bp, synthetic construct
  source 1..20: mol_type = other DNA
gtgccattggtagagtagga

SEQ ID NO: 127 - DNA, 20 bp, synthetic construct
  source 1..20: mol_type = other DNA
gtgagctccctaaagaacct

SEQ ID NO: 128 - DNA, 20 bp, synthetic construct
  source 1..20: mol_type = other DNA
ggtttgccaccagtacaggg

SEQ ID NO: 129 - DNA, 20 bp, synthetic construct
  source 1..20: mol_type = other DNA
tccagcttctccacatcaat

SEQ ID NO: 130 - DNA, 20 bp, synthetic construct
  source 1..20: mol_type = other DNA
gaacttgcctgcttccagct

SEQ ID NO: 131 - DNA, 20 bp, synthetic construct
  source 1..20: mol_type = other DNA
acactctggatgagtttgtc

SEQ ID NO: 132 - DNA, 20 bp, synthetic construct
  source 1..20: mol_type = other DNA
gggcacccatgacactctgg

SEQ ID NO: 133 - DNA, 20 bp, synthetic construct
  source 1..20: mol_type = other DNA
acttgtccagtgctccaggt

SEQ ID NO: 134 - DNA, 20 bp, synthetic construct
  source 1..20: mol_type = other DNA
cccaatccttgcagtcatgg

SEQ ID NO: 135 - DNA, 20 bp, synthetic construct
  source 1..20: mol_type = other DNA
agcacagcctgcatgtcctc

SEQ ID NO: 136 - DNA, 20 bp, synthetic construct
  source 1..20: mol_type = other DNA
caaactggtgtcagagcctg

SEQ ID NO: 137 - DNA, 20 bp, synthetic construct
  source 1..20: mol_type = other DNA
gcagcaccctccaaactggt

SEQ ID NO: 138 - DNA, 20 bp, synthetic construct
  source 1..20: mol_type = other DNA
tggttaggttggacccatgg

SEQ ID NO: 139 - DNA, 20 bp, synthetic construct
  source 1..20: mol_type = other DNA
gcccaggttgtctcagccca

SEQ ID NO: 140 - DNA, 20 bp, synthetic construct
  source 1..20: mol_type = other DNA
tccctctccagatactgagg

SEQ ID NO: 141 - DNA, 20 bp, synthetic construct
  source 1..20: mol_type = other DNA
acagacaacccaatggcagg

SEQ ID NO: 142 - DNA, 20 bp, synthetic construct
  source 1..20: mol_type = other DNA
gtctccagaactgagcaggg

SEQ ID NO: 143 - DNA, 20 bp, synthetic construct
  source 1..20: mol_type = other DNA
ccttaattcctgtctgaggc

SEQ ID NO: 144 - DNA, 20 bp, synthetic construct
  source 1..20: mol_type = other DNA
cagaatacagtgcccaggcc

SEQ ID NO: 145 - DNA, 20 bp, synthetic construct
  source 1..20: mol_type = other DNA
cccaggcccttgctcagaat

SEQ ID NO: 146 - DNA, 20 bp, synthetic construct
  source 1..20: mol_type = other DNA
gcactcatccctggctggct

SEQ ID NO: 147 - DNA, 20 bp, synthetic construct
  source 1..20: mol_type = other DNA
gattacagggcaaggccaca

SEQ ID NO: 148 - DNA, 20 bp, synthetic construct
  source 1..20: mol_type = other DNA
gccctggatgtggcaaaaga

SEQ ID NO: 149 - DNA, 20 bp, synthetic construct
  source 1..20: mol_type = other DNA
aaggaagtcagagggagggc

SEQ ID NO: 150 - DNA, 20 bp, synthetic construct
  source 1..20: mol_type = other DNA
caggcccaaacaggaggctc

SEQ ID NO: 151 - DNA, 20 bp, synthetic construct
  source 1..20: mol_type = other DNA
atgcccagacccaggcccaa

SEQ ID NO: 152 - DNA, 20 bp, synthetic construct
  source 1..20: mol_type = other DNA
ctgaggcacagcaccaaggc

SEQ ID NO: 153 - DNA, 20 bp, synthetic construct
  source 1..20: mol_type = other DNA
gccagaccaggaaggagcct

SEQ ID NO: 154 - DNA, 20 bp, synthetic construct
  source 1..20: mol_type = other DNA
tcaggactttgcctctttcc

SEQ ID NO: 155 - DNA, 20 bp, synthetic construct
  source 1..20: mol_type = other DNA
gctttagagatttgctaccc

SEQ ID NO: 156 - DNA, 20 bp, synthetic construct
  source 1..20: mol_type = other DNA
gcccagcctcagaatgattc

SEQ ID NO: 157 - DNA, 20 bp, synthetic construct
  source 1..20: mol_type = other DNA
cctctgaacccagtggagga

SEQ ID NO: 158 - DNA, 20 bp, synthetic construct
  source 1..20: mol_type = other DNA
gcctgggtttattggagggt

SEQ ID NO: 159 - DNA, 20 bp, synthetic construct
  source 1..20: mol_type = other DNA
gccagcacagccaagagtgg

SEQ ID NO: 160 - DNA, 20 bp, synthetic construct
  source 1..20: mol_type = other DNA
gggagtggaaggaaggagcc

SEQ ID NO: 161 - RNA, 20 nt, synthetic construct
  source 1..20: mol_type = other RNA
  misc_difference 1: note = May be thymine
  misc_difference 2: note = May be thymine
  misc_difference 3: note = May be thymine
  misc_difference 6: note = May be thymine
  misc_difference 15: note = May be thymine
  misc_difference 18: note = May be thymine
tttgctcccagggctagtgg

SEQ ID NO: 162 - RNA, 20 nt, synthetic construct
  source 1..20: mol_type = other RNA
  misc_difference 3: note = May be thymine
  misc_difference 5: note = May be thymine
  misc_difference 7: note = May be thymine
  misc_difference 11: note = May be thymine
  misc_difference 17: note = May be thymine
gctctatggatggagatggc

SEQ ID NO: 163 - RNA, 20 nt, synthetic construct
  source 1..20: mol_type = other RNA
  misc_difference 15: note = May be thymine
  misc_difference 17: note = May be thymine
  misc_difference 20: note = May be thymine
cccaagcccacagctctcct

SEQ ID NO: 164 - RNA, 20 nt, synthetic construct
  source 1..20: mol_type = other RNA
  misc_difference 1: note = May be thymine
  misc_difference 3: note = May be thymine
  misc_difference 6: note = May be thymine
  misc_difference 15: note = May be thymine
  misc_difference 19: note = May be thymine
tctgctgggaggcatgggtg

SEQ ID NO: 165 - RNA, 20 nt, synthetic construct
  source 1..20: mol_type = other RNA
  misc_difference 7: note = May be thymine
  misc_difference 11: note = May be thymine
ggagagtgagtacagccagc

SEQ ID NO: 166 - RNA, 20 nt, synthetic construct
  source 1..20: mol_type = other RNA
caggaccaaggacagagcac

SEQ ID NO: 167 - RNA, 20 nt, synthetic construct
  source 1..20: mol_type = other RNA
  misc_difference 2: note = May be thymine
  misc_difference 7: note = May be thymine
  misc_difference 11: note = May be thymine
  misc_difference 17: note = May be thymine
gtaggctgcatgagaatggg

SEQ ID NO: 168 - RNA, 20 nt, synthetic construct
  source 1..20: mol_type = other RNA
  misc_difference 1: note = May be thymine
  misc_difference 11: note = May be thymine
  misc_difference 15: note = May be thymine
tcaaggagcctaggtcacag

SEQ ID NO: 169 - RNA, 20 nt, synthetic construct
  source 1..20: mol_type = other RNA
  misc_difference 12: note = May be thymine
  misc_difference 14: note = May be thymine
gacccacagcctctcagacc

SEQ ID NO: 170 - RNA, 20 nt, synthetic construct
  source 1..20: mol_type = other RNA
  misc_difference 14: note = May be thymine
accacagaagaactgggagc

SEQ ID NO: 171 - RNA, 20 nt, synthetic construct
  source 1..20: mol_type = other RNA
  misc_difference 1: note = May be thymine
  misc_difference 3: note = May be thymine
  misc_difference 8: note = May be thymine
  misc_difference 10: note = May be thymine
  misc_difference 19: note = May be thymine
tgtcacctgtggaggacatc

SEQ ID NO: 172 - RNA, 20 nt, synthetic construct
  source 1..20: mol_type = other RNA
  misc_difference 4: note = May be thymine
  misc_difference 8: note = May be thymine
aggtcagtaaggcaggaagc

SEQ ID NO: 173 - RNA, 20 nt, synthetic construct
  source 1..20: mol_type = other RNA
  misc_difference 1: note = May be thymine
  misc_difference 4: note = May be thymine
tggtcagggacaaaggagag

SEQ ID NO: 174 - RNA, 20 nt, synthetic construct
  source 1..20: mol_type = other RNA
  misc_difference 4: note = May be thymine
gagtgaggaaaaggcaaggc

SEQ ID NO: 175 - RNA, 20 nt, synthetic construct
  source 1..20: mol_type = other RNA
  misc_difference 8: note = May be thymine
  misc_difference 9: note = May be thymine
  misc_difference 12: note = May be thymine
gagagggttagtgaccaggc

SEQ ID NO: 176 - RNA, 20 nt, synthetic construct
  source 1..20: mol_type = other RNA
  misc_difference 15: note = May be thymine
  misc_difference 19: note = May be thymine
aggcaagaccaaggtgggtg

SEQ ID NO: 177 - RNA, 20 nt, synthetic construct
  source 1..20: mol_type = other RNA
  misc_difference 1: note = May be thymine
  misc_difference 13: note = May be thymine
  misc_difference 17: note = May be thymine
  misc_difference 19: note = May be thymine
tgggacaggacatcagtgtg

SEQ ID NO: 178 - RNA, 20 nt, synthetic construct
  source 1..20: mol_type = other RNA
  misc_difference 17: note = May be thymine
  misc_difference 19: note = May be thymine
ggccaagacccagaggtgtg

SEQ ID NO: 179 - RNA, 20 nt, synthetic construct
  source 1..20: mol_type = other RNA
  misc_difference 4: note = May be thymine
  misc_difference 10: note = May be thymine
  misc_difference 13: note = May be thymine
aactgggagtgatgggcagc

SEQ ID NO: 180 - RNA, 20 nt, synthetic construct
  source 1..20: mol_type = other RNA
  misc_difference 14: note = May be thymine
gggccagggcagatggagag

SEQ ID NO: 181 - RNA, 20 nt, synthetic construct
  source 1..20: mol_type = other RNA
  misc_difference 19: note = May be thymine
agagacaaggagagggactg

SEQ ID NO: 182 - RNA, 20 nt, synthetic construct
  source 1..20: mol_type = other RNA
  misc_difference 16: note = May be thymine
  misc_difference 20: note = May be thymine
cccaggagcagcaggtggat

SEQ ID NO: 183 - RNA, 20 nt, synthetic construct
  source 1..20: mol_type = other RNA
gcacccacaagacacaaggg

SEQ ID NO: 184 - RNA, 20 nt, synthetic construct
  source 1..20: mol_type = other RNA
  misc_difference 12: note = May be thymine
  misc_difference 15: note = May be thymine
caacaggaaaatcctagggc

SEQ ID NO: 185 - RNA, 20 nt, synthetic construct
  source 1..20: mol_type = other RNA
  misc_difference 1: note = May be thymine
  misc_difference 14: note = May be thymine
  misc_difference 15: note = May be thymine
tgggaggcaggagttgaggc

SEQ ID NO: 186 - RNA, 20 nt, synthetic construct
  source 1..20: mol_type = other RNA
  misc_difference 5: note = May be thymine
  misc_difference 8: note = May be thymine
  misc_difference 16: note = May be thymine
  misc_difference 18: note = May be thymine
  misc_difference 20: note = May be thymine
aggctgatacccaggtgtct

SEQ ID NO: 187 - RNA, 20 nt, synthetic construct
  source 1..20: mol_type = other RNA
  misc_difference 1: note = May be thymine
  misc_difference 8: note = May be thymine
tgggaaatagggaagaagga

SEQ ID NO: 188 - RNA, 20 nt, synthetic construct
  source 1..20: mol_type = other RNA
  misc_difference 1: note = May be thymine
  misc_difference 5: note = May be thymine
  misc_difference 11: note = May be thymine
  misc_difference 15: note = May be thymine
  misc_difference 18: note = May be thymine
tacctgacagtgagtgatgc

SEQ ID NO: 189 - RNA, 20 nt, synthetic construct
  source 1..20: mol_type = other RNA
  misc_difference 9: note = May be thymine
  misc_difference 11: note = May be thymine
  misc_difference 17: note = May be thymine
gacaaggctgtggcactgac

SEQ ID NO: 190 - RNA, 20 nt, synthetic construct
  source 1..20: mol_type = other RNA
  misc_difference 4: note = May be thymine
  misc_difference 8: note = May be thymine
  misc_difference 13: note = May be thymine
  misc_difference 17: note = May be thymine
gggtgcataaggtaggtgcc

SEQ ID NO: 191 - RNA, 20 nt, synthetic construct
  source 1..20: mol_type = other RNA
  misc_difference 5: note = May be thymine
  misc_difference 12: note = May be thymine
  misc_difference 17: note = May be thymine
  misc_difference 20: note = May be thymine
gggctgagggatgcagtagt

SEQ ID NO: 192 - RNA, 20 nt, synthetic construct
  source 1..20: mol_type = other RNA
  misc_difference 11: note = May be thymine
agagacaggatgagacaagc

SEQ ID NO: 193 - RNA, 20 nt, synthetic construct
  source 1..20: mol_type = other RNA
  misc_difference 7: note = May be thymine
  misc_difference 12: note = May be thymine
aagcagtcagctcagagaca

SEQ ID NO: 194 - RNA, 20 nt, synthetic construct
  source 1..20: mol_type = other RNA
  misc_difference 1: note = May be thymine
  misc_difference 6: note = May be thymine
  misc_difference 8: note = May be thymine
  misc_difference 13: note = May be thymine
tggagtgtggagtcaagccc

SEQ ID NO: 195 - RNA, 20 nt, synthetic construct
  source 1..20: mol_type = other RNA
  misc_difference 1: note = May be thymine
  misc_difference 7: note = May be thymine
  misc_difference 12: note = May be thymine
  misc_difference 17: note = May be thymine
tgaagctgagatgccatgcc

SEQ ID NO: 196 - RNA, 20 nt, synthetic construct
  source 1..20: mol_type = other RNA
  misc_difference 7: note = May be thymine
  misc_difference 11: note = May be thymine
  misc_difference 14: note = May be thymine
  misc_difference 17: note = May be thymine
accagctaggtgatcctgaa

SEQ ID NO: 197 - RNA, 20 nt, synthetic construct
  source 1..20: mol_type = other RNA
  misc_difference 10: note = May be thymine
cccagcagatggcaaagagg

SEQ ID NO: 198 - RNA, 20 nt, synthetic construct
  source 1..20: mol_type = other RNA
  misc_difference 9: note = May be thymine
  misc_difference 14: note = May be thymine
  misc_difference 19: note = May be thymine
accacaagtgaactgcactc

SEQ ID NO: 199 - RNA, 20 nt, synthetic construct
  source 1..20: mol_type = other RNA
  misc_difference 1: note = May be thymine
  misc_difference 13: note = May be thymine
  misc_difference 15: note = May be thymine
tgacaggcagagtgtgggca

SEQ ID NO: 200 - RNA, 20 nt, synthetic construct
  source 1..20: mol_type = other RNA
  misc_difference 2: note = May be thymine
  misc_difference 6: note = May be thymine
  misc_difference 13: note = May be thymine
  misc_difference 17: note = May be thymine
  misc_difference 19: note = May be thymine
ctgactagcagctccctctg

SEQ ID NO: 201 - RNA, 20 nt, synthetic construct
  source 1..20: mol_type = other RNA
  misc_difference 4: note = May be thymine
  misc_difference 8: note = May be thymine
  misc_difference 13: note = May be thymine
  misc_difference 14: note = May be thymine
  misc_difference 15: note = May be thymine
gcctgcatcacctttgccaa

SEQ ID NO: 202 - RNA, 20 nt, synthetic construct
  source 1..20: mol_type = other RNA
  misc_difference 3: note = May be thymine
  misc_difference 5: note = May be thymine
  misc_difference 6: note = May be thymine
  misc_difference 12: note = May be thymine
  misc_difference 15: note = May be thymine
ggtcttccaactggtggagg

SEQ ID NO: 203 - RNA, 20 nt, synthetic construct
  source 1..20: mol_type = other RNA
  misc_difference 3: note = May be thymine
  misc_difference 6: note = May be thymine
  misc_difference 9: note = May be thymine
  misc_difference 13: note = May be thymine
gatcctggtggctgagaacc

SEQ ID NO: 204 - RNA, 20 nt, synthetic construct
  source 1..20: mol_type = other RNA
  misc_difference 1: note = May be thymine
  misc_difference 10: note = May be thymine
  misc_difference 12: note = May be thymine
  misc_difference 13: note = May be thymine
tggcaggcatcttggagcac

SEQ ID NO: 205 - RNA, 20 nt, synthetic construct
  source 1..20: mol_type = other RNA
  misc_difference 5: note = May be thymine
  misc_difference 14: note = May be thymine
gccatcaagagcctgcaagg

SEQ ID NO: 206 - RNA, 20 nt, synthetic construct
  source 1..20: mol_type = other RNA
  misc_difference 1: note = May be thymine
  misc_difference 4: note = May be thymine
  misc_difference 7: note = May be thymine
  misc_difference 9: note = May be thymine
  misc_difference 15: note = May be thymine
tcctactctaccaatggcac

SEQ ID NO: 207 - RNA, 20 nt, synthetic construct
  source 1..20: mol_type = other RNA
  misc_difference 4: note = May be thymine
  misc_difference 5: note = May be thymine
  misc_difference 7: note = May be thymine
  misc_difference 8: note = May be thymine
  misc_difference 9: note = May be thymine
  misc_difference 17: note = May be thymine
aggttctttagggagctcac

SEQ ID NO: 208 - RNA, 20 nt, synthetic construct
  source 1..20: mol_type = other RNA
  misc_difference 4: note = May be thymine
  misc_difference 6: note = May be thymine
  misc_difference 9: note = May be thymine
  misc_difference 12: note = May be thymine
ccctgtactggtggcaaacc

SEQ ID NO: 209 - RNA, 20 nt, synthetic construct
  source 1..20: mol_type = other RNA
  misc_difference 2: note = May be thymine
  misc_difference 3: note = May be thymine
  misc_difference 6: note = May be thymine
  misc_difference 8: note = May be thymine
  misc_difference 17: note = May be thymine
attgatgtggagaagctgga

SEQ ID NO: 210 - RNA, 20 nt, synthetic construct
  source 1..20: mol_type = other RNA
  misc_difference 4: note = May be thymine
  misc_difference 18: note = May be thymine
  misc_difference 19: note = May be thymine
agctggaagcaggcaagttc

SEQ ID NO: 211 - RNA, 20 nt, synthetic construct
  source 1..20: mol_type = other RNA
  misc_difference 8: note = May be thymine
  misc_difference 11: note = May be thymine
  misc_difference 18: note = May be thymine
  misc_difference 20: note = May be thymine
gacaaactcatccagagtgt

SEQ ID NO: 212 - RNA, 20 nt, synthetic construct
  source 1..20: mol_type = other RNA
  misc_difference 7: note = May be thymine
  misc_difference 9: note = May be thymine
  misc_difference 12: note = May be thymine
  misc_difference 16: note = May be thymine
ccagagtgtcatgggtgccc

SEQ ID NO: 213 - RNA, 20 nt, synthetic construct
  source 1..20: mol_type = other RNA
  misc_difference 4: note = May be thymine
  misc_difference 12: note = May be thymine
  misc_difference 20: note = May be thymine
acctggagcactggacaagt

SEQ ID NO: 214 - RNA, 20 nt, synthetic construct
  source 1..20: mol_type = other RNA
  misc_difference 4: note = May be thymine
  misc_difference 8: note = May be thymine
  misc_difference 16: note = May be thymine
  misc_difference 17: note = May be thymine
ccatgactgcaaggattggg

SEQ ID NO: 215 - RNA, 20 nt, synthetic construct
  source 1..20: mol_type = other RNA
  misc_difference 8: note = May be thymine
  misc_difference 15: note = May be thymine
  misc_difference 17: note = May be thymine
  misc_difference 20: note = May be thymine
gaggacatgcaggctgtgct

SEQ ID NO: 216 - RNA, 20 nt, synthetic construct
  source 1..20: mol_type = other RNA
  misc_difference 6: note = May be thymine
  misc_difference 8: note = May be thymine
  misc_difference 17: note = May be thymine
  misc_difference 18: note = May be thymine
  misc_difference 19: note = May be thymine
caggctctgacaccagtttg

SEQ ID NO: 217 - RNA, 20 nt, synthetic construct
  source 1..20: mol_type = other RNA
  misc_difference 6: note = May be thymine
  misc_difference 7: note = May be thymine
  misc_difference 8: note = May be thymine
  misc_difference 15: note = May be thymine
  misc_difference 18: note = May be thymine
accagtttggagggtgctgc

SEQ ID NO: 218 - RNA, 20 nt, synthetic construct
  source 1..20: mol_type = other RNA
  misc_difference 4: note = May be thymine
  misc_difference 8: note = May be thymine
  misc_difference 15: note = May be thymine
ccatgggtccaacctaacca

SEQ ID NO: 219 - RNA, 20 nt, synthetic construct
  source 1..20: mol_type = other RNA
  misc_difference 1: note = May be thymine
  misc_difference 6: note = May be thymine
  misc_difference 16: note = May be thymine
tgggctgagacaacctgggc

SEQ ID NO: 220 - RNA, 20 nt, synthetic construct
  source 1..20: mol_type = other RNA
  misc_difference 3: note = May be thymine
  misc_difference 7: note = May be thymine
  misc_difference 9: note = May be thymine
  misc_difference 11: note = May be thymine
cctcagtatctggagaggga

SEQ ID NO: 221 - RNA, 20 nt, synthetic construct
  source 1..20: mol_type = other RNA
  misc_difference 3: note = May be thymine
  misc_difference 8: note = May be thymine
  misc_difference 9: note = May be thymine
  misc_difference 13: note = May be thymine
  misc_difference 14: note = May be thymine
  misc_difference 16: note = May be thymine
  misc_difference 18: note = May be thymine
  misc_difference 20: note = May be thymine
cctgccattgggttgtctgt

SEQ ID NO: 222 - RNA, 20 nt, synthetic construct
  source 1..20: mol_type = other RNA
  misc_difference 4: note = May be thymine
  misc_difference 7: note = May be thymine
  misc_difference 11: note = May be thymine
  misc_difference 12: note = May be thymine
  misc_difference 14: note = May be thymine
ccctgctcagttctggagac

SEQ ID NO: 223 - RNA, 20 nt, synthetic construct
  source 1..20: mol_type = other RNA
  misc_difference 4: note = May be thymine
  misc_difference 15: note = May be thymine
  misc_difference 16: note = May be thymine
gcctcagacaggaattaagg

SEQ ID NO: 224 - RNA, 20 nt, synthetic construct
  source 1..20: mol_type = other RNA
  misc_difference 5: note = May be thymine
  misc_difference 12: note = May be thymine
  misc_difference 14: note = May be thymine
  misc_difference 16: note = May be thymine
  misc_difference 17: note = May be thymine
  misc_difference 19: note = May be thymine
ggcctgggcactgtattctg

SEQ ID NO: 225 - RNA, 20 nt, synthetic construct
  source 1..20: mol_type = other RNA
  misc_difference 2: note = May be thymine
  misc_difference 3: note = May be thymine
  misc_difference 5: note = May be thymine
  misc_difference 17: note = May be thymine
attctgagcaagggcctggg

SEQ ID NO: 226 - RNA, 20 nt, synthetic construct
  source 1..20: mol_type = other RNA
  misc_difference 14: note = May be thymine
  misc_difference 18: note = May be thymine
agccagccagggatgagtgc

SEQ ID NO: 227 - RNA, 20 nt, synthetic construct
  source 1..20: mol_type = other RNA
  misc_difference 1: note = May be thymine
  misc_difference 3: note = May be thymine
  misc_difference 8: note = May be thymine
  misc_difference 9: note = May be thymine
  misc_difference 14: note = May be thymine
  misc_difference 16: note = May be thymine
  misc_difference 19: note = May be thymine
tgtggccttgccctgtaatc

SEQ ID NO: 228 - RNA, 20 nt, synthetic construct
  source 1..20: mol_type = other RNA
  misc_difference 1: note = May be thymine
  misc_difference 3: note = May be thymine
  misc_difference 4: note = May be thymine
  misc_difference 5: note = May be thymine
  misc_difference 6: note = May be thymine
  misc_difference 13: note = May be thymine
tcttttgccacatccagggc

SEQ ID NO: 229 - RNA, 20 nt, synthetic construct
  source 1..20: mol_type = other RNA
  misc_difference 5: note = May be thymine
  misc_difference 9: note = May be thymine
  misc_difference 11: note = May be thymine
  misc_difference 15: note = May be thymine
  misc_difference 16: note = May be thymine
  misc_difference 19: note = May be thymine
  misc_difference 20: note = May be thymine
gccctccctctgacttcctt

SEQ ID NO: 230 - RNA, 20 nt, synthetic construct
  source 1..20: mol_type = other RNA
  misc_difference 6: note = May be thymine
  misc_difference 9: note = May be thymine
  misc_difference 11: note = May be thymine
  misc_difference 12: note = May be thymine
  misc_difference 13: note = May be thymine
  misc_difference 19: note = May be thymine
gagcctcctgtttgggcctg

SEQ ID NO: 231 - RNA, 20 nt, synthetic construct
  source 1..20: mol_type = other RNA
  misc_difference 1: note = May be thymine
  misc_difference 2: note = May be thymine
  misc_difference 8: note = May be thymine
  misc_difference 12: note = May be thymine
  misc_difference 14: note = May be thymine
  misc_difference 20: note = May be thymine
ttgggcctgggtctgggcat

SEQ ID NO: 232 - RNA, 20 nt, synthetic construct
  source 1..20: mol_type = other RNA
  misc_difference 4: note = May be thymine
  misc_difference 5: note = May be thymine
  misc_difference 8: note = May be thymine
  misc_difference 11: note = May be thymine
  misc_difference 13: note = May be thymine
  misc_difference 17: note = May be thymine
gccttggtgctgtgcctcag

SEQ ID NO: 233 - RNA, 20 nt, synthetic construct
  source 1..20: mol_type = other RNA
  misc_difference 5: note = May be thymine
  misc_difference 8: note = May be thymine
  misc_difference 9: note = May be thymine
  misc_difference 12: note = May be thymine
  misc_difference 15: note = May be thymine
  misc_difference 17: note = May be thymine
aggctccttcctggtctggc

SEQ ID NO: 234 - RNA, 20 nt, synthetic construct
  source 1..20: mol_type = other RNA
  misc_difference 15: note = May be thymine
  misc_difference 18: note = May be thymine
ggaaagaggcaaagtcctga

SEQ ID NO: 235 - RNA, 20 nt, synthetic construct
  source 1..20: mol_type = other RNA
  misc_difference 4: note = May be thymine
  misc_difference 11: note = May be thymine
  misc_difference 13: note = May be thymine
  misc_difference 15: note = May be thymine
gggtagcaaatctctaaagc

SEQ ID NO: 236 - RNA, 20 nt, synthetic construct
  source 1..20: mol_type = other RNA
  misc_difference 4: note = May be thymine
  misc_difference 7: note = May be thymine
  misc_difference 8: note = May be thymine
  misc_difference 10: note = May be thymine
  misc_difference 16: note = May be thymine
gaatcattctgaggctgggc

SEQ ID NO: 237 - RNA, 20 nt, synthetic construct
  source 1..20: mol_type = other RNA
  misc_difference 1: note = May be thymine
  misc_difference 4: note = May be thymine
  misc_difference 9: note = May be thymine
  misc_difference 13: note = May be thymine
  misc_difference 14: note = May be thymine
tcctccactgggttcagagg

SEQ ID NO: 238 - RNA, 20 nt, synthetic construct
  source 1..20: mol_type = other RNA
  misc_difference 5: note = May be thymine
  misc_difference 10: note = May be thymine
accctccaataaacccaggc

SEQ ID NO: 239 - RNA, 20 nt, synthetic construct
  source 1..20: mol_type = other RNA
  misc_difference 5: note = May be thymine
  misc_difference 7: note = May be thymine
  misc_difference 8: note = May be thymine
  misc_difference 12: note = May be thymine
  misc_difference 14: note = May be thymine
  misc_difference 17: note = May be thymine
ccactcttggctgtgctggc

SEQ ID NO: 240 - RNA, 20 nt, synthetic construct
  source 1..20: mol_type = other RNA
  misc_difference 4: note = May be thymine
  misc_difference 7: note = May be thymine
  misc_difference 8: note = May be thymine
  misc_difference 11: note = May be thymine
  misc_difference 12: note = May be thymine
  misc_difference 17: note = May be thymine
ggctccttccttccactccc

SEQ ID NO: 241 - RNA, 20 nt, synthetic construct
  source 1..20: mol_type = other RNA
tttgctcccagggctagtgg

SEQ ID NO: 242 - RNA, 20 nt, synthetic construct
  source 1..20: mol_type = other RNA
gctctatggatggagatggc

SEQ ID NO: 243 - RNA, 20 nt, synthetic construct
  source 1..20: mol_type = other RNA
cccaagcccacagctctcct

SEQ ID NO: 244 - RNA, 20 nt, synthetic construct
  source 1..20: mol_type = other RNA
tctgctgggaggcatgggtg

SEQ ID NO: 245 - RNA, 20 nt, synthetic construct
  source 1..20: mol_type = other RNA
ggagagtgagtacagccagc

SEQ ID NO: 246 - RNA, 20 nt, synthetic construct
  source 1..20: mol_type = other RNA
caggaccaaggacagagcac

SEQ ID NO: 247 - RNA, 20 nt, synthetic construct
  source 1..20: mol_type = other RNA
gtaggctgcatgagaatggg

SEQ ID NO: 248 - RNA, 20 nt, synthetic construct
  source 1..20: mol_type = other RNA
tcaaggagcctaggtcacag

SEQ ID NO: 249 - RNA, 20 nt, synthetic construct
  source 1..20: mol_type = other RNA
gacccacagcctctcagacc

SEQ ID NO: 250 - RNA, 20 nt, synthetic construct
  source 1..20: mol_type = other RNA
accacagaagaactgggagc

SEQ ID NO: 251 - RNA, 20 nt, synthetic construct
  source 1..20: mol_type = other RNA
tgtcacctgtggaggacatc

SEQ ID NO: 252 - RNA, 20 nt, synthetic construct
  source 1..20: mol_type = other RNA
aggtcagtaaggcaggaagc

SEQ ID NO: 253 - RNA, 20 nt, synthetic construct
  source 1..20: mol_type = other RNA
tggtcagggacaaaggagag

SEQ ID NO: 254 - RNA, 20 nt, synthetic construct
  source 1..20: mol_type = other RNA
gagtgaggaaaaggcaaggc

SEQ ID NO: 255 - RNA, 20 nt, synthetic construct
  source 1..20: mol_type = other RNA
gagagggttagtgaccaggc

SEQ ID NO: 256 - RNA, 20 nt, synthetic construct
  source 1..20: mol_type = other RNA
aggcaagaccaaggtgggtg

SEQ ID NO: 257 - RNA, 20 nt, synthetic construct
  source 1..20: mol_type = other RNA
tgggacaggacatcagtgtg

SEQ ID NO: 258 - RNA, 20 nt, synthetic construct
  source 1..20: mol_type = other RNA
ggccaagacccagaggtgtg

SEQ ID NO: 259 - RNA, 20 nt, synthetic construct
  source 1..20: mol_type = other RNA
aactgggagtgatgggcagc

SEQ ID NO: 260 - RNA, 20 nt, synthetic construct
  source 1..20: mol_type = other RNA
gggccagggcagatggagag

SEQ ID NO: 261 - RNA, 20 nt, synthetic construct
  source 1..20: mol_type = other RNA
agagacaaggagagggactg

SEQ ID NO: 262 - RNA, 20 nt, synthetic construct
  source 1..20: mol_type = other RNA
cccaggagcagcaggtggat

SEQ ID NO: 263 - RNA, 20 nt, synthetic construct
  source 1..20: mol_type = other RNA
gcacccacaagacacaaggg

SEQ ID NO: 264 - RNA, 20 nt, synthetic construct
  source 1..20: mol_type = other RNA
caacaggaaaatcctagggc

SEQ ID NO: 265 - RNA, 20 nt, synthetic construct
  source 1..20: mol_type = other RNA
tgggaggcaggagttgaggc

SEQ ID NO: 266 - RNA, 20 nt, synthetic construct
  source 1..20: mol_type = other RNA
aggctgatacccaggtgtct

SEQ ID NO: 267 - RNA, 20 nt, synthetic construct
  source 1..20: mol_type = other RNA
tgggaaatagggaagaagga

SEQ ID NO: 268 - RNA, 20 nt, synthetic construct
  source 1..20: mol_type = other RNA
tacctgacagtgagtgatgc

SEQ ID NO: 269 - RNA, 20 nt, synthetic construct
  source 1..20: mol_type = other RNA
gacaaggctgtggcactgac

SEQ ID NO: 270 - RNA, 20 nt, synthetic construct
  source 1..20: mol_type = other RNA
gggtgcataaggtaggtgcc

SEQ ID NO: 271 - RNA, 20 nt, synthetic construct
  source 1..20: mol_type = other RNA
gggctgagggatgcagtagt

SEQ ID NO: 272 - RNA, 20 nt, synthetic construct
  source 1..20: mol_type = other RNA
agagacaggatgagacaagc

SEQ ID NO: 273 - RNA, 20 nt, synthetic construct
  source 1..20: mol_type = other RNA
aagcagtcagctcagagaca

SEQ ID NO: 274 - RNA, 20 nt, synthetic construct
  source 1..20: mol_type = other RNA
tggagtgtggagtcaagccc

SEQ ID NO: 275 - RNA, 20 nt, synthetic construct
  source 1..20: mol_type = other RNA
tgaagctgagatgccatgcc

SEQ ID NO: 276 - RNA, 20 nt, synthetic construct
  source 1..20: mol_type = other RNA
accagctaggtgatcctgaa

SEQ ID NO: 277 - RNA, 20 nt, synthetic construct
  source 1..20: mol_type = other RNA
cccagcagatggcaaagagg

SEQ ID NO: 278 - RNA, 20 nt, synthetic construct
  source 1..20: mol_type = other RNA
accacaagtgaactgcactc

SEQ ID NO: 279 - RNA, 20 nt, synthetic construct
  source 1..20: mol_type = other RNA
tgacaggcagagtgtgggca

SEQ ID NO: 280 - RNA, 20 nt, synthetic construct
  source 1..20: mol_type = other RNA
ctgactagcagctccctctg

SEQ ID NO: 281 - RNA, 20 nt, synthetic construct
  source 1..20: mol_type = other RNA
gcctgcatcacctttgccaa

SEQ ID NO: 282 - RNA, 20 nt, synthetic construct
  source 1..20: mol_type = other RNA
ggtcttccaactggtggagg

SEQ ID NO: 283 - RNA, 20 nt, synthetic construct
  source 1..20: mol_type = other RNA
gatcctggtggctgagaacc

SEQ ID NO: 284 - RNA, 20 nt, synthetic construct
  source 1..20: mol_type = other RNA
tggcaggcatcttggagcac

SEQ ID NO: 285 - RNA, 20 nt, synthetic construct
  source 1..20: mol_type = other RNA
gccatcaagagcctgcaagg

SEQ ID NO: 286 - RNA, 20 nt, synthetic construct
  source 1..20: mol_type = other RNA
tcctactctaccaatggcac

SEQ ID NO: 287 - RNA, 20 nt, synthetic construct
  source 1..20: mol_type = other RNA
aggttctttagggagctcac

SEQ ID NO: 288 - RNA, 20 nt, synthetic construct
  source 1..20: mol_type = other RNA
ccctgtactggtggcaaacc

SEQ ID NO: 289 - RNA, 20 nt, synthetic construct
  source 1..20: mol_type = other RNA
attgatgtggagaagctgga

SEQ ID NO: 290 - RNA, 20 nt, synthetic construct
  source 1..20: mol_type = other RNA
agctggaagcaggcaagttc

SEQ ID NO: 291 - RNA, 20 nt, synthetic construct
  source 1..20: mol_type = other RNA
gacaaactcatccagagtgt

SEQ ID NO: 292 - RNA, 20 nt, synthetic construct
  source 1..20: mol_type = other RNA
ccagagtgtcatgggtgccc

SEQ ID NO: 293 - RNA, 20 nt, synthetic construct
  source 1..20: mol_type = other RNA
acctggagcactggacaagt

SEQ ID NO: 294 - RNA, 20 nt, synthetic construct
  source 1..20: mol_type = other RNA
ccatgactgcaaggattggg

SEQ ID NO: 295 - RNA, 20 nt, synthetic construct
  source 1..20: mol_type = other RNA
gaggacatgcaggctgtgct

SEQ ID NO: 296 - RNA, 20 nt, synthetic construct
  source 1..20: mol_type = other RNA
caggctctgacaccagtttg

SEQ ID NO: 297 - RNA, 20 nt, synthetic construct
  source 1..20: mol_type = other RNA
accagtttggagggtgctgc

SEQ ID NO: 298 - RNA, 20 nt, synthetic construct
  source 1..20: mol_type = other RNA
ccatgggtccaacctaacca

SEQ ID NO: 299 - RNA, 20 nt, synthetic construct
  source 1..20: mol_type = other RNA
tgggctgagacaacctgggc

SEQ ID NO: 300 - RNA, 20 nt, synthetic construct
  source 1..20: mol_type = other RNA
cctcagtatctggagaggga

SEQ ID NO: 301 - RNA, 20 nt, synthetic construct
  source 1..20: mol_type = other RNA
cctgccattgggttgtctgt

SEQ ID NO: 302 - RNA, 20 nt, synthetic construct
  source 1..20: mol_type = other RNA
ccctgctcagttctggagac

SEQ ID NO: 303 - RNA, 20 nt, synthetic construct
  source 1..20: mol_type = other RNA
gcctcagacaggaattaagg

SEQ ID NO: 304 - RNA, 20 nt, synthetic construct
  source 1..20: mol_type = other RNA
ggcctgggcactgtattctg

SEQ ID NO: 305 - RNA, 20 nt, synthetic construct
  source 1..20: mol_type = other RNA
attctgagcaagggcctggg

SEQ ID NO: 306 - RNA, 20 nt, synthetic construct
  source 1..20: mol_type = other RNA
agccagccagggatgagtgc

SEQ ID NO: 307 - RNA, 20 nt, synthetic construct
  source 1..20: mol_type = other RNA
tgtggccttgccctgtaatc

SEQ ID NO: 308 - RNA, 20 nt, synthetic construct
  source 1..20: mol_type = other RNA
tcttttgccacatccagggc

SEQ ID NO: 309 - RNA, 20 nt, synthetic construct
  source 1..20: mol_type = other RNA
gccctccctctgacttcctt

SEQ ID NO: 310 - RNA, 20 nt, synthetic construct
  source 1..20: mol_type = other RNA
gagcctcctgtttgggcctg

SEQ ID NO: 311 - RNA, 20 nt, synthetic construct
  source 1..20: mol_type = other RNA
ttgggcctgggtctgggcat

SEQ ID NO: 312 - RNA, 20 nt, synthetic construct
  source 1..20: mol_type = other RNA
gccttggtgctgtgcctcag

SEQ ID NO: 313 - RNA, 20 nt, synthetic construct
  source 1..20: mol_type = other RNA
aggctccttcctggtctggc

SEQ ID NO: 314 - RNA, 20 nt, synthetic construct
  source 1..20: mol_type = other RNA
ggaaagaggcaaagtcctga

SEQ ID NO: 315 - RNA, 20 nt, synthetic construct
  source 1..20: mol_type = other RNA
gggtagcaaatctctaaagc

SEQ ID NO: 316 - RNA, 20 nt, synthetic construct
  source 1..20: mol_type = other RNA
gaatcattctgaggctgggc

SEQ ID NO: 317 - RNA, 20 nt, synthetic construct
  source 1..20: mol_type = other RNA
tcctccactgggttcagagg

SEQ ID NO: 318 - RNA, 20 nt, synthetic construct
  source 1..20: mol_type = other RNA
accctccaataaacccaggc

SEQ ID NO: 319 - RNA, 20 nt, synthetic construct
  source 1..20: mol_type = other RNA
ccactcttggctgtgctggc

SEQ ID NO: 320 - RNA, 20 nt, synthetic construct
  source 1..20: mol_type = other RNA
ggctccttccttccactccc

SEQ ID NO: 321 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
ccactagccctgggagcaaa

SEQ ID NO: 322 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Adenosine with phosphorothioate linkage at 3'
  modified_base 15: mod_base = OTHER; note = Thymidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gccatctccatccatagagc

SEQ ID NO: 323 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
aggagagctgtgggcttggg

SEQ ID NO: 324 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
cacccatgcctcccagcaga

SEQ ID NO: 325 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Adenosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gctggctgtactcactctcc

SEQ ID NO: 326 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Thymidine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gtgctctgtccttggtcctg

SEQ ID NO: 327 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Thymidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
cccattctcatgcagcctac

SEQ ID NO: 328 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Adenosine with phosphorothioate linkage at 3'
  modified_base 11: mod_base = OTHER; note = Guanosine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
ctgtgacctaggctccttga

SEQ ID NO: 329 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = Guanosine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ggtctgagaggctgtgggtc

SEQ ID NO: 330 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gctcccagttcttctgtggt

SEQ ID NO: 331 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Adenosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gatgtcctccacaggtgaca

SEQ ID NO: 332 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gcttcctgccttactgacct

SEQ ID NO: 333 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Thymidine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
ctctcctttgtccctgacca

SEQ ID NO: 334 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gccttgccttttcctcactc

SEQ ID NO: 335 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = Thymidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Adenosine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gcctggtcactaaccctctc

SEQ ID NO: 336 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = Guanosine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
cacccaccttggtcttgcct

SEQ ID NO: 337 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = Adenosine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
cacactgatgtcctgtccca

SEQ ID NO: 338 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Guanosine with phosphorothioate linkage at 3'
  modified_base 12: mod_base = OTHER; note = Guanosine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
cacacctctgggtcttggcc

SEQ ID NO: 339 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = Thymidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Adenosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gctgcccatcactcccagtt

SEQ ID NO: 340 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ctctccatctgccctggccc

SEQ ID NO: 341 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
cagtccctctccttgtctct

SEQ ID NO: 342 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = Guanosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
atccacctgctgctcctggg

SEQ ID NO: 343 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
cccttgtgtcttgtgggtgc

SEQ ID NO: 344 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = Thymidine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gccctaggattttcctgttg

SEQ ID NO: 345 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = Adenosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gcctcaactcctgcctccca

SEQ ID NO: 346 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = Guanosine with phosphorothioate linkage at 3'
  modified_base 10: mod_base = OTHER; note = Guanosine with phosphorothioate linkage at 3'
  modified_base 11: mod_base = OTHER; note = Guanosine with phosphorothioate linkage at 3'
  modified_base 12: mod_base = OTHER; note = Thymidine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
agacacctgggtatcagcct

SEQ ID NO: 347 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
tccttcttccctatttccca

SEQ ID NO: 348 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gcatcactcactgtcaggta

SEQ ID NO: 349 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gtcagtgccacagccttgtc

SEQ ID NO: 350 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ggcacctaccttatgcaccc

SEQ ID NO: 351 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
actactgcatccctcagccc

SEQ ID NO: 352 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Thymidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gcttgtctcatcctgtctct

SEQ ID NO: 353 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Adenosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
tgtctctgagctgactgctt

SEQ ID NO: 354 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gggcttgactccacactcca

SEQ ID NO: 355 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
ggcatggcatctcagcttca

SEQ ID NO: 356 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = Adenosine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
ttcaggatcacctagctggt

SEQ ID NO: 357 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Thymidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Adenosine with phosphorothioate linkage at 3'
  modified_base 12: mod_base = OTHER; note = Thymidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
cctctttgccatctgctggg

SEQ ID NO: 358 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gagtgcagttcacttgtggt

SEQ ID NO: 359 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
tgcccacactctgcctgtca

SEQ ID NO: 360 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = Guanosine with phosphorothioate linkage at 3'
  modified_base 8: mod_base = OTHER; note = Adenosine with phosphorothioate linkage at 3'
  modified_base 9: mod_base = OTHER; note = Guanosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
cagagggagctgctagtcag

SEQ ID NO: 361 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = Adenosine with phosphorothioate linkage at 3'
  modified_base 8: mod_base = OTHER; note = Adenosine with phosphorothioate linkage at 3'
  modified_base 9: mod_base = OTHER; note = Guanosine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ttggcaaaggtgatgcaggc

SEQ ID NO: 362 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = Adenosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
cctccaccagttggaagacc

SEQ ID NO: 363 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Thymidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = Guanosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ggttctcagccaccaggatc

SEQ ID NO: 364 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Adenosine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gtgctccaagatgcctgcca

SEQ ID NO: 365 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Guanosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ccttgcaggctcttgatggc

SEQ ID NO: 366 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = Thymidine with phosphorothioate linkage at 3'
  modified_base 8: mod_base = OTHER; note = Thymidine with phosphorothioate linkage at 3'
  modified_base 9: mod_base = OTHER; note = Guanosine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gtgccattggtagagtagga

SEQ ID NO: 367 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Adenosine with phosphorothioate linkage at 3'
  modified_base 13: mod_base = OTHER; note = Adenosine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gtgagctccctaaagaacct

SEQ ID NO: 368 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = Thymidine with phosphorothioate linkage at 3'
  modified_base 15: mod_base = OTHER; note = Adenosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
ggtttgccaccagtacaggg

SEQ ID NO: 369 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
tccagcttctccacatcaat

SEQ ID NO: 370 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gaacttgcctgcttccagct

SEQ ID NO: 371 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = Guanosine with phosphorothioate linkage at 3'
  modified_base 9: mod_base = OTHER; note = Guanosine with phosphorothioate linkage at 3'
  modified_base 10: mod_base = OTHER; note = Adenosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
acactctggatgagtttgtc

SEQ ID NO: 372 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = Adenosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Adenosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gggcacccatgacactctgg

SEQ ID NO: 373 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Thymidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
acttgtccagtgctccaggt

SEQ ID NO: 374 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Thymidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Thymidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
cccaatccttgcagtcatgg

SEQ ID NO: 375 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
agcacagcctgcatgtcctc

SEQ ID NO: 376 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
caaactggtgtcagagcctg

SEQ ID NO: 377 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Adenosine with phosphorothioate linkage at 3'
  modified_base 15: mod_base = OTHER; note = Adenosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gcagcaccctccaaactggt

SEQ ID NO: 378 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = Guanosine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = Guanosine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
tggttaggttggacccatgg

SEQ ID NO: 379 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Guanosine with phosphorothioate linkage at 3'
  modified_base 7: mod_base = OTHER; note = Guanosine with phosphorothioate linkage at 3'
  modified_base 8: mod_base = OTHER; note = Thymidine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gcccaggttgtctcagccca

SEQ ID NO: 380 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Thymidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Guanosine with phosphorothioate linkage at 3'
  modified_base 12: mod_base = OTHER; note = Adenosine with phosphorothioate linkage at 3'
  modified_base 13: mod_base = OTHER; note = Thymidine with phosphorothioate linkage at 3'
  modified_base 14: mod_base = OTHER; note = Adenosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
tccctctccagatactgagg

SEQ ID NO: 381 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 2'-O-methoxyethyladenosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
acagacaacccaatggcagg

SEQ ID NO: 382 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gtctccagaactgagcaggg

SEQ ID NO: 383 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Adenosine with phosphorothioate linkage at 3'
  modified_base 7: mod_base = OTHER; note = Thymidine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Thymidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ccttaattcctgtctgaggc

SEQ ID NO: 384 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Thymidine with phosphorothioate linkage at 3'
  modified_base 7: mod_base = OTHER; note = Adenosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
cagaatacagtgcccaggcc

SEQ ID NO: 385 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Thymidine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
cccaggcccttgctcagaat

SEQ ID NO: 386 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Thymidine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gcactcatccctggctggct

SEQ ID NO: 387 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Guanosine with phosphorothioate linkage at 3'
  modified_base 10: mod_base = OTHER; note = Guanosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gattacagggcaaggccaca

SEQ ID NO: 388 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = Guanosine with phosphorothioate linkage at 3'
  modified_base 8: mod_base = OTHER; note = Adenosine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Thymidine with phosphorothioate linkage at 3'
  modified_base 12: mod_base = OTHER; note = Guanosine with phosphorothioate linkage at 3'
  modified_base 13: mod_base = OTHER; note = Guanosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = Adenosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gccctggatgtggcaaaaga

SEQ ID NO: 389 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = Thymidine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Guanosine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
aaggaagtcagagggagggc

SEQ ID NO: 390 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethyladenosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = Adenosine with phosphorothioate linkage at 3'
  modified_base 10: mod_base = OTHER; note = Adenosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = Guanosine with phosphorothioate linkage at 3'
  modified_base 15: mod_base = OTHER; note = Adenosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
caggcccaaacaggaggctc

SEQ ID NO: 391 - DNA, 20 bp, synthetic construct
  source 1..20: mol_type = other DNA
  modified_base 1: mod_base = OTHER; note = 5' hydroxyl 2'-O-methoxyethyladenosine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 5-methyl-2'-O-methoxyethylcytidine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 5-methyl-2'-O-methoxyethylcytidine with phosphorothioate linkage at 3'
  modified_base 17: mod_base = OTHER; note = 5-methyl-2'-O-methoxyethylcytidine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 2'-O-methoxyethyladenosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
atgcccagacccaggcccaa

SEQ ID NO: 392 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 2'-O-methoxyethylthymidine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = Adenosine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = 5-methyl-2'-deoxycytidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
ctgaggcacagcaccaaggc

SEQ ID NO: 393 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = Guanosine with phosphorothioate linkage at 3'
  modified_base 12: mod_base = OTHER; note = Adenosine with phosphorothioate linkage at 3'
  modified_base 13: mod_base = OTHER; note = Adenosine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gccagaccaggaaggagcct

SEQ ID NO: 394 - DNA, 20 bp, synthetic construct
  source 1..20: mol_type = other DNA
  modified_base 1: mod_base = OTHER; note = 5' hydroxyl 2'-O-methoxyethylthymidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethyladenosine with phosphorothioate linkage at 3'
  modified_base 4: mod_base = OTHER; note = 2'-O-methoxyethylguanosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Adenosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = Thymidine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Guanosine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = 5-methyl-2'-deoxycytidine with phosphorothioate linkage at 3'
  modified_base 14: mod_base = OTHER; note = Thymidine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethylthymidine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
tcaggactttgcctctttcc

SEQ ID NO: 395 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Adenosine with phosphorothioate linkage at 3'
  modified_base 7: mod_base = OTHER; note = Guanosine with phosphorothioate linkage at 3'
  modified_base 8: mod_base = OTHER; note = Adenosine with phosphorothioate linkage at 3'
  modified_base 9: mod_base = OTHER; note = Guanosine with phosphorothioate linkage at 3'
  modified_base 10: mod_base = OTHER; note = Adenosine with phosphorothioate linkage at 3'
  modified_base 11: mod_base = OTHER; note = Thymidine with phosphorothioate linkage at 3'
  modified_base 12: mod_base = OTHER; note = Thymidine with phosphorothioate linkage at 3'
  modified_base 13: mod_base = OTHER; note = Thymidine with phosphorothioate linkage at 3'
  modified_base 14: mod_base = OTHER; note = Guanosine with phosphorothioate linkage at 3'
  modified_base 15: mod_base = OTHER; note = 5-methyl-2'-deoxycytidine with phosphorothioate linkage at 3'
  modified_base 16: mod_base = OTHER; note = 2'-O-methoxyethylthymidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 5-methyl-2'-O-methoxyethylcytidine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gctttagagatttgctaccc

SEQ ID NO: 396 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Guanosine with phosphorothioate linkage at 3'
  modified_base 7: mod_base = OTHER; note = 5-methyl-2'-deoxycytidine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Thymidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = Adenosine with phosphorothioate linkage at 3'
  modified_base 12: mod_base = OTHER; note = Guanosine with phosphorothioate linkage at 3'
  modified_base 13: mod_base = OTHER; note = Adenosine with phosphorothioate linkage at 3'
  modified_base 14: mod_base = OTHER; note = Adenosine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylthymidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gcccagcctcagaatgattc

SEQ ID NO: 397 - DNA, 20 bp, synthetic construct
  source 1..20: mol_type = other DNA
  modified_base 1: mod_base = OTHER; note = 5-methyl-5' hydroxyl 2'-O-methoxyethylcytidine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 2'-O-methoxyethylthymidine with phosphorothioate linkage at 3'
  modified_base 4: mod_base = OTHER; note = 5-methyl-2'-O-methoxyethylcytidine with phosphorothioate linkage at 3'
  modified_base 5: mod_base = OTHER; note = 2'-O-methoxyethylthymidine with phosphorothioate linkage at 3'
  modified_base 6: mod_base = OTHER; note = Guanosine with phosphorothioate linkage at 3'
  modified_base 7: mod_base = OTHER; note = Adenosine with phosphorothioate linkage at 3'
  modified_base 8: mod_base = OTHER; note = Adenosine with phosphorothioate linkage at 3'
  modified_base 9: mod_base = OTHER; note = 5-methyl-2'-deoxycytidine with phosphorothioate linkage at 3'
  modified_base 10: mod_base = OTHER; note = 5-methyl-2'-deoxycytidine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = Adenosine with phosphorothioate linkage at 3'
  modified_base 13: mod_base = OTHER; note = Guanosine with phosphorothioate linkage at 3'
  modified_base 14: mod_base = OTHER; note = Thymidine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethyladenosine
  misc_feature 1..5: note = RNA
  misc_feature 6..15: note = DNA
  misc_feature 16..20: note = RNA
cctctgaacccagtggagga

SEQ ID NO: 398 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethylthymidine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Guanosine with phosphorothioate linkage at 3'
  modified_base 7: mod_base = OTHER; note = Guanosine with phosphorothioate linkage at 3'
  modified_base 8: mod_base = OTHER; note = Thymidine with phosphorothioate linkage at 3'
  modified_base 9: mod_base = OTHER; note = Thymidine with phosphorothioate linkage at 3'
  modified_base 10: mod_base = OTHER; note = Thymidine with phosphorothioate linkage at 3'
  modified_base 11: mod_base = OTHER; note = Adenosine with phosphorothioate linkage at 3'
  modified_base 12: mod_base = OTHER; note = Thymidine with phosphorothioate linkage at 3'
  modified_base 13: mod_base = OTHER; note = Thymidine with phosphorothioate linkage at 3'
  modified_base 14: mod_base = OTHER; note = Guanosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
gcctgggtttattggagggt

SEQ ID NO: 399 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 5-methyl-2'-O-methoxyethylcytidine with phosphorothioate linkage at 3'
  modified_base 3: mod_base = OTHER; note = 5-methyl-2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = 5-methyl-2'-deoxycytidine with phosphorothioate linkage at 3'
  modified_base 7: mod_base = OTHER; note = Adenosine with phosphorothioate linkage at 3'
  modified_base 8: mod_base = OTHER; note = 5-methyl-2'-deoxycytidine with phosphorothioate linkage at 3'
  modified_base 9: mod_base = OTHER; note = Adenosine with phosphorothioate linkage at 3'
  modified_base 10: mod_base = OTHER; note = Guanosine with phosphorothioate linkage at 3'
  modified_base 11: mod_base = OTHER; note = 5-methyl-2'-deoxycytidine with phosphorothioate linkage at 3'
  modified_base 12: mod_base = OTHER; note = 5-methyl-2'-deoxycytidine with phosphorothioate linkage at 3'
  modified_base 13: mod_base = OTHER; note = Adenosine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethyladenosine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gccagcacagccaagagtgg

SEQ ID NO: 400 - DNA, 20 bp, synthetic construct
  source 1..20: mol_type = other DNA
  modified_base 1: mod_base = OTHER; note = 5' hydroxyl 2'-O-methoxyethylguanosine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylguanosine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = Guanosine with phosphorothioate linkage at 3'
  modified_base 9: mod_base = OTHER; note = Adenosine with phosphorothioate linkage at 3'
  modified_base 10: mod_base = OTHER; note = Adenosine with phosphorothioate linkage at 3'
  modified_base 11: mod_base = OTHER; note = Guanosine with phosphorothioate linkage at 3'
  modified_base 12: mod_base = OTHER; note = Guanosine with phosphorothioate linkage at 3'
  modified_base 13: mod_base = OTHER; note = Adenosine with phosphorothioate linkage at 3'
  modified_base 14: mod_base = OTHER; note = Adenosine with phosphorothioate linkage at 3'
  modified_base 15: mod_base = OTHER; note = Guanosine with phosphorothioate linkage at 3'
  modified_base 16: mod_base = OTHER; note = 2'-O-methoxyethylguanosine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylguanosine with phosphorothioate linkage at 3'
  modified_base 19: mod_base = OTHER; note = 5-methyl-2'-O-methoxyethylcytidine with phosphorothioate linkage at 3'
  modified_base 20: mod_base = OTHER; note = 5-methyl-2'-O-methoxyethylcytidine
  misc_feature 1..5: note = RNA
  misc_feature 6..15: note = DNA
  misc_feature 16..20: note = RNA
gggagtggaaggaaggagcc

SEQ ID NO: 401 - DNA, 20 bp, synthetic construct
  source 1..20: mol_type = other DNA
  modified_base 1: mod_base = OTHER; note = 2'-O-methoxyethylthymidine with phosphorothioate linkage at 3'
  modified_base 2: mod_base = OTHER; note = 2'-O-methoxyethylguanosine with phosphorothioate linkage at 3'
  modified_base 3: mod_base = OTHER; note = 2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethyladenosine with phosphorothioate linkage at 3'
  modified_base 6: mod_base = OTHER; note = Guanosine with phosphorothioate linkage at 3'
  modified_base 7: mod_base = OTHER; note = Thymidine with phosphorothioate linkage at 3'
  modified_base 8: mod_base = OTHER; note = Cytidine with phosphorothioate linkage at 3'
  modified_base 9: mod_base = OTHER; note = Thymidine with phosphorothioate linkage at 3'
  modified_base 10: mod_base = OTHER; note = Guanosine with phosphorothioate linkage at 3'
  modified_base 11: mod_base = OTHER; note = Adenosine with phosphorothioate linkage at 3'
  modified_base 12: mod_base = OTHER; note = Cytidine with phosphorothioate linkage at 3'
  modified_base 13: mod_base = OTHER; note = Guanosine with phosphorothioate linkage at 3'
  modified_base 14: mod_base = OTHER; note = Cytidine with phosphorothioate linkage at 3'
  modified_base 15: mod_base = OTHER; note = Cytidine with phosphorothioate linkage at 3'
  modified_base 16: mod_base = OTHER; note = 2'-O-methoxyethylcytidine with phosphorothioate linkage at 3'
  modified_base 17: mod_base = OTHER; note = 2'-O-methoxyethyladenosine with phosphorothioate linkage at 3'
  modified_base 18: mod_base = OTHER; note = 2'-O-methoxyethylthymidine with phosphorothioate linkage at 3'
  modified_base 19: mod_base = OTHER; note = 2'-O-methoxyethylcytidine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
tgcaagtctgacgcccatct

SEQ ID NO: 402 - DNA, 20 bp, synthetic construct
  source 1..20: mol_type = other DNA
  modified_base 1: mod_base = OTHER; note = 2'-O-methoxyethyladenosine with phosphorothioate linkage at 3'
  modified_base 2: mod_base = OTHER; note = 2'-O-methoxyethylcytidine with phosphorothioate linkage at 3'
  modified_base 3: mod_base = OTHER; note = 2'-O-methoxyethylcytidine with phosphorothioate linkage at 3'
  modified_base 4: mod_base = OTHER; note = 2'-O-methoxyethyladenosine with phosphorothioate linkage at 3'
  modified_base 5: mod_base = OTHER; note = 2'-O-methoxyethylguanosine with phosphorothioate linkage at 3'
  modified_base 6: mod_base = OTHER; note = Thymidine with phosphorothioate linkage at 3'
  modified_base 7: mod_base = OTHER; note = Guanosine with phosphorothioate linkage at 3'
  modified_base 8: mod_base = OTHER; note = Cytidine with phosphorothioate linkage at 3'
  modified_base 9: mod_base = OTHER; note = Adenosine with phosphorothioate linkage at 3'
  modified_base 10: mod_base = OTHER; note = Thymidine with phosphorothioate linkage at 3'
  modified_base 11: mod_base = OTHER; note = Thymidine with phosphorothioate linkage at 3'
  modified_base 12: mod_base = OTHER; note = Cytidine with phosphorothioate linkage at 3'
  modified_base 13: mod_base = OTHER; note = Adenosine with phosphorothioate linkage at 3'
  modified_base 14: mod_base = OTHER; note = Thymidine with phosphorothioate linkage at 3'
  modified_base 15: mod_base = OTHER; note = Thymidine with phosphorothioate linkage at 3'
  modified_base 16: mod_base = OTHER; note = 2'-O-methoxyethylcytidine with phosphorothioate linkage at 3'
  modified_base 17: mod_base = OTHER; note = 2'-O-methoxyethylguanosine with phosphorothioate linkage at 3'
  modified_base 18: mod_base = OTHER; note = 2'-O-methoxyethyladenosine with phosphorothioate linkage at 3'
  modified_base 19: mod_base = OTHER; note = 2'-O-methoxyethylguanosine with phosphorothioate linkage at 3'
  modified_base 20: mod_base = OTHER; note = 2'-O-methoxyethylthymidine
  misc_feature 1..5: note = RNA
  misc_feature 6..15: note = DNA
  misc_feature 16..20: note = RNA
accagtgcattcattcgagt